SEQUENCE LISTING (387 sequences)

SEQ ID NO: 1 - protein, 19 aa, Artificial Sequence
  Synthetic Construct
TFVYGGCRAKRNNFKSAED

SEQ ID NO: 2 - protein, 19 aa, Artificial Sequence
  Synthetic Construct
TFQYGGCMGNGNNFVTEKE

SEQ ID NO: 3 - protein, 19 aa, Artificial Sequence
  Synthetic Construct
PFFYGGCGGNRNNFDTEEY

SEQ ID NO: 4 - protein, 19 aa, Artificial Sequence
  Synthetic Construct
SFYYGGCLGNKNNYLREEE

SEQ ID NO: 5 - protein, 19 aa, Artificial Sequence
  Synthetic Construct
TFFYGGCRAKRNNFKRAKY

SEQ ID NO: 6 - protein, 19 aa, Artificial Sequence
  Synthetic Construct
TFFYGGCRGKRNNFKRAKY

SEQ ID NO: 7 - protein, 19 aa, Artificial Sequence
  Synthetic Construct
TFFYGGCRAKKNNYKRAKY

SEQ ID NO: 8 - protein, 19 aa, Artificial Sequence
  Synthetic Construct
TFFYGGCRGKKNNFKRAKY

SEQ ID NO: 9 - protein, 19 aa, Artificial Sequence
  Synthetic Construct
TFQYGGCRAKRNNFKRAKY

SEQ ID NO: 10 - protein, 19 aa, Artificial Sequence
  Synthetic Construct
TFQYGGCRGKKNNFKRAKY

SEQ ID NO: 11 - protein, 19 aa, Artificial Sequence
  Synthetic Construct
TFFYGGCLGKRNNFKRAKY

SEQ ID NO: 12 - protein, 19 aa, Artificial Sequence
  Synthetic Construct
TFFYGGSLGKRNNFKRAKY

SEQ ID NO: 13 - protein, 19 aa, Artificial Sequence
  Synthetic Construct
PFFYGGCGGKKNNFKRAKY

SEQ ID NO: 14 - protein, 19 aa, Artificial Sequence
  Synthetic Construct
TFFYGGCRGKGNNYKRAKY

SEQ ID NO: 15 - protein, 19 aa, Artificial Sequence
  Synthetic Construct
PFFYGGCRGKRNNFLRAKY

SEQ ID NO: 16 - protein, 19 aa, Artificial Sequence
  Synthetic Construct
TFFYGGCRGKRNNFKREKY

SEQ ID NO: 17 - protein, 19 aa, Artificial Sequence
  Synthetic Construct
PFFYGGCRAKKNNFKRAKE

SEQ ID NO: 18 - protein, 19 aa, Artificial Sequence
  Synthetic Construct
TFFYGGCRGKRNNFKRAKD

SEQ ID NO: 19 - protein, 19 aa, Artificial Sequence
  Synthetic Construct
TFFYGGCRAKRNNFDRAKY

SEQ ID NO: 20 - protein, 19 aa, Artificial Sequence
  Synthetic Construct
TFFYGGCRGKKNNFKRAEY

SEQ ID NO: 21 - protein, 19 aa, Artificial Sequence
  Synthetic Construct
PFFYGGCGANRNNFKRAKY

SEQ ID NO: 22 - protein, 19 aa, Artificial Sequence
  Synthetic Construct
TFFYGGCGGKKNNFKTAKY

SEQ ID NO: 23 - protein, 19 aa, Artificial Sequence
  Synthetic Construct
TFFYGGCRGNRNNFLRAKY

SEQ ID NO: 24 - protein, 19 aa, Artificial Sequence
  Synthetic Construct
TFFYGGCRGNRNNFKTAKY

SEQ ID NO: 25 - protein, 19 aa, Artificial Sequence
  Synthetic Construct
TFFYGGSRGNRNNFKTAKY

SEQ ID NO: 26 - protein, 19 aa, Artificial Sequence
  Synthetic Construct
TFFYGGCLGNGNNFKRAKY

SEQ ID NO: 27 - protein, 19 aa, Artificial Sequence
  Synthetic Construct
TFFYGGCLGNRNNFLRAKY

SEQ ID NO: 28 - protein, 19 aa, Artificial Sequence
  Synthetic Construct
TFFYGGCLGNRNNFKTAKY

SEQ ID NO: 29 - protein, 19 aa, Artificial Sequence
  Synthetic Construct
TFFYGGCRGNGNNFKSAKY

SEQ ID NO: 30 - protein, 19 aa, Artificial Sequence
  Synthetic Construct
TFFYGGCRGKKNNFDREKY

SEQ ID NO: 31 - protein, 19 aa, Artificial Sequence
  Synthetic Construct
TFFYGGCRGKRNNFLREKE

SEQ ID NO: 32 - protein, 19 aa, Artificial Sequence
  Synthetic Construct
TFFYGGCRGKGNNFDRAKY

SEQ ID NO: 33 - protein, 19 aa, Artificial Sequence
  Synthetic Construct
TFFYGGSRGKGNNFDRAKY

SEQ ID NO: 34 - protein, 19 aa, Artificial Sequence
  Synthetic Construct
TFFYGGCRGNGNNFVTAKY

SEQ ID NO: 35 - protein, 19 aa, Artificial Sequence
  Synthetic Construct
PFFYGGCGGKGNNYVTAKY

SEQ ID NO: 36 - protein, 19 aa, Artificial Sequence
  Synthetic Construct
TFFYGGCLGKGNNFLTAKY

SEQ ID NO: 37 - protein, 19 aa, Artificial Sequence
  Synthetic Construct
SFFYGGCLGNKNNFLTAKY

SEQ ID NO: 38 - protein, 19 aa, Artificial Sequence
  Synthetic Construct
TFFYGGCGGNKNNFVREKY

SEQ ID NO: 39 - protein, 19 aa, Artificial Sequence
  Synthetic Construct
TFFYGGCMGNKNNFVREKY

SEQ ID NO: 40 - protein, 19 aa, Artificial Sequence
  Synthetic Construct
TFFYGGSMGNKNNFVREKY

SEQ ID NO: 41 - protein, 19 aa, Artificial Sequence
  Synthetic Construct
PFFYGGCLGNRNNYVREKY

SEQ ID NO: 42 - protein, 19 aa, Artificial Sequence
  Synthetic Construct
TFFYGGCLGNRNNFVREKY

SEQ ID NO: 43 - protein, 19 aa, Artificial Sequence
  Synthetic Construct
TFFYGGCLGNKNNYVREKY

SEQ ID NO: 44 - protein, 19 aa, Artificial Sequence
  Synthetic Construct
TFFYGGCGGNGNNFLTAKY

SEQ ID NO: 45 - protein, 19 aa, Artificial Sequence
  Synthetic Construct
TFFYGGCRGNRNNFLTAEY

SEQ ID NO: 46 - protein, 19 aa, Artificial Sequence
  Synthetic Construct
TFFYGGCRGNGNNFKSAEY

SEQ ID NO: 47 - protein, 19 aa, Artificial Sequence
  Synthetic Construct
PFFYGGCLGNKNNFKTAEY

SEQ ID NO: 48 - protein, 19 aa, Artificial Sequence
  Synthetic Construct
TFFYGGCRGNRNNFKTEEY

SEQ ID NO: 49 - protein, 19 aa, Artificial Sequence
  Synthetic Construct
TFFYGGCRGKRNNFKTEED

SEQ ID NO: 50 - protein, 19 aa, Artificial Sequence
  Synthetic Construct
PFFYGGCGGNGNNFVREKY

SEQ ID NO: 51 - protein, 19 aa, Artificial Sequence
  Synthetic Construct
SFFYGGCMGNGNNFVREKY

SEQ ID NO: 52 - protein, 19 aa, Artificial Sequence
  Synthetic Construct
PFFYGGCGGNGNNFLREKY

SEQ ID NO: 53 - protein, 19 aa, Artificial Sequence
  Synthetic Construct
TFFYGGCLGNGNNFVREKY

SEQ ID NO: 54 - protein, 19 aa, Artificial Sequence
  Synthetic Construct
SFFYGGCLGNGNNYLREKY

SEQ ID NO: 55 - protein, 19 aa, Artificial Sequence
  Synthetic Construct
TFFYGGSLGNGNNFVREKY

SEQ ID NO: 56 - protein, 19 aa, Artificial Sequence
  Synthetic Construct
TFFYGGCRGNGNNFVTAEY

SEQ ID NO: 57 - protein, 19 aa, Artificial Sequence
  Synthetic Construct
TFFYGGCLGKGNNFVSAEY

SEQ ID NO: 58 - protein, 19 aa, Artificial Sequence
  Synthetic Construct
TFFYGGCLGNRNNFDRAEY

SEQ ID NO: 59 - protein, 19 aa, Artificial Sequence
  Synthetic Construct
TFFYGGCLGNRNNFLREEY

SEQ ID NO: 60 - protein, 19 aa, Artificial Sequence
  Synthetic Construct
TFFYGGCLGNKNNYLREEY

SEQ ID NO: 61 - protein, 19 aa, Artificial Sequence
  Synthetic Construct
PFFYGGCGGNRNNYLREEY

SEQ ID NO: 62 - protein, 19 aa, Artificial Sequence
  Synthetic Construct
PFFYGGSGGNRNNYLREEY

SEQ ID NO: 63 - protein, 19 aa, Artificial Sequence
  Synthetic Construct
MRPDFCLEPPYTGPCVARI

SEQ ID NO: 64 - protein, 21 aa, Artificial Sequence
  Synthetic Construct
ARIIRYFYNAKAGLCQTFVYG

SEQ ID NO: 65 - protein, 22 aa, Artificial Sequence
  Synthetic Construct
YGGCRAKRNNYKSAEDCMRTCG

SEQ ID NO: 66 - protein, 22 aa, Artificial Sequence
  Synthetic Construct
PDFCLEPPYTGPCVARIIRYFY

SEQ ID NO: 67 - protein, 19 aa, Artificial Sequence
  Synthetic Construct
TFFYGGCRGKRNNFKTEEY

SEQ ID NO: 68 - protein, 19 aa, Artificial Sequence
  Synthetic Construct
KFFYGGCRGKRNNFKTEEY

SEQ ID NO: 69 - protein, 19 aa, Artificial Sequence
  Synthetic Construct
TFYYGGCRGKRNNYKTEEY

SEQ ID NO: 70 - protein, 19 aa, Artificial Sequence
  Synthetic Construct
TFFYGGSRGKRNNFKTEEY

SEQ ID NO: 71 - protein, 20 aa, Artificial Sequence
  Synthetic Construct
CTFFYGCCRGKRNNFKTEEY

SEQ ID NO: 72 - protein, 20 aa, Artificial Sequence
  Synthetic Construct
TFFYGGCRGKRNNFKTEEYC

SEQ ID NO: 73 - protein, 20 aa, Artificial Sequence
  Synthetic Construct
CTFFYGSCRGKRNNFKTEEY

SEQ ID NO: 74 - protein, 20 aa, Artificial Sequence
  Synthetic Construct
TFFYGGSRGKRNNFKTEEYC

SEQ ID NO: 75 - protein, 19 aa, Artificial Sequence
  Synthetic Construct
PFFYGGCRGKRNNFKTEEY

SEQ ID NO: 76 - protein, 19 aa, Artificial Sequence
  Synthetic Construct
TFFYGGCRGKRNNFKTKEY

SEQ ID NO: 77 - protein, 19 aa, Artificial Sequence
  Synthetic Construct
TFFYGGKRGKRNNFKTEEY

SEQ ID NO: 78 - protein, 19 aa, Artificial Sequence
  Synthetic Construct
TFFYGGCRGKRNNFKTKRY

SEQ ID NO: 79 - protein, 19 aa, Artificial Sequence
  Synthetic Construct
TFFYGGKRGKRNNFKTAEY

SEQ ID NO: 80 - protein, 19 aa, Artificial Sequence
  Synthetic Construct
TFFYGGKRGKRNNFKTAGY

SEQ ID NO: 81 - protein, 19 aa, Artificial Sequence
  Synthetic Construct
TFFYGGKRGKRNNFKREKY

SEQ ID NO: 82 - protein, 19 aa, Artificial Sequence
  Synthetic Construct
TFFYGGKRGKRNNFKRAKY

SEQ ID NO: 83 - protein, 19 aa, Artificial Sequence
  Synthetic Construct
TFFYGGCLGNRNNFKTEEY

SEQ ID NO: 84 - protein, 19 aa, Artificial Sequence
  Synthetic Construct
TFFYGCGRGKRNNFKTEEY

SEQ ID NO: 85 - protein, 19 aa, Artificial Sequence
  Synthetic Construct
TFFYGGRCGKRNNFKTEEY

SEQ ID NO: 86 - protein, 19 aa, Artificial Sequence
  Synthetic Construct
TFFYGGCLGNGNNFDTEEE

SEQ ID NO: 87 - protein, 19 aa, Artificial Sequence
  Synthetic Construct
TFQYGGCRGKRNNFKTEEY

SEQ ID NO: 88 - protein, 19 aa, Artificial Sequence
  Synthetic Construct
YNKEFGTFNTKGCERGYRF

SEQ ID NO: 89 - protein, 19 aa, Artificial Sequence
  Synthetic Construct
RFKYGGCLGNMNNFETLEE

SEQ ID NO: 90 - protein, 19 aa, Artificial Sequence
  Synthetic Construct
RFKYGGCLGNKNNFLRLKY

SEQ ID NO: 91 - protein, 19 aa, Artificial Sequence
  Synthetic Construct
RFKYGGCLGNKNNYLRLKY

SEQ ID NO: 92 - protein, 22 aa, Artificial Sequence
  Synthetic Construct
KTKRKRKKQRVKIAYEEIFKNY

SEQ ID NO: 93 - protein, 15 aa, Artificial Sequence
  Synthetic Construct
KTKRKRKKQRVKIAY

SEQ ID NO: 94 - protein, 17 aa, Artificial Sequence
  Synthetic Construct
RGGRLSYSRRFSTSTGR

SEQ ID NO: 95 - protein, 10 aa, Artificial Sequence
  Synthetic Construct
RRLSYSRRRF

SEQ ID NO: 96 - protein, 16 aa, Artificial Sequence
  Synthetic Construct
RQIKIWFQNRRMKWKK

SEQ ID NO: 97 - protein, 19 aa, Artificial Sequence
  Synthetic Construct
TFFYGGSRGKRNNFKTEEY

SEQ ID NO: 98 - protein, 59 aa, Artificial Sequence
  Synthetic Construct
MRPDFCLEPPYTGPCVARIIRYFYNAKAGLCQTFVYGGCRAKRNNFKSAEDCMRTCGGA

SEQ ID NO: 99 - protein, 19 aa, Artificial Sequence
  Synthetic Construct
TFFYGGCRGKRNNFKTKEY

SEQ ID NO: 100 - protein, 19 aa, Artificial Sequence
  Synthetic Construct
RFKYGGCLGNKNNYLRLKY

SEQ ID NO: 101 - protein, 19 aa, Artificial Sequence
  Synthetic Construct
TFFYGGCRAKRNNFKRAKY

SEQ ID NO: 102 - protein, 35 aa, Artificial Sequence
  Synthetic Construct
NAKAGLCQTFVYGGCLAKRNNFESAEDCMRTCGGA

SEQ ID NO: 103 - protein, 24 aa, Artificial Sequence
  Synthetic Construct
YGGCRAKRNNFKSAEDCMRTCGGA

SEQ ID NO: 104 - protein, 22 aa, Artificial Sequence
  Synthetic Construct
GLCQTFVYGGCRAKRNNFKSAE

SEQ ID NO: 105 - protein, 20 aa, Artificial Sequence
  Synthetic Construct
LCQTFVYGGCEAKRNNFKSA

SEQ ID NO: 106 - skipped

SEQ ID NO: 107 - protein, 19 aa, Artificial Sequence
  Synthetic Construct
TFFYGGSRGKRNNFKTEEY

SEQ ID NO: 108 - protein, 19 aa, Artificial Sequence
  Synthetic Construct
RFFYGGSRGKRNNFKTEEY

SEQ ID NO: 109 - protein, 19 aa, Artificial Sequence
  Synthetic Construct
RFFYGGSRGKRNNFKTEEY

SEQ ID NO: 110 - protein, 19 aa, Artificial Sequence
  Synthetic Construct
RFFYGGSRGKRNNFRTEEY

SEQ ID NO: 111 - protein, 19 aa, Artificial Sequence
  Synthetic Construct
TFFYGGSRGKRNNFRTEEY

SEQ ID NO: 112 - protein, 19 aa, Artificial Sequence
  Synthetic Construct
TFFYGGSRGRRNNFRTEEY

SEQ ID NO: 113 - protein, 20 aa, Artificial Sequence
  Synthetic Construct
CTFFYGGSRGKRNNFKTEEY

SEQ ID NO: 114 - protein, 20 aa, Artificial Sequence
  Synthetic Construct
TFFYGGSRGKRNNFKTEEYC

SEQ ID NO: 115 - protein, 20 aa, Artificial Sequence
  Synthetic Construct
CTFFYGGSRGRRNNFRTEEY

SEQ ID NO: 116 - protein, 20 aa, Artificial Sequence
  Synthetic Construct
TFFYGGSRGRRNNFRTEEYC

SEQ ID NO: 117 - DNA, 57 bp, Artificial Sequence
  Synthetic Construct
acctttttctatggcggcagccgtggcaaacgcaacaatttcaagaccgaggagtat

SEQ ID NO: 118 - protein, 21 aa, Artificial Sequence
  Synthetic Construct
KIAGKIAKIAGKIAKIAGKIA

SEQ ID NO: 119 - protein, 14 aa, Artificial Sequence
  Synthetic Construct
RPRPNYRPRPIYRP

SEQ ID NO: 120 - protein, 6 aa, Artificial Sequence
  Synthetic Construct
KRNKNN

SEQ ID NO: 121 - protein, 5 aa, Artificial Sequence
  Synthetic Construct
  MOD_RES (1)..(1): Xaa is benzyloxycarbonyl-glycine
  MOD_RES (4)..(4): Xaa is aspartyl(OBu-t)
  MISC_FEATURE (5)..(5): amidation
XWMXF

SEQ ID NO: 122 - protein, 4 aa, Artificial Sequence
  Synthetic Construct
  MOD_RES (1)..(1): Xaa is butyloxycarbonyl-tryptophan
  MISC_FEATURE (4)..(4): amidation
XLDF

SEQ ID NO: 123 - protein, 6 aa, Artificial Sequence
  Synthetic Construct
  MOD_RES (1)..(1): Xaa is succinyl-tyrosine
  MOD_RES (6)..(6): phenethylamide termination
XMGWMD

SEQ ID NO: 124 - protein, 6 aa, Artificial Sequence
  Synthetic Construct
  MOD_RES (1)..(1): Xaa is t-butyloxycarbonyl-(sulfo-Tyr)
  MOD_RES (5)..(5): Xaa is Nle
  MOD_RES (6)..(6): Xaa is Asp 2-phenylethyl ester
XMGWXX

SEQ ID NO: 125 - protein, 5 aa, Artificial Sequence
  Synthetic Construct
  MOD_RES (1)..(1): Xaa is t-butyloxycarbonyl-sulfotyrosine
  MOD_RES (2)..(2): Xaa is norleucine
  MOD_RES (5)..(5): Xaa is aspartyl-2-phenylethyl ester
XXGYX

SEQ ID NO: 126 - protein, 10 aa, Artificial Sequence
  Synthetic Construct
  MOD_RES (1)..(1): Xaa is 9-fluorenylmethoxycarbonyl-arginine
XGIVEQCCTS

SEQ ID NO: 127 - protein, 39 aa, Artificial Sequence
  Synthetic Construct
  MISC_FEATURE (39)..(39): amidation
HGEGTFTSDLSKQMEEEAVRLFIEWLKNGGPSSGAPPPS

SEQ ID NO: 128 - protein, 39 aa, Artificial Sequence
  Synthetic Construct
  MOD_RES (1)..(1): Xaa is His, Arg or Tyr
  MOD_RES (2)..(2): Xaa is Ser, Gly, Ala or Thr
  MOD_RES (3)..(3): Xaa is Asp or Glu
  MOD_RES (6)..(6): Xaa is Phe, Tyr or Nal
  MOD_RES (7)..(7): Xaa is Thr or Ser
  MOD_RES (8)..(8): Xaa is Ser or Thr
  MOD_RES (9)..(9): Xaa is Asp or Glu
  MOD_RES (10)..(10): Xaa is Leu, Ile, Val, pGly or Met
  MOD_RES (14)..(14): Xaa is Leu, Ile, pGly, Val or Met
  MOD_RES (22)..(22): Xaa is Phe, Tyr or Nal
  MOD_RES (23)..(23): Xaa is Ile, Val, Leu, pGly, t-BuG or Met
  MOD_RES (24)..(24): Xaa is Glu or Asp
  MOD_RES (25)..(25): Xaa is Trp, Phe, Tyr or Nal
  MOD_RES (31)..(31): Xaa is Pro, HPro, 3Hyp, 4Hyp, TPro, N-alkylglycine, N-alkyl-pGly or N-alkylalanine
  MOD_RES (36)..(38): Xaa is Pro, HPro, 3Hyp, 4Hyp, TPro, N-alkylglycine, N-alkyl-pGly or N-alkylalanine
  MISC_FEATURE (39)..(39): C-terminus can be amidated or hydroxylated
  MOD_RES (39)..(39): Xaa is Ser, Thr or Tyr
XXXGTXXXXXSKQXEEEAVRLXXXXLKNGGXSSGAXXXX

SEQ ID NO: 129 - protein, 5 aa, Artificial Sequence
  Synthetic Construct
  MOD_RES (3)..(3): Xaa is Pro, HPro, 3Hyp, 4Hyp, TPro, N-alkylglycine, N-alkyl-pGly or N-alkylalanine
  MISC_FEATURE (5)..(5): C-terminus can be amidated or hydroxylated
GGXSS

SEQ ID NO: 130 - protein, 6 aa, Artificial Sequence
  Synthetic Construct
  MOD_RES (3)..(3): Xaa is Pro, HPro, 3Hyp, 4Hyp, TPro, N-alkylglycine, N-alkyl-pGly or N-alkylalanine
  MISC_FEATURE (6)..(6): C-terminus can be amidated or hydroxylated
GGXSSG

SEQ ID NO: 131 - protein, 7 aa, Artificial Sequence
  Synthetic construct
  MOD_RES (3)..(3): Xaa is Pro, HPro, 3Hyp, 4Hyp, TPro, N-alkylglycine, N-alkyl-pGly or N-alkylalanine
  MISC_FEATURE (7)..(7): C-terminus can be amidated or hydroxylated
GGXSSGA

SEQ ID NO: 132 - protein, 8 aa, Artificial Sequence
  Synthetic Construct
  MOD_RES (3)..(3): Xaa is Pro, HPro, 3Hyp, 4Hyp, TPro, N-alkylglycine, N-alkyl-pGly or N-alkylalanine
  MISC_FEATURE (8)..(8): C-terminus can be amidated or hydroxylated
  MOD_RES (8)..(8): Xaa is Pro, HPro, 3Hyp, 4Hyp, TPro, N-alkylglycine, N-alkyl-pGly or N-alkylalanine
GGXSSGAX

SEQ ID NO: 133 - protein, 9 aa, Artificial Sequence
  Synthetic Construct
  MOD_RES (3)..(3): Xaa is Pro, HPro, 3Hyp, 4Hyp, TPro, N-alkylglycine, N-alkyl-pGly or N-alkylalanine
  MOD_RES (8)..(9): Xaa is Pro, HPro, 3Hyp, 4Hyp, TPro, N-alkylglycine, N-alkyl-pGly or N-alkylalanine
  MISC_FEATURE (9)..(9): C-terminus can be amidated or hydroxylated
GGXSSGAXX

SEQ ID NO: 134 - protein, 10 aa, Artificial Sequence
  Synthetic Construct
  MOD_RES (3)..(3): Pro, HPro, 3Hyp, 4Hyp, TPro, N-alkylglycine, N-alkyl-pGly or N-alkylalanine
  MOD_RES (8)..(10): Pro, HPro, 3Hyp, 4Hyp, TPro, N-alkylglycine, N-alkyl-pGly or N-alkylalanine
  MISC_FEATURE (10)..(10): C-terminus can be amidated or hydroxylated
GGXSSGAXXX

SEQ ID NO: 135 - protein, 5 aa, Artificial Sequence
  Synthetic Construct
  MOD_RES (3)..(3): Xaa is Pro, HPro, TPro, or N-methylalanine
  MISC_FEATURE (5)..(5): C-terminus can be amidated or hydroxylated
GGXSS

SEQ ID NO: 136 - protein, 6 aa, Artificial Sequence
  Synthetic Construct
  MOD_RES (3)..(3): Xaa is Pro HPro, TPro or N-methylalanine
  MISC_FEATURE (6)..(6): C-terminus can be amidated or hydroxylated
GGXSSG

SEQ ID NO: 137 - protein, 7 aa, Artificial Sequence
  Synthetic Construct
  MOD_RES (3)..(3): Xaa is Pro, HPro, TPro or N-methylalanine
  MISC_FEATURE (7)..(7): C-terminus can be amidated or hydroxylated
GGXSSGA

SEQ ID NO: 138 - protein, 8 aa, Artificial Sequence
  Synthetic Construct
  MOD_RES (3)..(3): Xaa is Pro, HPro, TPro or N-methylalanine
  MISC_FEATURE (8)..(8): C-terminus can be amidated or hydroxylated
  MOD_RES (8)..(8): Xaa is Pro, HPro, TPro or N-methylalanine
GGXSSGAX

SEQ ID NO: 139 - protein, 9 aa, Artificial Sequence
  Synthetic Construct
  MOD_RES (3)..(3): Xaa is Pro, HPro, TPro or N-methylalanine
  MOD_RES (8)..(9): Xaa is Pro, HPro, TPro or N-methylalanine
  MISC_FEATURE (9)..(9): C-terminus can be amidated or hydroxylated
GGXSSGAXX

SEQ ID NO: 140 - protein, 10 aa, Artificial Sequence
  Synthetic Construct
  MOD_RES (3)..(3): Xaa is Pro, HPro, TPro or N-methylalanine
  MOD_RES (8)..(10): Xaa is Pro, HPro, TPro or N-methylalanine
  MISC_FEATURE (10)..(10): C-terminus can be amidated or hydroxylated
GGXSSGAXXX

SEQ ID NO: 141 - protein, 5 aa, Artificial Sequence
  Synthetic Construct
  MOD_RES (3)..(3): Xaa is Pro, HPro, 3Hyp, 4Hyp, TPro, N-alkylglycine, N-allyl-pGly, or N-alkylalanine
  MISC_FEATURE (28)..(28): C-terminus can be amidated or hydroxylated
GGXSS

SEQ ID NO: 142 - protein, 6 aa, Artificial Sequence
  Synthetic Construct
  MOD_RES (3)..(3): Xaa is Pro, HPro, 3Hyp, 4Hyp, TPro, N-alkylglycine, N-alkyl-pGly, or N-alkylalanine
  MISC_FEATURE (6)..(6): C-terminus can be amidated or hydroxylated
GGXSSG

SEQ ID NO: 143 - protein, 7 aa, Artificial Sequence
  Synthetic Construct
  MOD_RES (3)..(3): Xaa is Pro, HPro, 3Hyp, 4Hyp, TPro, N-alkylglycine, N-alkyl-pGly, or N-alkylalanine
  MISC_FEATURE (7)..(7): C-terminus can be amidated or hydroxylated
GGXSSGA

SEQ ID NO: 144 - protein, 8 aa, Artificial Sequence
  Synthetic Construct
  MOD_RES (3)..(3): Xaa is Pro, HPro, 3Hyp, 4Hyp, TPro, N-alkylglycine, N-alkyl-pGly, or N-alkylalanine
  MISC_FEATURE (8)..(8): C-terminus can be amidated or hydroxylated
  MOD_RES (8)..(8): Xaa is Ala or Gln
GGXSSGAX

SEQ ID NO: 145 - protein, 9 aa, Artificial Sequence
  Synthetic Construct
  MOD_RES (3)..(3): Xaa is Pro, HPro, 3Hyp, 4Hyp, TPro, N-alkylglycine, N-alkyl-pGly, or N-alkylalanine
  MOD_RES (8)..(9): Xaa is Pro, HPro, 3Hyp, 4Hyp, TPro, N-alkylglycine, N-alkyl-pGly, or N-alkylalanine
  MISC_FEATURE (9)..(9): C-terminus can be amidated or hydroxylated
GGXSSGAXX

SEQ ID NO: 146 - protein, 10 aa, Artificial Sequence
  Synthetic Construct
  MOD_RES (3)..(3): Xaa is Pro, HPro, 3Hyp, 4Hyp, TPro, N-alkylglycine, N-alkyl-pGly, or N-alkylalanine
  MOD_RES (8)..(10): Xaa is Pro, HPro, 3Hyp, 4Hyp, TPro, N-alkylglycine, N-alkyl-pGly, or N-alkylalanine
  MISC_FEATURE (10)..(10): C-terminus can be amidated or hydroxylated
GGXSSGAXXX

SEQ ID NO: 147 - protein, 11 aa, Artificial Sequence
  Synthetic Construct
  MOD_RES (3)..(3): Xaa is Pro, HPro, 3Hyp, 4Hyp, TPro, N-alkylglycine, N-alkyl-pGly, or N-alkylalanine
  MOD_RES (8)..(11): Xaa is Pro, HPro, 3Hyp, 4Hyp, TPro, N-alkylglycine, N-alkyl-pGly, or N-alkylalanine
  MISC_FEATURE (11)..(11): C-terminus can be amidated or hydroxylated
GGXSSGAXXXX

SEQ ID NO: 148 - protein, 40 aa, Artificial Sequence
  Synthetic Construct
  MOD_RES (14)..(14): Xaa is Arg, Leu, Ile, or Met
  MOD_RES (20)..(20): Xaa is His, Arg, or Lys
  MOD_RES (40)..(40): Xaa is Arg-OH, -OH, -NH2 or Lys-OH
HGEGTFTSDLSKQXEEEAVXLFIEWLKNGGPSSGAPPPSX

SEQ ID NO: 149 - protein, 32 aa, Artificial Sequence
  Synthetic Construct
  MISC_FEATURE (6)..(6): Xaa is Phe or Tyr
  MISC_FEATURE (14)..(14): Xaa is Met, Ile or Leu
  MISC_FEATURE (20)..(20): Xaa is Lys
  MISC_FEATURE (30)..(30): Xaa is Gly or is absent
  MISC_FEATURE (32)..(32): Xaa is Arg or is absent
HGEGTXTSDLSKQXEEEAVXLFIEWLKNGXPX

SEQ ID NO: 150 - protein, 31 aa, Artificial Sequence
  Synthetic Construct
  MOD_RES (29)..(31): May be absent
HAEGTFTSDVSSYLEGQAAKEFIAWLVKGRG

SEQ ID NO: 151 - protein, 21 aa, Artificial Sequence
  Synthetic Construct
  MOD_RES (1)..(1): Xaa is H2N, H2N-Ser, H2N-Val-Ser, H2N-Asp-Val-Ser, or one of SEQ ID NOs:152-158
  MOD_RES (18)..(18): Xaa is Lys or Arg
  MOD_RES (21)..(21): Xaa is NH2, OH, Gly-NH2, or Gly-OH
XSYLEGQAAKEFIAWLVXGRX

SEQ ID NO: 152 - protein, 4 aa, Artificial Sequence
  Synthetic Construct
  MISC_FEATURE (1)..(1): NH2 modification
SDVS

SEQ ID NO: 153 - protein, 5 aa, Artificial Sequence
  Synthetic Construct
  MISC_FEATURE (1)..(1): NH2 modification
TSDVS

SEQ ID NO: 154 - protein, 6 aa, Artificial Sequence
  Synthetic Construct
FTSDVS

SEQ ID NO: 155 - protein, 7 aa, Artificial Sequence
  Synthetic Construct
  MISC_FEATURE (1)..(1): NH2 modification
TFTSDVS

SEQ ID NO: 156 - protein, 8 aa, Artificial Sequence
  Synthetic Construct
  MISC_FEATURE (1)..(1): NH2 modification
GTFTSDVS

SEQ ID NO: 157 - protein, 9 aa, Artificial Sequence
  Synthetic Construct
  MISC_FEATURE (1)..(1): NH2 modification
EGTFTSDVS

SEQ ID NO: 158 - protein, 10 aa, Artificial Sequence
  Synthetic Construct
  MISC_FEATURE (1)..(1): NH2 modification
AEGTFTSDVS

SEQ ID NO: 159 - protein, 28 aa, Artificial Sequence
  Synthetic Construct
  MOD_RES (28)..(28): Xaa is Lys, Lys-Gly, or Lys-Gly-Arg
HAEGTFTSDVSSYLEGQAAKEFIAWLVX

SEQ ID NO: 160 - protein, 30 aa, Artificial Sequence
  Synthetic Construct
HAEGTFTSDVSSYLEGQAAKEFIAWLVKGR

SEQ ID NO: 161 - protein, 31 aa, Artificial Sequence
  Synthetic Construct
  MOD_RES (1)..(1): Xaa is His, D-His, desamino-His, 2-amino-His, beta-hydroxy-His, homohistidine, alpha-fluoromethyl-His or alpha-methyl-His
  MOD_RES (2)..(2): Xaa is Met, Asp, Lys, Thr, Leu, Asn, Gln, Phe, Val or Tyr
  MOD_RES (15)..(15): Xaa is Glu, Gln, Ala, Thr, Ser or Gly
  MOD_RES (21)..(21): Xaa is Glu, Gln, Ala, Thr, Ser or Gly
  MOD_RES (31)..(31): Xaa is NH2 or Gly-OH
XXEGTFTSDVSSYLXGQAAKXFIAWLVKGRX

SEQ ID NO: 162 - protein, 31 aa, Artificial Sequence
  Synthetic Construct
  MOD_RES (1)..(1): Xaa is 4-imidazopropionyl (desamino-histidyl), 4-imidazoacetyl, or 4-imidazo-alpha, alpha-dimethyl-acetyl
  MOD_RES (20)..(20): Xaa is  Lys or Arg
  MOD_RES (28)..(28): Xaa is Lys(R2), where R2 is C6-10 unbranched acyl or absent
  MOD_RES (31)..(31): Xaa is Gly-OH or NH2
XAEGTFTSDVSSYLEGQAAXEFIAWLVXGRX

SEQ ID NO: 163 - protein, 31 aa, Artificial Sequence
  Synthetic Construct
  MOD_RES (2)..(2): Xaa is Gly, Ala, Val, Leu, Ile, Ser, or Thr
  MOD_RES (5)..(5): Xaa is Asp, Glu, Arg, Thr, Ala, Lys, or His
  MOD_RES (6)..(6): Xaa is His, Trp, Phe, or Tyr
  MOD_RES (10)..(10): Xaa is Leu, Ser, Thr, Trp, His, Phe, Asp, Val, Tyr, Glu, or Ala
  MOD_RES (16)..(16): Xaa is Gly, Asp, Glu, Gln, Asn, Lys, Arg, Cys, or Cya
  MOD_RES (17)..(17): Xaa is His, Asp, Lys, Glu, or Gln
  MOD_RES (18)..(18): Xaa is Glu, His, Ala, or Lys
  MOD_RES (20)..(20): Xaa is Asp, Lys, Glu, or His
  MOD_RES (21)..(21): Xaa is Ala, Glu, His, Phe, Tyr, Trp, Arg, or Lys
  MOD_RES (25)..(25): Xaa is Ala, Glu, Asp, Ser, or His
  MOD_RES (27)..(27): Xaa is Asp, Arg, Val, Lys, Ala, Gly, or Glu
  MOD_RES (28)..(28): Xaa is Glu, Lys, or Asp
  MOD_RES (29)..(29): Xaa is Thr, Ser, Lys, Arg, Trp, Tyr, Phe, Asp, Gly, Pro, His, or Glu
  MOD_RES (30)..(30): Xaa is Arg, Glu, or His
  MOD_RES (31)..(31): Xaa is Lys, Arg, Thr, Ser, Glu, Asp, Trp, Tyr, Phe, His, NH2, Gly, Gly-Pro, or Gly-Pro-NH2, or is deleted
HXEGXXTSDXSSYLEXXXAXXFIAXLXXXXX

SEQ ID NO: 164 - protein, 31 aa, Artificial Sequence
  Synthetic Construct
  MOD_RES (2)..(2): Xaa is Gly, Ala, Val, Leu, Ile, Ser, or Thr
  MOD_RES (6)..(6): Xaa is His, Trp, Phe, or Tyr
  MOD_RES (10)..(10): Xaa is Leu, Ser, Thr, Trp, His, Phe, Asp, Val, Glu, or Ala
  MOD_RES (16)..(16): Xaa is Gly, Asp, Glu, Gln, Asn, Lys, Arg, Cys, or Cya
  MOD_RES (17)..(17): Xaa is His, Asp, Lys, Glu, or Gln
  MOD_RES (20)..(20): Xaa is Asp, Lys, Glu, or His
  MOD_RES (24)..(24): Xaa is Ala, Glu, Asp, Ser, or His
  MOD_RES (29)..(29): Xaa is Thr, Ser, Lys, Arg, Trp, Tyr, Phe, Asp, Gly, Pro, His, or Glu
  MOD_RES (31)..(31): Xaa is Lys, Arg, Thr, Ser, Glu, Asp, Trp, Tyr, Phe, His, NH2, Gly, Gly-Pro, Gly-Pro-NH2, or is deleted
HXEGTXTSDXSSYLEXXAAXEFIXWLVKXRX

SEQ ID NO: 165 - protein, 31 aa, Artificial Sequence
  Synthetic Construct
  MOD_RES (2)..(2): Xaa is Gly, Ala, Val, Leu, Ile, Ser, or Thr
  MOD_RES (16)..(16): Xaa is Gly, Asp, Glu, Gln, Asn, Lys, Arg, Cys, or Cya
  MOD_RES (17)..(17): Xaa is His, Asp, Lys, Glu, or Gln
  MOD_RES (21)..(21): Xaa is Ala, Glu, His, Phe, Tyr, Trp, Arg, or Lys
  MOD_RES (24)..(24): Xaa is Ala, Glu, Asp, Ser, or His
  MOD_RES (31)..(31): Xaa is Lys, Arg, Thr, Ser, Glu, Asp, Trp, Tyr, Phe, His, -NH2, Gly, Gly-Pro, or Gly-Pro-NH2, or is deleted
HXEGTFTSDVSSYLEXXAAKXFIXWLVKGRX

SEQ ID NO: 166 - protein, 31 aa, Artificial Sequence
  Synthetic Construct
  MOD_RES (1)..(1): Xaa is L-His, D-His, desamino-His, 2amino-His, beta-hydroxy-His, homo-His, alpha-fluoromethyl-His or alpha-methyl-His
  MOD_RES (2)..(2): Xaa is Gly, Ala, Val, Leu, Ile, Ser or Thr
  MOD_RES (16)..(16): Xaa is Asp, Glu, Gln, Asn, Lys, Arg, Cys or Cya
  MOD_RES (31)..(31): Xaa is -NH2 or Gly(OH)
XXEGTFTSDVSSYLEXQAAKEFIAWLVKGRX

SEQ ID NO: 167 - protein, 31 aa, Artificial Sequence
  Synthetic Construct
  MOD_RES (1)..(1): Xaa is L-His, D-His, desamino-His, 2-amino-His, beta-hydroxy-His, homohistidine, alpha-fluoromethyl-His or alpha-methyl-His
  MOD_RES (2)..(2): Xaa is Ala, Gly, Val, Leu, Ile, Ser or Thr
  MOD_RES (6)..(6): Xaa is Phe, Trp or Tyr
  MOD_RES (10)..(10): Xaa is Val, Trp, Ile, Leu, Phe or Tyr
  MOD_RES (12)..(12): Xaa is Ser, Trp, Tyr, Phe, Lys, Ile, Leu or Val
  MOD_RES (13)..(13): Xaa is Tyr, Trp or Phe
  MOD_RES (14)..(14): Xaa is Leu, Phe, Tyr or Trp
  MOD_RES (16)..(16): Xaa is Gly, Glu, Asp or Lys
  MOD_RES (19)..(19): Xaa is Ala, Val, Ile or Leu
  MOD_RES (21)..(21): Xaa is Glu, Ile or Ala
  MOD_RES (24)..(24): Xaa is Ala or Glu
  MOD_RES (27)..(27): Xaa is Val or Ile
  MOD_RES (31)..(31): Xaa is Gly, His, NH2, or is absent
XXEGTXTSDXSXXXEXQAXKXFIXWLXKGRX

SEQ ID NO: 168 - protein, 31 aa, Artificial Sequence
  Synthetic Construct
  MOD_RES (1)..(1): Xaa is L-His, D-His, desamino-His, 2-amino-His, beta-hydroxy-His, homohistidine, alpha-fluoromethyl-His or alpha-methyl-His
  MOD_RES (2)..(2): Xaa is Gly, Ala, Val, Leu, Ile, Ser or Thr
  MOD_RES (10)..(10): Xaa is Val, Phe, Tyr or Trp
  MOD_RES (12)..(12): Xaa is Ser, Tyr, Trp, Phe, Lys, Ile, Leu or Val
  MOD_RES (16)..(16): Xaa is Gly, Glu, Asp or Lys
  MOD_RES (19)..(19): Xaa is Ala, Val, Ile or Leu
  MOD_RES (27)..(27): Xaa is Val or Ile
  MOD_RES (31)..(31): Xaa is Gly, NH2 or is absent
XXEGTFTSDXSXYLEXQAXKEFIAWLXKGRX

SEQ ID NO: 169-171 - skipped (3 entries)

SEQ ID NO: 172 - protein, 10 aa, Artificial Sequence
  Synthetic Construct
  MOD_RES (1)..(1): Xaa is N-acetyl-3-(3-quinolyl)alanine
  MOD_RES (2)..(2): Xaa is 3-(4-chlorophenyl)alanine
  MOD_RES (3)..(3): Xaa is 3-(3-pyridyl)alanine
  MOD_RES (5)..(5): Xaa is 3-(4-pyrazinylcarbonylaminocyclohexyl) alanine
  MOD_RES (6)..(6): Xaa is N(epsilon)picolinoyllysine
  MOD_RES (10)..(10): amination
XXXSXXVRPA

SEQ ID NO: 173 - protein, 10 aa, Artificial Sequence
  Synthetic Construct
  MOD_RES (1)..(1): Xaa is pyroglutamine
  MOD_RES (10)..(10): amination
XHWSYWLRPG

SEQ ID NO: 174 - protein, 5 aa, Artificial Sequence
  Synthetic Construct
  MOD_RES (1)..(1): Xaa is pyroglutamine
  MOD_RES (5)..(5): methyl ester modification
XHWSY

SEQ ID NO: 175 - skipped

SEQ ID NO: 176 - protein, 4 aa, Artificial Sequence
  Synthetic construct
  MOD_RES (4)..(4): amination
YPLG

SEQ ID NO: 177 - protein, 4 aa, Artificial Sequence
  Synthetic Construct
YPLG

SEQ ID NO: 178 - protein, 4 aa, Artificial Sequence
  Synthetic Construct
  MOD_RES (4)..(4): amination
YPKG

SEQ ID NO: 179 - protein, 4 aa, Artificial Sequence
  Synthetic Construct
  MOD_RES (4)..(4): amination
YPWG

SEQ ID NO: 180 - protein, 4 aa, Artificial Sequence
  Synthetic Construct
  MISC_FEATURE (1)..(4): Cyclic Peptide
  MOD_RES (1)..(1): Xaa is 7-aminoheptanoyl-phenylalanine
XWKT

SEQ ID NO: 181 - protein, 4 aa, Artificial Sequence
  Synthetic Peptide
  MISC_FEATURE (1)..(4): Cyclic Peptide
  MOD_RES (1)..(1): Xaa is 7-aminoheptanoylphenylalanine
  MOD_RES (4)..(4): Xaa is benzylthreonine
XYKX

SEQ ID NO: 182 - protein, 6 aa, Artificial Sequence
  Synthetic Peptide
  MISC_FEATURE (1)..(6): Cyclic Peptide
  MOD_RES (1)..(1): Xaa is beta-methyl-N-benzylglycyine
XFWKTF

SEQ ID NO: 183 - protein, 6 aa, Artificial Sequence
  Synthetic Construct
  MISC_FEATURE (1)..(6): Cyclic Peptide
  MOD_RES (1)..(1): Xaa is N-benzylglycine
XFWKTF

SEQ ID NO: 184 - protein, 6 aa, Artificial Sequence
  Synthetic Construct
  MISC_FEATURE (1)..(6): Cyclic Peptide
PFWKTF

SEQ ID NO: 185 - protein, 6 aa, Artificial Sequence
  Synthetic Construct
  MISC_FEATURE (1)..(6): Cyclic Peptide
  MOD_RES (2)..(2): Xaa is thiomethyl-phenylalanine
PXWKTF

SEQ ID NO: 186 - protein, 8 aa, Artificial Sequence
  Synthetic Construct
  MOD_RES (7)..(7): Xaa is Pen
  MISC_FEATURE (8)..(8): C-terminus is amidated
FCFWKTXT

SEQ ID NO: 187 - protein, 7 aa, Artificial Sequence
  Synthetic Construct
  MISC_FEATURE (2)..(6): Cyclic Peptide
  MOD_RES (3)..(3): Xaa is tyrosyltryptophyl
  MOD_RES (6)..(6): Xaa is penicillamine
  MOD_RES (7)..(7): amidation
FCXKTXT

SEQ ID NO: 188 - protein, 7 aa, Artificial Sequence
  Synthetic Construct
  MISC_FEATURE (2)..(6): Cyclic Peptide
  MOD_RES (2)..(2): Xaa is cysteinyltyrosyl
  MOD_RES (4)..(4): Xaa is orinthyl
  MOD_RES (6)..(6): Xaa is penicillamine
  MISC_FEATURE (7)..(7): amidation
FXWXTXT

SEQ ID NO: 189-190 - skipped (2 entries)

SEQ ID NO: 191 - protein, 8 aa, Artificial Sequence
  Synthetic Construct
  MOD_RES (8)..(8): Xaa is N-naphthylalanine amide
FCYWKTCX

SEQ ID NO: 192 - protein, 10 aa, Artificial Sequence
  Synthetic Construct
  MISC_FEATURE (1)..(10): Cyclic Peptide
  MISC_FEATURE (10)..(10): Amidation
EHFRWGKPVG

SEQ ID NO: 193 - protein, 6 aa, Artificial Sequence
  Synthetic Construct
  MISC_FEATURE (6)..(6): Amidation
KHFRWG

SEQ ID NO: 194 - skipped

SEQ ID NO: 195 - protein, 7 aa, Artificial Sequence
  Synthetic Construct
  MOD_RES (1)..(1): Xaa is acetyl-norleucine
  MISC_FEATURE (2)..(7): Cyclic Peptide
  MOD_RES (5)..(5): Xaa is beta-methyltryptophan
  MISC_FEATURE (7)..(7): amidation
XDHFRXK

SEQ ID NO: 196 - protein, 8 aa, Artificial Sequence
  Synthetic Construct
  MOD_RES (1)..(1): Xaa is acetylnorleucine
  MISC_FEATURE (8)..(8): Amidation
XEHFRWGK

SEQ ID NO: 197 - protein, 12 aa, Artificial Sequence
  Synthetic Construct
  MOD_RES (3)..(3): Xaa is norleucine
  MOD_RES (6)..(6): Xaa is 2′-napthylalanine
  MOD_RES (12)..(12): Amidation
YVXGPXRWDRFG

SEQ ID NO: 198 - protein, 4 aa, Artificial Sequence
  Synthetic Construct
  MISC_FEATURE (1)..(4): Cyclic Peptide
  MISC_FEATURE (1)..(4): 4-(aminomethyl)phenylacetic acid modification
FWKT

SEQ ID NO: 199 - protein, 4 aa, Artificial Sequence
  Synthetic Construct
  MISC_FEATURE (1)..(4): Cyclic Peptide
  MISC_FEATURE (1)..(4): 3-(aminomethyl)phenylacetic acid modification
FWKT

SEQ ID NO: 200 - protein, 4 aa, Artificial Sequence
  Synthetic Construct
  MISC_FEATURE (1)..(4): Cyclic Peptide
  MISC_FEATURE (1)..(4): 2-(aminomethyl)phenylacetic acid modification
FWKT

SEQ ID NO: 201-202 - skipped (2 entries)

SEQ ID NO: 203 - protein, 10 aa, Artificial Sequence
  Synthetic Construct
  MISC_FEATURE (10)..(10): Amidation
ADVGHVFLRF

SEQ ID NO: 204 - protein, 4 aa, Artificial Sequence
  Synthetic Construct
  MISC_FEATURE (4)..(4): Amidation
APGW

SEQ ID NO: 205 - protein, 9 aa, Artificial Sequence
  Synthetic Construct
  MISC_FEATURE (9)..(9): Amidation
AWQDLNSAW

SEQ ID NO: 206 - protein, 9 aa, Artificial Sequence
  Synthetic Construct
  MISC_FEATURE (9)..(9): Amidation
AMSFYFPRM

SEQ ID NO: 207 - protein, 6 aa, Artificial Sequence
  Synthetic Construct
  MISC_FEATURE (6)..(6): Amidation
RAPYFV

SEQ ID NO: 208 - protein, 5 aa, Artificial Sequence
  Synthetic Construct
  MISC_FEATURE (5)..(5): Amidation
RYIRF

SEQ ID NO: 209 - protein, 8 aa, Artificial Sequence
  Synthetic Construct
  MISC_FEATURE (8)..(8): C-terminus is amidated
ARPYSFGL

SEQ ID NO: 210 - protein, 5 aa, Artificial Sequence
  Synthetic Construct
  MISC_FEATURE (5)..(5): Amidation
NGIWY

SEQ ID NO: 211 - protein, 7 aa, Artificial Sequence
  Synthetic Construct
  MISC_FEATURE (7)..(7): Amidation
DYRPLQF

SEQ ID NO: 212 - protein, 8 aa, Artificial Sequence
  Synthetic Construct
  MISC_FEATURE: Cyclic Peptide
NTSFTPRL

SEQ ID NO: 213 - protein, 4 aa, Artificial Sequence
  Synthetic Construct
  MISC_FEATURE (1)..(1): Dansylation
  MISC_FEATURE (4)..(4): Amidation
PQRF

SEQ ID NO: 214 - protein, 4 aa, Artificial Sequence
  Synthetic Construct
  MISC_FEATURE (4)..(4): Amidation
FMRF

SEQ ID NO: 215 - protein, 12 aa, Artificial Sequence
  Synthetic Construct
  MISC_FEATURE (2)..(2): Ala is D-Ala
  MISC_FEATURE (12)..(12): Amidation
YAGFMKKKFMRF

SEQ ID NO: 216 - protein, 9 aa, Artificial Sequence
  Synthetic Construct
  MISC_FEATURE (9)..(9): Amidation
DPKEDFMRF

SEQ ID NO: 217 - protein, 5 aa, Artificial Sequence
  Synthetic Construct
  MISC_FEATURE (1)..(1): Desamino modification
  MOD_RES (3)..(3): Xaa is norleucyl
  MISC_FEATURE (5)..(5): Amidation
YFXRF

SEQ ID NO: 218 - protein, 9 aa, Artificial Sequence
  Synthetic Construct
  MISC_FEATURE (9)..(9): Amidation
GAHKNYLRF

SEQ ID NO: 219 - protein, 7 aa, Artificial Sequence
  Synthetic Construct
  MISC_FEATURE (7)..(7): Amidation
KSAYMRF

SEQ ID NO: 220 - skipped

SEQ ID NO: 221 - protein, 7 aa, Artificial Sequence
  Synthetic Construct
  MISC_FEATURE (7)..(7): Amidation
SDNFMRF

SEQ ID NO: 222 - protein, 8 aa, Artificial Sequence
  Synthetic Construct
  MISC_FEATURE (8)..(8): Amidation
SDPNFLRF

SEQ ID NO: 223 - protein, 7 aa, Artificial Sequence
  Synthetic Construct
  MISC_FEATURE (7)..(7): Amidation
SKPYMRF

SEQ ID NO: 224 - protein, 9 aa, Artificial Sequence
  Synthetic Construct
  MISC_FEATURE (9)..(9): Amidation
TPAEDFMRF

SEQ ID NO: 225 - protein, 4 aa, Artificial Sequence
  Synthetic Construct
  MISC_FEATURE (4)..(4): Amidation
FPRF

SEQ ID NO: 226 - protein, 7 aa, Artificial Sequence
  Synthetic Construct
  MISC_FEATURE (7)..(7): Amidation
GDPFLRF

SEQ ID NO: 227 - protein, 11 aa, Artificial Sequence
  Synthetic Construct
  MISC_FEATURE (11)..(11): Amidation
GLTPNMNSLFF

SEQ ID NO: 228 - protein, 6 aa, Artificial Sequence
  Synthetic Constuct
GLLDLK

SEQ ID NO: 229 - protein, 5 aa, Artificial Sequence
  Synthetic Construct
  MISC_FEATURE (5)..(5): Amidation
GYIRF

SEQ ID NO: 230 - protein, 7 aa, Artificial Sequence
  Synthetic Construct
  MOD_RES (1)..(1): Xaa is H-tryptophan
  MISC_FEATURE (7)..(7): Amidation
XREMSVW

SEQ ID NO: 231 - protein, 7 aa, Artificial Sequence
  Synthetic Construct
  MISC_FEATURE (7)..(7): Amidation
KPSFVRF

SEQ ID NO: 232 - protein, 9 aa, Artificial Sequence
  Synthetic Construct
  MISC_FEATURE (9)..(9): Amidation
LPPGPLPRP

SEQ ID NO: 233 - protein, 4 aa, Artificial Sequence
  Synthetic Construct
  MISC_FEATURE (4)..(4): Amidation
LFRF

SEQ ID NO: 234 - protein, 8 aa, Artificial Sequence
  Synthetic Construct
  MISC_FEATURE (8)..(8): Amidation
FDAFTTGF

SEQ ID NO: 235 - protein, 4 aa, Artificial Sequence
  Synthetic Construct
  MISC_FEATURE (4)..(4): Amidation
FTRF

SEQ ID NO: 236 - protein, 4 aa, Artificial Sequence
  Synthetic Construct
  MISC_FEATURE (4)..(4): Amidation
PFRF

SEQ ID NO: 237 - protein, 7 aa, Artificial Sequence
  Synthetic Construct
  MOD_RES (1)..(1): Xaa is pyroglutamyl
  MISC_FEATURE (7)..(7): Amidation
XDPFLRF

SEQ ID NO: 238 - protein, 8 aa, Artificial Sequence
  Synthetic Construct
  MOD_RES (1)..(1): Xaa is pyroglutamyl
  MISC_FEATURE (8)..(8): Amidation
XLNFSTGW

SEQ ID NO: 239 - protein, 5 aa, Artificial Sequence
  Synthetic Construct
  MOD_RES (1)..(1): Xaa is pyroglutamyl
  MISC_FEATURE (5)..(5): Amidation
XLGRF

SEQ ID NO: 240 - protein, 10 aa, Artificial Sequence
  Synthetic Construct
  MOD_RES (1)..(1): Xaa is pyroglutamyl
  MISC_FEATURE (10)..(10): Amidation
XLTFTPNWGS

SEQ ID NO: 241 - protein, 7 aa, Artificial Sequence
  Synthetic Construct
  MOD_RES (1)..(1): Xaa is pyroglutamyl
  MISC_FEATURE (7)..(7): Amidation
XWLKGRF

SEQ ID NO: 242 - protein, 9 aa, Artificial Sequence
  Synthetic Construct
  MISC_FEATURE (9)..(9): Amidation
SADPNFLRF

SEQ ID NO: 243-245 - skipped (3 entries)

SEQ ID NO: 246 - protein, 7 aa, Artificial Sequence
  Synthetic Construct
  MISC_FEATURE (7)..(7): Amidation
SDPFLRF

SEQ ID NO: 247 - protein, 8 aa, Artificial Sequence
  Synthetic Construct
  MISC_FEATURE (8)..(8): Amidation
SDRNFLRF

SEQ ID NO: 248 - skipped

SEQ ID NO: 249 - protein, 7 aa, Artificial Sequence
  Synthetic Construct
  MISC_FEATURE (7)..(7): Amidation
SEPYLRF

SEQ ID NO: 250 - protein, 6 aa, Artificial Sequence
  Synthetic Construct
TKQELE

SEQ ID NO: 251 - protein, 8 aa, Artificial Sequence
  Synthetic Construct
  MISC_FEATURE (8)..(8): Amidation
TNRNFLRF

SEQ ID NO: 252 - protein, 13 aa, Artificial Sequence
  Synthetic Construct
VFQNQFKGIQGRF

SEQ ID NO: 253 - protein, 12 aa, Artificial Sequence
  Synthetic Construct
  MISC_FEATURE (12)..(12): Amidation
VPNDWAHFRGSW

SEQ ID NO: 254 - protein, 7 aa, Artificial Sequence
  Synthetic Construct
  MISC_FEATURE (7)..(7): Amidation
YFAFPRQ

SEQ ID NO: 255 - skipped

SEQ ID NO: 256 - protein, 8 aa, Artificial Sequence
  Synthetic Construct
  MOD_RES (7)..(7): Xaa is (3S)(amino)-5-(carbonylmethyl)-2,3-dihydro-1,5-benzothiazepin-4(5 H)-one
RPPGFSXR

SEQ ID NO: 257 - skipped

SEQ ID NO: 258 - protein, 11 aa, Artificial Sequence
  Synthetic Construct
  MISC_FEATURE (1)..(1): palmitoylation
  MISC_FEATURE (11)..(11): hydroxylation
GKRPPGFSPFR

SEQ ID NO: 259 - protein, 10 aa, Artificial Sequence
  Synthetic Construct
  MISC_FEATURE (1)..(7): Cyclicle Peptide
  MISC_FEATURE (1)..(1): t-butyloxycarbonylation
  MISC_FEATURE (2)..(2): Xaa is t-butylseryl
  MISC_FEATURE (5)..(5): Xaa is t-butylseryl
  MISC_FEATURE (6)..(6): Xaa is t-butylthreonyl
CXNLXXCVLG

SEQ ID NO: 260 - protein, 7 aa, Artificial Sequence
  Synthetic Construct
YGWMDFG

SEQ ID NO: 261 - protein, 6 aa, Artificial Sequence
  Sythnthetic Construct
  MISC_FEATURE (1)..(1): Acetylation
RFWINK

SEQ ID NO: 262 - skipped

SEQ ID NO: 263 - protein, 4 aa, Artificial Sequence
  Synthetic Construct
KKGE

SEQ ID NO: 264 - protein, 4 aa, Artificial Sequence
  Synthetic Construct
  MISC_FEATURE (1)..(1): Xaa is 3-carboxysalsolinol-Gly
XGFL

SEQ ID NO: 265 - protein, 6 aa, Artificial Sequence
  Synthetic Construct
  MISC_FEATURE (1)..(6): Cyclic Peptide
KYMGFP

SEQ ID NO: 266 - protein, 5 aa, Artificial Sequence
  Synthetic Construct
  MISC_FEATURE (5)..(5): Xaa is psi(thiomethylene)leucine
YAGFX

SEQ ID NO: 267 - protein, 6 aa, Artificial Sequence
  Synthetic Construct
  MISC_FEATURE (1)..(6): Cyclic Peptide
KYGGFL

SEQ ID NO: 268-271 - skipped (4 entries)

SEQ ID NO: 272 - protein, 5 aa, Artificial Sequence
  Synthetic Construct
  MISC_FEATURE (1)..(1): t-butyloxycarbonylation
  MISC_FEATURE (4)..(5): psi(thioamide) bond
  MISC_FEATURE (5)..(5): benzyl ester modification
YGGFL

SEQ ID NO: 273 - protein, 5 aa, Artificial Sequence
  Synthetic Construct
  MISC_FEATURE (2)..(5): Cyclic Peptide
  MISC_FEATURE (4)..(5): psi(thiomethylene) bond
YKGFL

SEQ ID NO: 274 - protein, 5 aa, Artificial Sequence
  Synthetic Construct
  MOD_RES (4)..(4): Xaa is (4-nitro)phenylalanine
  MISC_FEATURE (5)..(5): Amidation
YGGXL

SEQ ID NO: 275 - protein, 5 aa, Artificial Sequence
  Synthetic Construct
  MOD_RES (3)..(3): Xaa is Sar
  MOD_RES (4)..(4): Xaa is (4-nitro)phenylalanine
  MISC_FEATURE (5)..(5): Amidation
YGXXL

SEQ ID NO: 276 - protein, 6 aa, Artificial Sequence
  Synthetic Construct
  MISC_FEATURE (6)..(6): Xaa is O-glucosylserinamide
YTGFLX

SEQ ID NO: 277 - skipped

SEQ ID NO: 278 - protein, 5 aa, Artificial Sequence
  Synthetic Construct
  MISC_FEATURE (5)..(5): Hydroxylation
YVGFA

SEQ ID NO: 279 - protein, 9 aa, Artificial Sequence
  Synthetic Construct
  MOD_RES (4)..(4): Xaa is phenylalaninamide
  MOD_RES (6)..(6): Xaa is phenylalaninamide
YAGXPXGAY

SEQ ID NO: 280 - protein, 5 aa, Artificial Sequence
  Synthetic Construct
  MISC_FEATURE (5)..(5): S-ethyl ester modification
YAGFC

SEQ ID NO: 281 - protein, 4 aa, Artificial Sequence
  Synthetic Construct
  MISC_FEATURE (2)..(2): Xaa is alanylglycyl
  MISC_FEATURE (4)..(4): S-butyl ester modification
YXFC

SEQ ID NO: 282 - protein, 5 aa, Artificial Sequence
  Synthetic Construct
  MOD_RES (4)..(4): Xaa is 4-nitrophenylalanine
  MISC_FEATURE (5)..(5): Amidation
YRGXP

SEQ ID NO: 283 - protein, 4 aa, Artificial Sequence
  Synthetic Construct
  MISC_FEATURE (4)..(4): Amidation
YRFF

SEQ ID NO: 284 - protein, 4 aa, Artificial Sequence
  Synthetic Construct
  MOD_RES (2)..(2): Xaa is methionyl(O)
  MOD_RES (4)..(4): Xaa is ethylphenylalanine-2-acetylhydrazide
YXGX

SEQ ID NO: 285 - protein, 4 aa, Artificial Sequence
  Synthetic Construct
  MISC_FEATURE (4)..(4): Dodecane modification
RPKP

SEQ ID NO: 286 - skipped

SEQ ID NO: 287 - protein, 5 aa, Artificial Sequence
  Synthetic Construct
  MISC_FEATURE (1)..(1): (phenylmethoxy)carbonylation
NCCPR

SEQ ID NO: 288 - protein, 4 aa, Artificial Sequence
  Synthetic Construct
  MISC_FEATURE (1)..(1): Acetylation
  MISC_FEATURE (4)..(4): Amidation
MPRG

SEQ ID NO: 289 - skipped

SEQ ID NO: 290 - protein, 5 aa, Artificial Sequence
  Synthetic Construct
  MISC_FEATURE (5)..(5): Amidation
NSFRY

SEQ ID NO: 291 - protein, 78 aa, Artificial Sequence
  Synthetic Construct
  MOD_RES (38)..(38): Xaa is epsilon-Ahx
  MOD_RES (77)..(77): Xaa is epsilon-Ahx
GRGDSGRKKRRQRRRPPQGRGDSGRKKRRQRRRPPQKXCGRGDSGRKKRRQRRRPPQGRGDSGRKKRRQRRRPPQKXC

SEQ ID NO: 292 - protein, 32 aa, Artificial Sequence
  Synthetic Construct
NANPNANPNANPNANPNANPNANPNANPNANP

SEQ ID NO: 293 - protein, 31 aa, Artificial Sequence
  Synthetic Construct
GPGGAGPGGAGPGGAGPGGAGPGGAGPGGAG

SEQ ID NO: 294 - protein, 18 aa, Artificial Sequence
  Synthetic Construct
FGEFGEFGEYGEFGEFGD

SEQ ID NO: 295 - protein, 30 aa, Artificial Sequence
  Synthetic Construct
  MISC_FEATURE (2)..(2): Xaa is Hyp
  MISC_FEATURE (5)..(5): Xaa is Hyp
  MISC_FEATURE (8)..(8): Xaa is Hyp
  MISC_FEATURE (11)..(11): Xaa is Hyp
  MISC_FEATURE (14)..(14): Xaa is Hyp
  MISC_FEATURE (17)..(17): Xaa is Hyp
  MISC_FEATURE (20)..(20): Xaa is Hyp
  MISC_FEATURE (23)..(23): Xaa is Hyp
  MISC_FEATURE (25)..(25): Xaa is Hyp
  MISC_FEATURE (26)..(26): Xaa is Hyp
  MISC_FEATURE (28)..(28): Xaa is Hyp
  MISC_FEATURE (29)..(29): Xaa is Hyp
PXGPXGPXGPXGPXGPXGPXGPXGPXGPXG

SEQ ID NO: 296 - protein, 30 aa, Artificial Sequence
  Synthetic Construct
PPGPPGPPGPPGPPGPPGPPGPPGPPGPPG

SEQ ID NO: 297 - protein, 26 aa, Artificial Sequence
  Synthetic Construct
  MISC_FEATURE (1)..(1): Acetylation
  MOD_RES (3)..(3): Xaa is Hyp
  MOD_RES (6)..(6): Xaa is Hyp
  MOD_RES (9)..(9): Xaa is Hyp
  MOD_RES (15)..(15): Xaa is Hyp
  MOD_RES (18)..(18): Xaa is Hyp
  MOD_RES (21)..(21): Xaa is Hyp
  MOD_RES (24)..(24): Xaa is Hyp
  MISC_FEATURE (26)..(26): C-terminal is CONH2
GPXGPXGPXGPWGPXGPXGPXGPXGG

SEQ ID NO: 298 - protein, 26 aa, Artificial Sequence
  Synthetic Construct
  MISC_FEATURE (1)..(1): Acetylation
  MOD_RES (3)..(3): Xaa is Hyp
  MOD_RES (6)..(6): Xaa is Hyp
  MOD_RES (9)..(9): Xaa is Hyp
  MOD_RES (12)..(12): Xaa is Hyp
  MOD_RES (15)..(15): Xaa is Hyp
  MOD_RES (18)..(18): Xaa is Hyp
  MOD_RES (21)..(21): Xaa is Hyp
  MOD_RES (24)..(24): Xaa is Hyp
  MISC_FEATURE (26)..(26): C-terminal is CONH2
GPXGPXGPXGWXGPXGPXGPXGPXGG

SEQ ID NO: 299 - protein, 14 aa, Artificial Sequence
  Synthetic Construct
  MISC_FEATURE (1)..(1): Acetylation
  MOD_RES (13)..(14): Xaa is beta-Ala
LARLLARLLARLXX

SEQ ID NO: 300 - protein, 21 aa, Artificial Sequence
  Synthetic Construct
  MISC_FEATURE (1)..(1): Acetylation
  MISC_FEATURE (21)..(21): C-terminus is CONH2
LSLLLSLLSLLLSLLSLLLSL

SEQ ID NO: 301 - protein, 20 aa, Artificial Sequence
  Synthetic Construct
  MISC_FEATURE (1)..(1): Acetylation
  MISC_FEATURE (20)..(20): Aldehyde terminated
AAVALLPAVLLALLAPDEVD

SEQ ID NO: 302 - protein, 20 aa, Artificial Sequence
  Synthetic Construct
  MISC_FEATURE (1)..(1): Acetylation
  MISC_FEATURE (20)..(20): Aldehyde terminated
AAVALLPAVLLALLAPYVAD

SEQ ID NO: 303 - protein, 15 aa, Artificial Sequence
  Synthetic Construct
  MISC_FEATURE (1)..(1): Acetylation
  MOD_RES (6)..(6): Xaa is Aib
  MISC_FEATURE (15)..(15): C-terminus is amidated
PEWLRXGVTFPGYIT

SEQ ID NO: 304 - protein, 15 aa, Artificial Sequence
  Synthetic Construct
  MISC_FEATURE (1)..(1): Acetylation
  MISC_FEATURE (15)..(15): C-terminus is amidated
WGHGHGHGPGHGHGH

SEQ ID NO: 305 - protein, 17 aa, Artificial Sequence
  Synthetic Construct
  MISC_FEATURE (1)..(1): Acetylation
  MISC_FEATURE (17)..(17): C-terminus is amidated
WEAQAREALAKEAQARA

SEQ ID NO: 306 - protein, 13 aa, Artificial Sequence
  Synthetic Construct
  MISC_FEATURE (1)..(1): Acetylation
CNANPNANPNANP

SEQ ID NO: 307 - skipped

SEQ ID NO: 308 - protein, 13 aa, Artificial Sequence
  Synthetic Construct
RRRRYGSRRRRRY

SEQ ID NO: 309 - protein, 18 aa, Artificial Sequence
  Synthetic Construct
  MISC_FEATURE (3)..(11): disulfide bond
RLCRIVVIRVCRDDDDEE

SEQ ID NO: 310 - protein, 18 aa, Artificial Sequence
  Synthetic Construct
  MISC_FEATURE (3)..(11): disulfide bond
RLCRIVVIRVCRSDDDEE

SEQ ID NO: 311 - protein, 24 aa, Artificial Sequence
  Synthetic Construct
  MISC_FEATURE (1)..(1): Benzyloxycarbonylation
  MISC_FEATURE (24)..(24): Methyl ester termination
GPPGPPGPPGPPGPPGPPGPPGPP

SEQ ID NO: 312 - protein, 19 aa, Artificial Sequence
  Synthetic Construct
  MOD_RES (1)..(1): Xaa is cyclohexylalanine
  MISC_FEATURE (3)..(4): epsilon(COCH2S) bond
  MOD_RES (18)..(18): Xaa is cyclohexylalanine
XPRGGGGGDYEPIPEEYXE

SEQ ID NO: 313 - protein, 17 aa, Artificial Sequence
  Synthetic Construct
  MOD_RES (2)..(2): Xaa is epsilon-aminocaproyl acid residue
  MISC_FEATURE (5)..(5): C10 caprinoyl acid modification
  MISC_FEATURE (8)..(8): C10 caprinoyl acid modification
  MISC_FEATURE (12)..(12): C10 caprinoyl acid modification
  MISC_FEATURE (15)..(15): C10 caprinoyl acid modification
CXWKKKKKKKKKYKKKK

SEQ ID NO: 314 - protein, 9 aa, Artificial Sequence
  Synthetic Construct
FLPIVGAKL

SEQ ID NO: 315 - protein, 15 aa, Artificial Sequence
  Synthetic Construct
EILIFWSKDIGYSFT

SEQ ID NO: 316 - protein, 23 aa, Artificial Sequence
  Synthetic Construct
KKKKKKKKKKKKKKKKGRGDSPC

SEQ ID NO: 317 - skipped

SEQ ID NO: 318 - protein, 4 aa, Artificial Sequence
  Synthetic Construct
LSKL

SEQ ID NO: 319 - protein, 15 aa, Artificial Sequence
  Synthetic Construct
  MISC_FEATURE (1)..(1): N-tert-butyloxycarbonylation
  MOD_RES (4)..(4): Xaa is aminoisobutyryl
  MOD_RES (11)..(11): Xaa is aminoisobutyryl
  MOD_RES (15)..(15): Xaa is aminoisobutyryl
  MISC_FEATURE (15)..(15): Methyl ester termination
VALXVALVALXVALX

SEQ ID NO: 320 - protein, 8 aa, Artificial Sequence
  Synthetic Construct
MYFGGGGG

SEQ ID NO: 321 - protein, 17 aa, Artificial Sequence
  Synthetic Construct
  MOD_RES (7)..(7): Xaa is alpha-aminoisobutyryl
  MOD_RES (11)..(11): Xaa is alpha-aminoisobutyryl
  MOD_RES (17)..(17): Amidation
FGGFTGXRKSXRKLADQ

SEQ ID NO: 322 - protein, 4 aa, Artificial Sequence
  Synthetic Construct
RGDT

SEQ ID NO: 323 - protein, 4 aa, Artificial Sequence
  Synthetic Construct
AEYG

SEQ ID NO: 324 - protein, 4 aa, Artificial Sequence
  Synthetic Construct
AYEG

SEQ ID NO: 325 - protein, 5 aa, Artificial Sequence
  Synthetic Construct
AVGVP

SEQ ID NO: 326 - protein, 4 aa, Artificial Sequence
  Synthetic Construct
EAYG

SEQ ID NO: 327 - protein, 4 aa, Artificial Sequence
  Synthetic Construct
EYAG

SEQ ID NO: 328 - protein, 5 aa, Artificial Sequence
  Synthetic Construct
GVGVP

SEQ ID NO: 329 - protein, 4 aa, Artificial Sequence
  Synthetic Construct
HDSG

SEQ ID NO: 330 - protein, 5 aa, Artificial Sequence
  Synthetic Construct
  MISC_FEATURE (5)..(5): Ethyl ester termination
YEAGG

SEQ ID NO: 331 - protein, 5 aa, Artificial Sequence
  Synthetic Construct
LGGVG

SEQ ID NO: 332 - protein, 4 aa, Artificial Sequence
  Synthetic Construct
LLFP

SEQ ID NO: 333 - protein, 4 aa, Artificial Sequence
  Synthetic Construct
KYYK

SEQ ID NO: 334 - protein, 4 aa, Artificial Sequence
  Synthetic Construct
FAEG

SEQ ID NO: 335 - protein, 4 aa, Artificial Sequence
  Synthetic Construct
FEAG

SEQ ID NO: 336 - protein, 5 aa, Artificial Sequence
  Synthetic Construct
  MISC_FEATURE (1)..(1): Xaa is sacroyl
  MISC_FEATURE (5)..(5): Aminoethylaminocarbonylmethyl(N-methyl)amino- co-alpha, omega-bis(oxiranylmethyl)poly(ethylene glycol) modification
XGFLG

SEQ ID NO: 337 - protein, 4 aa, Artificial Sequence
  Synthetic Construct
YAEG

SEQ ID NO: 338 - protein, 4 aa, Artificial Sequence
  Synthetic Construct
YEAG

SEQ ID NO: 339 - protein, 5 aa, Artificial Sequence
  Synthetic Construct
YIGSR

SEQ ID NO: 340 - protein, 5 aa, Artificial Sequence
  Synthetic Construct
VGGVG

SEQ ID NO: 341 - protein, 13 aa, Artificial Sequence
  Synthetic Construct
PKLLKTFLSKWIG

SEQ ID NO: 342 - protein, 14 aa, Artificial Sequence
  Synthetic Construct
PSGFYLKLDPRNFN

SEQ ID NO: 343 - skipped

SEQ ID NO: 344 - protein, 5 aa, Artificial Sequence
  Synthetic Construct
  MISC_FEATURE (1)..(1): (99m)Tc labeled
AGGCG

SEQ ID NO: 345 - protein, 5 aa, Artificial Sequence
  Synthetic Construct
  MISC_FEATURE (1)..(1): (99m)Tc labeled
AGGCL

SEQ ID NO: 346 - protein, 5 aa, Artificial Sequence
  Synthetic Construct
  MISC_FEATURE (1)..(1): (99m)Tc labeled
ASSCG

SEQ ID NO: 347 - protein, 14 aa, Artificial Sequence
  Synthetic Construct
TMKIIPFNRTLIGG

SEQ ID NO: 348 - protein, 5 aa, Artificial Sequence
  Synthetic Construct
VPGVG

SEQ ID NO: 349 - DNA, 180 bp, Artificial Sequence
  Synthetic Construct
atgagaccagatttctgcctcgagccgccgtacactgggccctgcaaagctcgtatcatccgttacttctacaatgcaaaggcaggcctgtgtcagaccttcgtatacggcggctgcagagctaagcgtaacaacttcaaatccgcggaagactgcatgcgtacttgcggtggtgcttag

SEQ ID NO: 350 - protein, 30 aa, Artificial Sequence
  Synthetic Construct
  MISC_FEATURE (1)..(5): This sequence may encompass 1-6 ′Gly-Gly-Gly-Gly-Ser′ repeating units
GGGGSGGGGSGGGGSGGGGSGGGGSGGGGS

SEQ ID NO: 351-357 - skipped (7 entries)

SEQ ID NO: 358 - protein, 5 aa, Artificial Sequence
  Synthetic Construct
SSSSG

SEQ ID NO: 359 - protein, 13 aa, Artificial Sequence
  Synthetic Construct
  MOD_RES (1)..(1): Xaa is L-pyroglutamic acid (pE)
  MISC_FEATURE (13)..(13): Optionally -OH terminated
XLYENKPRRPYIL

SEQ ID NO: 360-362 - skipped (3 entries)

SEQ ID NO: 363 - protein, 6 aa, Artificial Sequence
  Synthetic Construct
RRPYIL

SEQ ID NO: 364 - skipped

SEQ ID NO: 365 - protein, 7 aa, Artificial Sequence
  Synthetic Construct
  MISC_FEATURE (1)..(1): Acetylation
KRRPYIL

SEQ ID NO: 366 - protein, 7 aa, Artificial Sequence
  Synthetic Construct
  MISC_FEATURE (1)..(1): Acetylation
  MOD_RES (5)..(5): Xaa is D-Tyr
  MISC_FEATURE (7)..(7): Optionally -OH terminated
KRRPXIL

SEQ ID NO: 367 - protein, 8 aa, Artificial Sequence
  Synthetic Construct
  MOD_RES (1)..(1): Xaa is L-pyroglutamic acid
XKRRPYIL

SEQ ID NO: 368 - protein, 6 aa, Artificial Sequence
  Synthetic Construct
  MISC_FEATURE (1)..(1): Maleimido hexanoic acid (MHA) modification
  MISC_FEATURE (6)..(6): Optionally -OH terminated
RRPYIL

SEQ ID NO: 369 - protein, 6 aa, Artificial Sequence
  Synthetic Construct
  MISC_FEATURE (1)..(1): beta-mercapto-maleimido hexanoic acid (beta-mercaptoMHA) modification
RRPYIL

SEQ ID NO: 370 - protein, 13 aa, Artificial Sequence
  Synthetic Construct
  MOD_RES (1)..(1): Glu is L-pyroglutamic acid (pE)
  MISC_FEATURE (13)..(13): C-terminus is hydroxylated
ELYENKPRRPYIL

SEQ ID NO: 371 - protein, 6 aa, Artificial Sequence
  Synthetic Construct
  MISC_FEATURE (1)..(1): N-terminal MHA group
  MISC_FEATURE (6)..(6): C-terminus is hydroxylated
RRPYIL

SEQ ID NO: 372 - skipped

SEQ ID NO: 373 - protein, 30 aa, Artificial Sequence
  Synthetic Construct
  MISC_FEATURE (1)..(1): Xaa is Hyp
  MISC_FEATURE (4)..(4): Xaa is Hyp
  MISC_FEATURE (7)..(7): Xaa is Hyp
  MISC_FEATURE (10)..(10): Xaa is Hyp
  MISC_FEATURE (13)..(13): Xaa is Hyp
  MISC_FEATURE (16)..(16): Xaa is Hyp
  MISC_FEATURE (19)..(19): Xaa is Hyp
  MISC_FEATURE (22)..(22): Xaa is Hyp
  MISC_FEATURE (25)..(25): Xaa is Hyp
  MISC_FEATURE (28)..(28): Xaa is Hyp
XPGXPGXPGXPGXPGXPGXPGXPGXPGXPG

SEQ ID NO: 374 - skipped

SEQ ID NO: 375 - protein, 39 aa, Artificial Sequence; identical to SEQ ID NO: 127
  Synthetic Construct

SEQ ID NO: 376 - protein, 39 aa, Artificial Sequence
  Synthetic Construct
  MISC_FEATURE (39)..(39): Maleimido hexanoic acid (MHA) modification
HGEGTFTSDLSKQMEEEAVRLFIEWLKNGGPSSGAPPPK

SEQ ID NO: 377 - protein, 39 aa, Artificial Sequence
  Synthetic Construct
HGEGTFTSDLSKQMEEEAVRLFIEWLKNGGPCSGAPPPS

SEQ ID NO: 378 - DNA, 129 bp, Artificial Sequence
  Synthetic Construct
ctggttccgcgtggatccacctttttctatggcggcagccgtggcaaacgcaacaatttcaagaccgaggagtatagctgctccctgcctcagaccagtggcctgcagaagccagagagctgaattccc

SEQ ID NO: 379 - protein, 40 aa, Artificial Sequence
  Synthetic Construct
LVPRGSTFFYGGSRGKRNNFKTEEYSCSLPQTSGLQKPES

SEQ ID NO: 380 - protein, 6 aa, Artificial Sequence
  Synthetic Construct
LVPRGS

SEQ ID NO: 381 - skipped

SEQ ID NO: 382 - DNA, 562 bp, Artificial Sequence
  Synthetic Construct
atgggcagcagccatcatcatcatcatcacagcagcggcctggtgccgcgcggcagccatacctttttctatggcggcagccgtggcaaacgcaacaatttcaagaccgaggagtatatggtaccgatccagaaagttcaggatgacaccaaaaccctgatcaagaccattgttacccgtatcaacgacatttctcacacgcagtctgtatctgccaagcagcgtgttactggcttggacttcattccgggtcttcacccgattctgtctttgtctaagatggaccagactctggcagtttatcaacaggttctgaccagcctgccgatctcaaaacgtactgcagatcgccaacgacctggagaacctgcgtgacctgctgcatctgctggccttctctaagagctgctctctgccgcagacctctggcctgcagaagccggagagcctggatggcgttctggaagcctctctgtactctactgaggtggtggctttgagccgtctgcagggctctctgcaggacattcttcaacagttggatgttagcccggaatgctag

SEQ ID NO: 383 - protein, 186 aa, Artificial Sequence
  Synthetic Construct
MGSSHHHHHHSSGLVPRGSHTFFYGGSRGKRNNFKTEEYMVPIQKVQDDTKTLIKTIVTRINDISHTQSVSAKQRVTGLDFIPGLHPILSLSKMDQTLAVYQQVLTSLPSQNVLQIANDLENLRDLLHLLAFSKSCSLPQTSGLQKPESLDGVLEASLYSTEVVALSRLQGSLQDILQQLDVSPEC

SEQ ID NO: 384 - protein, 167 aa, Homo sapiens
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 385 - skipped

SEQ ID NO: 386 - protein, 45 aa, Artificial Sequence
  Synthetic Construct
PPGPPGPPGPPGPPGPPGPPGPPGPPGPPGPPGPPGPPGPPGPPG

SEQ ID NO: 387 - protein, 78 aa, Artificial Sequence
  Synthetic Construct
  MISC_FEATURE (39)..(40): Maleimido hexanoic acid (MHA) linker
  MISC_FEATURE (59)..(60): Maleimido propionic acid (MPA) linker
  MISC_FEATURE (78)..(78): Hydroxylation
HGEGTFTSDLSKQMEEEAVRLFIEWLKNGGPSSGAPPPKTFFYGGSRGKRNNFKTEEYCTFFYGGSRGKRNNFKTEEY

(19) United States
(12) Patent Application Publication
(10) Pub. No.: US 2016/0263235 A1
(43) Pub. Date: Sep. 15, 2016

(54) PEPTIDE THERAPEUTIC CONJUGATES AND USES THEREOF

(71) Applicant: ANGIOCHEM INC., MONTREAL (CA)

(72) Inventors: JEAN-PAUL CASTAIGNE, MONT-ROYAL (CA); MICHEL DEMEULE, BEACONSFIELD (CA); CHRISTIAN CHE, LONGUEUIL (CA); CARINE THIOT, PREAUX (FR); CATHERINE GAGNON, MONTREAL-NORD (CA); BETTY LAWRENCE, BOLTON (CA)

(21) Appl. No.: 14/696,193

(22) Filed: Apr. 24, 2015

Related U.S. Application Data
(63) Continuation of application No. 13/133,002, filed on Aug. 9, 2011, now abandoned.
(60) Provisional application No. 61/200,947, filed on Dec. 5, 2008.

(51) Int. Cl. A61K 47/48; A61K 38/22; C07K 14/575

(52) U.S. Cl. CPC A61K 47/48246; C07K 14/57563; A61K 38/2278; C07K 14/5759; A61K 38/2264

(57) ABSTRACT

The present invention features a compound having the formula A-X-B, where A is peptide vector capable of enhancing transport of the compound across the blood-brain barrier or into particular cell types, X is a linker, and B is a peptide therapeutic. The compounds of the invention can be used to treat any disease for which the peptide therapeutic is useful.

### BACKGROUND OF THE INVENTION

The invention relates to compounds including a peptide therapeutic bound to a peptide vector and uses thereof.

Peptides, such as peptide hormones, have found a variety of therapeutic uses. One of the challenges in treatment of patients using peptides is to ensure delivery of peptides to the desired tissue.

In particular, delivery to brain tissues is often reduced or prevented by the blood-brain barrier (BBB).

In the development of a new therapy for brain pathologies, the blood-brain barrier (BBB) is considered a major obstacle for the potential use of drugs for treating disorders of the central nervous system (CNS). The global market for CNS drugs was $68 billion in 2006, which was roughly half that of global market for cardiovascular drugs, even though in the United States, nearly twice as many people suffer from CNS disorders as from cardiovascular diseases. The reason for this imbalance is, in part, that more than 98% of all potential CNS drugs do not cross the BBB. In addition, more than 99% of worldwide CNS drug development is devoted solely to CNS drug discovery, and less than 1% is directed to CNS drug delivery. This may explain the lack of therapeutic options available for major neurological diseases.

The brain is shielded against potentially toxic substances by the presence of two barrier systems: the BBB and the blood-cerebrospinal fluid barrier (BCSFB). The BBB is considered to be the major route for the uptake of serum ligands since its surface area is approximately 5000-fold greater than that of BCSFB. The brain endothelium, which constitutes the BBB, represents the major obstacle for the use of potential drugs against many disorders of the CNS. As a general rule, only small lipophilic molecules may pass across the BBB, i.e., from circulating systemic blood to brain. Many drugs that have a larger size or higher hydrophobicity show high efficacy in CNS targets but are not efficacious in animals as these drugs cannot effectively cross the BBB. Thus, peptide and protein therapeutics are generally excluded from transport from blood to brain, owing to the negligible permeability of the brain capillary endothelial wall to these drugs. Brain capillary endothelial cells (BCECs) are closely sealed by tight junctions, possess few fenestrae and few endocytic vesicles as compared to capillaries of other organs. BCECs are surrounded by extracellular matrix, astrocytes, pericytes, and microglial cells. The close association of endothelial cells with the astrocyte foot processes and the basement membrane of capillaries are important for the development and maintenance of the BBB properties that permit tight control of blood-brain exchange.

Thus, there exists a need for improved delivery of peptide therapeutics to tissues, including tissues protected by the BBB.

### SUMMARY OF THE INVENTION

We have developed compounds that include (a) a peptide such as a peptide therapeutic (e.g., any peptide therapeutic described herein) and (b) a peptide vector. These compounds are useful in treating any disorder where increased transport of the peptide therapeutic across the BBB or into particular cell types is desired. In one particular example, the compound includes a GLP-1 agonist as a peptide therapeutic, which may be used to treat metabolic disorders such as diabetes and obesity. The peptide vector is capable of transporting the peptide therapeutic either across the blood-brain barrier (BBB) or into a particular cell type (e.g., liver, lung, kidney, spleen, and muscle). Surprisingly, we have shown that lower doses of exemplary peptide therapeutics, exendin-4 analogs, when conjugated to a peptide vectors as described herein, are effective in treating glycemia. Because the conjugates are targeted across the BBB or to particular cell types, therapeutic efficacy can be achieved using lower doses or less frequent dosing as compared to unconjugated peptide therapeutics, thus reducing the severity of or incidence of side effects and/or increasing efficacy. The compound may also exhibit increased stability, improved pharmacokinetics, or reduced degradation in vivo, as compared to the unconjugated peptide therapeutic.

Accordingly, in a first aspect the invention features a compound having the formula:

A-X-B

where A is a peptide vector capable of being transported across the blood-brain barrier (BBB) or into a particular cell type (e.g., liver, lung, kidney, spleen, and muscle), X is a linker, and B is a peptide therapeutic (e.g., a peptide therapeutic described herein). The transport across the BBB or into the cell may be increased by at least 10%, 25%, 50%, 75%, 100%, 200%, 500%, 750%, 1000%, 1500%, 2000%, 5000%, or 10,000%. The compound may be substantially pure. The compound may be formulated with a pharmaceutically acceptable carrier (e.g., any described herein).

In another aspect, the invention features methods of making the compound A-X-B. In one embodiment, the method includes conjugating the peptide vector (A) to a linker (X), and conjugating the peptide vector-linker (A-X) to a peptide therapeutic (B), thereby forming the compound A-X-B. In another embodiment, the method includes conjugating the peptide therapeutic (B) to a linker (X), and conjugating the peptide therapeutic/linker (X-B) to a peptide vector (A), thereby forming the compound A-X-B. In another embodiment, the method includes conjugating the peptide vector (A) to a peptide therapeutic (B), where either A or B optionally include a linker (X), to form the compound A-X-B.

In another aspect, the invention features a nucleic acid molecule that encodes the compound A-X-B, where the compound is a polypeptide. The nucleic acid molecule may be operably linked to a promoter and may be part of a nucleic acid vector. The vector may be in a cell, such as a prokaryotic cell (e.g., bacterial cell) or eukaryotic cell (e.g., yeast or mammalian cell, such as a human cell).

In another aspect, the invention features methods of making a compound of the formula A-X-B, where A-X-B is a polypeptide. In one embodiment, the method includes expressing a nucleic acid vector of the previous aspect in a cell to produce the polypeptide; and purifying the polypeptide.

In another aspect, the invention features a method of treating (e.g., prophylactically) a subject having a metabolic disorder. The method includes administering a compound of the first aspect in an amount sufficient to treat the disorder (e.g., where the peptide therapeutic is suitable for treating a metabolic disorder). In certain embodiments, the metabolic disorder is diabetes (e.g., Type I or Type II), obesity, diabetes as a consequence of obesity, hyperglycemia, dyslipidemia, hypertriglyceridemia, syndrome X, insulin resistance, impaired glucose tolerance (IGT), diabetic dyslipidemia, hyperlipidemia, a cardiovascular disease, or hypertension.

In another aspect, the invention features a method of reducing food intake by, or reducing body weight of, a subject. The method includes administering a compound of the first aspect of the invention (e.g., where the peptide therapeutic that reduces food intake) to a subject in an amount sufficient to reduce food intake or reduce body weight. The subject may be overweight, obese, or bulimic.

In another aspect, the invention features a method of treating (e.g., prophylactically) a disorder selected from the group consisting of anxiety, movement disorder, aggression, psychosis, seizures, panic attacks, hysteria, sleep disorders, Alzheimer's disease, and Parkinson's disease. The method includes administering a compound of the first aspect of the invention to a subject in an amount sufficient to treat or prevent the disorder.

The invention also features a method of increasing neurogenesis in a subject. The method includes administering a compound of the first aspect to a subject. The subject may desire, or may be in need of neurogenesis. In certain embodiments, the subject may be suffering from a disease or disorder of the central nervous system such as Parkinson's Disease, Alzheimer's Disease, Huntington's Disease, ALS, stroke, ADD, and neuropsychiatric syndromes. In other embodiments, the increase in neurogenesis can improve learning or enhance neuroprotection.

In another aspect, the invention features a method for converting liver stem/progenitor cells into functional pancreatic cells; preventing beta-cell deterioration and stimulation of β-cell proliferation; treating obesity; suppressing appetite and inducing satiety; treating irritable bowel syndrome; reducing the morbidity and/or mortality associated with myocardial infarction and stroke; treating acute coronary syndrome characterized by an absence of Q-wave myocardial infarction; attenuating post-surgical catabolic changes; treating hibernating myocardium or diabetic cardiomyopathy; suppressing plasma blood levels of norepinepherine; increasing urinary sodium excretion, decreasing urinary potassium concentration; treating conditions or disorders associated with toxic hypervolemia, e.g., renal failure, congestive heart failure, nephrotic syndrome, cirrhosis, pulmonary edema, and hypertension; inducing an inotropic response and increasing cardiac contractility; treating polycystic ovary syndrome; treating respiratory distress; improving nutrition via a non-alimentary route, i.e., via intravenous, subcutaneous, intramuscular, peritoneal, or other injection or infusion; treating nephropathy; treating left ventricular systolic dysfunction (e.g., with abnormal left ventricular ejection fraction); inhibiting antro-duodenal motility (e.g., for the treatment or prevention of gastrointestinal disorders such as diarrhea, postoperative dumping syndrome and irritable bowel syndrome, and as premedication in endoscopic procedures; treating critical illness polyneuropathy (CIPN) and systemic inflammatory response syndrome (SIRS; modulating triglyceride levels and treating dyslipidemia; treating organ tissue injury caused by reperfusion of blood flow following ischemia; or treating coronary heart disease risk factor (CHDRF) syndrome in a subject by administering and effective amount of a compound of the first aspect.

In another aspect, the invention features a method of treating (e.g., prophylactically) a cancer, a neurodegenerative disease, or a lysosomal storage disorder (e.g., any disease described herein). The method includes administering to a subject a compound of the first aspect (e.g., where the peptide therapeutic can be used to treat the disease or disorder) in an amount sufficient to treat the disease or disorder.

In any of the methods involving administration of a compound to a subject, the amount sufficient may be less than 90%, 75%, 50%, 40%, 30%, 20%, 15%, 10%, 5%, 4%, 3%, 2%, 1%, or 0.1% of the amount required for an equivalent dose of the peptide therapeutic (e.g., any described herein) when not conjugated to the peptide vector. The amount sufficient may reduce a side effect (e.g., vomiting, nausea, or diarrhea) as compared to administration of an effective amount of the peptide therapeutic when not conjugated to the peptide vector. The subject may be a mammal such as a human.

In any of the above aspects, the peptide vector may be a polypeptide substantially identical to any of the sequences set Table 1, or a fragment thereof. In certain embodiments, the peptide vector has a sequence of Angiopep-1 (SEQ ID NO:67), Angiopep-2 (SEQ ID NO:97), Angiopep-3 (SEQ ID NO:107), Angiopep-4a (SEQ ID NO:108), Angiopep-4b (SEQ ID NO:109), Angiopep-5 (SEQ ID NO:110), Angiopep-6 (SEQ ID NO:111), or Angiopep-7 (SEQ ID NO:112)). The peptide vector or conjugate may be efficiently transported into a particular cell type (e.g., any one, two, three, four, or five of liver, lung, kidney, spleen, and muscle) or may cross the mammalian BBB efficiently (e.g., Angiopep-1, -2, -3, -4a, -4b, -5, and -6). In another embodiment, the peptide vector or conjugate is able to enter a particular cell type (e.g., any one, two, three, four, or five of liver, lung, kidney, spleen, and muscle) but does not cross the BBB efficiently (e.g., a conjugate including Angiopep-7). The peptide vector may be of any length, for example, at least 6, 7, 8, 9, 10, 11, 12, 13, 14, 15, 16, 17, 18, 19, 20, 21, 25, 35, 50, 75, 100, 200, or 500 amino acids, or any range between these numbers. In certain embodiments, the peptide vector is 10 to 50 amino acids in length. The polypeptide may be produced by recombinant genetic technology or chemical synthesis.

TABLE 1
Exemplary Peptide Vectors
SEQ
ID
NO:

| | |
|---|---|
| 1 | T F V Y G G C R A K R N N F K S A E D |
| 2 | T F Q Y G G C M G N G N N F V T E K E |
| 3 | P F F Y G G C G G N R N N F D T E E Y |
| 4 | S F Y Y G G C L G N K N N Y L R E E E |
| 5 | T F F Y G G C R A K R N N F K R A K Y |
| 6 | T F F Y G G C R G K R N N F K R A K Y |
| 7 | T F F Y G G C R A K K N N Y K R A K Y |
| 8 | T F F Y G G C R G K K N N F K R A K Y |
| 9 | T F Q Y G G C R A K R N N F K R A K Y |
| 10 | T F Q Y G G C R G K K N N F K R A K Y |
| 11 | T F F Y G G C L G K R N N F K R A K Y |
| 12 | T F F Y G G S L G K R N N F K R A K Y |
| 13 | P F F Y G G C G G K K N N F K R A K Y |
| 14 | T F F Y G G C R G K G N N Y K R A K Y |
| 15 | P F F Y G G C R G K R N N F L R A K Y |
| 16 | T F F Y G G C R G K R N N F K R E K Y |
| 17 | P F F Y G G C R A K K N N F K R A K E |
| 18 | T F F Y G G C R G K R N N F K R A K D |
| 19 | T F F Y G G C R A K R N N F D R A K Y |
| 20 | T F F Y G G C R G K K N N F K R A E Y |
| 21 | P F F Y G G C G A N R N N F K R A K Y |
| 22 | T F F Y G G C G G K K N N F K T A K Y |
| 23 | T F F Y G G C R G N R N N F L R A K Y |
| 24 | T F F Y G G C R G N R N N F K T A K Y |
| 25 | T F F Y G G S R G N R N N F K T A K Y |
| 26 | T F F Y G G C L G N G N N F K R A K Y |
| 27 | T F F Y G G C L G N R N N F L R A K Y |
| 28 | T F F Y G G C L G N R N N F K T A K Y |
| 29 | T F F Y G G C R G N G N N F K S A K Y |
| 30 | T F F Y G G C R G K K N N F D R E K Y |
| 31 | T F F Y G G C R G K R N N F L R E K E |
| 32 | T F F Y G G C R G K G N N F D R A K Y |
| 33 | T F F Y G G S R G K G N N F D R A K Y |
| 34 | T F F Y G G C R G N G N N F V T A K Y |
| 35 | P F F Y G G C G G K G N N Y V T A K Y |
| 36 | T F F Y G G C L G K G N N F L T A K Y |
| 37 | S F F Y G G C L G N K N N F L T A K Y |
| 38 | T F F Y G G C G G N K N N F V R E K Y |
| 39 | T F F Y G G C M G N K N N F V R E K Y |
| 40 | T F F Y G G S M G N K N N F V R E K Y |
| 41 | P F F Y G G C L G N R N N Y V R E K Y |
| 42 | T F F Y G G C L G N R N N F V R E K Y |
| 43 | T F F Y G G C L G N K N N Y V R E K Y |
| 44 | T F F Y G G C G G N G N N F L T A K Y |
| 45 | T F F Y G G C R G N R N N F L T A E Y |
| 46 | T F F Y G G C R G N G N N F K S A E Y |
| 47 | P F F Y G G C L G N K N N F K T A E Y |
| 48 | T F F Y G G C R G N R N N F K T E E Y |
| 49 | T F F Y G G C R G K R N N F K T E E D |
| 50 | P F F Y G G C G G N G N N F V R E K Y |
| 51 | S F F Y G G C M G N G N N F V R E K Y |
| 52 | P F F Y G G C G G N G N N F L R E K Y |
| 53 | T F F Y G G C L G N G N N F V R E K Y |
| 54 | S F F Y G G C L G N G N N Y L R E K Y |
| 55 | T F F Y G G S L G N G N N F V R E K Y |
| 56 | T F F Y G G C R G N G N N F V T A E Y |
| 57 | T F F Y G G C L G K G N N F V S A E Y |
| 58 | T F F Y G G C L G N R N N F D R A E Y |
| 59 | T F F Y G G C L G N R N N F L R E E Y |
| 60 | T F F Y G G C L G N K N N Y L R E E Y |
| 61 | P F F Y G G C G G N R N N Y L R E E Y |
| 62 | P F F Y G G S G G N R N N Y L R E E Y |
| 63 | M R P D F C L E P P Y T G P C V A R I |
| 64 | A R I I R Y F Y N A K A G L C Q T F V Y G |
| 65 | Y G G C R A K R N N Y K S A E D C M R T C G |
| 66 | P D F C L E P P Y T G P C V A R I I R Y F Y |
| 67 | T F F Y G G C R G K R N N F K T E E Y |
| 68 | K F F Y G G C R G K R N N F K T E E Y |
| 69 | T F Y Y G G C R G K R N N Y K T E E Y |
| 70 | T F F Y G G S R G K R N N F K T E E Y |
| 71 | C T F F Y G C C R G K R N N F K T E E Y |
| 72 | T F F Y G G C R G K R N N F K T E E Y C |
| 73 | C T F F Y G S C R G K R N N F K T E E Y |
| 74 | T F F Y G G S R G K R N N F K T E E Y C |
| 75 | P F F Y G G C R G K R N N F K T E E Y |
| 76 | T F F Y G G C R G K R N N F K T K E Y |
| 77 | T F F Y G G K R G K R N N F K T E E Y |
| 78 | T F F Y G G C R G K R N N F K T K R Y |
| 79 | T F F Y G G K R G K R N N F K T A E Y |
| 80 | T F F Y G G K R G K R N N F K T A G Y |
| 81 | T F F Y G G K R G K R N N F K R E K Y |
| 82 | T F F Y G G K R G K R N N F K R A K Y |
| 83 | T F F Y G G C L G N R N N F K T E E Y |
| 84 | T F F Y G C G R G K R N N F K T E E Y |
| 85 | T F F Y G G R C G K R N N F K T E E Y |
| 86 | T F F Y G G C L G N G N N F D T E E E |
| 87 | T F Q Y G G C R G K R N N F K T E E Y |
| 88 | Y N K E F G T F N T K G C E R G Y R F |
| 89 | R F K Y G G C L G N M N N F E T L E E |
| 90 | R F K Y G G C L G N K N N F L R L K Y |
| 91 | R F K Y G G C L G N K N N Y L R L K Y |
| 92 | K T K R K R K K Q R V K I A Y E E I F K N Y |
| 93 | K T K R K R K K Q R V K I A Y |
| 94 | R G G R L S Y S R R F S T S T G R |
| 95 | R R L S Y S R R R F |
| 96 | R Q I K I W F Q N R R M K W K K |
| 97 | T F F Y G G S R G K R N N F K T E E Y |
| 98 | M R P D F C L E P P Y T G P C V A R I |
| | I R Y F Y N A K A G L C Q T F V Y G G |
| | C R A K R N N F K S A E D C M R T C G G A |
| 99 | T F F Y G G C R G K R N N F K T K E Y |
| 100 | R F K Y G G C L G N K N N Y L R L K Y |
| 101 | T F F Y G G C R A K R N N F K R A K Y |
| 102 | N A K A G L C Q T F V Y G G C L A K R N N F |
| | E S A E D C M R T C G G A |
| 103 | Y G G C R A K R N N F K S A E D C M R T C G |
| | G A |
| 104 | G L C Q T F V Y G G C R A K R N N F K S A E |
| 105 | L C Q T F V Y G G C E A K R N N F K S A |
| 107 | T F F Y G G S R G K R N N F K T E E Y |
| 108 | R F F Y G G S R G K R N N F K T E E Y |
| 109 | R F F Y G G S R G K R N N F K T E E Y |
| 110 | R F F Y G G S R G K R N N F R T E E Y |
| 111 | T F F Y G G S R G K R N N F R T E E Y |
| 112 | T F F Y G G S R G R R N N F R T E E Y |
| 113 | C T F F Y G G S R G K R N N F K T E E Y |
| 114 | T F F Y G G S R G K R N N F K T E E Y C |
| 115 | C T F F Y G G S R G R R N N F R T E E Y |
| 116 | T F F Y G G S R G R R N N F R T E E Y C |

Polypeptides Nos. 5, 67, 76, and 91, include the sequences of SEQ ID NOS: 5, 67, 76, and 91, respectively, and are amidated at the C-terminus.
Polypeptides Nos. 107, 109, and 110 include the sequences of SEQ ID NOS: 97, 109, and 110, respectively, and are acetylated at the N-terminus.

In any of the above aspects, the peptide vector may include an amino acid sequence having the formula:

X1 -X2-X3-X4-X5-X6-X7-X8-X9-X10-X11-X12-X13-X14-X15-X16-X17-X18-X19

where each of X1-X19 (e.g., X1-X6, X8, X9, X11-X14, and X16-X19) is, independently, any amino acid (e.g., a naturally occurring amino acid such as Ala, Arg, Asn, Asp, Cys, Gln, Glu, Gly, His, Ile, Leu, Lys, Met, Phe, Pro, Ser, Thr, Trp, Tyr, and Val) or absent and at least one (e.g., 2 or 3) of X1, X10, and X15 is arginine. In some embodiments, X7 is Ser or Cys; or X10 and X15 each are independently Arg or Lys. In some embodiments, the residues from X1 through X19, inclusive, are substantially identical to any of the amino acid sequences of any one of SEQ ID NOS:1-105 and 107-116 (e.g., Angiopep-1, Angiopep-2, Angiopep-3, Angiopep-4a, Angiopep-4b, Angiopep-5, Angiopep-6, and Angiopep-7). In some embodiments, at least one (e.g., 2, 3, 4, or 5) of the amino acids X1-X19 is Arg. In some embodiments, the polypeptide has one or more additional cysteine residues at the N-terminal of the polypeptide, the C-terminal of the polypeptide, or both.

In any of the above aspects, the peptide therapeutic may be selected from the group consisting of antimicrobial or antibiotic peptides, gastrointestinal peptides, pancreatic peptides, peptide hormones, hypothalamic hormones, pituitary hormones, and neuropeptides. The gastrointestinal or pancreatic peptide may be a cholecystokinin, gastrin, glucagon, epidermal growth factor, vasoactive intestinal peptide (VIP), insulin, or a GLP-1 agonist. The hypothalamic or pituitary hormone may be a pituitary hormone-releasing hormone (e.g., corticotropin-releasing hormone, gonadotropin-releasing hormone, growth hormone-releasing hormone, and thyrotropin-releasing hormone (TRH), a pituitary hormone release inhibiting hormone (e.g., MSH release inhibiting hormone and somatostatin), pro-opiomelanocortin or an analog or derivative (e.g., cleavage product) thereof (e.g., adrenocorticotropic hormone (ACTH), α-endorphin, β-endorphin, γ-endorphin, β-lipotropin, γ-lipotropin, and melanocyte-stimulating hormone), growth hormones, thyrotropin, vasotocin, and oxytocin. The neuropeptide may be any of angiotensin, bombesin, bradykinin, calcitonin, a cholecystokinin, delta sleep inducing peptide, galanin, gastric inhibitory polypeptide, gastrin, neuropeptide Y, neurotensin, an opioid peptide (e.g., a dynorphin, an endorphin, an enkephalin, and a nociceptin), vasoactive intestinal peptides, secretin, tachykinin, and vasopressin. Other peptide hormones include adiponectins, adrenomedullins, ghrelin, gonadotropins, inhibins, natriuretic peptides, parathyroid hormone (PTH) and parathyroid hormone related peptide (PTHrP), peptide YY, thymosin, and relaxins. In other embodiments, the peptide therapeutic is a distintegrin. In certain embodiments, the peptide therapeutic is not a nutriceutical, an antibody, an antibody fragment such as an Fv fragment, F(ab)2, F(ab)2′, or Fab, a cellular toxin, an endotoxin, an exotoxin, or an anti-angiogenic compound such as a tyrosine kinase inhibitor or VEGF inhibitor. Other peptide therapeutics include disintegrins, endothelins, and secretory protein inhibitor proteins. The peptide therapeutic may an analog or fragment of any of these peptides (e.g., any described herein). In certain embodiments, the analog or fragment has the same biological activity as the parent peptide.

In any of the above aspects, the peptide therapeutic may be a GLP-1 agonist. The GLP-1 agonist may GLP-1, exendin-4, exendin-3, or analog or fragment thereof (e.g., any analog or fragment described herein). In particular embodiments, the GLP-1 agonist is an exendin-4 analog selected from the group consisting of [Lys³⁹]exendin-4 and [Cys³²]exendin-4.

In particular embodiments, the peptide conjugated to the vector is selected from the group consisting of leptin, monomethyl auristatin E (MMAE), diphtheria toxin, botunilum toxin, tetanus toxin, pertussis toxin, staphylococcus enterotoxins, toxin shock syndrome toxin TSST-1, adenylate cyclase toxin, shiga toxin, cholera enterotoxin, endostatin, catechins, chemokine IP-10, inhibitors of matrix metalloproteinase (MMPIs), anastellin, vironectin, antithrombin, herceptin, avastin, panitumumab, a green fluorescent protein, a His tag protein, galactosidase, luciferase, peroxidase and phosphatase.

In certain embodiments of any of the above aspects, the peptide vector or peptide therapeutic is modified (e.g., as described herein). The peptide may be amidated, acetylated, or both. Such modifications may be at the amino or carboxy terminus of the polypeptide. The polypeptide may also include peptidomimetics (e.g., those described herein) of any of the polypeptides described herein. The polypeptide may be in a multimeric form, for example, dimeric form (e.g., formed by disulfide bonding through cysteine residues).

In certain embodiments, the peptide vector or peptide therapeutic has an amino acid sequence described herein with at least one amino acid substitution (e.g., 2, 3, 4, 5, 6, 7, 8, 9, 10, 11, or 12 substitutions), insertion, or deletion. The polypeptide may contain, for example, 1 to 12, 1 to 10, 1 to 5, or 1 to 3 amino acid substitutions, for example, 1 to 10 (e.g., to 9, 8, 7, 6, 5, 4, 3, 2) amino acid substitutions. The amino acid substitution(s) may be conservative or non-conservative. For example, the peptide vector may have an arginine at one, two, or three of the positions corresponding to positions 1, 10, and 15 of the amino acid sequence of any of SEQ ID NO:1, Angiopep-1, Angiopep-2, Angiopep-3, Angiopep-4a, Angiopep-4b, Angiopep-5, Angiopep-6, and Angiopep-7. In certain embodiments, the GLP-1 agonist may have a cysteine or lysine substitution or addition at any position (e.g., a lysine substitution at the N- or C-terminal position, or a cysteine substitution at the position corresponding to amino acid 32 of the exendin-4 sequence).

In any of the above aspects, the compound may specifically exclude a polypeptide including or consisting of any of SEQ ID NOS:1-105 and 107-116 (e.g., Angiopep-1, Angiopep-2, Angiopep-3, Angiopep-4a, Angiopep-4b, Angiopep-5, Angiopep-6, and Angiopep-7). In some embodiments, the polypeptides and conjugates of the invention exclude the polypeptides of SEQ ID NOs:102, 103, 104, and 105.

In any of the above aspects, the linker (X) may be any linker known in the art or described herein. In particular embodiments, the linker is a covalent bond (e.g., a peptide bond), a chemical linking agent (e.g., those described herein), an amino acid or a peptide (e.g., 2, 3, 4, 5, 8, 10, or more amino acids). In certain embodiments, the linker has the formula:

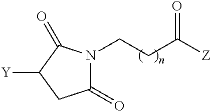

where n is an integer between 2 and 15 (e.g., 2, 3, 4, 5, 6, 7, 8, 9, 10, 11, 12, 13, 14, or 15); and either Y is a thiol on A and Z is a primary amine on B or Y is a thiol on B and Z is a primary amino on A.

By “peptide vector” is meant a compound or molecule such as a polypeptide or a polypeptide mimetic that can be transported into a particular cell type (e.g., liver, lungs, kidney, spleen, or muscle) or across the BBB. The vector may be attached to (covalently or not) or conjugated to peptide therapeutic and thereby may be able to transport the peptide therapeutic into a particular cell type or across the BBB. In certain embodiments, the vector may bind to receptors present on cancer cells or brain endothelial cells and thereby be transported into the cancer cell or across the BBB by transcytosis. The vector may be a molecule for which high levels of transendothelial transport may be obtained, without affecting the cell or BBB integrity. The vector may be a polypeptide or a peptidomimetic and may be naturally occurring or produced by chemical synthesis or recombinant genetic technology.

By “peptide therapeutic” is meant any polypeptide sequence or fragment thereof having at least one biological activity. As used herein, the term “peptide therapeutic” excludes leptin, monomethyl auristatin E (MMAE), diphtheria toxin, botunilum toxin, tetanus toxin, pertussis toxin, staphylococcus enterotoxins, toxin shock syndrome toxin TSST-1, adenylate cyclase toxin, shiga toxin, cholera enterotoxin, endostatin, catechins, chemokine IP-10, inhibitors of matrix metalloproteinase (MMP1s), anastellin, vironectin, antithrombin, herceptin, avastin, panitumumab, a green fluorescent protein, a His tag protein, galactosidase, luciferase, peroxidase and phosphatase.

By “GLP-1 agonist” is meant any compound capable of activating a GLP-1 receptor (e.g., a mammalian or human GLP-1 receptor). Agonists can include peptides or small molecule compounds (e.g., any of those described herein). Assays for determining whether a particular compound is a GLP-1 agonist are known in the art and described herein.

By “treating” a disease, disorder, or condition in a subject is meant reducing at least one symptom of the disease, disorder, or condition by administrating a therapeutic agent to the subject.

By “treating prophylactically” a disease, disorder, or condition in a subject is meant reducing the frequency of occurrence of (e.g., preventing) a disease, disorder or condition by administering a therapeutic agent to the subject.

In one example, a subject who is being treated for a metabolic disorder is one who a medical practitioner has diagnosed as having such a condition. Diagnosis may be performed by any suitable means, such as those described herein. A subject in whom the development of diabetes or obesity is being treated prophylactically may or may not have received such a diagnosis. One in the art will understand that subject of the invention may have been subjected to standard tests or may have been identified, without examination, as one at high risk due to the presence of one or more risk factors, such as family history, obesity, particular ethnicity (e.g., African Americans and Hispanic Americans), gestational diabetes or delivering a baby that weighs more than nine pounds, hypertension, having a pathological condition predisposing to obesity or diabetes, high blood levels of triglycerides, high blood levels of cholesterol, presence of molecular markers (e.g., presence of autoantibodies), and age (over 45 years of age). An individual is considered obese when their weight is 20% (25% in women) or more over the maximum weight desirable for their height. An adult who is more than 100 pounds overweight, is considered to be morbidly obese. Obesity is also defined as a body mass index (BMI) over 30 kg/m².

By “a metabolic disorder” is meant any pathological condition resulting from an alteration in a subject's metabolism. Such disorders include those resulting from an alteration in glucose homeostasis resulting, for example, in hyperglycemia. According to this invention, an alteration in glucose levels is typically an increase in glucose levels by at least 5%, 10%, 20%, 30%, 40%, 50%, 60%, 70%, 80%, 90%, or even 100% relative to such levels in a healthy individual. Metabolic disorders include obesity and diabetes (e.g., diabetes type I, diabetes type II, MODY, and gestational diabetes), satiety, and endocrine deficiencies of aging.

By “reducing glucose levels” is meant reducing the level of glucose by at least 10%, 20%, 30%, 40%, 50%, 60%, 70%, 80%, 90%, 95%, or 100% relative to an untreated control. Desirably, glucose levels are reduced to normoglycemic levels, i.e., between 150 to 60 mg/dL, between 140 to 70 mg/dL, between 130 to 70 mg/dL, between 125 to 80 mg/dL, and preferably between 120 to 80 mg/dL. Such reduction in glucose levels may be obtained by increasing any one of the biological activities associated with the clearance of glucose from the blood (e.g., increase insulin production, secretion, or action).

By “subject” is meant a human or non-human animal (e.g., a mammal).

By “increasing GLP-1 receptor activity” is meant increasing the level of receptor activation measured using standard techniques (e.g., cAMP activation) by, for example, at least 10%, 20%, 50%, 75%, 100%, 200%, or 500% as compared to an untreated control.

By “equivalent dosage” is meant the amount of a compound of the invention required to achieve the same molar amount of the peptide therapeutic (e.g., a GLP-1 agonist) in the compound of the invention, as compared to the unconjugated peptide therapeutic. For example, the equivalent dosage of 1.0 μg exendin-4 is about 1.6 μg of the [Lys³⁹-MHA]exendin-4/Angiopep-2-Cys-NH₂ conjugate described herein.

By a polypeptide which is “efficiently transported across the BBB” is meant a polypeptide that is able to cross the BBB at least as efficiently as Angiopep-6 (i.e., greater than 38.5% that of Angiopep-1 (250 nM) in the in situ brain perfusion assay described in U.S. patent application Ser. No. 11/807,597, filed May 29, 2007, hereby incorporated by reference). Accordingly, a polypeptide which is “not efficiently transported across the BBB” is transported to the brain at lower levels (e.g., transported less efficiently than Angiopep-6).

By a polypeptide or compound which is “efficiently transported to a particular cell type” is meant that the polypeptide or compound is able to accumulate (e.g., either due to increased transport into the cell, decreased efflux from the cell, or a combination thereof) in that cell type to at least a 10% (e.g., 25%, 50%, 100%, 200%, 500%, 1,000%, 5,000%, or 10,000%) greater extent than either a control substance, or, in the case of a conjugate, as compared to the unconjugated agent. Such activities are described in detail in International Application Publication No. WO 2007/009229, hereby incorporated by reference.

Other features and advantages of the invention will be apparent from the following Detailed Description, the drawings, and the claims.

### BRIEF DESCRIPTION OF THE DRAWINGS

FIG. 1 is table and schematic diagram showing exendin-4 and the exendin-4 analogs used in experiments described herein.

FIG. 2 is a schematic diagram of the synthetic scheme used to conjugate Cys-AngioPep2, Angiopep-2-Cys-NH₂, and Angiopep-1 to [Lys³⁹-MHA]exendin-4.

FIG. 3 is a schematic diagram of the synthetic scheme used to conjugate [Cys³²]exendin-4 to (maleimido propionic acid (MPA))-Angiopep-2, (maleimido hexamoic acid (MHA))-Angiopep-2, and (maleimido undecanoic acid (MUA))-Angiopep-2.

FIG. 4 is a graph showing transport of exendin-4 and exendin-4/Angiopep-2 across the BBB. The total amount in the brain, along with the amounts in the capillaries and the parenchyma are shown.

FIG. 5 is a graph showing increase in weight of (ob/ob) mice following administration of a control, exendin-4, or the [Lys³⁹-MHA]exendin-4/Angiopep-2-Cys-NH₂ conjugate (Exen-An2). Both exendin-4 and Ex-An2 were observed to reduce weight gain as compared to the animals receiving the control.

FIG. 6 is a graph showing total food consumption by (ob/ob) mice, where the mice were administered a control, exendin-4, or the Exen-An2. Both exendin-4 and Exen-An2 were observed to reduce food intake as compared to the animals receiving the control.

FIG. 7 is a graph showing reduction in glycemia following administration of two doses of exendin-4 (3 μg/kg and 30 μg/kg) and equivalent doses of Exen-An2 (4.8 μg/kg and 48 μg/kg). A similar reduction in glycemia at the lower dose of Exen-An2, as compared to the higher dose of exendin-4, was observed. During this experiment, one mouse in the control group died at day 12.

FIG. 8A is a schematic diagram showing the structure of an Exendin-4-Angiopep-2 dimer conjugate (Ex4(Lys39(MHA))-AN2-AN2). The compound has the structure HGEGTFTSDLSKQMEEEAVRLFIEWLKNGGPSSGAPPPK(MHA)-TFFYGGSRGKRNNFKTEEYC-(MPA)-TFFYGGSRGKRNNFKTEEY-OH, where MHA is maleimido hexanoic acid and MPA is maleimido propionic acid.

FIG. 8B is a schematic structure of an Exendin-4-scramble-Angiopep-2 (Ex4(Cys32)-ANS4 (N-Term) or Exen-S4) that was used a control. This compound has the structure HGEGTFTSDLSKQMEEEAVRLFIEWLKNGGPCSGAPPPS-(MHA)-GYKGERYRGFKETNFNTFS-OH, where MHA is maleimido hexanoic acid.

FIG. 9 is a graph showing the ability of Exendin-4, Exendin-4-Angiopep-2 conjugates, the Exen-S4, and Exendin-4 when conjugated conjugated to a dimeric form of Angiopep-2, to crosses the BBB.

FIG. 10 is a graph showing the ability of Exendin-4 and Exen-An2-An2 to reduce glycemia in mice.

FIGS. 11A and 11B are graphs showing tissue concentration in brain (FIG. 11A) and in pancreas (FIG. 11B) of Exendin-4 and Exen-4-An-2.

FIG. 12 is a graph showing dose-response of insulin secretion in response to either Exendin-4 or Exen-An2 in RIN-m5F pancreas cells.

FIGS. 13A and 13B are chromatograms showing the Leptin-AN2 (C11) conjugate before (FIG. 13A) and after (FIG. 13B) purification.

FIG. 14 is a chromatogram showing the results of purification of the Leptin-AN2 (C11) conjugate.

FIG. 15 is a graph showing uptake of the C3, C6, and C11 Leptin-AN2 conjugates into the brain, capillaries, and parenchyma using the in situ brain perfusion assay.

FIGS. 16A and 16B are graphs showing in situ brain perfusion of the leptin₁₁₆₋₁₃₀ and the Leptin-AN2 (C11) conjugate in lean mice and diet induced obese (DIO) mice (FIG. 16A) and plasma levels of leptin in lean mice and DIO mice (FIG. 16B).

FIGS. 17A and 17B are graphs showing food intake in mice receiving a control injection (saline), leptin₁₁₆₋₁₃₀, or the Leptin-AN2 (C11) conjugate after either four hours (FIG. 17A) or 15 hours (FIG. 17B).

FIG. 18 is a graph showing weight gain over a six-day period in mice receiving a control, leptin₁₁₆₋₁₃₀, or the Leptin-AN2 (C11) conjugate.

FIG. 19 is a graph showing weight gain over a ten-day period in ob/ob mice receiving a control, leptin₁₁₆₋₁₃₀, or the leptin-AN2 (C11) conjugate by daily IP injection over a period of six days.

FIG. 20 is a schematic diagram showing the GST tagged Angiopep construct.

FIG. 21 is a schematic diagram showing the PCR strategy used to generate the Angiopep-2-leptin₁₁₆₋₁₃₀ fusion protein.

FIG. 22 is a chromatogram showing purification of the GST-Angiopep2 on a GSH-sepharose column

FIGS. 23A-23C show a western blot (FIG. 23A), a UV spectrum from a liquid chromatography experiment (FIG. 23B), and a mass spectrum (FIG. 23C) of the recombinant Angiopep-2 peptide.

FIG. 24 is a graph showing uptake of the synthetic and recombinant forms of Angiopep-2 in the in situ brain perfusion assay.

FIG. 25 is a graph showing uptake of GST, GST-Angiopep-2, GST-leptin₁₁₆₋₁₃₀, and GST-Angiopep-2-leptin₁₁₆₋₁₃₀ into the parenchyma in the in situ brain perfusion assay.

FIG. 26 is a schematic diagram showing the His-tagged-mouse leptin and His-tagged-Angiopep-2-mouse leptin fusion protein.

FIG. 27 is an image of a gel showing purification of the His-tagged mouse leptin and the human leptin sequence.

FIG. 28 is the sequence of human leptin precursor. Amino acids 22-167 of this sequence form the mature leptin peptide.

FIGS. 29A and 29B are exemplary purification schemes for His-tagged leptin (mouse) and the His-tagged Angiopep-2-leptin conjugate.

FIG. 30 is photograph of a gel showing successful small-scale expression of the leptin and Angiopep-2-leptin conjugate.

FIG. 31 is a schematic diagram and picture of a gel showing that two products resulted from thrombing cleavage of the His-tagged conjugate.

FIG. 32 is a graph showing uptake of leptin and the Angiopep-2-leptin fusion protein into the parenchyma of DIO mice.

FIG. 33 is a graph showing the effect of recombinant leptin on the weight of ob/ob mice.

FIG. 34 is a graph showing the change in weight in DIO mice recieving a control, leptin, His-tagged mouse letpin, or the His-tagged Angiopep-2-leptin conjugate.

FIGS. 35A and 35B are chromatograms showing the ECMS-Neurotensin compound (ECMS-NT) before (FIG. 35A) and after (FIG. 35B) purification using the analytical method described in the examples.

FIG. 36 is a chromatogram showing purification of ECMS-NT on an AKTA-explorer with column filled with 30 ml of 30RPC resin.

FIGS. 37A and 37B are chromatograms showing Neurotensin Angiopep-2-Cys amide conjugate (NT-AN2Cys-NH₂ or NT-An2) before (FIG. 37A) and after (FIG. 37B) purification using the analytical method described in the examples.

FIG. 38 is a chromatogram showing purification of NT-An2 on an AKTA-explorer with column filled with 30 ml of 30RPC resin.

FIG. 39 is a graph showing hypothermia induction by NT-An2. Mice received saline (control), NT (1 mg/kg) or NT-An2 at 2.5 mg/kg or 5.0 mg/kg (equivalent to 1 and 2 mg/kg doses of NT). Rectal temperature was monitored 90 minutes following intravenous injection.

FIG. 40 is a graph showing the effect of body temperature in mice upon administration of 5, 15, or 20 mg/kg of NT-An2.

FIG. 41 is a graph showing the effect of body temperature in mice upon administration of 5, 10, or 20 mg/kg of a different preparation of NT-An2.

FIG. 42 is a graph showing in situ brain perfusion of NT and NT-An2. Following iodination, mice brains were perfused in the carotid artery with either [¹²⁵I]-NT or the [¹²⁵I]-NT-An2 derivative in Krebs buffer for the indicated times. After the indicated times, brains were further perfused for 30 sec to washout the excess of both compound. Both [¹²⁵I]-NT or [¹²⁵I]-NT-An2 derivative in brain were quantified using a beta counter. Results are expressed in terms of brain volume of distribution (ml/100 g) as a function of time.

FIG. 43 is a graph showing brain compartmentation of NT and NT-An2 after in situ brain perfusion as described for FIG. 40. Brain capillary depletion was performed using Dextran following standard procedures. Both [¹²⁵I]-NT or [¹²⁵I]-NT-An2 derivative present in brain, capillaries, and parenchyma were quantified and volume of distribution (ml/100 g/2 min) is reported.

FIG. 44 is a graph showing body temperature of mice receiving a bolus 5 mg/kg injection of the NT-An2, followed one hour later by a 2.5 hour infusion of NT-An2 at a rate of 5 mg/kg/30 min (i.e., 10 mg/kg/hr).

FIG. 45 is a graph showing body temperature of a rat receiving an intravenous bolus injection of 20 mg/kg NT-An2, followed immediately by a 20 mg/kg/hr infusion of NT-An2 for 3.5 hours.

FIG. 46 is a graph showing body temperature of mice receiving an intravenous bolus injection of 20 mg/kg NT-An2, followed immediately by a 20 mg/kg/hr infusion of NT-An2, which was increased to 40 mg/kg/hr after 2.5 hours.

FIG. 47 is a graph showing body temperature of rats receiving an intravenous bolus injection of 20 mg/kg NT-An2, followed immediately by a 20 mg/kg/hr infusion of NT-An2.

FIG. 48 is a graph showing body temperature of ratings receiving an intravenous bolus injection of 40 mg/kg NT-An2, followed immediately by a 40 mg/kg/hr infusion of NT-An2. This resulted in sustained reduction in body temperature for the 12 hour duration of the experiment.

FIG. 49 is a graph showing latency in the hot plate test in mice of the paw licking response in control mice (left), mice receiving 20 mg/kg NT-An2 (center), and mice receiving 1 mg/kg buprenorphine (right) just prior to and 15 minutes following administration of the compound.

FIG. 50 is a graph showing body temperature of mice receiving a bolus intravenous 7.5 mg/kg injection of NT(8-13), Ac-Lys-NT(8-13), Ac-Lys-[D-Tyr¹¹]NT(8-13), pGlu-NT(8-13), or a control. From among these analogs, Ac-Lys-[D-Tyr¹¹]NT(8-13) was observed to produce the greatest reduction in body temperature.

FIG. 51 is a graph showing body temperature of mice receiving a bolus intravenous injection of a control, NT, NT-An2, NT(8-13)-An2, and Ac-Lys-[D-Tyr¹¹]NT(8-13)-An2. The greatest reduction in body temperature was observed for NT-An2 and Ac-Lys-[D-Tyr¹¹]NT(8-13)-An2 conjugates.

FIG. 52 is a graph showing body temperature of mice receiving a bolus intravenous injection of Ac-Lys-[D-Tyr¹¹]NT(8-13) (1 mg/kg) or Ac-Lys-[D-Tyr¹¹]NT(8-13)-An2 (6.25 mg/kg). The An2 conjugated molecule was observed to reduce body temperature to a greater extent that the unconjugated molecule.

FIG. 53 is a graph showing body temperature of a mouse receiving a 6.25 mg/kg bolus intravenous injection of the Ac-Lys-[D-Tyr¹¹]NT(8-13)-An2 conjugate followed 60 minutes later by a 6.25 mg/kg/hr infusion of the conjugate.

FIG. 54 is a graph showing binding of radiolabeled NT ([³H]-NT) to HT29 cells that express the NTSR1 in the presence or absence of 40 nM of NT at 4° C. or 37° C.

FIG. 55 is a graph showing binding of [³H]-NT to HT29 cells in the presence of NT at concentrations ranging from 0.4 nM to 40 nM.

FIG. 56 is graph showing binding of [³H]-NT to HT29 cells in the presence of NT or Ac-Lys-[D-Tyr¹¹]NT(8-13).

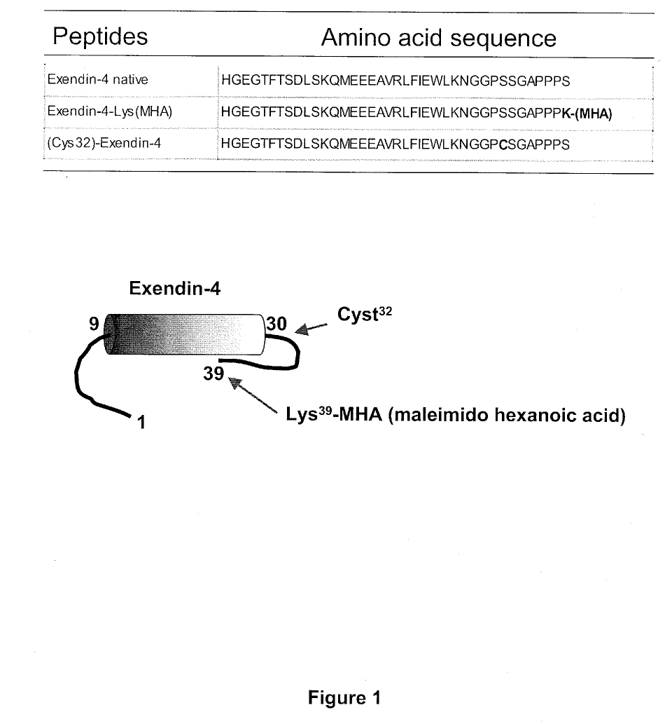

### DETAILED DESCRIPTION

We have developed peptide therapeutic conjugates having an enhanced ability to cross the blood-brain barrier (BBB) or to enter particular cell type(s) (e.g., liver, lung, kidney, spleen, and muscle) as exemplified by conjugates of peptide vectors to the exemplary peptide therapeutics, exendin-4, leptin, and neurotensin, and analogs thereof. The peptide conjugates of the invention thus include a therapeutic peptide and a peptide vector that enhance transport across the BBB.

Surprisingly, we have also shown that lower doses of the compounds of the invention, as compared to unconjugated GLP-1 agonists, are effective in treating GLP-1 related disorders, including a reduction in glycemia. By administering lower doses of the conjugated peptides, side effects such as vomiting, nausea, and diarrhea observed with the unconjugated agonists can be reduced or eliminated. Alternatively, increased efficacy at higher doses may be obtained.

The peptide therapeutic can be any peptide having biological known in the art and including peptides such as those described below. Particular GLP-1 agonists include exendin-4, GLP-1, and exendin-3 fragments, substitutions (e.g., conservative or nonconservative substitutions, or substitutions of non-naturally occurring amino acids), and chemical modifications to the amino acid sequences (e.g., those described herein). Peptide therapuetics, including GLP-1 agonists, are described in detail below.

### Peptide Therapeutics

Any peptide known in the art may be conjugated to a peptide vector of the invention. The peptide may be a mammalian peptide such as mouse, rat, or human peptide, or may be a nonmammalian peptide. Exemplary peptides are described below.

### Antimicrobial or Antibiotic Peptides

In certain embodiments, the peptide therapeutic is an antimicrobial or antibiotic peptide. The conjugate may be used to treat an infection such as a bacterial infection (e.g., any known in the art). Antimicrobial peptides include (KIAGKIA)₃ peptide, Apis mellifera abaecin protein, Ala19-magainin 2 amide, Ala(8,13,18)-magainin 2 amide, plant α-thionin protein, wheat α1-purothionin protein, anoplin, antimicrobial hybrid peptide CM15, antimicrobial peptide ESF39A, antimicrobial peptide LL-37, antimicrobial peptide V4, apidaecin, apoE(133-162), Hyas araneus arasin 1, aurein 1.2 peptide, aurein 2.2 peptide, aurein 2.3 peptide, Bac7(1-35) peptide, bactericidal permeability increasing protein, β lysin, Bombina orientalis BLP-7 protein, bombinin H2, BTM-P1 peptide, Anura caerin 1.1, Cavia CAP11 protein, CAP18 lipopolysaccharide-binding protein, cathelicidin antimicrobial peptide, cathelicidin, cationic antimicrobial protein 57, cationic antimicrobial protein CAP 37, CEC(dir)-CEC(ret) protein, cecropin A, cecropin A(1-7)melittin(2-9), cecropin A(1-8)magainin 2(1-12), cecropin C, Cecropins, chrysophsin, chicken CMAP27 protein, D-V13A(D) peptide, D-V13K(D) peptide, DC-1 peptide, DC-2 peptide, DC-2R peptide, Aesculus hippocastanum Ah-AMP1 protein, human α-defensin 5, human α-defensin 6, mouse cryptdin 4, defensin NP-1, defensin NP-3a, human DEFT1P protein, human neutrophil peptide 1, human neutrophil peptide 2, human neutrophil peptide 3, human neutrophil peptide 4, rat neutrophil peptide 3, neutrophil peptide 5, Oryctolagus cuniculus NP-1 protein, RK-1 peptide, mouse BD-6 protein, rat β defensin-1 protein, β-Defensin, human β-defensin 28, human β-defensin 3, mouse β-defensin-12 protein, human β-defensin-27, human β-defensin-5 protein, human β-defensin-6 protein, rat Bin1b protein, bovine neutrophil β-defensin 12, human DEFB-109 peptide, human DEFB1 protein, mouse Defb1 protein, mouse Defb14 protein, mouse Defb2 protein, human DEFB4 protein, mouse Defb4 protein, mouse Defb5 protein, mouse Defb7 protein, mouse Defb8 protein, mouse Defr1 protein, chicken gallinacin-8 protein, chicken gallinacin-9 protein, gramicidin, gramicidin A, gramicidin B, gramicidin C, gramicidin D, gramicidin S, hPAB-βprotein, lingual antimicrobial peptide, mouse Spag11 protein, mouse Tdl protein, mouse CRS4C protein, human DEFB118 protein, defensin NP-2, Aeshna cyanea defensin protein, deoxypheganomycin D, gallerimycin, chicken gallinacin 1 protein, chicken gallinacin 2 protein, Phaseolus limensis limenin protein, peptide NP-3b, peptide NP-5, Phaseolus coccineus phaseococcin protein, human retrocyclin-2, rhesus theta defensin-2, rhesus theta defensin-3, Oryctolagus cuniculus RK-1 protein, Macaca mulatta RTD-2 protein, Macaca mulatta RTD-3 protein, scorpine, Picea abies SPI1 protein, Lycopersicon esculentum tgas118, theta-defensin, dermaseptin, dermaseptin K4S4(1-16)a, dermaseptin K4S4(1-28), dermcidins, desferri-ferricrocin, Epinephelus coioides epinecidin-1, eremomycin aglycone, Evonymous europa lectin, chicken fowlicidin-3, Zea mays γ-zeathionin proteins, Ginkgo biloba ginkbilobin-2 protein, gomesin, Staphylococcus haemolyticus gonococcal growth inhibitor protein, mouse Hamp2 protein, Amblyomma hebraeum hebraein protein, Pyrrhocoris apterus hemiptericin protein, hepcidin, hevein, Dhvar-5, dhvar4 peptide, HTN1 protein, HTN3 protein, P-113D, Apis mellifera hymenoptaecin protein, Impatiens balsamina lb-AMP4 peptide, indolicidin, CP10A indolicidin derivative, IsCT-P peptide, Boophilus microplus Ixodidin, K4-S4(1-13)a, K6L5WP peptide, Raja kenojei kenojeinin I, karabemycin, kutapressin, Lachesana tarabaevi latarcin 2a, levitide, liver-expressed antimicrobial peptide 2, Lachesana tarabaevi Ltc1 peptide, Lachesana tarabaevi Ltc2a peptide, Litoria maculatin-1.1 protein, magainin A, magainin B, magainin G, magainin H, magainins, Bombina maxima maximin 9, MBI-27 protein, melitten or analog thereof (e.g., (4-aminobutanoyl)melittin, (5-aminopentanoyl)melittin, azidosalicylylmelittin, cecropin A(1-8)melittin(1-18), cecropin A(1-8)melittin(1-2), dioleoylmelittin, DNC-melitten, glycylmelittin, hecate 1, hecate-chorionic gonadotropin β-subunit conjugate, melittin(8-26), N-methylanthraniloyl melitten, prepromelittin, promelittin, and tetraacetylmelitten), modelin 1, modelin 5, Bombyx mori moricin protein, Myp30 peptide, myticin, mytilin, neuramide, neutrophil basic proteins, novispirin G10, octyl-cecropin(1-7)melittin(2-9), Odorrana grahami odorranain-NR, omiganan pentahydrochloride, Oxyuranus microlepidotus omwaprin protein, Oncorhyncin III, ovispirin, P18 antimicrobial peptide, P19(8) antimicrobial peptide, P19(9-B) antimicrobial peptide, paracelsin, paracelsin E, parasin I, Litopenaeus setiferus penaeidin-4 protein, peptide 399, peptide-Gly-Leu-amide, Peptite 2000, pexiganan, polymyxins, colistin, colbiocin, colistimethate, colistin heptapeptide, colistin nonapeptide, EuβI, deacylpolymyxin B, lubasporin, paenimyxin, pelargonoyl cyclic decapeptide polymyxin M(1), polymagma, polymyxin B, auricularum, corti-biciron, cortisporin, cyclo(diaminobutyryl-diaminobutyryl-phenylalanyl-leucyl-diaminobutyryl-diaminobutyryl-threonyl), dexapolyspectran, diaminobutyryl-cyclo(diaminobutyryl-diaminobutyryl-phenylalanyl-leucyl-diaminobutyryl-diaminobutyryl-threonyl), maxitrol, panotile, pelargonoyl-cyclic decapeptide polymyxin B(1), polydexa, polymyxin B nonapeptide, polyspectran OS, pulpomixine, septomixine, sP-B compound, sP-Bpy compound, sP-Bw compound, Uniroid, polymyxin B(1), polymyxin S(1), polymyxin T(1), polypeptin, UCB 630, polyphemusin I, polyphemusin II, porcine myeloid antibacterial peptide 23, PR 11 proline-arginine-rich peptide, PR 26, PR 39, prohepcidin, protegrin-1, protegrin-2, protegrin-3, protegrin-4, protegrin-5, Pseudis paradoxa pseudin-2 protein, purothionin, Pyrrhocoris apterus pyrrhocoricin protein, RACA 854, RIN 25 peptide, RL-37 peptide, RPRPNYRPRPIYRP peptide, SB 37, SC5 synthetic antimicrobial peptide, Shiva 11, Shiva 3, Shiva-1, stomoxyn, T22 protein, tachystatin A, Pyrularia pubera THI1 protein, thionins, bovine tracheal antimicrobial peptide, wheat TthV protein, WLBU2 peptide, WS22-N-amide, xenopsin precursor fragment (XPF), antimicrobial peptide IB-367, PGAa antimicrobial peptide, antibacterial polypeptide LCI, antibiotic 2928, antibiotic 5590, antibiotic A 19009, antibiotic AFC-BC11, antibiotic G0069A, ampullosporin, actinomycin HKI 0155, actinotiocin, antrimycin, aplasmomycin, aramycin, argimicin A, auromomycin, auromycin, azinomycin B, azinothricin, azureomycin A, azureomycin B, bacillomycin D, berninamycin A, berninamycin B, berninamycin C, berninamycin D, biphenomycin A, biphenomycin C, cairomycin A, cairomycin B, cairomycin C, chandramycin, cycloheptamycin, cypemycin, cystaurimycin, diperamycin, 2-imidazoledistamycin, chloroacetyldistamycin, distamycin-DAPI, distamycin-EDTA-iron(II), M-bromoacetyldistamycin, permethyldistamycin A, stallimycin, thioformyldistamycin, duramycin, duramycin B, duramycin C, echomycin A, echomycin B, echomycin C, enomycin, enramycin, ficellomycin, gardimycin, globomycin, histidinomycin, hodydamycin, janiemycin, janthinocin A, janthinocin B, janthinocin C, japonicin 1, japonicin 2, kuwaitimycin, lavendomycin, longicatenamycin, macracidmycin, macromomycin B, macromomycin I protein, macromomycin II protein, macromomycin protein, malioxamycin, muraymycin A1, muraymycin A3, muraymycin C1, napsamycin B, napsamycin C, napsamycin D, neoberninamycin, nilemycin, pacidamycin 1, pacidamycin 3, pacidamycin 5, pantomycin, phenomycin, sideromycins, siomycin, siomycin A, siomycin D1, sohbumycin, sporamycin, sporangiomycin, stendomycidin, stendomycin, sulfomycin, syriamycin, takaokamycin, telomycin, termicin, thioxamycin, trichosporin B-Ia, trichosporin B-IIIa, trichosporin B-IIIb, trichosporin B-IIIc, trichosporin B-IIId, trichosporin B-V, trichosporin B-VIa, tritrypticin, tsushimycin, tyrothricin, vancomycin B, yemenimycin, zelkovamycin, zwittermicin A, 3-(1-methyl-4-(1-methyl4-(1-methyl-4-(8-(2′-carboxamidoethyloxy)-7-methoxy-1,2,3,11a-tetrahydro-5H-pyrrolo(2,1-c)(1,4)benzodiazepin-5-one)pyrrole-2-carboxamido)pyrrole-2-carboxamido)pyrrole-2-carboxamido)propionamidine, AR-1-144, dien-microgonotropen-c, distamin, Distal, distel(2+), FCE 24561, FCE 25450A, FCE 26644, FCE 27164, FCE 27266, FCE 27784, MEN 10710, MEN 10716, microgonotropen L2, microgonotropen pentaazapentabutylamine, MT 12, MT 17, N-(2-(diethylamino)ethyl)-1-methyl-4-(1-methyl-4-(4-formamido-1-methylimidazole-2-carboxamido)pyrrole-2-carboxamido)imidazole-2-carboxamide, PNU 151807, PNU 153429, PNU 157977, tallimustine, tren-microgonotropen-b, edeine, edeine A, edeine B, edeine D, edeine F, and octapeptin antibiotics.

### Gastrointestinal or Pancreatic Peptides and Peptide Hormones

In certain embodiments, the peptide therapeutic is a gastrointestinal or pancreatic hormone. Gastrointestinal hormones include cholecystokinin, gastrin, glucagon, epidermal growth factor, and vasoactive intestinal peptide (VIP). Other gastrointestinal and pancreatic peptides include glucagon and glucagon-like peptides. Pancreatic peptides include insulin and somatostatin. Analogs of these peptides are described below. Other gastrointestinal and pancreatic hormones include pancreastatin, pancreastatin(33-49), pancreastatin-16, pancreastatin-29, and pancreastatin-52, pancreatic polypeptide, pancreatic polypeptide (31-36), Torpedo marmorata gut PLY, pancreatic eicosapeptide, avian pancreatic polypeptide, salmon pancreatic polypeptide, human PPY protein, human PPY2 protein, skin peptide tyrosine-tyrosine, glicentin, glicentin (1-16), glicentin(62-69), glicentin-related pancreatic peptide, Glucagon-Like Peptide 2, ALX-0600, glucagon-like peptide-2(3-33), glucagon-like-immunoreactivity, lysyl-arginyl-asparaginyl-lysyl-asparaginyl-asparagine, oxyntomodulin, oxyntomodulin (19-37), and Nle(27)-oxyntomodulin.

Cholecystokinins

In certain embodiments, the gastrointestinal peptide is cholecystokinin or an analog thereof. Cholecystokinin analogs include cholescystokinin, 4-(biotin-epsilon-(aminohexanoyl)oxymethyl)-3-nitrobenzoyl-glycyl-(propionypornithinyl-epsilon-aminohexanoyl-cholecystokinin, 4-alanyloxymethyl-3-nitrobenzoyl-epsilon-aminohexanoyl-cholecystokinin, A 68552, ARL 15849XX, BC 197, BC 264, benzyloxycarbonyl-glycyl-tryptophyl-methionyl-aspartyl(OBu-t)-phenylalaninamide, butyloxycarbonyl-tryptophyl-leucyl-aspartyl-phenylalaninamide, sincalide, (3-azido-4-hydroxy-5-iodobenzimidyl)-CCK-8, 8-sulfocholecystokinin octapeptide, acetylcholecystokinin C-terminal heptapeptide, AR C15849KF, Bolton Hunter-cholecystokinin nonapeptide, Bolton Hunter-cholecystokinin octapeptide, cholecystokinin(26-32), rhodamine-Tyr-Gly-Nle(28,31) phenethyl ester-cholecystokinin(26-32), Tyr-Gly-Nle(28,31) phenethyl ester-cholecystokinin(26-32), cholecystokinin(26-33), cholecystokinin(26-33) sulfated amide, I-Tyr-Gly-(Nle(28,31),4-NO₂-Phe33-cholecystokinin(26-33), I-Tyr-Gly-Nle(28,31)-cholecystokinin(26-33), N-acetyl-norleucine(28,31)-cholecystokinin(26-33), N-α-hydroxysulfonyl-Nle(28,31)-cholecystokinin(26-33), Tyr-Gly-(Nle(28,31),4-NO₂-Phe33)cholecystokinin(26-33), cholecystokinin(27-33), t-butyloxycarbonyl-cholecystokinin(27-33), tert-butyloxycarbonyl-Nle(28,31)-cholecystokinin(27-33), cholecystokinin hexapeptide, desNH₂-Tyr-cholecystokinin octapeptide, cholecystokinin pentapeptide, Tyr27-cholecystokinin-pancreozymin, desaminopancreozymin octapeptide, desulfated sincalide, FPL 14294, indium DTPA-Glu-G-CCK8, JMV 167, JMV 170, JMV 179, JMV 180, JMV 182, JMV 236, JMV 320, JMV 332, JMV 81, N-(4-(4′-azido-3′-iodophenylazo)benzoyI)-3-aminopropionyl-CCK-8, propionyl CCK octapeptide sulfate, pGlu-sincalide, Phe(CH₂SO₃Na)(2)-sincalide, SNF 8702, SNF 8814, SNF 8906, succinyl-tyrosyl-methionyl-glycyl-tryptophyl-methionyl-aspartyl-phenethylamide, SUT 8701, t-butyloxycarbonyl-(sulfo-Tyr)-Met-Gly-Trp-Nle-Asp 2-phenylethyl ester, tert-butyloxycarbonylcholecystokinin-8, CCK 15, cholecystokinin(1-14), cholecystokinin(10-20), biotinyl-Tyr-Gly-(Thr28-Nle31)-cholecystokinin(25-33), Thr28-Nle31-cholecystokinin(25-33), Tyr25-Nle(28,31)-cholecystokinin(25-33), 2-(4-azidosalicylamido)-1,3-dithiopropionate(Thr28-Ahx31)-cholecystokinin(25-33), indium-DOTA(0)-Asp26-Nle(28,31)-cholecystokinin(26-33), iodo-Tyr-Gly-Nle(28,31)-Bpa33-cholecystokinin(26-33), iodo-Tyr-Gly-Nle(28,31)-Bpa(29-33)-cholecystokinin(26-33), benzoyloxycarbonyl-cholecystokinin(27-32) amide, cholecystokinin(27-32)-amide, cholecystokinin(29-33)-amide, butyloxycarbonyl-cholecystokinin(31-33) amide, Thr34-Ahx37-cholecystokinin(31-39), cholecystokinin 10 C-terminal fragment, cholecystokinin 12 C-terminal fragment, cholecystokinin 21, cholecystokinin 22 C-terminal fragment, cholecystokinin 33(10-20), cholecystokinin 39, cholecystokinin 5-pentagastrin, cholecystokinin 58, cholecystokinin 8, cholecystokinin 9, cholecystokinin C-terminal flanking peptide, cholecystokinin precursor C-terminal pentapeptide, Gly-cholecystokinin, cholecystokinin-J, desulfated benzyloxycarbonyl cholecystokinin(26-33), dimyristoylmercaptoglycero-N(α)maleoyl-β-alanyl(Thr,Nle)-CCK-9, JMV 176, MP 2286, MP 2288, pBC 264, preprocholecystokinin, procholecystokinin, Ro 23-7014, SNF 8815, SNF 9007, sulfated cholecystokinin 15, t-butyloxycarbonyl-sulfotyrosyl-norleucyl-glycyl-tyrosyl-aspartyl-2-phenylethyl ester, U 67827E, and V-9-M cholecystokinin nonapeptide.

Epidermal Growth Factor

In certain embodiments, the peptide peptide therapeutic is epidermal growth factor (EGF) or an analog thereof. Such peptides include ¹¹¹In-DTPA-hEGF, ⁶⁸Ga-DOTA-hEGF, biotinyl EGF, biregulin, chicken CALEB protein, E 6010, E1-INT, EGF-genistein, Mouse Emr4 protein, EGF(1-45), EGF(1-48), EGF(1-53), Cys-50₃H(33,42)-EGF(32-48), EGF(33-42), [Cys(Acm)20,31] epidermal growth factor (20-31), EGF precursor, Lys(3)-Tyr(22)-EGF, EGF-dextran-tyrosine conjugate, EGF-dextran conjugate, S(1-5) EGF-like protein, EGF-ricin complex, epigen, epiregulin, C. elegans fat3 protein, human FAT3 protein, rat FAT3 protein, sea urchin fibropellin protein, gigantoxin I, Herdmania momus HmEGFL-1 protein, C. elegans Lin-3 protein, mouse Ly64 protein, Drosophila oep protein, peptabody-EGF, Pseudomonas exotoxin-epidermal growth factor conjugate, Drosophila spi protein, human TDGF1 protein, mouse TDGF1 protein, ^{99m}Tc-HYNIC-human EGF, ^{99m}Tc EGF, and Lys-6-urogastrone.

Glucagon

In certain embodiments, the peptide therapeutic is glucagon or an analog thereof. Such peptides include proglucagon, (desHis1,desPhe6,Glu9)-glucagon-NH₂, γ-L-glutamoyl(Na-hexadecanoyl)-R(34-7)GLP-1(37), glucagon(1-17), glucagon(1-21), glucagon(1-6), glucagon(19-29), desHis(1)-glucagon amide, 12-(N(6)-(4-azidophenylamidino)Lys)-glucagon, 2-nitro-4-azidophenylsulfenyl-glucagon, carboxy-Me-Met(27)-glucagon, desHis(1)-(N(ε)-phenylthiocarbamoyl-Lys(12))-glucagon, desHis(1)-Tyr(22)-glucagon, di-(δ-(5-nitro-2-pyrimidyl)Orn)(17,18)-glucagon, fluorescein-Trp(25) glucagon, homoArg(12)-glucagon, Met-sulfoxide(27)-glucagon, N(α)-citraconyl glucagon, N(α)-malto-Me-Met(27)-glucagon, N(α)-trinitrophenyl-His(1)-homo-Arg(12)-glucagon, oxindolyl-Ala(25)-glucagon, protamine zinc-glucagon, thiol-Trp(2) glucagon, Tyr(22)-glucagon, desHis(1)-Nle(9)-Ala(11,16)-glucagon-amide, desHis(1)-Glu(9)-glucagonamide, imidazopropionyl(7)-arginyl(26)-N(ε)-octanoyl-lysyl(34)-glucaon-like peptide-1(7-37)-OH, iodoglucagon, N(α)-biotinyl-N-(epsilon)-acetimidoglucagon, N(α)-carbamylglucagon, N(α)-ε-acetylglucagon, N(α)-maltoglucagon, N(ε)-acetimidoglucagon, Ala(1)-N(ε)-acetimidoglucagon, desHis(1)-N(ε)-acetimidoglucagon, Phe(1)-N(ε)-acetimidoglucagon, N(ε)-decanoylglucagon, N(ε)-hexanoylglucagon, N-4-azido-2-nitrophenylglucagon, N-trinitrophenylglucagon, nitroglucagon, proglucagon(111-160), S 23521, Trp-S-glucagon dimer, and S-methylglucagon.

Vasoactive Intestinal Peptides

In certain embodiments, the peptide therapeutic is vasoactive intestinal peptide or an analog thereof. Such peptides include vasoactive intestinal peptide precursor, (Bz2-K24)-vasoactive intestinal peptide, (VIP-neurotensin) hybrid antagonist, Arg(15,20,21)-Leu(17)-VIP-Gly-Lys-Arg-NH₂, aviptadil, iodinated vasoactive intestinal peptide, peptide histidine valine 42, PG 97-269, preprovasoactive intestinal peptide, preprovasoactive intestinal peptide(111-122), Ro 24-9981, Ro 25-1392, Ro 25-1553, stearyl-Nle(17)-neurotensin(6-11)VIP(7-28), stearyl-norleucine(17)-vasoactive intestinal peptide, ^{99m}Tc tricarbonyl VD5 peptide, ^{99m}Tc tricarbonyl VD4 peptide, ^{99m}Tc tricarbonyl VP05 peptide, TP 3654, TP3982, vasoactive intestinal peptide(1-11), vasoactive intestinal peptide(1-12), vasoactive intestinal peptide(1-16), Lys(15)-Arg(16)-Leu(27)-vasoactive intestinal peptide(1-7)-GRF(8-27), lysyl(15)-arginyl(16)-leucyl(27)-vasoactive intestinal peptide(1-7)-growth hormone-releasing factor(8-27), vasoactive intestinal peptide(10-28), vasoactive intestinal peptide(11-28)-NH₂, vasoactive intestinal peptide(22-28), vasoactive intestinal peptide(4-11), vasoactive intestinal peptide(6-23), vasoactive intestinal peptide(6-28), vasoactive intestinal peptide(7-11), vasoactive intestinal peptide precursor, (N-Ac-His(1)-Nle(17)-Arg(20,21)-Ala(26))-vasoactive intestinal peptide, 17-norleucine-vasoactive intestinal peptide, 4-azidobenzoyl-vasoactive intestinal peptide, 4-chloro-Phe(6)-Leu(17)-vasoactive intestinal peptide, 4-Cl-Phe-vasoactive intestinal peptide, Ac-(Lys(12,14)-Nle(17)-Val(26)-Thr(28))-vasoactive intestinal peptide, Cys(2)-vasoactive intestinal peptide, Gly-vasoactive intestinal peptide, iodo-Tyr(10)-Met sulfoxide(17)-vasoactive intestinal peptide, Lys(1)-Pro(2,5)-Leu(17)-vasoactive intestinal peptide, Lys(1)-Pro(2,5)-Arg(3,4)-Tyr(6)-vasoactive intestinal peptide, N-succinimidyl 4-fluorobenzoate-Arg(8,15,21)-Leu(17)-vasoactive intestinal peptide, Arg(15,20,21)-Leu(17)-vasoactive intestinal peptide-GRR, vasoactive intestinal peptide-neurotensin hybrid, and N-hexanoyl-vasoactive intestinal polypeptide.

Insulin

In certain embodiments, the peptide therapeutic is insulin or an analog thereof. Such peptides include proinsulin, (A-C-B) human proinsulin, 9-fluorenylmethoxycarbonyl-arginyl-glycyl-isoleucyl-valyl-glutamyl-glutaminyl-cysteinyl-cysteinyl-threonyl-serine, C-Peptide, des(27-31)-C-peptide, des(1-21)preproinsulin, ((2-sulfo)-9-fluorenylmethoxycarbonyl)3-insulin, 2,4-dinitrophenol-insulin A chain-fluorescein conjugate, 2-(4-azidosalicylamido)ethyl-1,3-dithiopropionate insulin, acetylinsulin, Albulin, α-2-macroglobulin-insulin complex, amphioxus insulin-like peptide, ATP-insulin conjugates, B-insulin, B22 Glu desB30 insulin, B27 Lys destripeptide insulin, B29-biotin insulin, basal insulin, benzoylphenylalanine(B25)insulin, bis(9-fluorenylmethoxycarbonyl)insulin, BSA-insulin-chlorin e(6) conjugate, cholera toxin B-insulin conjugate, colloidal gold-insulin complex, DKP-insulin, DP 432, Exubera, glargine, glucose-insulin-potassium cardioplegic solution, glycerol-insulin, hexyl-insulin monoconjugate 2, Humalog Mix25, C. elegans ins-1 protein, insulin B(20-30), insulin B(22-30), insulin B(9-23), insulin B(9-30), insulin B-chain tetrapeptide amide B22-B25, insulin covalent aggregate, insulin crosslinked to the catalytic fragment A of diphtheria toxin, insulin detemir, insulin dimer, insulin fragment A(14-21)-B(17-30), insulin glulisine, mouse insulin I, insulin LISPRO, (2,4,6-trinitrophenyl)sulfonyl(Al)-insulin, (2-nitro-4-azidophenyl)(A1)-insulin, (2-nitro-4-azidophenyl)-Gly(B29)-insulin, (2-nitro-4-azidophenylacetyl) (B29)-insulin, (2-nitro-4-azidophenylacetyl)(B2)-desPhe(B1)-insulin, (2-nitro-4-trimethylammoniophenyl)(A1)-insulin, (2-nitro-azidophenylacetyl)(B1)-insulin, (B1)-desPhe-insulin, 2,7-diaminosuberoyl-N(α)(A1)-N(ε)(B29)-insulin, 3,5-diiodo-Tyr(A19)-insulin, 3-(N-phenylacetyl)-insulin, 3-iodo-Tyr(A14)-insulin, 4-(azidophenylacetyl)-2,4-diaminobutyric acid(A1)-insulin, 4-azido-2-nitrophenyl-insulin, 4-azidobenzoyl(B29)-insulin, 4-fluorophenylalanine(A19)-insulin, 4-succinylamidophenylarabinopyranoside-insulin, 4-succinylamidophenylglucopyranoside-insulin, 4-succinylamidophenylmannopyranoside-insulin, 6-(4-fluorobenzoyl)aminohexanoylphenylalanyl(B1)-insulin, A(27)-B-insulin-like growth factor I insulin, adipoyl-Arg(B22)-insulin, Aib21 B-chain-insulin, Aib22 B-chain-insulin, Ala(A1)-insulin, Ala(B0)-insulin, Ala(B24)-insulin, Arg(B0)-insulin, Arg(B22)-insulin, Arg(B29)-insulin, Arg(B31)-insulin, Arg(B31,B32)-insulin, Asn(21)-diethylamide(A)-insulin, Asn(B10)-insulin, Asn(B12)-insulin, AsnNH₂(A21)-insulin, Asp(B10)-insulin, Asp(B28)-insulin, Asp(B9)-Glu(B27)-insulin, Asp-imide(A21)-insulin, azoisobutyryl-insulin, B1-(4-azidosalicyloyl)-(B1-biocytin,B2-lysine)-insulin, biotinyl-insulin, bis(tert-butyloxycarbonyl)desoctapeptide-phenylhydrazide-insulin, butyrimidylpyridine disulfide-insulin, carbonyl-bis-Met,N(al),N(epsilon)(B29)-insulin, carboxymethyl-His-insulin, citraconyl-insulin, depot-insulin, des(heptapeptide)(B24-B30)-insulin, des(hexapeptide)(B25-30)-Ala(B23)-insulin, des(pentapeptide)(B1-B5)-insulin, des(tetrapeptide)(B1-B4)-insulin, des(tetrapeptide)(B27-B30)-insulin, desAla-insulin, desAsn(21)-Cys(20)-2,2,2-trifluoroethylamide(A)-insulin, desAsn21-Cys20-ethylamide(A)-insulin, desAsn21-Cys20-isopropylamide(A)-insulin, desAsn(21)-CysNH₂(20)(A)-insulin, desAsn(A21)-desAla(B30)-insulin, desGly(1A)-N-((trimethylammonio)acetyl)Ile(2A)-insulin, desGly(A1)-insulin, desGly(A1)-desPhe(B1)-insulin, desPhe(B1)-desVal(B2)-insulin, desamido (A21)-insulin, desamido (B3) insulin, desamido-insulin, deshexapeptide(B25-B30)-insulin, desoctapeptide-insulin, despentapeptide (B26-B30)-HisNH₂(B25)-insulin, despentapeptide (B26-B30)-PheNH₂(B25)-insulin, despentapeptide (B26-B30)-TyrNH₂(B25)-insulin, despentapeptide(B26-B30)-insulin, dethiobiotinyl-insulin, diacetyl(A1-B29)-insulin, dicarbain(A7,B7)-insulin, dicitraconyl Gly(A1)Phe(B1)-insulin, dihydroxycyclohexylene-insulin, disulfide-desAla(B30)-insulin, dodecoy(A1-B29)-insulin, fluorescein-isothiocyanated-insulin, fluoresceinthiocarbamyl(B29)-insulin, Gln(A8)-insulin, Gln(B13)-insulin, Gln(B30)-insulin, Glu(21) B-chain-insulin, Glu(A8)-insulin, Gly(A1)-insulin, glycosylated insulin, hexamethylester-insulin, His(B16)-insulin, iodo-insulin, isophane insulin, Leu(A19)-insulin, Leu(A3)-insulin, Leu(B24)-insulin, Leu(B24,B25)-insulin, Leu(B25)-insulin, Leu(B30)-insulin, Long-Acting Insulin, bovine insulatard, human insulatard, porcine isulatard, Lys(B29)-(N(E)-myristoyl)-des(b30) insulin, methoxy-insulin, N(α)(B1)-biotinyl-epsilon-aminocaproyl-insulin, N(E)-palmitoyl Lys(B29)-insulin, N,N-bis(methylsulfonylethoxycarbonyl)-insulin, N-Me-Ile(2A)-insulin, N-Me-Val(3A)-insulin, N-methylpyridinium insulin, neutral insulin, Nle(A2)-insulin, Nle(B17)-insulin, octadeutero-Phe(B1)-octadeutero-Val(B2)-insulin, Phe(A14)-insulin, Phe(A19)-insulin, Phe(B1)-insulin, Phe(B24)-insulin, polyethylene glycol(B1)-insulin, Pro(B21)-insulin, Sar(9A)-insulin, Ser(A6,A11)-insulin, Ser(B24)-insulin, Ser(B25)-insulin, single chain des(B30)-insulin, tetrakis(3-nitro-Tyr)-insulin, tri-Lys-insulin, trifluoroacetyl-insulin, triphthaloyl-insulin, tris(N-methylpyridinium)-insulin, Trp(A1)-insulin, Trp(A19)-insulin, Trp(B1)-insulin, Ultratard Insulin, W(B28),P(B29)-insulin, zebrafish insulin-a protein, zebrafish insulin-b protein, insulin-dextran, Panulirus argus insulin-like protein 6-kDa, insulin-related factor, iodo(A14-tyrosyl)insulin, Leydig insulin-like protein, Lys(B29)(N-carboxynonadecanoyl)-des(B30) human insulin, Lys(B29)-N(ε)-lithocholoyl-gamma-Glu des(b30) insulin, N(ε)B29-(4-azidosalicyloyl) insulin, N(ε)B29-(4-azidotetrafluorobenzoyl-biocytinyl) insulin, NBC-insulin, NBI6024, neutral protamine lispro, NovoSol Basal insulin, preproinsulin, sulfated beef insulin, thyroxyl-insulin, methionyl-lysylproinsulin, miniproinsulin, N-(ε29),N-(ε59)-bis(methylsulfonylethoxycarbonyl)proinsulin, proinsulin(46-70), des(31,32)-proinsulin, des(Lys(64),Arg(65)) proinsulin, des(23-63)-proinsulin, and proinsulin-E. coli tryptophan E chimeric polypeptide.

GLP-1 Agonists

In particular embodiments, the peptide therapeutic is a GLP-1 agonist. Particular GLP-1 agonists include GLP-1, exendin-4, and analogs thereof. Exemplary analogs are described below.

Exendin-4 and Exendin-4 Analogs

Exendin-4 and exendin-4 analogs can also be used in the compositions, methods, and kits of the invention. The compounds of the invention can include fragments of the exendin-4 sequence. Exendin-4 has the sequence.

His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Pro-Ser-NH₂

Particular exendin-4 analogs include those having a cysteine substitution (e.g., [Cys³²]exendin-4) or a lysine substitution (e.g., [Lys³⁹]exendin-4). Other exendin-4 analogs include (2-sulfo-9-fluorenylmethoxycarbonyl)3-exendin-4 and fluorescein-Trp25-exendin-4.

Exendin analogs are also described in U.S. Pat. No. 7,157,555 and include those of the formula:

X₁-X₂-X₃-Gly-Thr-X₄-X₅-X₆-X₇-X₈-Ser-Lys-Gln-X₉-Glu-
Glu-Glu-Ala-Val-Arg-Leu-X₁₀-X₁₁-X₁₂-X₁₃-Leu-Lys-
Asn-Gly-Gly-X₁₄-Ser-Ser-Gly-Ala-X₁₅-X₁₆-X₁₇-X₁₈-Z

where X₁ is His, Arg or Tyr; X₂ is Ser, Gly, Ala or Thr; X₃ is Asp or Glu; X₄ is Phe, Tyr or Nal; X₅ is Thr or Ser; X₆ is Ser or Thr; X₇ is Asp or Glu; X₈ is Leu, Ile, Val, pGly or Met; X₉ is Leu, Ile, pGly, Val or Met; X₁₀ is Phe, Tyr, or Nal; X₁₁ is Ile, Val, Leu, pGly, t-BuG or Met; X₁₂ is Glu or Asp; X₁₃ is Trp, Phe, Tyr, or Nal; X₁₄, X₁₅, X₁₆ and X₁₇ are independently Pro, HPro, 3Hyp, 4Hyp, TPro, N-alkylglycine, N-alkyl-pGly or N-alkylalanine; X₁₈ is Ser, Thr, or Tyr; and Z is OH or NH₂ (e.g., with the proviso that the compound is not exendin-3 or exindin-4.)

Preferred N-alkyl groups for N-alkylglycine, N-alkyl-pGly and N-alkylalanine include lower alkyl groups (e.g., C₁₋₆ alkyl or C₁₋₄ alkyl).

In certain embodiments, X₁ is His or Tyr (e.g., His). X₂ can be Gly. X₉ can be Leu, pGly, or Met. X₁₃ can be Trp or Phe. X₄ can be Phe or Nal; X₁₁ can be Ile or Val, and X₁₄, X₁₅, X₁₆ and X₁₇ can be independently selected from Pro, HPro, TPro, or N-alkylalanine (e.g., where N-alkylalanine has a N-alkyl group of 1 to about 6 carbon atoms). In one aspect, X₁₅, X₁₆, and X₁₇ are the same amino acid residue. X₁₈ may be Ser or Tyr (e.g., Ser). Z can be —NH₂.

In other embodiments, X₁ is His or Tyr (e.g., His); X₂ is Gly; X₄ is Phe or Nal; X₉ is Leu, pGly, or Met; X₁₀ is Phe or Nal; X₁₁ is Ile or Val; X₁₄, X₁₅, X₁₆, and X₁₇ are independently selected from Pro, HPro, TPro, or N-alkylalanine; and X₁₈ is Ser or Tyr, (e.g., Ser). Z can be —NH₂.

In other embodiments, X₁ is His or Arg; X₂ is Gly; X₃ is Asp or Glu; X₄ is Phe or napthylalanine; is Thr or Ser; X₆ is Ser or Thr; X₇ is Asp or Glu; X₈ is Leu or pGly; X₉ is Leu or pGly; X₁₀ is Phe or Nal; X₁₁ is Ile, Val, or t-butyltylglycine; X₁₂ is Glu or Asp; X₁₃ is Trp or Phe; X₁₄, X₁₅, X₁₆, and X₁₇ are independently Pro, HPro, TPro, or N-methylalanine; X₁₈ is Ser or Tyr: and Z is —OH or —NH₂ (e.g., where the compound is not exendin-3 or exendin-4). Z can be —NH₂.

In another embodiment, X₉ is Leu, Ile, Val, or pGly (e.g., Leu or pGly) and X₁₃ is Phe, Tyr, or Nal (e.g., Phe or Nal). These compounds can exhibit advantageous duration of action and be less subject to oxidative degradation, both in vitro and in vivo, as well as during synthesis of the compound.

Other exendin analogs also described in U.S. Pat. Nos. 7,157,555 and 7,223,725, include compounds of the formula:

X₁-X₂-X₃-Gly-X₅-X₆-X₇-X₈-X₉-X₁₀-X₁₁-X₁₂-X₁₃-X₁₄-
X₁₅-X₁₆-X₁₇-Ala-X₁₉-X₂₀-X₂₁-X₂₂-X₂₃-X₂₄-X₂₅-X₂₆-
X₂₇-X₂₈-Z₁

where X₁ is His, Arg, or Tyr; X₂ is Ser, Gly, Ala, or Thr; X₃ is Asp or Glu; X₅ is Ala or Thr; X₆ is Ala, Phe, Tyr, or Nal; X₇ is Thr or Ser; X₈ is Ala, Ser, or Thr; X₉ is Asp or Glu; X₁₀ is Ala, Leu, Ile, Val, pGly, or Met; X₁₁ is Ala or Ser; X₁₂ is Ala or Lys; X₁₃ is Ala or Gln; X₁₄ is Ala, Leu, Ile, pGly, Val, or Met; X₁₅ is Ala or Glu; X₁₆ is Ala or Glu; X₁₇ is Ala or Glu; X₁₉ is Ala or Val; X₂₀ is Ala or Arg; X₂₁ is Ala or Leu; X₂₂ is Phe, Tyr, or Nal; X₂₃ is Ile, Val, Leu, pGly, t-BuG, or Met; X₂₄ is Ala, Glu, or Asp; X₂₅ is Ala, Trp, Phe, Tyr, or Nal; X₂₆ is Ala or Leu; X₂₇ is Ala or Lys; X₂₈ is Ala or Asn; Z₁ is —OH, —NH₂, Gly-Z₂, Gly-Gly-Z₂, Gly-Gly-X₃₁-Z₂, Gly-Gly-X₃₁-Ser-Z₂, Gly-Gly-X₃₁-Ser-Ser-Z₂, Gly-Gly-X₃₁-Ser-Ser-Gly-Z₂, Gly-Gly-X₃₁-Ser-Ser-Gly-Ala-Z₂, Gly-Gly-X₃₁-Ser-Ser-Gly-Ala-X₃₆—Z₂, Gly-Gly-X₃₁-Ser-Ser-Gly-Ala-X₃₆—X₃₇—Z₂ or Gly-Gly-X₃₁-Ser-Ser-Gly-Ala-X₃₆—X₃₇—X₃₈—Z₂; X₃₁, X₃₆, X₃₇, and X₃₈ are independently Pro, HPro, 3Hyp, 4Hyp, TPro, N-alkylglycine, N-alkyl-pGly or N-alkylalanine; and Z₂ is -OH or -NH₂ (e.g., provided that no more than three of X₅, X₆, X₈, X₁₀, X₁₁, X₁₂, X₁₃, X₁₄, X₁₅, X₁₆, X₁₇, X₁₉, X₂₀, X₂₁ , X₂₄, X₂₅, X₂₆, X₂₇ and X₂₈ are Ala). Preferred N-alkyl groups for N-alkylglycine, N-alkyl-pGly and N-alkylalanine include lower alkyl groups of 1 to about 6 carbon atoms (e.g., 1 to 4 carbon atoms).

In certain embodiments, X₁ is His or Tyr (e.g., His). X₂ can be Gly. X₁₄ can be Leu, pGly, or Met. X₂₅ can be Trp or Phe. In some embodiments, X₆ is Phe or Nal, X₂₂ is Phe or Nal, and X₂₃ is Ile or Val. X₃₁, X₃₆, X₃₇, and X₃₈ can be independently selected from Pro, HPro, TPro, and N-alkylalanine. In certain embodiments, Z₁ is —NH₂ or Z₂ is —NH₂.

In another embodiment, X₁ is His or Tyr (e.g., His); X₂ is Gly; X₆ is Phe or Nal; X₁₄ is Leu, pGly, or Met; X₂₂ is Phe or Nal; X₂₃ is Ile or Val; X₃₁, X₃₆, X₃₇, and X₃₈ are independently selected from Pro, HPro, TPro, or N-alkylalanine. In particular embodiments, Z₁ is —NH₂.

In another embodiment, X₁ is His or Arg; X₂ is Gly or Ala; X₃ is Asp or Glu; X₅ is Ala or Thr; X₆ is Ala, Phe, or naphthylalanine; X₇ is Thr or Ser; X₈ is Ala, Ser, or Thr; X₉ is Asp or Glu; X₁₀ is Ala, Leu, or pGly; X₁₁ is Ala or Ser; X₁₂ is Ala or Lys; X₁₃ is Ala or Gln; X₁₄ is Ala, Leu, or pGly; X₁₅ is Ala or Glu; X₁₆ is Ala or Glu; X₁₇ is Ala or Glu; X₁₉ is Ala or Val; X₂₀ is Ala or Arg; X₂₁ is Ala or Leu; X₂₂ is Phe or Nal; X₂₃ is Ile, Val or t-BuG; X₂₄ is Ala, Glu or Asp; X₂₅ is Ala, Trp or Phe; X₂₆ is Ala or Leu; X₂₇ is Ala or Lys; X₂₈ is Ala or Asn; Z₁ is —OH, —NH₂, Gly-Z₂, Gly-Gly-Z₂, Gly-Gly-X₃₁-Z₂, Gly-Gly X₃₁-Ser-Z₂, Gly-Gly-X₃₁ Ser-Ser-Z₂, Gly-Gly-X₃₁ Ser-Ser-Gly-Z₂, Gly-Gly-X₃₁ Ser-Ser-Gly Ala-Z₂, Gly-Gly-X₃₁ Ser-Ser-Gly-Ala-X₃₆—Z₂, Gly-Gly-X₃₁-Ser-Ser-Gly-Ala-X₃₆—X₃₇—Z₂, Gly-Gly-X₃₁-Ser-Ser-Gly-Ala-X₃₆—X₃₇—X₃₈—Z₂; X₃₁, X₃₆, X₃₇ and X₃₈ being independently Pro HPro, TPro or N-methylalanine; and Z₂ being —OH or —NH₂ (e.g., provided that no more than three of X₃, X₅, X₆, X₈, X₁₀, X₁₁^{,} X₁₂^{,} X₁₃^{,} X₁₄^{,} X₁₅, X₁₆, X₁₇, X₁₉, X₂₀, X₂₁, X₂₄, X₂₅, X₂₆, X₂₇ and X₂₈ are Ala).

In yet another embodiment, X₁₄ is Leu, Ile, Val, or pGly (e.g., Leu or pGly), and X₂₅ is Phe, Tyr or Nal (e.g., Phe or Nal).

Exendin analogs described in U.S. Pat. No. 7,220,721 include compounds of the formula:

X₁-X₂-X₃-X₄-X-₅-X₆-X₇-X₈-X₉-X₁₀-X₁₁-X₁₂-X₁₃-X₁₄-
X₁₅-X₁₆-X₁₇-Ala-X₁₉-X₂₀-X₂₁-X₂₂-X₂₃-X₂₄-X₂₅-X₂₆-
X₂₇-X₂₈-Z₁

where X₁ is His, Arg, Tyr, Ala, Norval, Val, or Norleu; X₂ is Ser, Gly, Ala, or Thr; X₃ is Ala, Asp, or Glu; X₄ is Ala, Norval, Val, Norleu, or Gly; X₅ is Ala or Thr; X₆ is Phe, Tyr or Nal; X₇ is Thr or Ser; X₈ is Ala, Ser or Thr; X₉ is Ala, Norval, Val, Norleu, Asp, or Glu; X₁₀ is Ala, Leu, Ile, Val, pGly, or Met; X₁₁ is Ala or Ser; X₁₂ is Ala or Lys; X₁₃ is Ala or Gln; X₁₄ is Ala, Leu, Ile, pGly, Val, or Met; X₁₅ is Ala or Glu; X₁₆ is Ala or Glu; X₁₇ is Ala or Glu; X₁₉ is Ala or Val; X₂₀ is Ala or Arg; X₂₁ is Ala or Leu; X₂₂ is Phe, Tyr, or Nal; X₂₃ is Ile, Val, Leu, pGly, t-BuG, or Met; X₂₄ is Ala, Glu, or Asp; X₂₅ is Ala, Trp, Phe, Tyr, or Nal; X₂₆ is Ala or Leu; X₂₇ is Ala or Lys; X₂₈ is Ala or Asn; Z₁ is —OH, —NH₂, Gly-Z₂, Gly-Gly-Z₂, Gly-Gly-X₃₁—Z₂, Gly-Gly-X₃₁-Ser-Z₂, Gly-Gly-X₃₁-Ser-Ser-Z₂, Gly-Gly-X₃₁-Ser-Ser-Gly-Z₂, Gly-Gly-X₃₁ Ser-Ser-Gly-Ala-Z₂, Gly-Gly-X₃₁-Ser-Ser-Gly-Ala-X₁₃—Z₂, Gly-Gly-X₃₁ Ser-Ser-Gly-Ala-X₃₆-X₃₇—Z₂, Gly-Gly X₃₁ Ser Ser Gly Ala X₃₆ X₃₇ X₃₁ —Z₂ or Gly Gly X₃₁ Ser Ser Gly Ala X₃₆ X₃₇ X₃₈ X₃₉—Z₂ ; where X₃₁, X₃₆, X₃₇, and X₃₈ are independently Pro, HPro, 3Hyp, 4Hyp, TPro, N-alkylglycine, N-alkyl-pGly, or N-alkylalanine; and Z₂ is —OH or —NH₂ (e.g., provided that no more than three of X₃, X₄, X₅, X₈, X₉, X₁₀, X₁₁, X₁₂, X₁₃, X₁₄, X₁₅, X₁₆, X₁₇, X₁₉, X₂₀, X₂₁, X₂₄, X₂₅, X₂₆, X₂₇ and X₂₈ are Ala and/or provided also that, if X₁ is His, Arg, or Tyr, then at least one of X₃, X₄ and X₉ is Ala).

Particular examples of exendin-4 analogs include exendin-4(1-30), exendin-4(1-30) amide, exendin-4(1-28) amide, [Leu¹⁴,Phe²⁵]exendin-4 amide, [Leu¹⁴,Phe²⁵]exendin-4(1-28) amide, and [Leu¹⁴,Ala²²,Phe²⁵]exendin-4(1-28) amide.

U.S. Pat. No. 7,329,646 describes exendin-4 analogs having the general formula:

His-Gly-Glu-Gly-Thr-Phe-Thr-Ser-Asp-Leu-Ser-Lys-
Gln-X₁₄-Glu-Glu-Glu-Ala-Val-X₂₀-Leu-Phe-Ile-Glu-
Trp-Leu-Lys-Asn-Gly-Gly-Pro-Ser-Ser-Gly-Ala-Pro-
Pro-Pro-Ser-X₄₀.

where X₁₄ is Arg, Leu, Ile, or Met; X₂₀ is His, Arg, or Lys; X₄₀ is Arg-OH, —OH, —NH₂ or Lys-OH. In certain embodiments, when X₁₄ is Met and X₂₀ is Arg, X₄₀ cannot be —NH₂. Other exendin-4 derivatives include [(Ile/Leu/Met)¹⁴,(His/Lys)²⁰,Arg⁴⁰]exendin-4; [(not Lys/not Arg)¹²,(not Lys/not Arg)²⁰,(not Lys/not Arg)²⁷,Arg⁴⁰]exendin-4; and [(not Lys/not Arg)²⁰,Arg⁴⁰]exendin-4. Particular exendin-4 analogs include [Lys²⁰,Arg⁴⁰]exendin-4,[His²⁰,Arg⁴⁰]exendin-4; and [Leu ¹⁴,Lys²⁰,Arg⁴⁰]exendin-4.

The invention may also use truncated forms of exendin-4 or any of the exendin analogs described herein. The truncated forms may include deletions of 1, 2, 3, 4, 5, 6, 7, 8, 9, 10, 11, 12, 13, 14, 15, 16, 17, 18, 19, or 20 amino acids from the N-terminus, from the C-terminus, or a combination thereof. Particular exendin-4 fragments include Exendin-4(1-31). Other fragments of exendin-4 are described in U.S. Patent Application Publication No. 2007/0037747 and have the formula:

His-Gly-Glu-Gly-Thr-X₆-Thr-Ser-Asp-Leu-Ser-Lys-
Gln-X₁₄-Glu-Glu-Glu-Ala-Val-X₂₀-Leu-Phe-Ile-Glu-
Trp-Leu-Lys-Asn-Gly-X₃₀-Pro-X₃₂

where X₆ is Phe or Tyr, X₁₄ is Met, Ile or Leu, X₂₀ is Lys; X₃₀ is Gly or is absent; and X₃₂ is Arg or is absent.

GLP-1 and GLP-1 Analogs

The GLP-1 agonist used in the compositions, methods, and kits of the invention can be GLP-1 or a GLP-1 analog. In certain embodiments, the GLP-1 analog is a peptide, which can be truncated, may have one or more substitutions of the wild type sequence (e.g., the human wild type sequence), or may have other chemical modifications. GLP-1 agonists can also be non-peptide compounds, for example, as described in U.S. Pat. No. 6,927,214. Particular analogs include BIM 51077, LY307161, LY548806, CJC-1131, Liraglutide, glucagon-like peptide 1(1-36)amide, glucagon-like peptide 1(1-37), glucagon-like peptide 1(7-36), Ala³⁶-glucagon-like peptide 1(7-36), glucagon-like peptide 1(7-36)amide, Ser(8)-glucagon-like peptide 1(7-36)amide, glucagon-like peptide 1(7-37), N-acetyl-glucagon-like peptide-1(7-36)amide, N-pyroglutamyl-glucagon-like peptide-1(7-36)amide, and glucagon-like peptide-1(9-36)-amide.

The GLP-1 analog can be truncated form of GLP-1. The GLP-1 peptide may be truncated by 1, 2, 3, 4, 5, 6, 7, 8, 9, 10, 11, 12, 13, 15, 20, or more residues from its N-terminus, its C-terminus, or a combination thereof. In certain embodiments, the truncated GLP-1 analog is the GLP-1(7-34), GLP-1(7-35), GLP-1(7-36), or GLP-1(7-37) human peptide or the C-terminal amidated forms thereof.

In other embodiments of the invention, modified forms of truncated GLP-1 peptides are used. Exemplary analogs are described in U.S. Pat. No. 5,545,618 and have the amino acid sequence:

His-Ala-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Lys-(Gly)-(Arg)-(Gly)

where (Gly), (Arg), and (Gly) are present or absent depending on indicated chain length, with at least one modification selected from the group consisting of (a) substitution of a neutral amino acid, Arg, or a D form of Lys for Lys at position 26 and/or 34 and/or a neutral amino acid, Lys, or a D form of Arg for Arg at position 36; (b) substitution of an oxidation-resistant amino acid for Trp at position 31; (c) substitution according to at least one of: Tyr for Val at position 16; Lys for Ser at position 18; Asp for Glu at position 21; Ser for Gly at position 22; Arg for Gln at position 23; Arg for Ala at position 24; and Gln for Lys at position 26; (d) a substitution comprising at least one of an alternative small neutral amino acid for Ala at position 8; an alternative acidic amino acid or neutral amino acid for Glu at position 9; an alternative neutral amino acid for Gly at position 10; and an alternative acidic amino acid for Asp at position 15; and (e) substitution of an alternative neutral amino acid or the Asp or N-acylated or alkylated form of His for His at position 7. With respect to modifications (a), (b), (d), and (e), the substituted amino acids may be in the D form. The amino acids substituted at position 7 can also be the N-acylated or N-alkylated amino acids. Exemplary GLP-1 analogs include [D-His³⁷]GLP-1(7-37), [Tyr]GLP-1(7-37), [N-acetyl-His⁷]GLP-1(7-37), [N-isopropyl-His⁷]GLP-1(7-37), [D-Ala⁸]GLP-1(7-37), [D-Glu⁹]GLP-1(7-37), [Asp⁹]GLP-1(7- 37), [D-Asp⁹]GLP-1(7-37), [D-Phe¹⁰]GLP-1(7-37), [Ser²²,Arg²³,Arg²⁴,Gln²⁶]GLP-1(7-37), and [Ser⁸,Gln⁹,Tyr¹⁸,Lys¹⁸,Asp²¹]GLP-1(7-37).

Other GLP-1 fragments are described in U.S. Pat. No. 5,574,008 have the formula:

R₁-Ser-Tyr-Leu-Glu-Gly-Gln-Ala-Ala-Lys-Glu-Phe-
Ile-Ala-Trp-Leu-Val-X-Gly-Arg-R₂

where R₁ is H₂N; H₂N-Ser; H₂N-Val-Ser; H₂N-Asp-Val-Ser; H₂N-Ser-Asp-Val-Ser; H₂N-Thr-Ser-Asp-Val-Ser; H₂N-Phe-Thr-Ser-Asp-Val-Ser; H₂N-Thr-Phe-Thr-Ser-Asp-Val-Ser; H₂N-Gly-Thr-Phe-Thr-Ser-Asp-Val-Ser; H₂N-Glu-Gly-Thr-Phe-Thr-Ser-Asp-Val-Ser; or H₂N-Ala-Glu-Gly-Thr-Phe-Thr-Ser-Asp-Val-Ser; X is Lys or Arg; and R₂ is NH₂, OH, Gly-NH₂, or Gly-OH.

Other GLP-1 analogs, described in U.S. Pat. No. 5,118,666, include the sequence His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-X, where X is Lys, Lys-Gly, or Lys-Gly-Arg.

GLP-1 analogs also include peptides of the formula: H₂N—X—CO—R₁, where R₁ is OH, OM, or —NR₂R₃; M is a pharmaceutically acceptable cation or a lower branched or unbranched alkyl group (e.g., C₁₋₆ alkyl); R₂ and R₃ are independently selected from the group consisting of hydrogen and a lower branched or unbranched alkyl group (e.g., C₁₋₆ alkyl); X is a peptide comprising the sequence His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg; NH₂ is the amine group of the amino terminus of X; and CO is the carbonyl group of the carboxy terminus of X; acid addition salts thereof; and the protected or partially protected derivatives thereof. These compounds may have insulinotropic activity exceeding that of GLP-1(1-36) or GLP-1(1-37).

Other GLP-1 analogs are described in U.S. Pat. No. 5,981,488 and have the formula:

R₁-X-Glu-Gly-Thr-Phe-Thr-Ser-Asp-Val-Ser-Ser-
Tyr-Leu-Y-Gly-Gln-Ala-Ala-Lys-Z-Phe-Ile-Ala-Trp-
Leu-Val-Lys-Gly-Arg-R₂

where R₁ is His, D-His, desamino-His, β-amino-His, 6-hydroxy-His, homohistidine, α-fluoromethyl-His, or α-methyl-His; X is Met, Asp, Lys, Thr, Leu, Asn, Gln, Phe, Val, or Tyr; Y and Z are independently selected from Glu, Gln, Ala, Thr, Ser, and Gly; and R₂ is selected from NH₂ and Gly-OH (e.g., provided that, if R₁ is His, X is Val, Y is Glu, and Z is Glu, then R₂ is NH₂).

Other GLP-1 analogs are described in U.S. Pat. No. 5,512,549 and have the formula:

R₁-Ala-Glu-Gly-Thr-Phe-Thr-Ser-Asp-Val-Ser-Ser-
Tyr-Leu-Glu-Gly-Gln-Ala-Ala-Xaa-Glu-Phe-Ile-Ala-
Trp-Leu-Val-Lys(R₂)-Gly-Arg-R₃

where R₁ is 4-imidazopropionyl (desamino-histidyl), 4-imidazoacetyl, or 4-imidazo-α, adimethyl-acetyl; R₂, which is bound to the side chain of the Lys (e.g., through the ε amino group), is C₆₋₁₀ unbranched acyl or is absent; R₃ is Gly-OH or NH₂; and Xaa is Lys or Arg.

Still other GLP-1 analogs are described in U.S. Pat. No. 7,084,243. In one embodiment, the GLP-1 analog has the formula:

His-X₈-Glu-Gly-X₁₁-X₁₂-Thr-Ser-Asp-X₁₆-Ser-Ser-
Tyr-Leu-Glu-X₂₂-X₂₃-X₂₄-Ala-X₂₆-X₂₇-Phe-Ile-Ala-
X₃₁-Leu-X₃₃-X₃₄-X₃₅-X₃₆-R

where X₈ is Gly, Ala, Val, Leu, Ile, Ser, or Thr; X₁₁ is Asp, Glu, Arg, Thr, Ala, Lys, or His; X₁₂ is His, Trp, Phe, or Tyr; X₁₆ is Leu, Ser, Thr, Trp, His, Phe, Asp, Val, Tyr, Glu, or Ala; X₂₂ is Gly, Asp, Glu, Gln, Asn, Lys, Arg, Cys, or Cya; X₂₃ is His, Asp, Lys, Glu, or Gln; X₂₄ is Glu, His, Ala, or Lys; X₂₆ is Asp, Lys, Glu, or His; X₂₇ is Ala, Glu, His, Phe, Tyr, Trp, Arg, or Lys; X₃₀ is Ala, Glu, Asp, Ser, or His; X₃₃ is Asp, Arg, Val, Lys, Ala, Gly, or Glu; X₃₄ is Glu, Lys, or Asp; X₃₅ is Thr, Ser, Lys, Arg, Trp, Tyr, Phe, Asp, Gly, Pro, His, or Glu; X₃₆ is Arg, Glu, or His; R is Lys, Arg, Thr, Ser, Glu, Asp, Trp, Tyr, Phe, His, —NH₂, Gly, Gly-Pro, or Gly-Pro-NH₂, or is deleted (e.g., provided that the polypeptide does not have the sequence of GLP-1(7-37)OH or GLP-1(7-36)-NH₂ and provided that the polypeptide is not Gly⁸-GLP-1(7-37)OH, Gly⁸-GLP-1(7-36)NH₂, Val⁸-GLP-1(7-37)OH, Val⁸-GLP-1(7-36)NH₂, Leu⁸-GLP-1(7-37)OH, Leu⁸-GLP-1(7-36)NH₂, Ile⁸-GLP-1(7-37)OH, Ile⁸-GLP-1(7-36)NH₂, Ser⁸-GLP-1(7-37)OH, Ser⁸-GLP-1(7-36)NH₂, Thr⁸-GLP-1(7-37)OH, or Thr⁸-GLP-1(7-36)NH₂, Ala¹¹-Glp-1(7-37)OH, Ala¹¹-Glp-1(7-36)NH₂, Ala¹⁶-Glp-1(7-37)OH, Ala¹⁶-Glp-1(7-36)NH₂, Ala²⁷-Glp-1(7-37)OH, Ala²⁷-Glp-1(7-36)NH₂, Ala²⁷-Glp-1(7-37)OH, Ala²⁷-Glp-1(7-36)NH₂, Ala³³-Glp-1(7-37)OH, or Ala³³-Glp-1(7-36)NH₂).

In another embodiment, the polypeptide has the amino acid sequence:

His-X₈-Glu-Gly-Thr-X₁₂-Thr-Ser-Asp-X₁₆-Ser-Ser-Tyr-
Leu-Glu-X₂₂-X₂₃-Ala-Ala-X₂₆-Glu-Phe-Ile-X₃₀-Trp-
Leu-Val-Lys-X₃₅-Arg-R

where X₈ is Gly, Ala, Val, Leu, Ile, Ser, or Thr; X₁₂ is His, Trp, Phe, or Tyr; X₁₆ is Leu, Ser, Thr, Trp, His, Phe, Asp, Val, Glu, or Ala; X₂₂ is Gly, Asp, Glu, Gln, Asn, Lys, Arg, Cys, or Cya; X₂₃ is His, Asp, Lys, Glu, or Gln; X₂₆ is Asp, Lys, Glu, or His; X₃₀ is Ala, Glu, Asp, Ser, or His; X₃₅ is Thr, Ser, Lys, Arg, Trp, Tyr, Phe, Asp, Gly, Pro, His, or Glu; R is Lys, Arg, Thr, Ser, Glu, Asp, Trp, Tyr, Phe, His, —NH₂, Gly, Gly-Pro, Gly-Pro-NH₂, or is deleted, (e.g., provided that the polypeptide does not have the sequence of GLP-1(7-37)OH or GLP-1(7-36)-NH₂ and provided that the polypeptide is not Gly⁸-GLP-1(7-37)OH, Gly⁸-GLP-1(7-36)NH₂, Val⁸-GLP-1(7-37)OH, Val⁸-GLP-1(7-36)NH₂, Leu⁸-GLP-1(7-37)OH, Leu⁸-GLP-1(7-36)NH₂, Ile⁸-GLP-1(7-37)OH, Ile⁸-GLP-1(7-36)NH₂, Ser⁸-GLP-1(7-37)OH, Ser⁸-GLP-1(7-36)NH₂, Thr⁸-GLP-1(7-37)OH, Thr⁸-GLP-1(7-36)NH₂, Ala¹⁶-GLP(7-37)OH, or Ala¹⁶-GLP-1(7-36)NH₂).

In another embodiment, the polypeptide has the amino acid sequence:

His-X₈-Glu-Gly-Thr-Phe-Thr-Ser-Asp-Val-Ser-Ser-Tyr-
Leu-Glu-X₂₂-X₂₃-Ala-Ala-Lys-X₂₇-Phe-Ile-X₃₀-Trp-
Leu-Val-Lys-Gly-Arg-R

where X₈ is Gly, Ala, Val, Leu, Ile, Ser, or Thr; X₂₂ is Gly, Asp, Glu, Gln, Asn, Lys, Arg, Cys, or Cya; X₂₃ is His, Asp, Lys, Glu, or Gln; X₂₇ is Ala, Glu, His, Phe, Tyr, Trp, Arg, or Lys X₃₀ is Ala, Glu, Asp, Ser, or His; R is Lys, Arg, Thr, Ser, Glu, Asp, Trp, Tyr, Phe, His, —NH₂, Gly, Gly-Pro, or Gly-Pro-NH₂, or is deleted (e.g., provided that the polypeptide does not have the sequence of GLP-1(7-37)OH or GLP-1(7-36)NH₂ and provided that the polypeptide is not Gly⁸-GLP-1(7-37)OH, Gly⁸-GLP-1(7-36)NH₂, Val⁸-GLP-1(7-37)OH, Val⁸-GLP-1(7-36)NH₂, Leu⁸-GLP-1(7-37)OH, Leu⁸-GLP-1(7-36)NH₂, Ile⁸-GLP-1(7-37)OH, Ile⁸-GLP-1(7-36)NH₂, Ser⁸-GLP-1(7-37)OH, Ser⁸-GLP-1(7-36)NH₂, Thr⁸-GLP-1(7-37)OH, Thr⁸-GLP-1(7-36)NH₂, Ala¹⁸-GLP-1(7-37)OH, Ala¹⁶-Glp-1(7-36) NH₂, Glu²⁷-Glp-1(7-37)OH, or Glu²⁷-Glp-1(7-36)NH₂.

In another embodiment, the polypeptide has the amino acid sequence:

X₇-X₈-Glu-Gly-Thr-Phe-Thr-Ser-Asp-Val-Ser-Ser-Tyr-
Leu-Glu-X₂₂-Gln-Ala-Ala-Lys-Glu-Phe-Ile-Ala-Trp-
Leu-Val-Lys-Gly-Arg-R

where X₇ is L-His, D-His, desamino-His, 2amino-His, 8-hydroxy-His, homo-His, α-fluoromethyl-His or α-methyl-His; X₈ is Gly, Ala, Val, Leu, Ile, Ser or Thr (e.g., Gly, Val, Leu, Ile, Ser, or Thr); X₂₂ is Asp, Glu, Gln, Asn, Lys, Arg, Cys, or Cya, and R is —NH₂ or Gly(OH).

In another embodiment, the GLP-1 compound has an amino acid other than alanine at position 8 and an amino acid other than glycine at position 22. Specific examples of GLP-1 compounds include [Glu²²]GLP-1(7-37)OH, [Asp²²]GLP-1(7-37)OH, [Arg²²]GLP-1(7-37)OH, [Lys²²]GLP-1(7-37)OH, [Cya²²]GLP-1(7-37)OH, [Val⁸,Glu²²]GLP-1(7-37)OH, [Val⁸,Asp²²]GLP-1(7-37)OH, [Val⁸,Arg²²]GLP-1(7-37)OH, [Val⁸,Lys²²]GLP-1(7-37)OH, [Val⁸,Cya²²]GLP-1(7-37)OH, [Gly⁸,Glu²²]GLP-1(7-37)OH, [Gly⁸,Asp²²]GLP-1(7-37)OH, [Gly⁸,Arg²²]GLP-1(7-37)OH, [Gly⁸,Lys²²]GLP-1(7-37)OH, [Gly⁸,Cya²²]GLP-1(7-37)OH, [Glu²²]GLP-1(7-36)NH₂, [Asp²²]GLP-1(7-36)NH₂, [Arg²²]GLP-1(7-36)NH₂, [Lys²²]GLP-1(7-36)NH₂, [Cya²²]GLP-1(7-36)NH₂, [Val⁸,Glu²²]GLP-1(7-36)NH₂, [Val⁸,Asp²²]GLP-1(7-36)NH₂, [Val⁸,Arg²²]GLP-1(7-36)NH₂, [Val⁸,Lys²²]GLP-1(7-36)NH₂, [Val⁸,Cya²²]GLP-1(7-36)NH₂, [Gly⁸,Glu²²]GLP-1(7-36)NH₂, [Gly⁸,Asp²²]GLP-1(7-36)NH₂, [Gly⁸,Arg²²]GLP-1(7-36)NH₂, [Gly⁸,Lys²²]GLP-1(7-36)NH₂, [Gly⁸,Cya²²]GLP-1(7-36)NH₂, [Val⁸,Lys²³GLP-1(7-37)OH, [Val⁸,Ala²⁷]GLP-1(7-37)OH, [Val⁸,Glu³⁰]GLP-1(7-37)OH, [Gly⁸,Glu³⁰]GLP-1(7-37)OH, [Val⁸,His³⁵]GLP-1(7-37)OH, [Val⁸,His³⁷]GLP-1(7-37)OH, [Val⁸,Glu²²,Lys²³]GLP-1(7-37)OH, [Val⁸,Glu²²,Glu²]GLP-1(7-37)OH, [Val⁸,Glu²²,Ala²⁷]GLP-1(7-37)OH, [Val⁸,Gly³⁴,Lys³⁵]GLP-1(7-37)OH, [Val⁸,His³⁷]GLP-1(7-37)OH, [Gly⁸,His³⁷]GLP-1(7-37)OH.

Other GLP-1 analogs are described in U.S. Pat. No. 7,101,843 and include those having the formula:

X₇-X₈-Glu-Gly-Thr-X₁₂-Thr-Ser-Asp-X₁₆-Ser-X₁₈-X₁₉-
X₂₀-Glu-X₂₂-Gln-Ala-X₂₅-Lys-X₂₇-Phe-Ile-X₃₀-Trp-
Leu-X₃₃-Lys-Gly-Arg-X₃₇

wherein: X₇ is L-His, D-His, desamino-His, 2-amino-His, β-hydroxy-His, homohistidine, α-fluoromethyl-His, or α-methyl-His; X₈ is Ala, Gly, Val, Leu, Ile, Ser, or Thr; X₁₂ is Phe, Trp, or Tyr; X₁₆ is Val, Trp, Ile, Leu, Phe, or Tyr; X₁₈ is Ser, Trp, Tyr, Phe, Lys, Ile, Leu, or Val; X₁₉ is Tyr, Trp, or Phe; X₂₀ is Leu, Phe, Tyr, or Trp; X₂₂ is Gly, Glu, Asp, or Lys; X₂₅ is Ala, Val, Ile, or Leu; X₂₇ is Glu, Ile, or Ala; X₃₀ is Ala or Glu X₃₃ is Val, or Ile; and X₃₇ is Gly, His, NH₂, or is absent (e.g., provided that the compound does not have the sequence GLP-1(7-37)OH, GLP-1(7-36)-NH₂, [Gly⁸]GLP-1(7-37)OH, [Gly⁸]GLP-1(7-36)NH₂, [Val⁸]GLP-1(7-37)OH, [Val⁸]GLP-1(7-36)NH₂, [Leu⁸]GLP-1(7-37)OH, [Leu⁸]GLP-1(7-36)NH₂, [Ile⁸]GLP-1(7-37)OH, [Ile⁸]GLP-1(7-36)NH₂, [Ser⁸]GLP-1(7-37)OH, [Ser⁸]GLP-1(7-36)NH₂, [Thr⁸]GLP-1(7-37)OH, [Thr⁸]GLP-1(7-36)NH₂, [Val⁸,Tyr¹²]GLP-1(7-37)OH, [Val⁸,Tyr¹²]GLP-1(7-36)NH₂, [Val⁸,Tyr¹⁶]GLP-1(7-37)OH, [Val⁸,Tyr¹⁶]GLP-1(7-36)NH₂, [Val⁸,Glu²²]GLP-1(7-37)OH, [Val⁸,Glu²²]GLP-1(7-36)NH₂, [Gly⁸,G²²]GLP-1(7-37)OH, [Gly⁸,Glu²²]GLP-1(7-36)NH₂, [Val⁸,Asp²²]GLP-1(7-37)OH, [Val⁸,Asp²²]GLP-1(7-36)NH₂, [Gly⁸,Asp²²]GLP-1(7-37)OH, [Gly⁸,Asp²²]GLP-1(7-36)NH₂, [Val⁸,Lys²²]GLP-1(7-37)OH, [Val⁸,Lys²²]GLP-1(7-36)NH₂, [Gly⁸,Lys²²]GLP-1(7-37)OH, [Gly⁸,Lys²²]GLP-1(7-36)NH₂, [Leu⁸,Glu²²]GLP-1(7-37)OH, [Leu⁸,Glu²²]GLP-1(7-36)NH₂, [Ile⁸,Glu²²]GLP-1(7-37)OH, [Ile⁸,Glu²²]GLP-1(7-36)NH₂, [Leu⁸,Asp²²]GLP-1(7-37)OH, [Leu⁸,Asp²²]GLP-1(7-36)NH₂, [Ile⁸,Asp²²]GLP-1(7-37)OH, [Ile⁸,Asp²²]GLP-1(7-36)NH₂, [Leu⁸,Lys²²]GLP-1(7-37)OH, [Leu⁸,Lys²²]GLP-1(7-36)NH₂, [Ile⁸,Lys²²]GLP-1(7-37)OH, [Ile⁸,Lys²²]GLP-1(7-36)NH₂, [Ser⁸,Glu²²]GLP-1(7-37)OH, [Ser⁸,Glu²²]GLP-1(7-36)NH₂, [Thr⁸,Glu²²]GLP-1(7-37)OH, [Thr⁸,Glu²²]GLP-1(7-36)NH₂, [Ser⁸,Asp²²]GLP-1(7-37)OH, [Ser⁸,Asp²²]GLP-1(7-36)NH₂, [Thr⁸,Asp²²]GLP-1(7-37)OH, [Thr⁸,Asp²²]GLP-1(7-36)NH₂, [Ser⁸,Lys²]GLP-1(7-37)OH, [Ser⁸,Lys²²]GLP-1(7-36)NH₂, [Thr⁸,Lys²²]GLP-1(7-37)OH, [Thr⁸,Lys²²]GLP-1(7-36)NH₂, [Glu²²]GLP-1(7-37)OH, [Glu²]GLP-1(7-36)NH₂, [Asp²²]GLP-1(7-37)OH, [Asp²²]GLP-1(7-36)NH₂, [Lys²²]GLP-1(7-37)OH, [Lys²²]GLP-1(7-36)NH₂, [Val⁸,Ala²⁷]GLP-1(7-37)OH, [Val⁸,Glu²²,Ala²⁷]GLP-1(7-37)OH, [Val⁸,Glu³⁰]GLP-1(7-37)OH, [Val⁸,Glu³⁰]GLP-1(7-36)NH₂, [Gly⁸,Glu³⁰]GLP-1(7-37)OH, [Gly⁸,Glu³⁰]GLP-1(7-36)NH₂, [Leu⁸,Glu³⁰]GLP-1(7-37)OH, [Leu⁸,Glu³⁰]GLP-1(7-36)NH₂, [Ile⁸,Glu³⁰]GLP-1(7-37)OH, [Ile⁸,Glu³⁰]GLP-1(7-36)NH₂, [Ser⁸,Glu³⁰]GLP-1(7-37)OH, [Ser⁸,Glu³⁰]GLP-1(7-36)NH₂, [Thr⁸,Glu³⁰]GLP-1(7-37)OH, [Thr⁸,Glu³⁰]GLP-1(7-36)NH₂, [Val⁸,His³⁷]GLP-1(7-37)OH, [Val⁸,His³⁷]GLP-1(7-36)NH₂, [Gly⁸,His³⁷]GLP-1(7-37)OH, [Gly⁸,His³⁷]GLP-1(7-36)NH₂, [Leu⁸,His³⁷]GLP-1(7-37)OH, [Leu⁸,His³⁷]GLP-1(7-36)NH₂, [Ile⁸,His³⁷]GLP-1(7-37)OH, [Ile⁸,His³⁷]GLP-1(7-36)NH₂, [Ser⁸,His³⁷]GLP-1(7-37)OH, [Ser⁸,His³⁷]GLP-1(7-36)NH₂, [Thr⁸,His³⁷]GLP-1(7-37)OH, [Thr⁸,His³⁷]GLP-1(7-36)NH₂).

Other GLP-1 analogs described in U.S Pat. No. 7,101,843 have the formula:

X₇-X₈-Glu-Gly-Thr-Phe-Thr-Ser-Asp-X₁₆-Ser-X₁₈-Tyr-
Leu-Glu-X₂₂-Gln-Ala-X₂₅-Lys-Glu-Phe-Ile-Ala-Trp-
Leu-X₃₃-Lys-Gly-Arg-X₃₇

wherein: X₇ is L-His, D-His, desamino-His, 2-amino-His, β-hydroxy-His, homohistidine, α-fluoromethyl-His, or α-methyl-His; X₈ is Gly, Ala, Val, Leu, Ile, Ser, or Thr; X₁₆ is Val, Phe, Tyr, or Trp; X₁₈ is Ser, Tyr, Trp, Phe, Lys, Ile, Leu, or Val; X₂₂ is Gly, Glu, Asp, or Lys; X₂₅ is Ala, Val, Ile, or Leu; X₃₃ is Val or Ile; and X₃₇ is Gly, NH₂, or is absent (e.g., provided that the GLP-1 compound does not have the sequence of GLP-1(7-37)OH, GLP-1(7-36)-NH₂, [Gly⁸]GLP-1(7-37)OH, [GlylGLP-1(7-36)NH₂, [Val⁸]GLP-1(7-37)OH, [Val⁸]GLP-1(7-36)NH₂, [Leu⁸]GLP-1(7-37)OH, [Leu⁸]GLP-1(7-36)NH₂, [Ile⁸]GLP-1(7-37)OH, [Ile⁸]GLP-1(7-36)NH₂, [Ser⁸]GLP-1(7-37)OH, [Ser⁸]GLP-1(7-36)NH₂, [Thr⁸]GLP-1(7-37)OH, [Thr⁸]GLP-1(7-36)NH₂, [Val⁸-Tyr¹⁶]GLP-1(7-37)OH, [Val⁸-Tyr¹⁶]GLP-1(7-36)NH₂, [Val⁸,Glu²²]GLP-1(7-37)OH, [Val⁸,Glu²²]GLP-1(7-36)NH₂, [Gly⁸,Glu²²]GLP-1(7-37)OH, [Gly⁸,Glu²²]GLP-1(7-36)NH₂, [Val⁸,Asp²²]GLP-1(7-37)OH, [Val⁸,Asp²²]GLP-1(7-36)NH₂,[Gly⁸,Asp²²]GLP-1(7-37)OH, [Gly⁸,Asp²²]GLP-1(7-36)NH₂, [Val⁸,Lys²²]GLP-1(7-37)OH, [Val⁸,Lys²²]GLP-1(7-36)NH₂, [GIY⁸,Lys²²]GLP-1(7-37)OH, [Gly⁸,Lys²²]GLP-1(7-36)NH₂, [Leu⁸,Glu²²]GLP-1(7-37)OH, [Leu⁸,Glu²²]GLP-1(7-36)NH₂, [Ile⁸,Glu²²]GLP-1(7-37)OH, [Ile⁸,Glu²²]GLP-1(7-36)NH₂, [Leu⁸,Asp²²]GLP1(7-37)OH, [Leu⁸,Asp²²]GLP-1(7-36)NH₂, [Ile⁸,Asp²²]GLP-1(7-37)OH, [Ile⁸,Asp²²]GLP-1(7-36)NH₂, [Leu⁸,Lys²²]GLP-1(7-37)OH, [Leu⁸,Lys²²]GLP-1(7-36)NH₂, [Ile⁸,Lys²²]GLP-1(7-37)OH, [Ile⁸,Lys²²]GLP-1(7-36)NH₂, [Ser⁸,Glu²²]GLP-1(7-37)OH, [Ser⁸,Glu²²]GLP-1(7-36)NH₂, [Thr⁸,Glu²²]GLP-1(7-37)OH, [Thr⁸,Glu²²]GLP-1(7-36)NH₂, [Serₛ,Asp²²]GLP-1(7-37)OH, [Ser⁸,Asp²²]GLP-1(7-36)NH₂, [Thr⁸,Asp²²]GLP-1(7-37)OH, [Thr⁸,Asp²²]GLP-1(7-36)NH₂, [Ser⁸,Lys²²]GLP-1(7-37)OH, [Ser⁸,Lys²²]GLP-1(7-36)NH₂, [Thr⁸,Lys²²]GLP-1(7-37)OH, [Thr⁸,Lys²²]GLP-1(7-36)NH₂, [Glu²²]GLP-1(7-37)OH, [Glu²²]GLP-1(7-36)NH₂, [Asp²²]GLP-1(7-37)OH, [Asp²²]GLP-1(7-36)NH₂, [Lys²²]GLP-1(7-37)OH, [Lys²²]GLP-1(7-36)NH₂).

GLP-1 analogs are also described in U.S. Pat. No. 7,238,670 and have the structure:

A-X₁-X₂-X₃-X₄-X₅-X₆-X₇-X₈-X₉-Y—Z—B

where each of X₁₋₉ is a naturally or nonnaturally occurring amino acid residue; Y and Z are amino acid residues; and one of the substitutions at the α-carbon atoms of Y and Z may each independently be substituted with a primary substituent group selected from the group consisting of hydrogen, alkyl, cycloalkyl, cycloalkylalkyl, heterocyclylalkyl, arylalkyl and heteroarylalkyl, heterocyclylalkyl said primary substituent optionally being substituted with a secondary substituent selected from a cycloalkyl, heterocyclyl, aryl, or heteroaryl group; any of said primary or secondary substituents may further be substituted with one or more of H, alkyl, cycloalkyl, arylalkyl, aryl, heterocyclyl, heteroaryl, alkenyl, alkynyl, halo, hydroxy, mercapto, nitro, cyano, amino, acylamino, azido, guanidino, amidino, carboxyl, carboxamido, carboxamido alkyl, formyl, acyl, carboxyl alkyl, alkoxy, aryloxy, arylalkyloxy, heteroaryloxy, heterocycleoxy, acyloxy, mercapto, mercapto alkyl, mercaptoaryl, mercapto acyl, halo, cyano, nitro, azido, amino, guanidino alkyl, guanidino acyl, sulfonic, sulfonamido, alkyl sulfonyl, aryl sulfonyl or phosphonic group; wherein, the primary or secondary substitutents may optionally be bridged by covalent bonds to form one or more fused cyclic or heterocyclic systems with each other; where, the other substitution at the alpha-carbon of Y may be substituted with H, C₁₋₆ alkyl, aminoalkyl, hydroxyalkyl or carboxyalkyl; where the other substitution at the alpha-carbon of Z may be substituted with hydrogen, C₁₋₁₂ alkyl, aminoalkyl, hydroxyalkyl, or carboxyalkyl;

A and B are optionally present, where A is present and A is H, an amino acid or peptide containing from about 1-15 amino acid residues, an R group, an R—C(O) (amide) group, a carbamate group RO—C(O), a urea R₄R₅N—C(O), a sulfonamido R—SO₂, or R₄R₅N—SO₂; where R is selected from the group consisting of hydrogen, C₁₋₁₂ alkyl, C₃₋₁₀ cycloalkyl, cycloalkylalkyl, heterocyclyl, heterocycloalkyl, aryl, heteroaryl, arylalkyl, aryloxyalkyl, heteroarylalkyl, and heteroaryloxyalkyl; R₄ and R₅ are each independently selected from the group consisting of H, alkyl, cycloalkyl, cycloalkylalkyl, heterocyclyl, heterocycloalkyl, aryl, heteroaryl, arylalkyl, aryloxyalkyl, heteroarylalkyl, and heteroaryloxyalky; where the α-amino group of X₁ is substituted with H or an alkyl group, said alkyl group may optionally form a ring with A; where B is present and B is OR₁, NR₁R₂, or an amino acid or peptide containing from 1 to 15 amino acid residues (e.g., 1 to 10 or 1 to 5) terminating at the C-terminus as a carboxamide, substituted carboxamide, an ester, a free carboxylic acid, or an amino-alcohol; where R₁ and R₂ are independently chosen from H, C₁₋₁₂ alkyl, C₃₋₁₀ cycloalkyl, cycloalkylalkyl, heterocyclyl, heterocycloalkyl, aryl, heteroaryl, arylalkyl, aryloxyalkyl, heteroarylalkyl or heteroaryloxyalkyl.

Exemplary substitutions on the α-carbon atoms of Y and Z include heteroarylarylmethyl, arylheteroarylmethyl, and biphenylmethyl forming biphenylalanine residues, any of which is also optionally substituted with one or more, hydrogen, alkyl, cycloalkyl, arylalkyl, aryl, heterocyclyl, heteroaryl, alkenyl, alkynyl, halo, hydroxy, mercapto, nitro, cyano, amino, acylamino, azido, guanidino, amidino, carboxyl, carboxamido, carboxamido alkyl, formyl, acyl, carboxyl alkyl, alkoxy, aryloxy, arylalkyloxy, heteroaryloxy, heterocycleoxy, acyloxy, mercapto, mercapto alkyl, mercaptoaryl, mercapto acyl, halo, cyano, nitro, azido, amino, guanidino alkyl, guanidino acyl, sulfonic, sulfonamido, alkyl sulfonyl, aryl sulfonyl and phosphonic group.

Other embodiments include isolated polypeptides where the other substitution at the α-carbon of Y is substituted with H, methyl, or ethyl; and where the other substitution at the α-carbon of Z is substituted with H, methyl, or ethyl.

Further embodiments include isolated polypeptides as described above where X₁ is naturally or non-naturally occurring amino acid residue in which one of the substitutions at the α-carbon is a primary substituent selected from the group consisting of heterocyclylalkyl, heteroaryl, heteroarylkalkyl and arylalkyl, said primary substituent optionally being substituted with secondary substituent selected from heteroaryl or heterocyclyl; and in which the other substitution at the α-carbon is H or alkyl; X₂ is naturally or nonnaturally occurring amino acid residue in which one of the substitutions at the α-carbon is an alkyl or cycloalkyl where the alkyl group may optionally form a ring with the nitrogen of X₂; and wherein the other substitution at the α-carbon is H or alkyl; X₃ is a naturally or nonnaturally occurring amino acid residue in which one of the substitutions at the α-carbon is a carboxyalkyl, bis-carboxyalkyl, sulfonylalkyl, heteroalkyl, or mercaptoalkyl; and where the other substitution at the α-carbon is hydrogen or alkyl; X₄ is a naturally or nonnaturally occurring amino acid residue in which the α-carbon is not substituted, or in which one of the substitutions at the α-carbon is aminoalkyl, carboxyalkyl heteroarylalkyl, or heterocycylalkyl; X₅ is a naturally or nonnaturally occurring amino acid residue in which one of the substitutions at the α-carbon is an alkyl or hydroxyalkyl, and in which the other substitution at the α-carbon is hydrogen or alkyl; X₆ is a naturally or nonnaturally occurring amino acid residue in which one of the substitutions at the α-carbon is C₁₋₁₂ alkyl, aryl, heteroaryl, heterocyclyl, cycloalkylalkyl, heterocyclylalkyl, arylalkyl, or heteroarylalkyl group, and the other substitution at the α-carbon is H or alkyl; X₇ is a naturally or nonnaturally occurring amino acid residue in which one of the substitutions at the α-carbon is a hydroxylalkyl group; X₈ is a naturally or nonnaturally occurring amino acid residue in which one of the substitutions at the α-carbon is C₁₋₁₂ alkyl, hydroxylalkyl, heteroarylalkyl, or carboxamidoalkyl, and the other substitution at the α-carbon is H or alkyl; X₉ is a naturally or nonnaturally occurring amino acid residue in which one of the substitutions at α-carbon is carboxylalkyl, bis-carboxylalkyl, carboxylaryl, sulfonylalkyl, carboxylamidoalkyl, or heteroarylalkyl; and where A is H, an amino acid or peptide containing from about 1 to about 5 amino acid residues, an R group, an R—C(O) amide group, a carbamate group RO—C(O), a urea R₄R₅N—C(O), a sulfonamido R—SO₂ or a R₄R₅N—SO₂.

In certain embodiments, X₁ is His, D-His, N-Methyl-His, D-N-Methyl-His, 4-ThiazolylAla, or D-4-ThiazolylAla; X₂ is Ala, D-Ala, Pro, Gly, D-Ser, D-Asn, Nma, D-Nma, 4-ThioPro, 4-Hyp, L-2-Pip, L-2-Azt, Aib, S- or R-Iva and Acc3; X₃ is Glu, N-Methyl-Glu, Asp, D-Asp, His, Gla, Adp, Cys, or 4-ThiazolyAla; X₄ is Gly, His, Lys, or Asp; X₅ is Thr, D-Thr, Nle, Met, Nva, or L-Aoc; X₆ is Phe, Tyr, Tyr(Bzl), Tyr(3-NO₂), Nle, Trp, Phe(penta-fluoro), D-Phe(penta-fluoro), Phe(2-fluoro), Phe(3-fluoro), Phe(4-fluoro), Phe(2,3-di-fluoro), Phe(3,4-di-fluoro), Phe(3,5-di-fluoro), Phe(2,6-di-fluoro), Phe(3,4,5-tri-fluoro), Phe(2-iodo), Phe(2-OH), Phe(2-OMe), Phe(3-OMe), Phe(3-cyano), Phe(2-chloro), Phe(2-NH₂), Phe(3-NH₂), Phe(4-NH₂), Phe(4-NO₂), Phe(4-Me), Phe(4-allyl), Phe(n-butyl), Phe(4-cyclohexyl), Phe(4-cyclohexyloxy), Phe(4-phenyloxy), 2-Nal, 2-pyridylAla, 4-thiazolylAla, 2-Thi, α-Me-Phe, D-α-Me-Phe, α-Et-Phe, D-α-Et-Phe, a-Me-Phe(2-fluoro), D-α-Me-Phe(2-fluoro), a-Me-Phe(2,3-di-fluoro), D-α-Me-Phe(2,3-di-fluoro), α-Me-Phe(2,6-di-fluoro), D-α-Me-Phe(2,6-di-fluoro), α-Me-Phe(penta-fluoro) and D-α-Me-Phe(penta-fluoro); is Thr, D-Thr, Ser, or hSer; X₈ is Ser, hSer, His, Asn, or α-Me-Ser; and X₉ is Asp, Glu, Gla, Adp, Asn, or His.

Additional embodiments include those where Y is Bip, D-Bip, L-Bip(2-Me), D-Bip(2-Me), L-Bip(2′-Me), L-Bip(2-Et), D-Bip(2-Et), L-Bip(3-Et), L-Bip(4-Et), L-Bip(2-n-propyl), L-Bip(2-n-propyl, 4-OMe), L-Bip(2-n-propyl,2′-Me), L-Bip(3-Me), L-Bip(4-Me), L-Bip(2,3-di-Me), L-Bip(2,4-di-Me), L-Bip(2,6-di-Me), L-Bip(2,4-di-Et), L-Bip(2-Me, 2′-Me), L-Bip(2-Et, 2′-Me), L-Bip(2-Et, 2′-Et), L-Bip(2-Me,4-OMe), L-Bip(2-Et,4-OMe), D-Bip(2-Et,4-OMe), L-Bip(3-OMe), L-Bip(4-OMe), L-Bip(2,4,6-tri-Me), L-Bip(2,3-di-OMe), L-Bip(2,4-di-OMe), L-Bip(2,5-di-OMe), L-Bip(3,4-di-OMe), L-Bip(2-Et,4,5-di-OMe), L-Bip(3,4-Methylene-di-oxy), L-Bip(2-Et, 4,5-Methylene-di-oxy), L-Bip(2-CH₂OH, 4-OMe), L-Bip(2-Ac), L-Bip(3-NH-Ac), L-Bip(4-NH-Ac), L-Bip(2,3-di-chloro), L-Bip(2,4-di-chloro), L-Bip(2,5-di-chloro), L-Bip(3,4-di-chloro), L-Bip(4-fluoro), L-Bip(3,4-di-fluoro), L-Bip(2,5-di-fluoro), L-Bip(3-n-propyl), L-Bip(4-n-propyl), L-Bip(2-iso-propyl), L-Bip(3-iso-propyl), L-Bip(4-iso-propyl), L-Bip(4-tert-butyl), L-Bip(3-phenyl), L-Bip(2-chloro), L-Bip(3-chloro), L-Bip(2-fluoro), L-Bip(3-fluoro), L-Bip(2-CF₃), L-Bip(3-CF₃), L-Bip(4-CF₃), L-Bip(3-NO₂), L-Bip(3-OCF₃), L-Bip(4-OCF₃), L-Bip(2-OEt), L-Bip(3-OEt), L-Bip(4-OEt), L-Bip(4-SMe), L-Bip(2-OH), L-Bip(3-OH), L-Bip(4-OH), L-Bip(2-CH₂—COOH), L-Bip(3-CH₂—COOH), L-Bip(4-CH₂-COOH), L-Bip(2-CH₂—NH₂), L-Bip(3-CH₂—NH₂), L-Bip(4-CH₂—NH₂), L-Bip(2-CH₂—OH), L-Bip(3-CH₂—OH), L-Bip(4-CH₂—OH), L-Phe[4-(1-propargyl)], L-Phe[4-(1-propenyl)], L-Phe[4-n-butyl], L-Phe[4-cyclohexyl], Phe(4-phenyloxy), L-Phe(penta-fluoro), L-2-(9,10-dihydrophenanthrenyl)-Ala, 4-(2-benzo(b)furan)-Phe, 4-(4-Dibenzofuran)-Phe, 4-(4-phenoxathiin)-Phe, 4-(2-Benzo(b)thiophene)-Phe, 4-(3-thiophene)-Phe, 4-(3-Quinoline)-Phe, 4-(2-naphthyl)-Phe, 4-(1-Naphthyl)-Phe, 4-(4-(3,5-dimethylisoxazole))-Phe, 4-(2,4-dimethoxypyrimidine)-Phe, homoPhe, Tyr(BzI), Phe(3,4-di-chloro), Phe(4-lodo), 2-Naphthyl-Ala, L-α-Me-Bip, or D-α-Me-Bip; Z is L-Bip, D-Bip, L-Bip(2-Me), D-Bip(2-Me), L-Bip(2′-Me), L-Bip(2-Et), D-Bip(2-Et), L-Bip(3-Me), L-Bip(4-Me), L-Bip(3-OMe), L-Bip(4-OMe), L-Bip(4-Et), L-Bip(2-n-propyl,2′-Me), L-Bip(2,4-di-Me), L-Bip(2-Me, 2′-Me), L-Bip(2-Me,4-OMe), L-Bip(2-Et, 4-OMe), D-Bip(2-Et,4-OMe), L-Bip(2,6-di-Me), L-Bip(2,4,6-tri-Me), L-Bip(2,3,4,5,-tetra-Me), L-Bip(3,4-di-OMe), L-Bip(2,5-di-OMe), L-Bip(3,4-Methylene-di-oxy), L-Bip(3-NH-Ac), L-Bip(2-iso-propyl), L-Bip(4-iso-propyl), L-Bip(2-Phenyl), L-Bip(4-Phenyl), L-Bip(2-fluoro), L-Bip(4-CF₃), L-Bip(4-OCF₃), L-Bip(2-OEt), L-Bip(4-OEt), L-Bip(4-SMe), L-Bip(2-CH₂—COOH), D-Bip(2-CH₂—COOH), L-Bip(2′-CH₂—COOH), L-Bip(3-CH₂—COOH), L-Bip(4-CH₂—COOH), L-Bip(2-CH₂-NH₂), L-Bip(3-CH₂-NH₂), L-Bip(4-CH₂—NH₂), L-Bip(2-CH₂—OH), L-Bip(3-CH₂—OH), L-Bip(4-CH₂—OH), L-Phe(3-Phenyl), L-Phe[4-n-Butyl], L-Phe[4-cyclohexyl], Phe(4-Phenyloxy), L-Phe(penta-fluoro), L-2-(9,10-DihydrophenanthrenyI)-Ala, 4-(3-Pyridyl)-Phe, 4-(2-Naphthyl)-Phe, 4-(1-naphthyl)-Phe, 2-naphthyl-Ala, 2-fluorenyl-Ala, L-α-Me-Bip, D-α-Me-Bip, L-Phe(4-NO₂), or L-Phe(4-lodo); A is H, acetyl, 6-Ala, Ahx, Gly, Asp, Glu, Phe, Lys, Nva, Asn, Arg, Ser, Thr, Val, Trp, Tyr, caprolactam, Bip, Ser(Bzl), 3-pyridylAla, Phe(4-Me), Phe(penta-fluoro), 4-methylbenzyl, 4-fluorobenzyl, n-propyl, n-hexyl, cyclohexylmethyl, 6-hydroxypentyl, 2-thienylmethyl, 3-thienylmethyl, penta-fluorobenzyl, 2-naphthylmethyl, 4-biphenylmethyl, 9-anthracenylmethyl, benzyl, (S)-(2-amino-3-phenyl)propyl, methyl, 2-aminoethyl, or (S)-2-aminopropyl; and B is OH, NH₂, Trp-NH₂, 2-naphthylAla-NH₂, Phe(penta-fluoro)-NH₂, Ser(Bzl)-NH₂, Phe(4-NO₂)—NH₂, 3-pyridylAla-NH₂, Nva-NH₂, Lys-NH₂, Asp-NH₂, Ser-NH₂, His-NH₂, Tyr-NH₂, Phe-NH₂, L-Bip-NH₂, D-Ser-NH₂, Gly-OH, .beta.-Ala-OH, GABA-OH, or APA-OH.

In certain embodiments, when A is not present, and X₁ is an R group, an R—C(O) (amide) group, a carbamate group RO—C(O), a urea R₄R₅N—C(O), a sulfonamido R—SO₂, or a R₄R₅N—SO₂; wherein R is H, C₁₋₁₂ alkyl, C₃₋₁₀ cycloalkyl, cycloalkylalkyl, heterocyclyl, heterocycloalkyl, aryl, heteroaryl, arylalkyl, aryloxyalkyl, heteroarylalkyl, heteroaryloxyalkyl, or heteroarylalkoxyalkyl; and where R₄ and R₅ are each independently H, C₁₋₁₂ alkyl, C₃₋₁₀ cycloalkyl, cycloalkylalkyl, heterocyclyl, heterocycloalkyl, aryl, heteroaryl, arylalkyl, aryloxyalkyl, heteroarylalkyl, or heteroaryloxyalky.

In certain embodiments, when B is not present and Z is OR₁, NR₁ R₂, or an amino-alcohol; where R₁ and R₂ are independently H, C₁₋₁₂ alkyl, C₃₋₁₀ cycloalkyl, cycloalkylalkyl, heterocycle, heterocycloalkyl, aryl, heteroaryl, arylalkyl, aryloxyalkyl, heteroarylalkyl, or heteroaryloxyalkyl. In certain embodiments, X₁ (where applicable), X₂, and X₃ are N—H or N-alkylated, (e.g., N-methylated) amino acid residues. The polypeptide may be a 10-mer to 15-mer and capable of binding to and activating the GLP-1 receptor.

Abbreviations

Nal=naphthylalanine

pGly=pentylglycine

t-BuG or =t-butylglycine

TPro=thioproline

HPro=homoproline

NmA=N-methylalanine

Cya=cysteic acid

Thi=β2-Thienyl-Ala

hSer=homoserine

Aib=a-aminoisobutyric acid

Bip=biphenylalanine

Nle=norleucine

Ahx=2-aminohexanoic acid

Nva=norvaline

### Hypothalamic and Pituitary Hormones

The peptide therapeutic can be a hypothalamic or pituitary hormone. These hormones include pituitary hormone-releasing hormones, pituitary hormone release inhibiting hormones, pro-opiomelanocortin, growth hormones, pituitary gonadotropins (e.g., those described herein), vasotocin, oxytocin, and vasopressins (e.g., those described herein). Other hypothalamic or pituitary hormones include Coturnix japonica gonadotropin-inhibitory hormone, lactotropin, rat luteinizing hormone release-inhibiting factor, melanin concentrating hormone precursor(109-129)-glycyl-glutamic acid, melanin-concentrating hormone precursor, melanin-concentrating-hormone precursor(129-145)-glutamyl-isoleucinamide, neurohormone C, prolactin-releasing peptide, human protein, rat DGF protein, melanin-concentrating hormone, melanin-concentrating hormone(2-17), Phe(13),Tyr(19)-melanin-concentrating hormone, nasohypophysial factor, decidual luteotropin, lysocorticone, sperm releasing substance, Bos taurus TSP 86-84 protein, coherin, hypophysin, hypostin, and fish somatolactin protein.

Pituitary Hormone-releasing Hormones

In certain embodiments, the peptide therapeutic is a pituitary hormone-releasing hormone or an analog thereof. Such hormones include corticotropin-releasing hormone, gonadotropin-releasing hormone, growth hormone-releasing hormone, and thyrotropin-releasing hormone (TRH).

Analogs of corticotropin-releasing hormone include α-helical corticotropin-releasing hormone, Tyr(3)-Pro(4)-Nle(18,21)-α-helical corticotropin releasing hormone(3-41), astressin, astressin B, cortagine, corticorelin ovine, Nle(21,38)-Arg(36)-corticotropen-releasing hormone, corticotropin releasing hormone(9-41), biotinyl-Ser(1)-corticotropin releasing hormone, Phe(12)-Nle(21,38)-corticotropin-releasing hormone(12-41), Phe(12)-Nle(21,38)-α-Me-Leu(37)-corticotropin-releasing hormone(12-41), Glu(20)-corticotropin-releasing hormone, iodo-Tyr(0)-corticotropin-releasing hormone, Nle(21)-iodo-Tyr(32)-corticotropin-releasing hormone, Pro(5)-corticotropin-releasing hormone, cyclo(31-34)(phenylalanyl(12)-norleucyl(21,28)-glutamyl(31)-lysyl(34))acetyl-corticotropin releasing factor(4-41), Phe(12)-Nle(21,38)-C(α-MeLeu(37))-H—R corticotropin-releasing factor(12-41), phenylalanyl corticotropin-releasing factor, phenylalanyl corticotropin-releasing factor(12-41), pro-corticotropin releasing hormone, prolyl-prolylisoleucine, human UCN2 protein, human UCN3 protein, mouse urocortin 2, and rat urocortin 3.

Analogs of gonadotropin-releasing hormone include (β-Asp(α-DEA))(6),Gln8-GnRH, (lysine(6)(1,3,8-trihydroxy-6-carboxyanthraquinone))GnRH, (Arg(6)-Trp(7)-Leu(8)-Pro(9)-NEt)GnRH, 9-hydroxyproline-gonadorelin, A 76154, AN 207, argtide, azaline, azo-GnRH tetanus toxoid conjugate, azo-LHRH-bovine serum albumin conjugate, azo-LHRH-tetanus toxoid conjugate, BIM 21009, bovine serum albumin-LHRH conjugate, Buserelin, cetrorelix, Lys(4)-Trp(6)-Glu(9)-cyclo(4-9)GnRH, dalarelin, detirelix, fertirelin, folligen, ganirelix, penicillamine-(tert-butyl)(6)-GnRH(1-9)nonapeptide ethylamide, (Ac-dehydro-Pro(1)-4-Cl-Phe(2)-Trp(3,6))-N-α-MeLeu(7)-GnRH, Ac(3,4)-dehydro-Pro(1)-4-fluoro-Phe(2)-Trp(3,6)-GnRH, Ac(4-Cl-Phe(1,2)-Trp(3)-Tyr(5)-Lys(6)-Ala(10))-GnRH, acetyl(4-azidobenzoyl)-Lys(1)-4-chloro-Phe(2),Trp(3),Arg(6),Ala(10)-GnRH, Trp(6)-N-Me-Leu(7)-Pro(9)-N-Et-GnRH, GnRH-hinge-MVP peptide, Lys(6)-GnRH-II, GnRH3-hinge-MVP, gonadorelin(6-D-Phe), Gonadorelin-like peptide, gonadotropin releasing hormone associated peptide, gonadotropin-releasing hormone-III, goserelin, histrelin, chimeric L-GnRH-PE66 protein, lamprey GnRH-I, Leuprolide, LHRH(1-10), LHRH(1-5), LHRH(1-6), GlyNH₂(6)-LHRH(1-6), LHRH(1-9), LHRH(2-10), Trp(6)-LHRH(2-10), desArg-LHRH cysteamide, desGly(10)-Ala(6)-LHRH ethylamide, Ala(6)-desGly(10)-LHRH propylamide, Ac-LHRH(5-10), (1,9)-nonapeptide N-Et-ProNH(2)(9)-Gln(cyclohexyl)(6)-desGlyNH₂(10)-LHRH, (3-(1H-pyrazol-3-yl))-Ala(2)-LHRH, (N)-Ac-3(2-naphthyl)Ala(1)-(4-Cl-Phe)(2)-Trp(3)-Arg(6)-Phe(7)-AlaNH₂(10)-LHRH, 1,6-cyclo(Ac-Glu(1)-Phe(2)-Trp(3)-Lys(6))-LHRH, 4-amino-Phe(6)-LHRH, 4-ClPhe(2)-Trp(3,6)-LHRH, Ac-(4-Cl-Phe(1,2)-Trp(3)-Lys(6)-Ala(10))-LHRH, Ac-2-Nal(1)-4-Cl-Phe(2)-3-Pal(3)-Arg(5)-4-methoxybenzoyl-2-ABA(6)-Ala(10)-LHRH, Ac-2-Nal(1)-4-Cl-Phe(2)-Trp(3)-Arg(Et2)(6)-Ala(10)-LHRH, Ac-2-Nal(1)-4-Cl-Phe(2)-Trp(3)-Ser(Rha)(6)-AzGlyNH₂(10)-LHRH, Ac-dehydro-Phe(1)-dehydro-4-Cl-Phe(2)-dehydro-Trp(3,6)-LHRH, Ac-dehydro-Pro(1)-4-Cl-Phe(2)-Trp(3,6)-LHRH, Ac-dehydro-Pro(1)-4-F-Phe(2)-Trp(3,6)-LHRH, Ac-delta(3)-Pro(1)-4-F-Phe(2)-Trp(3)-Lys(6)-LHRH, Ac-Nal(1)-4-Cl-Phe(2)-Pal(3,6)-LHRH, Ac-Nal(1)-4-Cl-Phe(2)-Trp(3)-Arg(6)-Trp(7)-Ala(10)-LHRH, Ac-Nal(1)-Cpa(2)-Pal(3,6)-Arg(5)-Ala(10)-LHRH, Ac-Nal(1)-Cpa(2)-Trp(3)-Arg(6)-Ala(10)-LHRH, Ac-Nal-Ala(1)-4-Cl-Phe(2)-Ser(Rha)(6)-LHRH, acetyl-2-(2-naphthyl)-Ala(1)-4-F-Phe(2)-Trp(3)-Arg(6)-LHRH, Ala(6)-LHRH, Ala(6)-desGly(l0)-LHRH, Ala(6)-Gly(10)-ethylamide-LHRH, Ala(6)-N-Et-ProNH₂(9)-iodo-LHRH, biotin-Lys(6)-LHRH, Boc-(BzI)Ser(1)-desHis(2)-Trp(6)-LHRH, cLeu(7)-LHRH, cyclohexyl-Ala(7)-LHRH, desHis(2)-Ala(6)-LHRH, desHis(2)-Ala(6)-N-Et-ProNH₂(9)-LHRH, desHis(2)-Leu(6)-LHRH, desTyr(5)-LHRH, Gln(1)-desHis(2)-Phe(6)-N-Et-ProNH₂(9)-LHRH, Gln(8)-LHRH, Gly(10)-LHRH, His(5)-Arg(6)-Trp(7)-Tyr(8)-LHRH, His(5)-Trp(7)-Tyr(8)-LHRH, His(5)-Tyr(6)-LHRH, His(6)-N-Et-ProNH₂(9)-LHRH, hydroxyprolyl(9)-LHRH, Leu(6)-LHRH, Leu(6)-desEt-GlyNH₂(10)-LHRH, Leu(6)-Leu(N-α-Me)(7)-N-Et-ProNH₂(9)-LHRH, Leu(6)-N-Et-GlyNH₂(10)-LHRH, Lys(6)-LHRH, Lys(6)-EGS-Lys(6)-LHRH LHRH, Lys(6)-N-Et-GlyNH₂(10)-LHRH, Lys(6)-N-Et-ProNH₂(9)-LHRH, Lys(8)-LHRH, lysine(6)-glutaryl-2-(hydroxymethyl)anthraquinone LHRH, N-(Ac)-Trp(1)-(4-Cl-Phe)(2)-Trp(3)-Trp(6)-AlaNH₂(10)-LHRH, N-Ac(2)-Nal(1)-4-Cl-Phe(2)-3-Pal(3)-Arg(5)-5-(4-methoxyphenyl)-5-oxo-2-aminopentanoic acid(6)-Ala(10)-LHRH, N—Ac-(4-Cl-Phe)(1,2)-Phe(3)-Arg(6)-AlaNH₂(10)-LHRH, N—Ac-(4-Cl-Phe)(1,2)-Trp(3)-Arg(6)-AlaNH₂(10)-LHRH, N—Ac-(4-F-Phe)(1)-(4-Cl-Phe)(2)-Trp(3,6)-AlaNH₂(10)-LHRH, N—Ac-2-Nal(1)-4-Cl-Phe(2)-3-Pal(3)-Arg(5)-Glu(6)-AlaNH₂(10)-LHRH, N—Ac-2-Nal(1)-4-Cl-Phe(2)-Trp(3)-Hci(6)-AlaNH₂(10)-LHRH, N—Ac-2-naphthyl-Ala(1)-4-chloro-Phe(2)-pyridyl-Ala(3)-nicotinyl-Lys(5,6)-isopropyl-Lys(8)-AlaNH₂(10)-LHRH, N—Ac-3(2-naphthyl)Ala(1)-Phe(2,3)-Arg(6)-Phe(7)-AlaNH₂(10)-LHRH, N—Ac-3-(2-dibenzofuranyl)-Ala(1)-LHRH, N—Ac-Ala(1)-(4-Cl-Phe)(2)-Trp(3,6)-LHRH, N—Ac-Gly(1)-(4-Cl-Phe)(2)-Trp(3,6)-LHRH, N—Ac-muramyl-Ala-iso-Glu-LysNH₂-LHRH, N—Ac-Nal(1)-4-Cl-Phe(2)-Trp(3)-Cit(6)-AlaNH₂(10)-LHRH, N—Ac-naphthyl(1)-(4-Cl-Phe)(2)-Trp(3)-Arg(6)-Ala(10)-LHRH, N—Ac—O-phenylTyr(1)-LHRH, N-Ac-Pro(1)-(4-Cl-Phe)(2)-(2-naphthyl-Ala)(3,6)-LHRH, N—Ac-Trp(1)-(4-Cl-Phe)(2)-Trp(3)-Arg(6)-Ala(10)-LHRH, N-acetyl-(4-chlorophenylalanyl)(1)-(4-chlorophenylalanyl)(2)-tryptophyl(3)-arginyl(6)-alanine(10)-LHRH, N-epsilon-azidobenzoyl-Lys(6)-LHRH, N-Et-AlaNH₂(6)-LHRH, pGlu(1)-4-Cl-Phe(2)-Trp(3,6)-LHRH, pGlu(1)-Phe(2)-Trp(3)-Lys(6)-LHRH, pGlu(1)-Phe(2)-Trp(3)-Ser(4)-N-ε-azidobenzoyl-Lys(6)-LHRH, pGlu(1)-Phe(2)-Trp(3,6)-LHRH, Phe(2)-Ala(6)-LHRH, Phe(2)-Leu(6)-LHRH, Phe(2)-N-ε-(2,4)-dinitrophenol-Lys(6)-LHRH, Phe(2)-Pro(3)-Phe(6)-LHRH, Phe(2)-Trp(3)-Phe(6)-LHRH, Phe(2)-Trp(6)-LHRH, Phe(5)-5-Ala(6)-LHRH, Phe(5)-δ-Ala(6)-N-Et-ProNH₂(9)-LHRH, Phe(6)-LHRH, Phe(7)-LHRH, Pro(1)-Phe(2)-Trp(3,6)-LHRH, rhodamine LHRH, rhodamine-Lys(6)-LHRH, Ser(6)-LHRH, Trp(6)-desGlyNH₂(10)-LHRH, Trp(7)-LHRH, Trp(8)-LHRH, (N)-Ac-(4-Cl-Phe)(1)-(4-Cl-Phe)(2)-Trp(3)-Phe(6)-AlaNH₂(10)-LHRH, (N)—Ac-Trp(1)-(4-Cl-Phe)(2)-Trp(3)-Phe(6)-AlaNH₂(10)-LHRH, lutrelin acetate, meterelin, MI 1544, MI 1892, N—Ac-(4-Cl-Phe)(1)-(4-C1-Phe)(2)-Trp(3)-Lys(6)-AlaNH₂(10)-LHRH, N-acetyl-3-(3-quinolypalanyl-3-(4-chlorophenyl)alanyl-3-(3-pyridypalanyl-seryl-3-(4-pyrazinylcarbonylaminocyclohexyl)alanyl-N(epsilon)picolinoyllysyl-valyl-arginyl-prolyl-alaninamide, Nafarelin, azaglycylnafarelin, Org 30850, orntide acetate, ovaprim, ovurelin, P-X 1544, P-X 1892, Ala(17)-phLHRH(14-36), rat porf-2 protein, pro-LHRH(14-69)OH, progonadoliberin I, pyroglutamyl-histidyl-tryptophyl-seryl-tyrosyl-tryptophyl-leucyl-arginyl-prolyl-glycinamide, pyroglutamyl-histidyl-tryptophyl-seryl-tyrosyl methyl ester, relisorm L, ricin A-GnRH conjugate, RS 15378, RS 18286, surfagon, T 107, triptorelin, deslorelin, triptorelin pamoate, Tryptal, Vaxstrate, Wy 40905, and Wy 43657.

Analogs of growth hormone-releasing hormone include CJC 1295, sermorelin, DBO 29, GRF-1PEG500, MZ 3-149, MZ 4-243, MZ 4-71, MZ 5-156, MZ-J-7-118, Nle(27)-somatotropin(1-29)amide, 27-Leu-somatotropin releasing hormone(1-29) amide, desNH₂Tyr(1)-Ala(2,15)-somatotropin releasing hormone(1-29)NH₂, N—Ac-Tyr(1)-Phe(2)-somatotropin releasing hormone(1-29)amide, N-acetyl-Tyr(1),Ala(2)-somatotropin releasing hormone(1-29)amide, N-acetyl-Tyr(1),Arg(2)-somatotropin releasing hormone(1-29)amide, N-acetyl-tyrosyl(1)-arginyl(2)-somatotropin releasing hormone(1-29)amide, Leu(27)-Ala(2)-somatotropin releasing hormone(1-29)NH₂, desNH₂Tyr(1)-Ala(15)-somatotropin releasing hormone(1-29)NH₂, Ala(15)-somatotropin-releasing hormone(1-29)amide, α-hydroxy-Gly(14)-Ala(15)-somatotropin-releasing hormone(1-29), Ala(2)-somatotropin-releasing hormone(1-29)amide, growth hormone releasing hormone-related peptide, Ala(15)-Leu(27)-growth hormone-releasing factor(1-32)amide, JI-38, JV 1-36, JV 1-38, JV 1-52, JV 1-53, pre-pro-growth hormone releasing factor, human Pro-GHRH(2-44) peptide, mouse pro-growth hormone releasing hormone, human Pro-Pro-hGHRH(1-44) peptide, Pro-Pro-hGHRH(1-44)-Gly-Gly-Cys, Ro 23-7861, somatotropin releasing factor 40, Leu(27)-somatotropin releasing factor 40, somatotropin releasing hormone(1-24)amide, somatotropin releasing hormone(1-26)amide, somatotropin releasing hormone(1-29), His(1)-I-Tyr(10)-Nle(27)-somatotropin releasing hormone(1-32)amide, Pro(15)-Leu(27)-somatotropin releasing hormone(1-32)NH₂, somatotropin releasing hormone(1-37), somatotropin releasing hormone(1-37)amide, Ala(34)-Ser(38)-Arg(40)-somatotropin releasing hormone(1-40)-OH, Ac-Tyr(1)-somatotropin releasing hormone(1-40)-OH, somatotropin releasing hormone(1-43), Leu(27)-somatotropin releasing hormone(1-44)-OH, somatotropin releasing hormone(1-44)amide, somatotropin releasing hormone(1-45), 27-Leu-45-Gly-somatotropin releasing hormone(1-45), Leu(27)-somatotropin releasing hormone(3-29)NH₂, N(a) biotinyl(1-44)amide somatotropin releasing hormone, 1-His-2-Ala-27-Nle-somatotropin releasing hormone(1-29), 1-N-MeTyr-27-Nle-28-Asn-somatotropin releasing hormone(1-29)NHEt, 2-Ala-somatotropin releasing-hormone(1-29), 3′-5′ somatotropin-releasing hormone(1-23), somatotropin-releasing hormone(1-29)-Gly-Gly-Gly-Gly-Cys-NH₂, somatotropin-releasing hormone(1-30)amide, Ala(2)-Leu(15)-Nle(27)-GABA(30)-somatotropin-releasing hormone(1-30)amide, Ile(2)-Ser(8)-Ala(15)-Leu(27)-Ser(28)-Hse(30)-somatotropin-releasing hormone(1-30)amide, isoAsp(8)-Leu(27)-somatotropin-releasing hormone(1-32)amide, His(1),Nle(27)-somatotropin-releasing hormone(1-32)amide, Ala(15,29)-somatotropin-releasing hormone(4-29)-OH, Leu(27)-somatotropin-releasing hormone(1-32) amide, Ala(2)-Nle(27)-GABA(30)-somatotropin-releasing hormone(1-30)-amide, Asp(8)-Leu(27)-somatotropin-releasing hormone(1-32)amide, tesamorelin, U 90349E, and lysyl(15)-arginyl(16)-leucyl(27)-vasoactive intestinal peptide(1-7)-growth hormone-releasing factor(8-27).

Analogs of TRH include 2-hydroxy-4-carboxybutyrylhistidyl-prolinamide, 3-(aminocarbonyl)-1-(3-(2-(aminocarbonyl)pyrrolidin-1-yl)-3-oxo-2-(((5-oxopyrrolidin-2-yl)carbonyl)amino)propyl)pyridinium, 5-oxoprolyl-2,4(5)-diiodohistidyl-prolinamide, 5-oxoprolyl-4(5)-iodohistidyl-prolinamide, pGlu-His-amphetamine, CG 3509, digipramine, DN 1417, fluorescein-TRH, Glp-asparagine-proline-D-tyrosine-D-tryptophanylamide, glutaminyl-pyroglutamyl-glutamyl-proline amide, JTP 2942, L-pyroglutamyl-L-histidyl-3,3-dimethylprolinamide, methyl pyroglutamyl-histidyl-pipecolate, MK 771, montirelin, N-(2-hydroxy-4-(isobutylcarbamoyl)butyryl)histidylprolinamide, rat pFQ7 protein, posatirelin, PR 546, prepro-thyrotropin releasing hormone(160-169), prepro-thyrotropin releasing hormone(25-50), prepro-thyrotropin releasing hormone(53-74), prepro-TRH, prepro-TRH(178-199), pro-thyrotropin releasing hormone, Nle(2)-Prot(3)-protirelin, Nve(2)-Prot(3)-protirelin, rat pSE14 protein, pyro(a-aminoadipyI)-histidyl-prolinamide, pyroglutamyl-((N3)-imidazolylmethyl)-histidyl-n-amylprolinamide, pyroglutamyl-glutamyl-proline amide, pyroglutamyl-histidyl-3-methylprolinamide, pyroglutamyl-histidyl-proline thioamide, pyroglutamyl-histidyl-proline-tyramine, pyroglutamyl-L-histidyl-L-pipecolic acid amide, pyroglutamyl-leucyl-prolinamide, pyroglutamyl-tyrosyl-prolylamide, TA 0910, TA 0910 acid-type, thyroliberin N-ethylamide, TRH 5-fluoroimidazole, TRH chloromethyl ketone, TRH diazomethyl ketone, CRM45 thyrotropin releasing hormone, 1-(methano-Glp(2,3))-TRH, 1-Me-TRH, 2,4-diiodoimidazole-TRH, 2,4-MePro(3)-TRH, 2-diazohistidinyl-TRH, 2-fluoromethylimidazole-TRH, 2-picolyl-TRH, 3-Me-TRH, 4(5)-nitroimidazole-TRH, 4-fluoroimidazole-TRH, beta-(pyrazolyl-1)-Ala(2)-TRH, deamido-TRH, Gly TRH, Gly-Lys-Arg-TRH, Leu(2)-Pip(3)-TRH, linear beta-Ala-TRH, nVal(2)-TRH, Pro-hydrazide-TRH, and 4-azidosalicylamide-TRH.

Pituitary Hormone Release Inhibiting Hormones

In certain embodiments the peptide therapeutic is a pituitary hormone release inhibiting hormone or an analog thereof. Such peptides include MSH release inhibiting hormones, somatostatin, or analog thereof.

Exemplary MSH relase inhibitng hormones include carbobenzoxyprolyl-leucyl-glycinamide, N-acetyl-prolyl-leucyl-glycinamide, pareptide, prolyl-leucyl-glycine, prolyl-leucyl-thiazolidine-2-carboxamide, tyrosyl-prolyl-leucyl-glycinamide, tyrosyl-prolyl-leucyl-glycine, tyrosyl-prolyl-lysyl-glycinamide, and tyrosyl-prolyl-tryptophyl-glycinamide.

Exemplary somatostatin analogs include angiopeptin, antrin, AOD 9604, AS 51, AS 52, BIM 23003, BIM 23034, BIM 23052, BIM 23120, BIM 23206, BIM 23268, BIM 23926, BIM 23A760, CGP 15425, CGP 23996, CH 275, CH 288, CMDTPA-Tyr3-octreotate, cyclo(7-aminoheptanoyl-phenylalanyl-tryptophyl-lysyl-threonyl), cyclo(7-aminoheptanoylphenylalanyl-tryptophyl-lysyl-benzylthreonyl), cyclo(aminoheptanoic acid-cyclo(cysteinyl-phenylalanyl-D-tryptophyl-lysyl-threonyl-cysteinyl)), cyclo(β-methyl-N-benzylglycyl-phenylalanyl-tryptophyl-lysyl-threonyl-phenylalanyl), cyclo(N-benzylglycyl-phenylalanyl- tryptophyl-lysyl-threonyl-phenylalanyl), cyclo(Pro-Phe-Trp-Lys-Thr-Phe), cyclo(prolyl-thiomethyl-phenylalanyl-tryptophyl-lysyl-threonyl-phenylalanyl), D,D-carbasomatostatin, DC 32-87, dihydrosomatostatin, JF-10-81, Lan-7, lanreotide, pentetreotide, Phe-Cys-Phe-Trp-Lys-Thr-Pen-Thr-NH₂, phenylalanyl-cyclo(cysteinyl-tyrosyltryptophyl-lysyl-threonyl-penicillamine)threoninamide, phenylalanyl-cyclo(cysteinyltyrosyl-tryptophyl-ornithyl-threonyl-penicillamine)threoninamide, prosomatostatin, prosomatostatin(29-92), prosomatostatin cryptic peptide, PTR 3173, PTR-3205, RC 161, San 201-456, sms-D70, SOM-230, desamino-Trp somatostatin(7-10), somatostatin 28, Leu(8)-Trp(22)-iodo-Tyr(25)-somatostatin 28, Nle(8)-somatostatin 28, Trp(22)-somatostatin 28, Tyr(7)-Gly(10)-somatostatin 28, somatostatin 28(1-12), Tyr(11)-somatostatin 14, Tyr(7)-Gly(10)-somatostatin 14, somatostatin 20, somatostatin 25, somatostatin 25(1-9), somatostatin 26, somatostatin 34, somatostatin RC 102, somatostatin(3-6), somatostatin(7-10), 4-NH₂-Phe(4)-Trp(8)-somatostatin, 5-fluoro-Trp(8)-somatostatin, 5-methoxy-Trp(8)-somatostatin, Ala(2)-Trp(8)-Cys(14)-somatostatin, Ala(3,14)-somatostatin, Ala(5)-Orn(9)-somatostatin, Ala(5)-Trp(8)-somatostatin, azidonitrobenzoyl-Lys(4)-iodo-Tyr(11)-somatostatin, azidonitrobenzoyl-Lys(9)-iodo-Tyr(11)-somatostatin, cyclic hexapeptide(Phe-Phe-Trp-Lys-Thr-Phe)-somatostatin, cyclo(desAla(1)-desGly(2)-S-COMe-homo-CysNH₂(3)-Trp(8)-desCys(14))-somatostatin, cyclo(Pro-dehydro-Phe-Trp-Lys-Thr-Phe) somatostatin, Cys(3)-somatostatin, de-Asn(5)-Trp(8)-Ser(13)-somatostatin, desAla(1)-somatostatin, desAla(1)-desGly(2)-Trp(8)-Asn(3,14)-somatostatin, desamino acid(1,2,5)-Glu(7)-Trp(8)-1Amp(9)-m-I-Tyr(11)-hhLys(12)-somatostatin, desAsn(5)-somatostatin, iodine-Tyr-somatostatin, iodo-Tyr(1)-somatostatin, N—Ac-desAla(1)-desGly(2)-4-Cl-Phe(6)-Trp(8)-somatostatin, N-Tyr(1)-somatostatin, nonapeptide-D-Trp(8)-somatostatin, octapeptide-Trp(8)-somatostatin, Phe(4)-somatostatin, Pro(2)-Met(13)-somatostatin, protamine zinc-somatostatin, seleno-Cys(3,14)-Trp(8)-somatostatin, Ser(13)-somatostatin, Trp(8)-somatostatin, Trp(8)-Cys(14)-somatostatin, Tyr(11)-somatostatin, Val(1)-Trp(8)-somatostatin, somatostatin-22, zebrafish sst1 protein, ^{99m}Tc depreotide, human thrittene protein, TT2-32, vapreotide, Woc4D, Wy 40770, Wy 40793, Wy 41747, (¹⁷⁷utetium-DOTA(0)Tyr3)octreotate, (DOTA(0)-Phe(1)-Tyr(3))octreotide, (PnAO-(D)Phe(1))octreotide, (^{99m}Tc-EDDA-tricine-HYNIC(0)-Nal(1)-Thr(8))octreotide, ¹¹¹In-DOTA-TOC, DOTA(0)-Tyr(3)-Thr(8)-¹¹¹In-octreotate, DTPA(0)-¹¹¹In-octreotide, DOTA(0)-Tyr(3)-¹⁷⁷Lu-octreotide, 3-Tyr-octreotide, 4-nitrobenzo-2-oxa-1,3-diazol-octreotide, DOTA-Tyr(3)-⁹⁰Y-octreotide, ^{99m}Tc-octreotide, copper 1,4,8,11-tetraazacyclotetradecane-N,N′,N”,N′″-tetraacetic acid-octreotide, copper-1,4,8,11-tetraazacyclotetradecane-N,N′,N″,N′″-tetraacetic acid-octreotate, desferrioxamine B-succinyl-phenylalanine(1)-octreotide, DOTA-Tyr(1)-octreotate, DTPA-benzyl-acetamido-D-Phe(1),Tyr(3)-octreotide, EE 581, Ga(111)-DOTATOC, “'In-octreotide, maltotriose-iodotyrosyl(3)-octreotate, CPTA-Phe(1)-octreotide, DOTA-Phe(1)-octreotide, DOTA-Tyr(3)-octreotide, iodoTyr(3)-octreotide, TETA-Phe(1)-octreotide, octreotide-conjugated paclitaxel, phenylalanyl-cysteinyl-tyrosyl-tryptophyl-lysyl-threonyl-cysteinyl-N-naphthylalanine amide, RC 121, SDZ 204-090, SDZ 215-811, SDZ 223228, SDZ CO 611, ^{99m}Tc-(EDDA-HYNIC)octreotate, ^{99m}Tc-6-(4-thioureabenzyl)-3,3,9,9-tetramethyl-4,8-diazaundecane-phenylalanine(1)-octreotide, ^{99m}Tc cyclopentadienyltricarbonyloctreotide, ^{99m}Tc hydrazinonicotinyl-Tyr(3)-octreotide, ^{99m}Tc hydrazinonicotinyl-Tyr(3)-Thr(8)-octreotide, ^{99m}Tc-tricine-hydrazinonicotinyl-Phe(1)-Tyr(3)-octreotide, Y-DOTA-t-GA-tate, Y-DOTAGA-tate, and yttrium cyclohexyldiethylenetriaminepentaacetic acid-octreotide.

Pro-Opiomelanocortin

In certain embodiments, the peptide thereaputic is pro-opiomelanocortin or a derivative (e.g., a cleavage product) or an analog thereof. Pro-opiomelanocortin can be cleaved to form, for example, adrenocorticotropic hormone (ACTH), endorphins (e.g., α-endorphin, β-endorphin, and γ-endorphin), β-lipotropin, γ-lipotropin, and melanocyte-stimulating hormones (e.g., α-MSH, β-MSH, and γ-MSH).

Pro-opiomeanocortin analogs include pro-opiomelanocortin(1-49), pro-opiomelanocortin(1-77), pro-opiomelanocortin amino-terminal glycopeptide, pro-opiomelanocortin joining peptide, pro-opiomelanocortin human joining peptide(77-109), pro-opiomelanocortin joining peptide(14-23), pro-opiomelanocortin joining peptide(77-97), and pro-opiomelanocortin joining peptide(79-108), and zebrafish POMC protein.

ACTH analogs include 41795-Ba, acethropan-S, ACTH(1-10), ACTH(1-14), ACTH(1-16), ACTH(1-17), (Na-(biotinyl-β-Ala1)-Lys17)-ACTH(1-17)-NH-(CH₂)₄-NH₂, bis(Cys(25))-ACTH(1-26), Cys-carboxamidomethyl(25)-ACTH(1-26), ACTH(1-32), ACTH(1-37), ACTH(1-38), Phe(2)-Nle(4)-ACTH(1-38), Phe(2)-Nle(4)-iodo-Tyr(23)-ACTH(1-38), ACTH(1-4), ACTH(1-24), ACTH(13-24), ACTH(17-39), ACTH(25-39), ACTH(27-39), ACTH(4-10), Phe(7)-ACTH(4-10), ACTH(4-11), ACTH(4-12), ACTH(4-7), Pro-Gly-Pro-ACTH(4-7), ACTH(4-9), ACTH(5-10), ACTH(5-14), ACTH(5-8), ACTH(6-9), ACTH(7-10), ACTH(7-16)NH₂, ACTH(7-38), ACTH(7-39), Phe(2)-Nle(4)-ACTH(1-24), ACTH(11-24), ACTH(15-24), ACTH(19-24), ACTH(5-24), ACTH(6-24), adrenocorticotropin zinc, 7-MeTrp(9)-cosyntropin, Ala(1)-Lys(17)-ACTH 4-amino-n-butylamide(1-17), ACTH α(1-18), Phe(7)-ACTH amide(1-10), ACTH amide(1-16), Gly(1)-ACTH amide(1-18), Ala(1)-ACTH amide(1-20), Gly(l)-ACTH amide a(1-18), (t-butyl-Trp)(9)-ACTH nonadecapeptidamide(1-19), (Trp(2,5,7-Bu(t)3)9)-ACTH nonadecapeptidamide(1-19), Aib(1)-Lys(17,18,19)-ACTH nonadecapeptide(1-19), 2,4-dinitro-5-azidophenylsulfenyl-ACTH, 2-nitro-4-azidophenylsulfenyl-ACTH, 2-nitro-5-azidophenylsulfenyl-ACTH, 2-nitrophenylsulfenyl-ACTH, biotinyl-ACTH, formylmethionyl-ACTH, nitrophenylsulphenyl-ACTH, thiolglycine-ACTH, actid, β-cell tropin, Ser(1)-Lys(17,18)-6-corticotropin(1-19)-nonadecapeptide, BIM 22015, corticotropin 4-10, corticotropin-like intermediate lobe peptide, ebiratide, glutamyl-histidyl-phenylalanyl-arginyl-tryptophyl-glycyl-lysyl-prolyl-valyl-glycinamide cyclic peptide, lysyl-histidyl-phenylalanyl-arginyl-tryptophyl-glycinamide, Org 2766, and Org 31433.

α-MSH analogs include DOTA-β-Ala(3)-Nle(4)-Asp(5)-Phe(7)-Lys(10)-¹¹¹In-α-MSH(3-10), 1,4,7,10-tetraazacyclododecane 1,4,7,10-tetraacetic acid (Cys(3,4,10),D-Phe(7))α-MSH(3-13), 1,4,7,10-tetraazacyclododecane 1,4,7,10-tetraacetic acid (ReO-acetyl(Cys(3,4,10),Phe(7),Arg(7))α-MSH(3-13)), ⁶⁴Cu-DOTA-NAPamide, ⁶⁸Ga-1,4,7,10-tetraazacyclododecane 1,4,7,10-tetraacetic acid (ReO-acetyl(Cys(3,4,10),Phe(7),Arg(7))α-MSH(3-13)), acetyl-norleucyl(4)-(aspartyl(5)-histidyl(6)-phenylalanyl(7)-arginyl(8)-tryptophyl(9)-lysyl(10))cyclo-a-MSH(4-10)amide, cyclic(2-7)-peptide acetyl-norleucyl-aspartyl-histidyl-phenylalanyl-arginyl-6-methyltryptophyl-lysinamide, acetylnorleucyl-glutamyl-histidyl-phenylalanyl-arginyl-tryptophyl-glycyl-lysinamide, ACTH(6-9), a-melanotropin hydrazide, Val(13)-α-MSH(1-13), (Nle(4),Phe(p-1)7)-α-MSH(1-13)amide, Ac-Nle(4)-cyclo(Asp(5)-Phe(7)-Lys(10))-α-MSH(4-10)amide, Ac-Nle(4)-Glu(gamma-4′-hydroxyanilide)(5)-Phe(7)-α-MSH(4-10)NH₂, acetyl-Nle(4)-AsP(5)-Phe(7)-α-MSH(4-11), Ac-Nle(4)-Orn(5)-Glu(8)-α-MSH(4-11)NH₂, Trp(7)-Ala(8)-Phe(10)-α-MSH(6-11)-amide, D-tryptophyl(7)-D-phenylalanyl(10)-α-MSH(6-11)amide, Val(13)-α-MSH(8-13), Ac-Nl4(4)-Orn(5)-Phe(7)-Glu(8)-α-MSH(4-11)NH₂, ¹²⁵I-Tyr(2)-Nle(4)-Phe(7)-α-MSH, Ac-cyclo(Cys4-Cys10)-α-MSH, Nle(4)-Phe(7)-(NAPS)Trp(9)-α-MSH, α-MSH-melphalan conjugate, AP 214, diphtheria toxin-related-α-MSH fusion toxin, Enkorten, Nle(4)-Asp(5)-Phe(7)-epsilon(DOTA)Lys(11)-gadolinium α-MSH(4-11), melanotan-II, Ac-(Nle(4)-Phe(7))α-MSH(4-9)NH₂, (2-Phe-4-Nle)α-MSH, (Ala-4′-azido-3′,5′-ditritio-Phe-nor-Val)α-MSH, 12-Bct-1-N(α)-dodecanoyl-Ser-4-Nle-7-Phe-α-MSH, 13-(4-azido-Phe)α-MSH, 2-(3,5-diiodo-Tyr)α-MSH, 2-Tyr-α-MSH, 4-half-Cys-10-half-Cys-α-MSH, 4-Nle-7-Phe-α-MSH, 4-Nle-α-MSH, N(a)-Bct-1-Ser-4-Nle-7-Phe-α-MSH, N(α)-chlorotriazinylaminofluorescein-1-Ser-4-Nle-7-Phe-α-MSH, N,O-diacetyl-Ser(1)-α-MSH, N-acetyl-MSH, PT-141, Phe(7)-α-MSH(5-10), DTPA-Nle(4)-Phe(7)-α-MSH, DTPA bis((Nle(4)-Phe(7))-α-MSH), 4-fluorobenzoyl-Nle(4)-Phe(7)-α-MSH, RMI 2001, RMI 2004, RMI 2005, and SHU 9119.

β-MSH analogs include 11-Mrp-14-Nal-18-Cys-22-Asp-β-MSH(11-22)NH₂, azidoiodo-β-MSH, β-MSH(5-22), β-MSH(5-8), β-MSH(6-8), Tyr(9)-β-MSH(9-18), Gly(10)-β-MSH, and Nle(7)-β-MSH.

β-endorphin analogs include α-N-acetyl β-endorphin(1-26), β-endorphin(1-18), β-endorphin(1-27), Gly(8)-β-endorphin(1-27)amide, Leu(8)-β-endorphin(1-27)amide, β-endorphin(1-5), β-endorphin(1-9), Ac-Glu(13)-Glu(22) methylamide-β-endorphin(13-22), β-endorphin(13-31), Ac-Val(15)-Lys(19) methylamide-β-endorphin(15-19), β-endorphin(2-16), β-endorphin(2-17), β-endorphin(2-9), 6-endorphin(28-31), β-endorphin(6-21), β-endorphin(6-31), 2-nitro-4-azidophenylsulfenyl-6-endorphin, Arg(9,19,24,28,29)-β-endorphin, Cys(11,26)-Phe(27)-Gly(31)-β-endorphin, Gln(8),Gly(31)-Gly-Gly-NH₂-β-endorphin, Leu(5)-β-endorphin, Trp(27)-β-endorphin, Trp(27)-β-endorphin-2-nitrophenylsulfenyl-chloride, Tyr(18)-Trp(27)-β-endorphin, Tyr(31)-β-endorphin, β-endorphinyl-thioglycine, β-neo-endorphin, desAsn(20)-β(c)-endorphin, desenkephalin-γ-endorphin, desacetyl β-endorphin(1-27), endorphin(1-20), endorphin(20-31), Ala-2-endorphin, glutamine-8β-endorphin, immunorphin, N-acetyl-6-endorphin, N-acetyl-β-endorphin(1-8), N-dimethyl β-endorphin, Org 31258, and Org 31318.

γ-MSH analogs include tyrosyl-valyl-norleucyl-glycyl-prolyl-2′-naphthylalanyl-arginyl-tryptophyl-aspartyl-arginyl-phenylalanyl-glycinamide, γ-MSH(15-26), Lys-γ(2) MSH, and Lys-γ(3) MSH.

β-lipotropin analogs include 1-(pyroglutamic acid)-β-lipotropin, β-lipotropin(60-65), β-lipotropin(78-91), 2-alanyl-69-homoarginine-β-lipotropin(61-69), Gln(9)-β-lipotropin, and lipormone.

Growth Hormones

In certain embodiments, the peptide therapeutic is a growth hormone or an analog thereof. Such peptides include synthetic 2-CAP protein, acceleratory factor from growth hormone, cataglykin, cyclo(phenylalanyl-tryptophyl-lysyl-threonyl-4-(aminomethyl)phenylacetic acid), cyclo-(phenylalanyl-tryptophyl-lysyl-threonyl-3-(aminomethyl)phenylacetic acid), cyclo-phenylalanyl-tryptophyl-lysyl-threonyl-2-(aminomethyl)phenylacetic acid, E 117 peptide, human G119R protein, human G120R protein, gamma-lactam(11) human growth hormone(6-13), Salmo salar growth hormone type I, bovine growth hormone, human HGH-V protein, human growth hormone (HGH), B 2036, HGH 22K, pegvisomant, somatotropin 20K, somatrem, YM 17798, HGH(1-15), HGH isohormone D, HGH isohormone E, des(1-6,14)-HGH, L 117 peptide, Met-HGH, methione-equine growth hormone, N(a)-acetylsomatotropin(7-13), pregrowth hormone, S-carbamidomethyl HGH, S-carboxymethylsomatotropin, salmon growth hormone type II, somatotropin(1-134), somatotropin(1-43), somatotropin(108-129), somatotropin(134-154), somatotropin(135-191), somatotropin(176-191), somatotropin(177-191), somatotropin(31-44), somatotropin(32-38), somatotropin(32-46), somatotropin(4-15), somatotropin(44-191), somatotropin(44-91), somatotropin(54-95), somatotropin(6-13), somatotropin(73-128) glycinamide, somatotropin(75-120), somatotropin fragment(77-107), somatotropin fragment(87-124), somatotropin fragment(96-133), somatotropin sulfoxide, Ala(165)-somatotropin, di-(4-azidophenacyl) (182,189)-somatotropin, glycosylated somatotropin, iodinated somatotropin, Leu(117)-Arg(119)-Asp(122)-somatotropin, Sometribove, and Somfasepor.

Thyrotropin

In certain embodiments, the peptide therapeutic is thyrotropin or analog thereof. Such peptides include human chorionic thyrotropin protein, dansyl thyrotropin, deglycosylated thyrotropin, exophthalmos producing substance, hTSHβ-CTPα protein, β subunit thyrotropin, thyrotropin-α-lactalbumin-daunomycin conjugate, and thyrotropin-daunomycin conjugate.

Vasotocin

In certain embodiments, the peptide therapeutic is vasotocin or an analog thereof. Such peptides include 1,4,7,10-tetraazacyclododecane-N,N′,N″N′″-tetraacetic acid-Lys(8)-vasotocin, atosiban, hydrin 1, hydrin 1′, hydrin 2, (β-mercapto-β,β-cyclopentamethylenepropionic acid)-O-methyl-Tyr(2)-Thr(4)-Orn(8)-Tyr(9)-NH₂ vasotocin, 1-(3-mercaptopropanoic acid)-8-Arg-vasotocin, 1-(β-mercapto-β,β-diethylpropionic acid)-(OEt-Tyr)(2)-Orn(8)-vasotocin, 1-(β-mercaptopropanoic acid)-8-Arg-9-(4-aminorhodaminyl-Phe)-vasotocin, 1-(β-mercaptopropionic acid)-8-Arg-9-(4-aminofluoresceinyl-Phe)-vasotocin, 1-deamino-4-Lys(azidobenzoyl)-8-Arg-vasotocin, 1-deamino-7-Lys-8-Arg-vasotocin, 1-deamino-arginine-vasotocin, 1-deamino-Lys(7)-(fluorescein)-Arg(8)-vasotocin, 1-desamino-(4-azidobenzoyl)Lys(7)-Arg(8)-vasotocin, 1-desamino-fluorescein-Lys(4)-Arg(8)-vasotocin, 1-desamino-OEt-Tyr(2)-Val(4)-Orn(8)-vasotocin, 1-desaminopenicillamyl-(Tyr-OMe)(2)-Orn(8)-vasotocin, 4-Leu-vasotocin, Asu(1,6)-Arg(8)-vasotocin, β-mercaptopropionic acid-8-Lys(N-ε-4-azidobenzoyl)-vasotocin, d(CH₂)₅—O-methyl-Tyr(2)-Thr(4)-N(δ)-propionyl-Orn(8)-Tyr(9)-NH₂vasotocin, d(CH2)5-O-methyl-Tyr(2)-Thr(4)-Orn(8)-desGly-NH₂(9)-vasotocin, dansyl-Lys(8)-vasotocin, deamino-1,6-dicarba-vasotocin, and Phe(2)-Orn(8)-vasotocin.

Oxytocin

In certain embodiments, the peptide therapeutic is oxytocin or analog thereof. Such peptides include 1,6-bis(L-α,β-diaminopropionic acid) oxytocin, 1-deamino-2-Trp-4-Val-8-Orn-OT, ANTAG I, ANTAG II, ANTAG III, asvatocin, carbetocin, desglycyl-carbetocin, desleucylglycine-carbetocin, conopressin G, Conus tulipa conopressin-T, deaminodicarba-Gly-oxytocin, deaminooxytocin, Ser(4)-deaminotocinamide, Ser(4)-deaminotocinoic acid, dicarbaoxytocin, F 314, F 327, F 372, F 382, Conus villepinii gamma-conopressin-vil, Glanduphen, glumitocin, isotocin, KB 5-21, mesotocin, Phe(2)-mesotocin, mono-6-deoxy-6-oxytocinyl-6-cyclodextrin 5, N-acetyloxytocin, nacartocin, oxypressin, (1-(2-hydroxy-3-mercaptopropionic acid))-Thr(4)-Gly(7)-oxytocin, (1-desaminopenicillamine-8-α-hydroxyisocaproic acid)-oxytocin, (4-ethyl-Phe)(2)-oxytocin, (8-α-hydroxyisocaproic acid)-oxytocin, (bromoacetylamino-Phe)(2)-deamino-oxytocin, (N(4),N(4)-dimethyl-Asn)(5)-oxytocin, 1-(β-mercapto-(β,β-cyclopentamethylene)propionic acid)-Phe(Me)(2)-Thr(4)-Orn(8)-oxytocin, 1-β-mercapto-β,β-diethylpropionic acid-(3,5-dibromo-Tyr)(2)-oxytocin, 1′-(1′-methyl-4′-thiopiperidine)acetic acid-oxytocin, 1′-(1′-thio-4′-methylcyclohexane)acetic acid-oxytocin, 1,6-a-Asu-oxytocin, 1,6-N-carbonyl-Lys-oxytocin, 1-(1-mercaptocyclohexaneacetic acid)-(OEt-Tyr)(2)-Orn(8)-oxytocin, 1-(2-hydroxy-3-mercaptopropionic acid)-oxytocin, 1-(β-mercapto-β,β-cyclopentamethylenepropionic acid)-Orn(8)-oxytocin, 1-(N-maleoyl-11-aminoundecanoyl)Cys-oxytocin, 1-(N-maleoyl-Gly)Cys-oxytocin, 1-α-mercaptoacetic acid-iso-Asn(5)-oxytocin, 1-β-mercapto-β,β-cyclopentamethylenepropionic acid-oxytocin, 1-β-mercapto-β,β-diethylpropionic acid-Leu(4)-oxytocin, 1-d(CH2)5-(2-O-methyl)Tyr-Thr(4)-Orn(8)-Tyr-NH₂(9)-oxytocin, 1-deaminopenicillamine-oxytocin, 1-deaminopenicillamyl-MeO-Tyr(2)-Thr(4)-oxytocin, 1-deaminopenicillamyl-Phe(2)-Thr(4)-oxytocin, 1-desamino-(O-Et-Tyr)(2)-oxytocin, 1-desamino-thio-Gly(9)-oxytocin, 1-desaminopenicillamyl-Leu(2)-oxytocin, 1-desaminopenicillamyl-MeO-Tyr(2)-oxytocin, 1-desaminopenicillamyl-Orn(8)-oxytocin, 1-desaminopenicillamyl-Phe(2)-oxytocin, 1-desaminopenicillamyl-Thr(4)-oxytocin, 1-penicillamyl-Leu(2)-oxytocin, 1-penicillamyl-O-MeTyr(2)-oxytocin, 1-penicillamyl-Phe(2)-Thr(4)-oxytocin, 2-L-dopa-oxytocin, 2-nitro-5-azidobenzoyl-Gly-oxytocin, 3,2′-di-Me-Phe(2)-oxytocin, 4-fluoro-Phe(2)-oxytocin, 7-(azetidine-2-carboxylic acid)-oxytocin, 7-(thiazolidine-4-carboxylic acid)-oxytocin, 9 α-aminoacetonitrile-oxytocin, Asp(5)-oxytocin, β mercapto-β,β-cyclopentamethylenepropionic acid-Trp(2)-Arg(8)-oxytocin, β-cyano-Ala(5)-oxytocin, d(CH2)5(1)-Tyr(OMe)(2)-Orn(8)-oxytocin, deamino-(8-α-hydroxyisocaproic acid)-oxytocin, deamino-(N-Me-Leu)(8)-oxytocin, deamino-1-carba-oxytocin, deamino-6-carba-oxytocin, desGlyNH₂(9)-oxytocin, desamino-(4-fluoro-Phe)(2)-oxytocin, di-Ala(1,6)-oxytocin, di-Ser(1,6)-oxytocin, Glu(4)-oxytocin, Glu(NHNH₂)(4)-oxytocin, Gly(4)-oxytocin, Gly(7)-oxytocin, Gly-Lys-Arg-oxytocin, GlyNH₂(10)-oxytocin, His(4)-oxytocin, Hmp(1)-Phe(2)-Hgn(4)-Dab(Ala)(8)-oxytocin, homo-Ser(4)-oxytocin, hydroxy-Thr(4)-oxytocin, Lys(8)-oxytocin, malamidic acid(5-β)-oxytocin, MePhe(2)-oxytocin, methyl oxytocin, Mpa(1)-cyclo(Glu(4)-Lys(8))-oxytocin, N-acetyl-2-O-methyl-Tyr-oxytocin, Pen(1)-(4-MePhe)(2)-Thr(4)-Orn(8)-oxytocin, Pen(1)-Phe(2)-Thr(4)-Orn(8)-oxytocin, penicillamine(1)-oxytocin, penicillamyl(1)-Leu(4)-oxytocin, penicillamyl(1)-Thr(4)-oxytocin, Phe(2)-Orn(8)-oxytocin, Pmp(1)-Trp(2)-Cys(6)-Arg(8)-oxytocin, propionylamino-Phe-deamino-oxytocin, Sar(7)-oxytocin, Thr(4)-Gly(7)-oxytocin, Thr(4)-N-MeAla(7)-oxytocin, Thr(4)-Sar(7)-oxytocin, tri-Gly-oxytocin, Trp(2)-oxytocin, Trp(8)-oxytocin, 1-β-mercapto-(β,β-cyclopentamethylene)propionic acid)-Tyr(OMe)(2)-Orn(8)-oxytocin, oxytocinoic acid dimethylamide, phasvatocin, preproconopressin, seritocin, syntometrine, tocinamide, Ser(4)-tocinamide, tocinoic acid, Ser(4)-tocinoic acid, and VAP 259.

### Neuropeptides

In certain embodiments, the peptide therapeutic is a neuropeptide or an analog thereof. Such peptides include angiotensin, bombesin, bradykinin, calcitonin, cholecystokinins (e.g., those described herein), gastric inhibitory polypeptide, gastrin, neuropeptide Y, neurotensin, opioid peptides, vasoactive intestinal peptides (e.g., those described herein), secretin, tachykinin, and vasopressin, or an analog thereof. Other neuropeptides include (Hyp(3))Met-callatostatin, 3-phenyllactyl-leucyl-arginyl-asparaginamide, 3-phenyllactyl-phenylalanyl-lysyl-alaninamide, 4-pyroglutamyl-glycyl-arginyl-phenylalaninamide, 5-HT-moduline, achacin, achatin I, Achatina cardio-excitatory peptide 1, achetakinin, achetakinin II, beetle adipokinetic hormone, adrenoregulin, ADVGHVFLRFamide, Aedes Head Peptide I, AF1 neuropeptide, AF2 neuropeptide, alanyl-prolyl-glycyl-tryptophanamide, alanyl-tryptophyl-glutaminyl-aspartyl-leucyl-asparagyl-seryl-alanyl-tryptophanamide, aldosterone secretion inhibitory factor, allatostatin, allatostatin A1, allatostatin A2, allatotropin, α-CDCP, α-conotoxin Epl, α-endopsychosin, ameletin, AMSFYFPRMamide, anglerfish peptide YG, Antho-RPamide II, Antho-RWamide I, Antho-RWamide II, antisecretory factor, RAPYFVamide, arginyl-tyrosyl-isoleucyl-arginyl-phenylalaninamide, arginylphenylalaninamide, ARPYSFGL-NH₂, asparaginyl-glycyl-isoleucyl-tryptophyl-tyrosinamide, DYRPLQFamide, baratin, mouse Bid3 protein, rat Bid3 protein, bombinakinin M, Bombyx mori bombyxin E1 protein, Bombyx mori bombyxin f1 protein, bombyxin II, bombyxin, buccalin, buccalin B, Bombina variegate Bv8 protein, rat Bv8 protein, calfluxin, callatostatin, callisulfakinin II, cardioacceleratory peptide 2b, carnosine, rat Cd81 protein, CERE, cerebral peptide 1, cerebral peptide 2, cerebrin prohormone, cionin, conorfamide, Conus spurius conorfamide-Sr2, Conus geographus contulakin-G protein, human CORT protein, cortistatin, cortistatin 14, cortistatin 29(1-13), Tyr10-cortistatin(14), cortistatin-8, crustacean cardioactive peptide, culekinin depolarizing peptide II, curtatoxin I, curtatoxin II, curtatoxin III, cyanea-RFamide I, cyanea-RFamide II, cyanea-RFamide III, cybernins, cyclo(alanine-(1-amino-1-cyclopentane)carbonyl), cyclo(asparaginyl-threonyl-seryl-phenylalanyl-threonyl-prolyl-arginyl-leucyl), cyclo(leucylglycine), dansyl-prolyl-glutaminyl-argininamide, dansyl-prolyl-glutaminyl-arginyl-phenylalaninamide, Bombyx mori DH-PBAN precursor protein, diapause hormone, diazepam-binding inhibitor(32-86), diazepam-binding inhibitor(51-70), mouse Dlgh3 protein, rat Dlgh3 protein, doublecortin protein, drebrin E, drebrins, drosulfakinin 1, sulfoTyr(4)-Leu(7)-drosulfakinin 1, DSIP-immunoreactive peptide, human DTNA protein, mouse DTNA protein, human DTNB protein, E021, ecdysiotropin, ectodermal-neural cortex 1 protein, F24 peptide, F39 peptide, FMRFamide, Ala2-YAGFMKKKFMRFamide, aspartyl-prolyl-lysyl-glutamyl-aspartyl-phenylalanyl-methyonyl-arginyl-phenylalanylamide, desamino-tyrosyl-phenylalanyl-norleucyl-arginyl-phenylalaninamide, GAHKNYLRFamide, KSAYMRFamide, Met-enkephalin-FMRFa chimeric peptide, neuropeptide DF2, SDNFMRFamide, SDPNFLRFamide, seryl-lysyl-prolyl-tyrosyl-methionyl-arginyl-phenylalaninamide, threonyl-prolyl-alanyl-glutamyl-aspartyl-phenylalnyl-methionyl-arginyl-phenylalanylamide, tryptophyl-norleucyl-arginyl-phenylalaninamide, FPRF amide, frequenin calcium sensor proteins, fulicin, fulyal, galanin-like peptide, gastrin-releasing peptide, gastrin releasing peptide(1-16), gastrin releasing peptide(14-27), Phe(25)-gastrin releasing peptide(18-27), Ala(24)-gastrin releasing peptide(20-26), Ala(6)-gastrin-releasing peptide 10, mouse Gcm1 protein, GDPFLRFamide, GFSamide, GLTPNMNSLFFamide, hypertrehalosemic hormone II, hypertrehalosemic peptide I, glycine-extended anglerfish peptide YG, glycyl-aminoisobutyryl-alanyl-aspartate, glycyl-leucyl-leucyl-aspartyl-leucyl-lysine, glycyl-tyrosyl-isoleucyl-arginyl-phenylalaninamide, GMM2, mouse Gnb2-rs1 protein, Asteroidea GSS protein, H-tryptophyl-arginyl-glutamyl-methionyl-seryl-valyl-tryptophylamide, WWamide-3, H-WWamide-1, Helix lucorum HCS1 protein, Helix lucorum HCS2 protein, Helix lucorum HDS2 protein, head activator peptide, hydra Arg(1)-Phe(5)-head activator peptide, hippocampal cholinergic neurostimulating peptide, histidine rich basic peptide (Aplysia), histidine rich basic peptide precursor (Aplysia), histidyl(7)-corazonin, holokinin 1, holokinin 2, Hydra Hym-176 protein, Hym-355 neuropeptide, hypertrehalosemic hormone, hypertrehalosemic neuropeptide, hypocretin-2-saporin conjugate, insulin-related neuropeptide, insulin-related peptide I, mouse intermedin protein, rat intermedin protein, KPSFVRFamide, Led OVM myotropic peptide, Led-CC-1 peptide, Led-CC-11 peptide, Led-MNP-I peptide, leucokinin 1, leucokinin 2, leucokinin 3, leucokinin 4, leucokinin I, leucomyosuppressin, leucosulfakinin, leucosulfakinin II, leucyl-prolyl-prolyl-glycyl-prolyl-leucyl-prolyl-arginyl-prolinamide, LFRFamide, Lom-AG-myotropin, 6-Phe-Ala(0)-Lom-MT-II, LUQIN, mouse Lynx1 protein, mouse Lynx2 protein, mandibular organ-inhibiting hormone 1, mandibular organ-inhibiting hormone 2, rat manserin, Mas-MIP I peptide, Mas-MIP II peptide, melanocyte-stimulating hormone, Met-callatostatin, desGly-desPro-Met-callatostatin, Hyp(2)-Met-callatostatin, methionine sulfoxide NPY, motilin, mu-agatoxin I, myokinin, myomodulin, myotropin II, N-acetyl-pituitary adenylate cyclase activity peptide 27, N-N-(4-azido-tetrafluorobenzoyl)-biocytinyloxyl-succinimide, neomyosuppressin, neurexophilin, human Neurod2 protein, mouse Neurod2 protein, rat Neurod2 protein, chicken NeuroM protein, neuromedin N-125, neuromedin S, neuromedin U, neuromedin U 25, neuromedin U 8, neuromedin U 9, neuronal membrane cytoskeletal protein 4.1, neuropeptide B, neuropeptide F, human neuropeptide S, neuropeptide SF, neuropeptide VF, mouse neuropeptide W, rat neuropeptide W, neurophysin, human AVP protein, VLDV neurophysin, neuroserpin, neurostenin, neurotensin-like immunoreactivity, NocII neuropeptide, NocIII neuropeptide, norbin, human NPVF protein, human NPW protein, rat Nrn protein, human NRN1 protein, orcokinin, Ala(13)-orcokinin, Ser(9)-orcokinin, Val(13)-orcokinin, orexins, mouse Pacsin1 protein, human PCDHA4 protein, mouse Pcdha4 protein, rat Pcdha4 protein, human PCDHA6 protein, mouse Pcdha6 protein, rat Pcdha6 protein, human PCLO protein, mouse Pclo protein, rat Pclo protein, mouse Pcp2 protein, human PCSK1N protein, Pea-CAH-II neuropeptide, Pea-PK-1, Pea-PK-2, pedal peptide, Pej-PDH-I peptide, Pej-PDH-II peptide, Penaeus japonicus Pej-SGP-IV protein, peptide I (Aplysia), peptide II (Aplysia), peptide tyrosine phenylalanine, peptide V, periplaneta-DP, periplanetin CC-1, perisulfakinin, periviscerokinin, periviscerokinin-2, pev-myomodulin, PFR(Tic)amide, phenylalanyl-aspartyl-alanyl-phenylalanyl-threonyl-threonyl-glycyl-phenylalanylamide, phenylalanyl-threonyl-arginyl-phenylalaninamide, Helicoverpa zea pheromone biosynthesis activating neuropeptide, pheromone biosynthesis-activating neuropeptide II, pheromone-biosynthesis-activating neuropeptide I, Pseudaletia pheromonotropin, phyllomedusin, pineal peptide E5, pituitary adenylate cyclase-activating polypeptide, human ADCYAP1 protein, mouse Adcyap1 protein, rat Adcyap1 protein, Arg(15,20,21)-Leu(17)-PACAP-Gly-Lys-Arg-NH₂, P66 peptide, PACAP-related peptide, pig pituitary adenylate cyclase-activating-peptide(1-38), pituitary adenylate-cyclase-activating peptide(6-27), pituitary adenylate-cyclase-activating-peptide(6-38), plaferon, PnTx4-3, Polyorchis penicillatus pol-RFamide neuropeptides protein, postural asymmetry factor, human prepro-26RFa protein, pro-aldosterone secretion inhibitory factor, proctolin, human PROK2 protein, mouse Prok2 protein, rat prokineticin 2, prolyl-phenylalanyl-arginyl-phenylalaninamide, mouse protocadherin β16, mouse Ptx3 homeodomain protein, pQDPFLRFamide, pyroglutamyl-leucyl-asparaginyl-phenylalanyl-seryl-threonyl-glycyl-tryptophanamide, pyroglutamyl-leucyl-glycyl-arginyl-phenylalaninamide, pyroglutamyl-leucyl-threonyl-phenylalanyl-threonyl-prolyl-asparaginyl-tryptophyl-glycyl-serinamide, pyroglutamyl-tryptophyl-leucyl-lysyl-glycyl-arginyl-phenylalaninamide, pyrokinin, human PYY2 protein, mouse Rac1 protein, RFamide peptide, mouse Rgs19ip1 protein, rat Rgs19ip1 protein, mouse Rgs19ip3 protein, human RHEB protein, mouse Rheb protein, rat Rheb protein, SADPNFLRFamide, SALMFamide 1, SALMFamide 2, SchistoFLRFamide, schistomyotropin-1, schistomyotropin-2, schistostatin, human SCN11A protein, mouse Scn11a protein, rat Scn11a protein, scorpion toxin AaH III, scorpion toxin AaH IT1, scorpion toxin AaH IT2, scorpion toxin AaH IT4, SDPFLRFamide, SDRNFLRFamide, secretoneurin, SEEPLY peptide, SEPYLRFamide, human SHC3 protein, mouse SHC3 protein, rat SHC3 protein, small cardioactive peptide A, small cardioactive peptide B, sodium-influx-stimulating peptide, type I sodium channel SP19 peptide, stannin, stichopin, SynGAP protein p135, rat TAFA5 peptide, Mouse TAT4 peptide, Tem-HrTH, rat TFF3 protein, threonyl-lysyl-glutaminyl-glutamyl-leucyl-glutamic acid, TNRNFLRFamide, triakontatetraneuropeptide, tuberoinfundibular peptide 39, Tx1 neurotoxin, Tx2 neurotoxin, Tx3 neurotoxin, urechistachykinin I, urechistachykinin II, Vaxl protein, VD1-RPD2 neuropeptide α1, VD1-RPD2 neuropeptide α2, VD1-RPD2 neuropeptide β, VD1-RPD2 neuropeptide prepro, VFQNQFKGIQGRF, VGF peptide, VGF protein, VPNDWAHFRGSWamide, Y-head activator-head activator bipeptide, and YFAFPRQamide.

Angiotensin

The peptide therapeutic can be angiotensin, angiotensinogen, or analog thereof. Exemplary angiotensin analogs include angiotensin A, angiotensin I, angiotensin I(1-7), angiotensin I(1-9), (β-(4-pyridyl-1-oxide)-Ala4)-angiotensin I, Arg10-angiotensin I, Asn1-Va15-Gly9-angiotensin 1, Asn1-Val5-His9-angiotensin I, desAsp1-angiotensin I, desLeu10-angiotensin I, Ile5-angiotensin I, Pro11-Ala12-angiotensin I, Sar1-angiotensin I, Sar1-(S-Me)Cys8-angiotensin, Sar1-Ala7-angiotensin I, Sar1-Ile5-α-Me-Ala(7)-angiotensin I, Sar(1)-Val(5)-N-Me-Ala(7)-angiotensin I, Sar(1,7)-Val(5)-angiotensin I, Val(5)-angiotensin I, Lys(11)-angiotensinogen(3-11), angiotensinogen (1-13), angiotensinogen(1-14), angiotensinogen(6-13), H 142, H 189, rat proangiotensin-12, D-Pro7-Ang-(1-7), des-angiotensin I renin substrate, des-aspartate-angiotensin I, angiotensin II, (2,4-dinitrophenyl)aminohexanoylangiotensin II, (6-biotinylamido)hexanoylangiotensin II, 7-Ala-angiotensin(1-7), Ile8-angiotensin I, angiotensin II(1-6), angiotensin II(1-7), angiotensin II(1-8), angiotensin II(2-7), angiotensin II(3-7), Phe4-angiotensin II(3-7), angiotensin II amide, Sar1-angiotensin II amide(1-7), (α-Me-Tyr)(4)-angiotensin II, 1-(4-azidobenzoic acid)-Ile8-angiotensin II, 1-hydantoic acid-Val5-Ala8-angiotensin II, 1-malyl-angiotensin II, 1-malyl-Leu(8)-angiotensin II, 1-N(4)-dimethyl-Asp-angiotensin II, 1-N-Suc-Val(5)-phenyl-Gly(8)-angiotensin II, 4-amino-Phe(6)-angiotensin II, Aib(1)-angiotensin II, Ala(7)-angiotensin II, Ala(7)-N-Me-Phe(8)-angiotensin II, Ala(8)-angiotensin II, Ala-Pro-Gly-angiotensin II, Asn(1)-Val(5)-angiotensin II, Asp(1)-Val(5)-angiotensin II, cyclo(Sar(1)-Cys(3)-Mpt(5))-angiotensin II, cyclo(Sar(1)-HCys(3)-Mpt(5))-angiotensin II, Cys(1,8)-angiotensin II, Cys(8)-angiotensin II, desAsp(1)-(N(2)-(3-carboxy-1-oxypropyl)-Arg)-Ile(5)-angiotensin II, desAsp(1)-desArg(2)-Ile(5)-angiotensin II, desAsp(1)-Me-Tyr(4)-angiotensin II, desPhe(8)-angiotensin II, His(2)-Ile(5)-angiotensin II, Ile(5)-MePro(7)-angiotensin II, Ile(8)-angiotensin II, iodo-Sar(1)-Tdf(8)-angiotensin II, Leu(8)-angiotensin II, Lys(2)-angiotensin II, N,N-dimethyl-Gly(1)-Ile(5)-angiotensin II, N-(1-octanoyl)-Ile(5)-Leu(8)-angiotensin II, N-a-(N-fluoresceinthiocarbamoyl)-Asp(1)-Ile(5)-angiotensin II, N-Me-Phe(8)-angiotensin II, Phe(4)-angiotensin II, Sar(1)-angiotensin II, Sar(1)-4-azido-Phe(8)-angiotensin II, Sar(1)-Ala(7)-angiotensin II, Sar(1)-Ala(7)-N-Me-Phe(8)-angiotensin II, Sar(1)-Car(4)-angiotensin II, Sar(1)-Car(8)-angiotensin II, Sar(1)-Gly(8)-angiotensin II, Sar(1)-hydroxy-Pro(7)-N-Me-Phe(8)-angiotensin II, Sar(1)-Ile(4)-Ile(8)-angiotensin II, Sar(1)-Ile(5)-angiotensin II, Sar(1)-Ile(5)-(4-azido)Phe(8)-angiotensin II, Sar(1)-Ile(5)-Gly(8)-angiotensin II, Sar(1)-Me-Thr(8)-angiotensin II, Sar(1)-Me-Tyr(4)-angiotensin II, Sar(1)-N-Me-Phe(8)-angiotensin II, Sar(1)-Phe(4)-angiotensin II, Sar(1)-Phe(4)-Ile(8)-angiotensin II, Sar(1)-Phe(8)-angiotensin II, Sar(1)-S-Me-Cys(8)-angiotensin II, Sar(1)-Ser(8)-angiotensin II, Sar(1)-Thr(8)-angiotensin II, Sar(1)-Val(5)-angiotensin II, Sar(1,7)-angiotensin II, Val(5)-Trp(8)-angiotensin II, desPhe(6)-angiotensin IV, 2-nitro-5-azidobenzoyl-angiotensin, Sar-Arg-Val-Tyr-Val-His-Pro-(2′,3′,4′,5′,6′-pentabromo)-pheangiotensin-II, arlalasin, cyclo(3,5)-(Sar(1)-Lys(3)-Glu(5)-Ile(8))ANG II, DD 3487, divalanal-angiotensin IV, divalinal-angiotensin IV, Ex 169, norLeu3-A(1-7), pentasarcosyl angiotensin II, phosphotyrosylangiotensin II, pseudoangiotensin II, sarleucin, triprolyl angiotensin II, Val(5)-Ala(8)-angiotensin II, Angiotensin III, 1-desarginine-angiotensin III, 4-Val-7-Trp-angiotensin III, 5-Ile-angiotensin III, Ile(7)-angiotensin III, Ile(8)-angiotensin III, Sar(1)-Ile(7)-angiotensin III, angiotensin pentapeptide, and crinia-angiotensin.

Bombesin

The peptide therapeutic can be bomesin or analog thereof. Exemparly bombesin analogs include ¹⁸F-FB-BBN-RGD, (phenylalanyl(6)-alanyl(11)-phenylalanyl(13)-Nle(14))Bn(6-14), ¹⁷⁷Lu-DOTA-8-aminooctanoylbombesin(7-14)-amide, ⁶⁴Cu-Pro1-Tyr4-DOTA-bombesin(1-14), ⁸⁶Y-Pro(1)-Tyr(4)-DOTA-bombesin(1-14), ^{99m}Tc-EDDA-HYNIC-(Lys 3)bombesin, acetyl-bombesin(7-14), AN 215, BIM 189, BIM 26226 Phe6-bombesin(6-13) methyl ester, Phe6-bombesin(6-13) propylamide, Tyr6-bombesin(6-13) methyl ester, Phe6-bombesin(6-13) ethylamide, D-Phe6-Leu13-ψ(CH₂NH)-Phe(14)-bombesin(6-14), D-Trp6-Leu13-ψ(CH₂NH)-Leu(14)-bombesin(6-14), Na16-Psi(13,14)-Phe14-bombesin(6-14), Phe6-Leu13-ψCCH₂NH)-Leu14-bombesin(6-14), Thr6-Leu13-ψ(CH₂NH)-Met14-bombesin(6-14), Tpi6-Leu13-ψ(CH₂NH)-Leu14-bombesin(6-14), Trp6-Leu13-ψ(CH₂NH)-Phe14-bombesin(6-14), Phe6-desMet14-bombesin(6-14)-ethylamide, Phe6-Cpa14-ψ(13-14)-bombesin(6-14)-NH₂, Phe6-Gln7-ψ(CHCH)Leu14-bombesin(6-14)-NH₂, bombesin(7-14), bombesin nonapeptide, Hca6-Leu13-ψ(CH₂N)-Tac14-bombesin(6-14), Tpi6-Leu13-ψ(CH₂N)-Tpi14-bombesin(6-14), N-(3-iodobenzoyl)glutamyl-desMet14-bombesin(8-13)-NH₂, Leu13-ψ(CH₂NH)-Leu14-bombesin, Lys3-bombesin, Phe12-Leu14-bombesin, Phe12-bombesin, ψ(13,14)-Leu14-bombesin, Tyr4-Phe12-bombesin, Tyr4-bombesin, bombestatin, Cu-DOTA-Lys3-bombesin, DOTA-PEG(4)-bombesin(7-14), DTPA-Prol-Tyr4-bombesin, human probombesin C-terminal peptide, JMV 1458, neuromedin C, Ala1-Leu9-ψ(CH₂NH)-Leu10-neuromedin C, Leu9-ψ(CH₂NH)-Leu10-neuromedin C, Re(H₂O)(CO)₃-diaminopropionic acid-SSS-bombesin(7-14)NH₂, ^{99m}Tc-demobesin 1, ^{99m}Tc-HYNIC-bombesin, ^{99m}Tc-Leu13-bombesin

Bradykinins

The peptide therapeutic can be bradykinin or an analog thereof. Exemplary bradykinin analogs include Ac-Orn-(Oic2,α-MePhe5,D-βNal7,Ile8)desArg9-bradykinin, Amolops loloensis amolopkinin protein, arginyl-prolyl-prolyl-glycyl-phenylalanyl-seryl-(3S)(amino)-5-(carbonylmethyl)-2,3-dihydro-1,5-benzothiazepin-4(5H)-one-arginine, B 3852, B 4146, B 4162, B 9340, B 9430, B 9858, B-9958, bradykinin(1-5), bradykinin(2-9), bradykinin(7-9), bradykinin chloromethyl ketone, (Thi-Ala)(5,8)-Phe(7)-bradykinin, 1-adamantanecarboxylic acid-Arg(0)-Hyp(3)-Thi(5,8)-Phe(7)-bradykinin, 4-iodo-Phe(5)-bradykinin, 4-nitro-Phe(5)-bradykinin, acetyl-Arg-Hyp(3)-Phe(7)-Leu(8)-bradykinin, Ala(1)-Thr(6)-bradykinin, Arg(0)-Trp(5)-Leu(8)-bradykinin, Arg-Hyp(3)-Phe(7)-Leu(8)-bradykinin, beta-homo-Pro(7)-bradykinin, cyclo(N-(epsilon-1)-Lys(1)-Gly(6))-bradykinin, cyclo-N(epsilon)-Lys-bradykinin, desArg(1)-bradykinin, desArg(9)-bradykinin, desPhe(8)-desArg(9)-bradykinin, desPro(3)-bradykinin, desArg(9)-Hyp(3)-bradykinin, Gly(6)-bradykinin, Gly-Leu-Met-Lysbradykinin, hydroxy-Pro(3)-bradykinin, Hyp(3)-Thi(5)-Tic(7)-Oic(8)-desArg(9)-bradykinin, Hyp(3)-TyrMe(8)-bradykinin, Ile(10)-Tyr(11)-bradykinin, Leu(8)-bradykinin, Leu(8)-desArg(9)-bradykinin, Leu-Ile-Ser-bradykinin, Met(1,5)-bradykinin, Met-Ile-Ser-bradykinin, Met-Lys-bradykinin, Phe(8)-ψ-CH₂NH-Arg(9)-bradykinin, Sar-(D-Phe(8))-desArg(9)-bradykinin, Thr(6)-bradykinin, Trp(5)-bradykinin, Trp(5)-Leu(8)-bradykinin, Tyr(8)-bradykinin, Tyr(Me)8-bradykinin, Tyr-bradykinin, Tyr-Arg-(Hyp(3)-Phe(7)-Leu(8))-bradykinin, Met-Ile-Ser-bradykinin-Leu, bradykininogen, bromobradykinin, cyclobradykinin, dansylbradykinin, rat desArg(11)-T-kinin, galanin(1-13)-bradykinin(2-9)-amide, desArg(10)-HOE 140, HOE 890307, HOE k86-4321, icatibant, JMV 1116, JMV 1465, JMV 1609, MAP4-RPPGF, methionyl-lysyl-bradykinin-serine, N-bromoacetylbradykinin, NPC 16731, NPC 17761, NPC 567, NPC 573, ornitho-kinin, Pam-Gly(-1)-Lys(0)-Arg(1)-Pro(2)-Pro(3)-Gly(4)-Phe(5)-Ser(6)-Pro(7)-Phe(8)-Arg(9)-OH, para-iodophenyl HOE 140, Protopolybia exigua protopolybiakinin-1, Protopolybia exigua protopolybiakinin-2, R 715, RMP 7, S 16118, Phyllomedusa bicolor sapo, T-kinin, Tyr-Lys-bradykinin, vespakinin-M, and vespakinin-X.

Calcitonin

The peptide therapeutic can be calcitonin, calcitonin gene-related peptide (CGRP), or an analog thereof. Such peptides include calcitonin gene-related peptide I, calcitonin gene-related peptide II, human calcitonin(9-32), (4-azidobenzoyl)-Arg(11,18)-Lys(14)-calcitonin, Arg-3-nitrophenylazido-Lys-calcitonin, Hse(32)-amide eel calcitonin, human desPhe(16)-calcitonin, human Gly(8)-calcitonin, human Val(8)-calcitonin, salmon Arg(11,18)-Lys(14)-calcitonin, salmon desLeu(16)-calcitonin, salmon desSer(2)-calcitonin, salmon Gly(8)-calcitonin, salmon Gly(8)-Ala(16)-desLeu(19)-calcitonin, salmon Gly(8)-desLeu(16)-Arg(24)-calcitonin, sardine calcitonin, CCP II, elcatonin, katacalcin, preprocalcitonin, procalcitonin, RG 12851, salmon calcitonin, salmon calcitonin(8-32), SB 205614, and t-butyloxycarbonyl-cyclo(cysteinyl-t-butylseryl-asparaginyl-leucyl-t-butylseryl-t-butylthreonyl-cysteinyl)-valyl- leucyl-glycine, ethylamide-Cys(2,7)-alpha-CGRP, CGRP(1-7), CGRP(19-37), CGRP(32-37), t-butyl-Cys(18)-CGRP(19-37), CGRP(23-37), CGRP(27-37), CGRP(28-37), CGRP(8-37), propionyllysyl(24)-CGRP(8-37), (acetylmethoxy)cysteinyl(2,7)-CGRP, Asu(2,7)-CGRP, prepro-CGRP, and pro-CGRP.

Delta Sleep-inducing Peptide

In certain embodiments, the peptide therapeutic is delta sleep-inducing peptide or an analog thereof. Such peptides include delta sleep-inducing peptide(1-4), delta sleep-inducing peptide(1-6), delta sleep-inducing peptide phosphate, isoAsp(5)-delta sleep-inducing peptide, N-Tyr-delta sleep-inducing peptide, omega-aminocaprylyl-delta sleep-inducing peptide, Trp(1)-delta sleep-inducing peptide, Ala(4)-delta-sleep-inducing peptide amine, cyclo-Gly-delta-sleep-inducing peptide, deltaran, and Deltran.

Galanin

The peptide therapeutic may be galanin or an analog thereof. Such peptide include gal(1-14)-(Abu 8) scy-I, human GAL protein, rat Gal protein, galanin(1-11)-amide, galanin(1-13)-spantide amide, galanin(1-15), Thr(6)-Trp(8,9)-galanin(1-15)-15-01, galanin(1-16), Sar(1)-Ala(12)-galanin(1-16)-amide, galanin(1-19), galanin(16-29), galanin(17-30), galanin(2-11)-amide, galanin(3-30), galanin message-associated peptide, galanin(1-14)-(aminobutyrate)SCY-I, galanin(1-13)-bradykinin-(2-9)-amide, galparan, M38 peptide, and M40, and transportan.

Gastric Inhibitory Polypeptide

The peptide therapeutic may be gastric inhibitory polypeptide (GIP) or an analog thereof. GIP analogs include GIP(1-14), GIP(1-39), GIP(1-42), GIP(3-42), GIP(7-42), ε-palmitoyl-Lys16-GIP, ε-palmitoyl-Lys37-GIP,Hyp3-GIP, Hyp3-palmitoylLys16-GIP, N-pyroglutamyl-ε-palmitoyllysyl(16)-GIP, N-pyroglutamyl-ε-palmitoyllysyl(37)-GIP, Pro(3)-GIP, and N-AcGIP(LysPAL37)).

Gastrin

The peptide therapeutic can be gastrin or an analog thereof. Exemplary gastrin analogs include 3-(3-iodo-4-hydroxyphenyl)propionyl(Leu15)gastrin-(5-17), APH070, big gastrin, dansylgastrin, desglugastrin, diiodograstrin, DM-gastrin, DP-gastrin, E1-INT, desulfonated-gastrin(2-17), Leu(15)-gastrin(2-17)-Gly, gastrin(4-17), gastrin 17, gastrin 34(1-14)-IgG hinge protein-gastrin 17(2-17), gastrin desulfonated, Leu15-gastrin heptadecapeptide, methoxine(15)-gastrin heptadecapeptide, Nle15-gastrin heptadecapeptide, gastrin hexapeptide, gastrin I, gastrin immunogen, Asp11-gastrin, Asp11-Phe12-gastrin, Phe12-gastrin, Glu-octagastrin, glycine-extended gastrin 17, IgG hinge protein-gastrin 17(2-17), iodogastrin, JMV 209, JMV 97, minigastrin, desTrp1-Asp5-Leu12-minigastrin, desTrp1-Nle12-minigastrin, nanogastrin, preprogastrin, progastrin(1-35), ^{99m}Tc-HYNIC(0)-Glu1-desGlu(2-6)-minigastrin, and tyrosyl-glycyl-tryptophyl-methionyl-aspartyl-phenylalanyl-glycine).

Neuropeptide Y

In certain embodiments, the peptide therapeutic is a neuropeptide Y or an analog thereof. Such peptides include bis(31-31′)((Cys(31),Nva(34))NPY(27-36)-NH₂), D-Trp(34)-neuropeptide Y, desamido-neuropeptide Y, EXBP 68, galanin-NPY chimeric peptide M32, galanin-NPY chimeric peptide M88, N(α)-((biotinylamido)hexanoyl)-neuropeptide Y, N(α)-biotinyl-neuropeptide Y, neuropeptide Y(1-27), neuropeptide Y(1-30), neuropeptide Y(13-36), neuropeptide Y(16-36), neuropeptide Y(17-36), neuropeptide Y(18-36), neuropeptide Y(2-36), neuropeptide Y(20-36), N-acetyl-(Leu(28,31))-neuropeptide Y(24-36) amide, Ac-(Leu(28,31))-neuropeptide Y(24-36), neuropeptide Y(26-36), desasparaginyl(29),tryptophyl(28,32)-neuropeptide Y(27-36), Tyr(27,36)-Thr(32)-neuropeptide Y(27-36), neuropeptide Y(3-36), bis(31-31′)(Cys(31)-Trp(32)-Nva(34))neuropeptide Y(31-36), Cys-neuropeptide Y(32-36) amide, cyclic (Lys(28)-Glu(32))-neuropeptide Y(Ac-25-36), neuropeptide Y C-terminal flanking peptide, N-acetyl-(Leu(17,28,31)Gln(19)Ala(20,23))-neuropeptide Y(13-36)amide, Ahx(5-17)-neuropeptide Y, Ahx(5-24),γ-Glu(2)-ε-Lys(30)-neuropeptide Y, desAA(7-24)-(Ala(5)-Aoc(6)-Trp(32))-neuropeptide Y, Leu(31)-Pro(34)-neuropeptide Y, N(ε,7)-biotinyl-Lys(7)-neuropeptide Y, Nle(4)-neuropeptide Y, Pro(34)-neuropeptide Y, Trp(32)-neuropeptide Y, NPY(28-36), fish pancreatic peptide Y, preproneuropeptide Y, proneuropeptide Y, propionyl-neuropeptide Y, PYX 1, PYX 2, WRYamide, and YM 42454.

Neurotensin

The peptide therapeutic may be neurotensin or analog thereof. Exemplary neurotensin analogs include (VIP-neurotensin) hybrid antagonist, acetylneurotensin(8-13), JMV 1193, KK13 peptide, neuromedin N, neuromedin N precursor, neurotensin(1-10), neurotensin(1-11), neurotensin(1-13), neurotensin(1-6), neurotensin(1-8), neurotensin(8-13), Asp(12)-neurotensin(8-13), Asp(13)-neurotensin(8-13), Lys(8)-neurotensin(8-13), N-methyl-Arg(8)-Lys(9)-neo-Trp(11)-neo-Leu(12)-neurotensin(8-13), neurotensin(9-13), neurotensin 69L, Arg(9)-neurotensin, azidobenzoyl-Lys(6)-Trp(11)-neurotensin, Gln(4)-neurotensin, iodo-Tyr(11)-neurotensin, iodo-Tyr(3)-neurotensin, N-α-(fluoresceinylthiocarbamyl)glutamyl(1)-neurotensin, Phe(11)-neurotensin, Ser(7)-neurotensin, Trp(11)-neurotensin, Tyr(11)-neurotensin, rat NT77, PD 149163, proneurotensin, stearyl-Nle(17)-neurotensin(6-11)VIP(7-28), ^{99m}Tc-NT-XI, TJN 950, and vasoactive intestinal peptide-neurotensin hybrid.

Opioid Peptides

The peptide therapeutic can be an opioid peptide. Exemplary opioid peptides include dynorphins, endorphins, enkephalins, and nociceptins, or an analog thereof. Other opioids include (F-G)NOC oFQ(1-13)-NH₂, (Nphe(1),Arg(14),Lys(15))N—OFQ NH₂, acetyl-arginyl-phenylalanyl-tryptophyl-isoleucyl-asparaginyl-lysine, cyclo(Cys(10,14))nociceptin(1-13) amide, cyclo(Cys(7,14))nociceptin(1-13) amide, cyclo(tyrosyl-ornithyl-phenylalanyl-aspartamide), deltorphin, deltorphin I, deltorphin II, Ala(2)-deltorphin I, Ala(2)-deltorphin II, Ile(5,6)-deltorphin II, Ala(2)-Cys(4)-deltorphin, Leu(2)-deltorphin, dermorphin, dermorphin-saporin, endomorphin 1, endomorphin 2, Dmt(1)-2-Nal(4)-endomorphin-1, Pro(2)-endomorphin-1, 1-Nal(4)-endomorphin-2, Dmt(1)-2-Nal(4)-endomorphin-2, prolyl(2)-endomorphin-2, FE 200665, FE 200666, nocistatin, opiomelanin, prepro-orphanin FQ(154-181), prepro-orphanin FQ(160-187), proorphanin, Tyr-W-MIF-1, and UFP-102.

Dynorphin and dynorphin analogs include 3-nitro-2-pyridinesulfenyl dynorphin derivative, arodyn, dynorphin(1-11), Ala(2)-dynorphin(1-11), Pro(10)-dynorphin(1-11), dynorphin(1-12), dynorphin(1-13), dynorphin(1-24), dynorphin(1-32), dynorphin(1-8), dynorphin(2-17), dynorphin(3-13), dynorphin A, dynorphin A(1-11)-amide, Pro(3)-dynorphin A(1-11)-amide, Ala(2)-Trp(4)-dynorphin A(1-13), Asn(2)-Trp(4)-dynorphin A(1-13), N-Met-Tyr(1)-dynorphin A(1-13), Tyr(14)-Leu(15)-Phe(16)-Asn(17)-Gly(18)-Pro(19)-dynorphin A(1-13), N-Met-Tyr(1)-dynorphin A(1-13)amide, dynorphin A(1-9), dynorphin A(2-12), dynorphin A(6-12), 4-aminocyclohexylcarbonyl(2-3)-dynorphin A amide(1-13), biocytin(13)-dynorphin A amide(1-13), Cys(2)-Cys(5)-MeArg(7)-Leu(8)-dynorphin A amide(1-9), N-methyl-Tyr(1)-4-nitro-Phe(4)-N-methyl-Arg(7)-Leu(8)-dynorphin A ethylamide(1-8), MeTyr(1)-MeArg(7)-Leu(8)-dynorphin A ethylamide(1-9), Ala(2)-desGly(3)-dynorphin A, desTyr(1)-desTrp(14)-desAsp(15)-desAsn(16)-desGlu(17)-dynorphin A, desTyr(1)-Gly(2)-dynorphin A, Dmt(1)-Tic(2)-dynorphin A, Nα-benzylTyr(1)-cyclo(Asp(5)-Dap(8))-dynorphin A-(1-11)-NH₂, cyclo(N,5)(Trp(3)-Trp(4)-Glu(5))-dynorphin A-(1-11)amide, dynorphin amide(1-10), Ala(2)-(5-F-Phe)(4)-dynorphin amide(1-13), dynorphin B, dynorphin B(1-13), dynorphin B(1-29), dynorphin B(5-9), dynorphin bridge peptide, E 2078, PL017-dynorphin A(6-17), pre-prodynorphin, and rimorphin.

Endorphins and endorphin analogs include adrenal opioid peptide E, α-endorphin, desTyr(1)-α-endorphin, α-neoendorphin, amidorphin, amidorphin(8-26), β-casomorphin 4027, human β-casomorphin 8 protein, β-casomorphin 11, β-casomorphin 4, β-casomorphin 5, β-casomorphin 7, β-casomorphin I, desTyr-β-casomorphin, β-casomorphin-4-nitroanilide, β-casomorphins, β-endorphin and analogs thereof (e.g., those described herein), Trp(3)-casomorphin, circulating opioid factor, CM 2-3, cytochrophin-4, δ-endorphin, humoral endorphin, desTyr(1)-γ-endorphin, historphin, kyotorphin, lysyl-lysyl-glycyl-glutamic acid, morphiceptin, Dmt(1)-Nal(3)-morphiceptin, N-Me-Phe(3)-morphiceptin, N-Me-Phe(3)-Pro(4)-morphiceptin, Val(4)-morphiceptin, N-acetyl-α-endorphin, N-acetyl-γ-endorphin, neo-kyotorphin, neokyotorphin(1-4), rimorphin, and rat Tyr-cav.

Enkephalins and enkephalin analogs include 3-carboxysalsolinol-Gly-Gly-Phe-Leu, Ala(2)-MePhe(4)-Gly(5)-enkephalin, Ala(2)-MePhe(4)-GlyNH₂(5)-enkephalin, biphalin, BW 942C, cyclo(lysyl-tyrosyl-methionyl-glycyl-phenylalanyl-prolyl), cysteinyldopaenkephalin, D-Ala2-D-Nle5-enkephalin-Arg-Phe, EK 209, enkelytin, Ala(2)-Nle(5)-enkephalin sulfonic acid, 2,6-dimethyl-Tyr(1)-Pen(2,5)-enkephalin, Ala(2)-cysteamine(5)-enkephalin, Ala(2)-N-pentyl-PheNH(4)-enkephalin, Ala(2)-N-Phe(4)-Gly-ol-enkephalin, Ala(2)-O-benzyl-Ser(5)-enkephalin, Ala(2)-ProNH₂(5)-enkephalin, Ala(2)-Val(5)-enkephalin, Ala(2)-ValNH₂(5)-enkephalin, AlaNH₂(5)-enkephalin, alanyl(2)-N-(2-(dimethylamino)ethyl)-N(α)-methyl-phenylalaninamide(4)-enkephalin, Cys(2)-CysNH₂(5)-enkephalin, Cys(2)-Pen(5)-enkephalin, dehydro-Ala(3)-enkephalin, dalargin, leucine enkephalin, leucine-2-alanine enkephalin (DADLE), 2-Ala-5-N-Et-Leu-enkephalinamide, azoenkephalin, destyrosyl-dalargin, Ala(2)-cyclopropyl-Phe(4)-enkephalin-Leu methyl ester, (Ala(2)-Cl-Phe(4))-enkephalin-Leu, Ala(2)-(cyclopropyl-Phe)(4)-enkephalin-Leu, Ala(2)-Arg(6)-enkephalin-Leu, Ala(2)-Cys(6)-enkephalin-Leu, Ala(2)-cystamine-dimer-enkephalin-Leu, Ala(2)-Me-Phe(4)-enkephalin-Leu, Ala(2)-melphalan methyl ester-enkephalin-Leu, Ala(2)-Ser(6)-enkephalin-Leu, N,N-diallyl-Ala(2)-enkephalin-Leu, N,N-diallyl-Ala(2)-bis(cystine)(6)-enkephalin-Leu, Ala(2)-enkephalin-Leu-polyethylene glycol, Ala(2)-enkephalinamide-Leu, Ala(2)-aminoethyl dimer-enkephalinamide-Leu, Ala(2)-N-(2-((4-azido-2-nitrophenyl)amino)N-ethyl(5))-enkephalinamide-Leu, de-Tyr(1)-Ala(2)-enkephalinamide-Leu, tyrosyl-alanyl-glycyl-phenylalanyl-psi(thiomethylene)leucine, cyclic leucine enkephalin, cyclo(lysyl-tyrosyl-glycyl-glycyl-phenylalanyl-leucyl), Arg(2)-Leu(5)-enkephalin, H-Tyr-cyclo-(N(δ)-Orn-Gly-Phe-Leu)-enkephalin, N-cyclo-Leu(5)-enkephalin, Ser(2)-Leu(5)-Thr(6)-enkephalin, enkephalin-azo-albumin, 4′-bromo-Phe(4)-enkephalin-Leu, 4-(hydroxyphenyl)azo-enkephalin-Leu, 4-hydroxycinnamoyl(1)-enkephalin-Leu, acetaldehyde-enkephalin-Leu, Arg(6)-enkephalin-Leu, Arg(6)-Phe(7)-enkephalin-Leu, Arg(6)-PheNH₂(7)-enkephalin-Leu, Arg(6,7)-enkephalin-Leu, cyclo-N(y)-diNH-butyryl-enkephalin-Leu, dehydro-Phe(4)-enkephalin-Leu, desTyr(1)-enkephalin-Leu, Gly-Pro-(Lys-Aib-Leu-Aib)(2)-OMe-enkephalin-Leu, Gly-Pro-(Lys-Pro-Pro-Pro)2-OMe-enkephalin-Leu, Gly-Pro-(Lys-Sar-Sar-Sar)(2)-OMe-enkephalin-Leu, NH₂(3)-enkephalin-Leu, sulfonated enkephalin-Leu, Gly(2)-ψ-(methyleneoxy)-Gly(3)-Leu(5)-enkephalinamide, Tyr(1)-ψ-(methyleneoxy)-Gly(2)-Leu(5)-enkephalinamide, enkephalinamide-Leu, cyclo(α,γ-dibutyric acid(2)-glutamyl(3))-enkephalinamide-Leu, Tyr sulfate(1)-enkephalinamide-Leu, ICI 154129, Leu-enkephalin-tyrosyl-arginyl-glycyl-phenylalanine ethyl ester, N,N-dibenzyl(Phe(p-NCS))(4)-leucine enkephalin, N,N-diallyl-tyrosyl-a-aminoisobutyric acid-phenylalanyl-leucine, phorphin, pro-enkephalin-Leu, t-butyloxycarbonyltyrosyl-glycyl-glycyl-phenylalanyl-psi(thioamide)leucyl benzyl ester, tyrosyl-cyclo(lysyl-glycyl-phenylalanyl-psi(thiomethylene)leucine), tyrosyl-glycyl-glycyl-(4-nitro)phenylalanyl-leucinamide, tyrosyl-glycyl-sarcosyl-(4-nitro)phenylalanyl-leucinamide, tyrosyl-threonyl-glycyl-phenylalanyl-leucyl-O-glucosylserinamide, Met(2)-Pro(5)-enkephalin, Met(2)-ProNH₂(5)(N(1,5)-glucopyranosyl)enkephalin, Met(2)-Thz(5)-GlyNH₂(3)-enkephalin, methionine enkephalin, adrenorphin, BAM 12P, BAM 18P, BAM 22P, BAM-20P, δ-Ala(2)-Met(5)-enkephalin, Met(2)-ProNH₂(5)-enkephalin, 4′-bromo-Phe(4)-enkephalin-Met, 5-amino-Val(2)-desGly(3)-enkephalin-Met, acetaldehyde-enkephalin-Met, Ala(2)-enkephalin-Met, Ala(2)-4-azido-Phe(4)-enkephalin-Met, Arg(6)-enkephalin-Met, Arg(6)-Gly(7)-Leu(8)-enkephalin-Met, Arg(6)-Gly(7)-Leu(8)-Lys(9)-enkephalin-Met, Arg(6)-Phe(7)-enkephalin-Met, Arg(6)-PheNH₂(7)-enkephalin-Met, Arg(6,7)-enkephalin-Met, desTyr(1)-enkephalin-Met, Lys(6)-enkephalin-Met, Lys(6)-Arg(7)-enkephalin-Met, sulfoxide-enkephalin-Met, Trp(4)-enkephalin-Met, Tyr-O-sulfateenkephalin-Met, Met(2)-Pro(5)-(N(1,5)-2,3,4,6-O-tetraacetylglycosyl)-enkephalinamide, Met-metazocine-enkephalinamide, Ala(2)-enkephalinamide-Met sulfoxide, Ala(2)-enkephalinamide-Met, Ala(2)-(penta-F-Phe)(4)-enkephalinamide-Met, Ala(2)-didehydro-Phe(4)-enkephalinamide-Met, Ala(2)-N-Me(5)-enkephalinamide-Met, Ala(2,3)-enkephalinamide-Met, Tyr sulfate(1)-Ala(2)-enkephalinamide-Met, Enkorten, Met-enkephalin-FMRFa chimeric peptide, Met-enkephalin-glycyl-tyrosine, Met-enkephalinamide, metkephamid, metkephamid acetate, nifalatide, peptide F, pro-Met-enkephalin, N-(1-(Cl—Ac)-3-methylbutyl)-PheNH₂(4)-enkephalin, Pen(2)-Cys(5)-enkephalin, Pen(2,5)-4′-iodo-Phe(4)-enkephalin, Pen(2,5)-4-chloro-Phe(4)-enkephalin, Pen(2,5)-Ala(3)-enkephalin, Thr(2)-Thz(5)-GlyNH₂(3)-enkephalin, Cys(0₂NH₂)(2)-enkephalin-Leu, Cys(O₂NH₂)(2)-enkephalin-Met, Thr(2)-4-azido-Phe(4)-Leu(5)-enkephalin-Thr, Ala(2,5)-enkephalinamide, cyclo(N,N′-carbonyl-lysyl(2,5))-enkephalinamide, Cys(2,5)-enkephalinamide, Met(2)-Hyp(5)galactopyranosyl-enkephalinamide, Met(2)-Hyp(5)glucopyranosyl-enkephalinamide, Met(2)-Pro(5)-(N(1,5))-galactopyranosyl-enkephalinamide, Pen(2)-Cys(5)-enkephalinamide, Thr(2)-delta(3)Pro(5)-enkephalinamide, FW 34569, H-tyrosyl-cyclo(cysteinyl-phenylalanyl-penicillaminyl)-OH, IVS 43, IVS 46, LY 164929, LY 190388, ONO 9902, D-Penicillamine (2,5)-Enkephalin, penicillaminyl(2,5)-phenylalanine(6)-enkephalin, peptide B, peptide E (adrenal medulla), preproenkephalin, proenkephalin, proenkephalin carboxyl-terminal peptide B, RX 783030, synenkephalin, tyrosyl-(valyl-glycyl-phenylalanyl-alanyl)-OH, tyrosyl-alanyl glycyl-phenylalaninamide-propyl-phenylalaninamide-glycyl-alanyl-tyrosine, tyrosyl-alanyl-glycyl-phenylalanyl-cysteine S-ethyl ester, tyrosyl-alanylglycyl-phenylalanyl-cysteine S-butyl ester, tyrosyl-arginyl-glycyl-4-nitrophenylalanyl-prolinamide, tyrosyl-arginyl-phenylalanyl-norvalylamide, tyrosyl-arginyl-phenylalanyl-phenylaninamide, tyrosyl-D-alanyl-glycyl-methylphenylalanyl-N-propylglycinamide, tyrosyl-methionyl(O)-glycyl-ethylphenylalanine-2-acetylhydrazide,

Nociceptins and nociceptin analogs include nociceptin(1-11), nociceptin(1-13) amide, Phe(1)ψ(CH₂13 O)Gly(2)-nociceptin(1-13), Phe(1)ψ(CH₂—NH)-Gly(2)-nociceptin(1-13)-NH₂, Phe(1)ψ(CH₂NH)-Gly(2)-nociceptin(1-17)-NH₂, Arg(14)-Lys(DTPA)(15)-nociceptin(1-17)amide, nociceptin(1-6), nociceptin orphanin FQ(1-17)OH, Arg(14)-Lys(15)-nociceptin, Tyr(1)-nociceptin, NPhe(1)-nociceptin-(1-13)-NH₂, (pF)Phe(4)-Aib(7,11)-Arg(14)-Lys(15)-nociceptin-amide, Nphe(1)-(pF)Phe(4)-Aib(7)-Arg(14)-Lys(15)-nociceptin-amide, Nphe(1)-(pF)Phe(4)-Aib(7,11)-Arg(14)-Lys(15)-nociceptin-amide, phenylalanyl(1)-psi(CH₂NH)-glycyl(2)-4-fluorophenylalanyl(4)-arginyl(14)-lysyl(15)-nociceptin-orphanin FQ-amide.

Secretin

The peptide therapeutic may be secretin, or an analog thereof. Such peptides include (ψ-4,5)-secretin, (rat secretin-27)-Gly-rhodamine, prosecretin, glycine secretin(1-27), secretin(1-6), secretin(21-27), secretin(4-27), Gln(9)-secretin(5-27), secretin(7-27), secretin releasing peptide, Tyr(10)-secretin, 27-deamido-secretin, Asp(3)-secretin, β-Asp(3)-secretin, Tyr(6)-secretin, Val(5)-secretin, technetium ^{99m}Tc-secretin, vasectrin I, vasectrin II, and vasectrin III.

Tachykinins

The peptide therapeutic may be tachykinin or an analog thereof. Exemplary tachykinin analogs include δ-preprotachykinin(111-126), callitachykinin I, callitachykinin II, carassin, Eledoisin, Bolton Hunter-eledoisin ligand, eledoisin(6-11), eledoisin(7-11), eledoisin C-terminal heptapeptide, substance P analog(eledoisin related peptide), gal(1-14)-(Abu 8) scy-I, hemokinin-1, Kassinin, Leucophaea maderae LemTRP-1 protein, neurokinin A, iodoacetyl-Bodipy-neurokinin A, MDL 28564, MEN 10456, lysyl3-glycyl8-R-lactam-leucine9-neurokinin A(3-10), Ala5-neurokinin A(4-10), β-Ala(8)-neurokinin A(4-10), Lys(5)-MeLeu(9)-Nle(10)-neurokinin A(4-10), Lys(5)-Tyr(12)(7)-MeLeu(9)-Nle(10)-neurokinin A(4-10), Nle(10)-neurokinin A(4-10), Trp(7)-β-Ala(8)-neurokinin A(4-10), Tyr(5)-Trp(6,8,9)-Arg(10)-neurokinin A(4-10), neurokinin A(4-10)-OH, neurokinin A(4-10), Tyr(5)-Trp(6,8,9)-Lys(10)-neurokinin A(4-10), Ala(5)-Aib(8)-Leu(10)-neurokinin A, iodoHis(2)-neurokinin A, iodoHis(3)-neurokinin A, Leu(3)-Ile(7)-neurokinin A, Leu(9)-neurokinin A, neurokinin A-bovine serum albumin conjugate, neurokinin A-OH, propionyl neurokinin A, neurokinin B, GR 138678, neurokinin B(4-10), β-Asp4-MePhe7-neurokinin B(4-10), I-His-MePhe7-neurokinin B, MePhe7-neurokinin B, Pro2-Trp(6,8)-Nle10-neurokinin B, neurokinin B-bovine serum albumin conjugate, neuromedin B, cyclic Cys(2,5)-neuromedin K, preprotachykinin B(50-79), neuropeptide K, PG-KII peptide, physalaemin, GR 82334, hylambatin, physalaemin C-terminal heptapeptide, LysS-Thr6-physalaemin, uperolein, mouse preproneurokinin-C, preprotachykinin, protachykinin, ranamargarin, ranatachykinin A, ranatachykinin B, ranatachykinin C, ranatachykinin D, scyliorhinin I, scyliorhinin II, scyliorhinin II(3-18), sialokinin I, sialokinin II, Substance P, arginyl-prolyl-lysyl-prolyl-dodecane, Bolton Hunter reagent-substance P conjugate, delta-Ava-Pro(9)-substance P(7-11), galanin(1-13)-spantide amide, galantide, GR 71251, GR 73632, NY 3238, NY 3640, propionyl-(Met(O₂)11)substance P(7-11), senktide, septide, acetyl-Arg6-septide, spantide, spantide II, spantide III, substance P(1-4), substance P(1-6), substance P(1-7), Pro(2)-Phe(7)-substance P(1-7), substance P(1-9), substance P(3-11), α-biotinyl-Lys(3)-substance P(3-11), substance P(3-4), substance P(4-11), β-Ala(4)-Sar(9)-Met(O₂)(11)-substance P(4-11), Pro4-Npa(7,9)-Phe11-substance P(4-11), Pro4-Trp(2,9,10)-substance P(4-11), Pro4-Trp(7,9)-substance P(4-11), Pro4-Trp(7,9)-LeuNH₂(11)-substance P(4-11), Pro4-Trp(7,9)-Nle11-substance P(4-11), Pro4-Trp(7,9)-PheNH₂(11)-substance P(4-11), Pro4-Trp(7,9,10)-substance P(4-11), Pro4-Trp(7,9,10)-Phe(11)-substance P(4-11), Pro4-Val8-Trp(7,9,10)-substance P(4-11), substance P(5-11), ArgS-Trp(7,9)-substance P(5-11), Arg5-Trp(7,9)-Nle11-substance P(5-11), Asp(5,6)-MePhe8-substance P(5-11), cyclo(11-5(ε))Lys5-substance P(5-11), Glp5-Glu(O-benzyl)(11)-substance P(5-11), N,N-diMe-Gln6-substance P(5-11), N-α-(desamino-3-iodotyrosyl)-8-N-Me-Phe-5,6-Asp-substance P(5-11), pGlu5-MePhe8-MeGly9-substance P(5-11), substance P(6-11), Ac(Arg6-Sar9-Met(O₂)(11))-substance P(6-11), Arg6-Trp(7,9)-Me-Phe8-substance P(6-11), Glp6-Glu(O-benzyl)(11)-substance P(6-11), Glp6-iodo-Tyr8-substance P(6-11), Glu6-substance P(6-11), Glu(G1c)(6)-substance P(6-11), N(1,6)(β-glucopyranosyl)Glu5-Pro9-substance P(6-11), N(α)-(3-iododesaminotyrosyl)-substance P(6-11), Orn6-substance P(6-11), pGlu6-substance P(6-11), pGlu6-N-MeLeu10-substance P(6-11), pGlu6-N-MePhe7-substance P(6-11), pGlu6-N-MePhe8-Aib9-substance P(6-11), pGlu6-Phe8-ψ-(methyleneoxy)-Gly9-substance P(6-11), Phe7-His9-substance P(6-11), Tyr6-D-Phe7-D-His9-substance P(6-11), Orn6-Glu(O-benzyl)(11)-substance P(6-11)-O-benzyl, substance P(7-11), β Ala4-Ser9-Met(O₂)(11)-substance P(4-11), Pro4-Trp(7,9,10)-LeuNH₂(11)-substance P(4-11), (3-iodo-4-hydroxyphenyl)propionic acid-substance P, 1,4,7,10-tetraazacyclododecane-1-glutaric acid-4,7,10-triacetic acid-arginyl(1)-substance P, 3-(4-hydroxy-3,5-diiodophenyl)propionic acid-substance P, α-biotinyl-Arg(1)-substance P, α-N-Arg(1)-ε-N-Lys(3)-di-(pyridoxal phosphate)-substance P, amino(4)-Phe(7)-substance P, aminoethyl(2)-Met(11)-substance P, Arg(1)-Cl₂-Phe(5)-Asn(6)-Trp(7,9)-Nle(11)-substance P, Arg(1)-Pro(2)-Phe(7)-His(9)-substance P, Arg(1)-Pro(2)-Trp(7,9)-LeuNH₂(11)-substance P, Arg(1)-Pro(2)-Trp(7,9)-substance P, Arg(1)-Pro(2)-Trp(7,9)-Leu(11)-substance P, Arg(1)-Trp(5,7,9)-Leu(11)-substance P, Arg(1)-Trp(7,9)-Leu(12)-substance P, Arg(3)-substance P, biotin-NTE-Arg(3)-substance P, biotinyl-apa-Pro(9)-MePhe(pBz)(10)-Trp(11)-substance P, Bpa(8)-substance P, cyclo(H-Glu-Phe-Phe-Gly-Leu-Met-NH(CH₂)₃-NH-)substance P, Cys(3,6)-Tyr(8)-Pro(9)-substance P, desArg(1)-substance P, ε-biotinyl-Lys(3)-substance P, Gly(12)-substance P, Gly(12)-Lys(13)-substance P, Indium-111-DTPA-Arg(1)-substance P, iodo-Tyr(8)-substance P, Leu(11),CH₂NH-(10-11)-substance P, methyl ester-substance P, N-spermine-Gln(5)-substance P, NleNH₂(11)-substance P, pGlu(5)-MePhe(8)-Sar(9)-substance P, Phe(5)-Trp(7,9)-Leu(11)-substance P, Phe(7)-substance P, Pro(2)-Phe(7)-Trp(9)-substance P, Pro(9)-substance P, Pro(9)-Met(O₂)(11)-substance P, prolyl(2)-tryptophan(7,9)-substance P, pyridoxal-phosphate(6)-Lys(3)-substance P, Sar(9)-Met(O₂)(11)-substance P, sulfoxide-substance P, Trp(9)-substance P, Tyr(0)-(4′-N3)Phe(8)-Nle(11)-substance P, Tyr(1)-Nle(11)-substance P, Tyr(8)-substance P, (3-iodo-4-hydroxyphenyl)propionic acid-N-hydroxysuccinimidyl ester-substance P, 3-(4-hydroxy-3,5-diiodophenyl)propionic acid-N-hydroxysuccinimidyl ester-substance P, substance P-metabolite 5-11, substance P-saporin, tryptophyl(7)-sendide, human TAC4 protein, and tachykinin neuropeptide γ.

Vasopressin

The peptide therapeutic may be vasopressin or a vasopressin analog. Exemplary vasopressin analogs include arginine vasopressin, (phenylmethoxy)carbonyl-asparaginyl-(cysteinyl)cysteinyl-prolyl-arginine, acetylmethionyl-prolyl-arginine, acetylmethionyl-prolyl-arginyl-glycinamide, arginine vasopressin(2-5), 2-naphthylalanine(3)-arginine vasopressin, acyclic argipressin(1-6), argipressin(1-7), (4-1′)-disulfide Cys(6)-argipressin(3-9), argipressin(4-8), argipressin(4-8) cysteinyl methyl ester, argipressin(4-9), (3-1′)-disulfide Cys(6)-argipressin(4-9), (2-1′)-disulfide Cys(6)-argipressin(5-8), (2-1′)-disulfide Cys(6)-argipressin(5-9), argipressin methylenedithioether, (1-(1-mercapto-4-methylcyclohexaneacetic acid)-Phe(2)-Ile(4))-argipressin, (1-(4-mercapto-1-methyl-4-piperidineacetic acid)-Phe(2)-Ile(4))-argipressin, (1-(4-mercapto-4-tetrahydrothiopyranoacetic acid)-Phe(2)-Ile(4))-argipressin, (1-(4-mercaptotetrahydropyranoacetic acid)-Phe(2)-Ile(4))-argipressin, (1-β-mercapto-β,β-cyclopentamethylenepropionic acid)-Sar(7)-argipressin, (1-mercapto-4-methylcyclohexaneacetic acid)(1)-argipressin, (1-mercapto-4-phenylcyclohexaneacetic acid)(1)-argipressin, (1-mercapto-cyclohexaneacetic acid)(1)-O-methyl-Tyr(2)-glutamic acid (γ-Gly-amide)(4)-argipressin, (1-mercaptocyclohexaneacetic acid)(1)-Ile(2)-Val(4)-argipressin, (3,4-dehydro-Pro)(7)-argipressin, (4-azido)Phe(3)-argipressin, (4-tert-butyl-1-mercaptocyclohexaneacetic acid)(1)-argipressin, (β-mercapto-β,β-cyclopentamethylenepropionic acid)(1)-Ile(2)-Ala(4)-argipressin, (methyl-alanyl(7))-argipressin, 1-(4-thio-4-tetrahydropyranoacetic acid)-O-Et-Tyr(2)-Val(4)-argipressin, 1-(4-thio-4-tetrahydrothiopyranoacetic acid)-O-Et-Tyr(2)-Val(4)-argipressin, 1-(β-mercapto-β,β-diethylpropionic acid)-argipressin, 1-deamino-3-(3′-pyridyl)-Ala(2)-argipressin, 1-deaminopentamethylene-Phe(2)-Ile(4)-argipressin, 3-mercapto-3-methylbutyryl(1)-MeTyr(2)-argipressin, Ala(10)-argipressin, Ala-Gly-argipressin, AlaNH₂(9)-argipressin, Asp(5)-argipressin, Asu(1,6)-argipressin, Asu(1,6)-Phe(4-N3)(3)-argipressin, β mercapto-β,β-cyclopentamethylenepropionic acid(1)-Tyr(2),Ile(4),Lys(9)(N(6)-fluoresceinylaminothiocarbonyl)-argipressin, β mercapto-β,β-cyclopentamethylenepropionic acid(1)-O-methyl-Tyr(2)-Lys-(N(epsilon)-biotinamidocaproate)NH₂(9)-argipressin, δ mercapto-δ,δ-cyclopentamethylenepropionic acid(1)-Ile(2,4)-Ala-NH₂(9)-argipressin, δ mercapto-β,β-cyclopentamethylenepropionic acid(1)-O-methyl-Tyr(2)-TyrNH₂(9)-argipressin, β, mercapto-δ,δ-cyclopentamethylene propionic acid(1),Tyr(2),Ile(4),Lys(9)(N(6)-tetramethylrhodamylaminothiocarbonyl)-argipressin, β-mercapto-β,β-cyclopentamethylenepropionic acid-Tyr(Me)(2)-Ala-NH₂(9)-argipressin, β-mercapto-β,β-cyclopentamethylenepropionic acid(1)-O-methyl-Tyr(2)-Val(4)-argipressin, β-mercapto-β,β-cyclopentamethylenepropionic acid(1)-Ile(2,4)-argipressin, β-mercapto-(δ,δ-cyclopentamethylenepropionic acid(1)-Ile(2)-Thr(4)-argipressin, β-mercapto-β,β-cyclopentamethylenepropionic acid(1)-O-methyl-Tyr(2)-LysNH₂(9)-argipressin, β-mercapto-β,β-cyclopentamethylenepropionic acid(1)-argipressin, β-mercapto-β,β-cyclopentamethylenepropionic acid(1)-Ile(2)-Abu(4)-argipressin, Cpa(1)-Phe(ethylene)Phe(2,3)-Val(4)-argipressin, d(CH₂)₅(1)-Tyr(Me)(2)-delta(3)Pro(7)-argipressin, d(CH₂)₅-O-ethyl-Tyr(2)-Val(4)-Tyr-NH₂(9)-argipressin, d(CH₂)₅-Phe(2,4)-argipressin, dCha(4)-argipressin, deamino(4-Dab(N(δ)-N-maleoyl-β-alanine)) argipressin, deaminopenicillamine(1)-O-methyl-Tyr(2)-argipressin, deaminopenicillamine(1)-Val(4)-argipressin, desGly-NH₂(9)-argipressin, glutamic acid (γ-N,N-diethylamide)(4)-argipressin, Gly(OH)9-argipressin, homo-argipressin, hydroxy-Pro(4)-argipressin, Mca(1)-I-Tyr(2)-Sar(7)-argipressin, Phe(2)-argipressin, Phe(2)-(4-azido)Phe(3)-argipressin, Pro(4)-argipressin, Pro(4)-hydroxy-Pro(7)-argipressin, Ser-Ala-argipressin, Thr(10)-Ser(11)-Ala(12)-argipressin, Val(4)-argipressin, (1-mercaptocyclohexaneacetic acid)(1)-O-ethyl-Tyr(2)-argipressin, (1-mercaptocyclohexaneacetic acid)(1)-Tyr(2)-Val(4)-argipressin, β-mercapto-β,β-cyclopentamethylenepropionic acid(1)-Val(4)-argipressin, deamino arginine vasopressin, DCDAVP, 1-deamino-4-Val-8-Arg-vasopressin, deamino(4-Thr-8-Arg)-vasopressin, deamino-homo-Arg-vasopressin, iodo-sarc-arginine-vasopressin, SK&F 100398, SK&F 101071, SK&F 101926, SK&F 103784, SK&F 105494, 1-(1-mercaptocyclohexaneacetic acid)-2-(O-methyl-L-tyrosine)-8-L-arginine-vasopressin, 1-(2-mercapto-2,2-(cyclopentamethylene)propionic acid)-2-(O-methyl)Tyr-8-Arg-vasopressin, 1-adamantanacetyl-2-(O-ethyl)Tyr-4-Val-6-aminobutyryl-8,9-Arg-vasopressin, 1-deamino-(2-(O-methyl)Tyr)-4-Val-8-Arg-vasopressin, 1-deamino-2-Phe-7-(3,4-dehydro)Pro-8-Arg-vasopressin, 1-deamino-4-(2-aminobutyric acid)-8-Arg-vasopressin, β-mercapto-β,β-cyclopentamethylenepropionic acid(1)-Phe(2)-Ile(4)-Arg(8)-Ala(9)-vasopressin, β-mercapto-β,β-cyclopentamethylenepropionic acid-Sar(7)-Arg(8)-vasopressin, d(CH₂)₅(1)-Tyr(OMe)(2)-Arg(8)-vasopressin, desGly(9)-phenylacetyl(1)-O-Et-Tyr(2)-Lys(6)-Arg(8)-vasopressin, desTyr(2-methyl)-4-Val-D-8-Arg-vasopressin, Mpa(1)-Aic(2)-Val(4)-Arg(8)-vasopressin, N,N-diethylamide 1-(1-mercaptocyclohexaneacetic acid)-2-O-methyl-Tyr-4-glutamic acid (γ-N,N-diethylamide)-8-Arg-vasopressin, N-acetyl-Arg(8)-vasopressin, N-acetyl-O-methyl-Tyr(2)-Arg(8)-vasopressin, Val-Asp-Arg(8)-vasopressin, human AVP protein, F-180 vasoconstrictor peptide, Glanduphen, iodo-lin-vasopressin, Lysine vasopressin, 1-deamino-triglycyl-8-lysine-vasopressin, 7-(azetidine-2-carboxylic acid)lysinevasopressin, felypressin, terlipressin, ((2-mercapto)propionic acid)(1)-(Lys-N(6)-biotin)(8)-vasopressin, (1 β-mercaptopropionic acid,8(ε-N-4-toluenesulfonyl)Lys)-vasopressin, (1-(2-hydroxy-3-mercaptopropanoic acid)-8-Lys)-vasopressin, (1-(2-mercapto)propionic acid)-N(6)-5-dimethylaminonaphthalene-1-sulfonyl-8-Lys-vasopressin, (1-2-mercapto)propionic acid-N(6)-carboxytetramethylrhodamine-8-Lys-vasopressin, (1-α-mercaptoacetic acid)-8-Lys-vasopressin, (1-β-mercapto-β,β-diethylpropionic acid-4-Leu)-8-Lys-vasopressin, (1-β-mercaptopropionic acid-8-Lys)-vasopressin, (1-γ-mercaptobutyric acid)-8-Lys-vasopressin, (3-(1,4-cyclohexadienyl)Ala-8-Lys)-vasopressin, (5-(N(4),N(4)-dimethyl-Asn)-8-Lys)-vasopressin, 1-(2-mercapto)propionic acid-N(6)-2-N-methylanthranilamide-8-Lys-vasopressin, 1-(3-mercapto)propionic acid-8-(N(6)-4-azidophenylamidino)lysine-vasopressin, 1-(β-mercapto-β,β-cyclopentamethylene propionic acid)-(O-ethyl)Tyr(2)-Val(4)-Lys(8)-N(6)-carboxytetramethylrhodamine-vasopressin, 1-β-mercapto-β,β-diethylpropionic acid-8-Lys-vasopressin, 1-deamino-(3-(4-azido-Phe))-8-Lys-vasopressin, 1-deamino-(8-lysine(N(6)-tetramethylrhodamylaminothiocarbonyl))-vasopressin, 1-deamino-Leu(4)-Lys(8)-vasopressin, 1-desamino-(8-rhodamine-Lys)-vasopressin, 1-penicillamine-2-O-meTyr-8-Lys-vasopressin, 3-(3-β-(2-thienyl)-Ala)-8-Lys-vasopressin, 4-Leu-8-Lys-vasopressin, 8-(4-hydroxyphenylpropionyl)-Lys(8)-vasopressin, 8-Lys-8-phenylpropionyl-vasopressin, 9-Ala-NH₂-Lys-vasopressin, 9-desGly-NH₂-Lys-vasopressin, 9-homo-Lys-vasopressin, deamino(8-Lys(N(ε)-N-maleoyl-(β-alanine)) vasopressin, Glu(NHNH₂)(4)-Lys(8)-vasopressin, Gly-Lys-Arg-8-Lys-vasopressin, N(epsilon)-tyrosyl-8-lysyl-vasopressin, N-(N-Gly-Gly)-8-Lys-vasopressin, N-α-Gly-Gly-Gly-8-Lys-9-desGlyNH₂-vasopressin, N-Gly-8-Lys-vasopressin, vasopressin,-(1-(2-mercapto)propionic acid)-N(6)-carboxyfluorescein-8-Lys-, Neo-lidocaton, ornipressin, 2-Gly-9-desGly-2-Phe-8-Orn-vasopressin, 2-Gly-9-desGly-4-Val-8-Orn-vasopressin, 9-desGly-(2-Phe-8-Orn)-vasopressin, Phe(2)-Ile(3)-Orn(8)-vasopressin,desGly(NH₂)(9)d(CH₂)₅-Tyr(Me)(2)-Thr(4)-Orn(8)-vasotocin, β-mercapto-β,β-cyclopentamethylenepropionic acid-Trp(2)-Phe(3)-Ile(4)-Arg(8)-oxytocin, pitressin tannate, preprovasopressin, pressinamide, pressinoic acid, proAVP hormone, (β-mercapto-(β,β)-cyclopentamethylenepropionic acid)-Phe(2)-Ile(4)-Ala(9)-vasopressin, β-mercapto-β,β-cyclopentamethylenepropionic acid(1)-O-ethyl-Tyr(2)-Val(4)-Cit(8)-vasopressin, desGly-vasopressin, ε-hydroxy-Nle(8)-vasopressin, homo-Nle(8)-vasopressin, and vasopressinase-altered vasopressin.

### Other Peptide Hormones

Other peptide hormones include adipokines, adrenomedullins, ghrelin, gonadotropins, inhibins, natriuretic peptides, parathyroid hormone (PTH) and parathyroid hormone related peptide (PTHrP), thymosin, relaxins, and analogs thereof. Peptide hormones also include BIM28163, GKN1 protein, C. elegans Ins-7 protein, mouse InsI5 protein, human intermedin protein, motilin, 13-Leu-motilin, ANQ 11125, atilmotin, biotinyl(Cys(23))motilin, Nle(13)-motilin, OHM 11526, Leu(13)-pMot(1-14), Prepromotilin, SK896, human obestatin, mouse obestatin, rat obestatin, osteocalcin, osteocalcin(37-49), Peptide PHI, Phe(4)-peptide PHI, peptide PHI-(1-27)-glycine, Gln(24)-PHI peptide, Arabidopsis RALF1 protein, RC-1139, sauvagine, Tremella brasiliensis tremerogen A-I protein, mouse urotensin II-related peptide, rat urotensin II-related peptide, urotensin, (Orn8)urotensin-II, preprourotensin II, urotensin I, urotensin II, Pen(5)-Trp(7)-Orn(8)-urotensin II(4-11), Cha(6)-urotensin II(4-11), human UTS2D protein, and Xenopus Xen-dorphin prohormone.

Adipokines

In certain embodiments, the peptide therapeutic is an adipokine or an analog thereof. Adipokines include adiponectin, leptin, and resistin. Adiponectins include human, mouse, and rat adiponectin. Leptins include leptin(116-130), leptin(22-56), leptin(57-92), leptin(93-105), LY396623, metreleptin, murine leptin analog, pegylated leptin, and methionyl human leptin. Resistins include human, mouse, and rat resistin.

Adrenomedullins

In certain embodiments, the peptide therapeutic is an adrenomedullin or an analog thereof. Such peptides include adrenomedullin(1-12), adrenomedullin(1-50), adrenomedullin(11-26), adrenomedullin(13-52), adrenomedullin(15-22), rat adrenomedullin(20-50), adrenomedullin(22-52), rat adrenomedullin(24-50), adrenomedullin(27-52), adrenomedullin precursor(45-92), adrenomedullin(16-31), adrenotensin, rat intermedin protein, proadrenomedullin, and prodepin.

Ghrelin

In certain embodiments, the peptide therapeutic is ghrelin or a ghrelin analog. Exemplary ghrelin analogs include Trp3-Arg5-ghrelin(1-5), BIM-28125, desGln14-ghrelin, des-n-octanoyl-ghrelin, human GHRL protein, RC-1291, and human exon 3-deleted preproghrelin.

Gonadotropins

In certain embodiments, the peptide therapeutic is a gonadotropin. Exemplary gonadotropins include carp gonadotropin, carp vitellogenic gonadotropin, Gestyl, PMSG-HCG, chorionic gonadotropin, AB1ER-CR-2XY, asialo-human chorionic gonadotropin, asialoagalacto-human chorionic gonadotropin, asialogalactochoriongonadotropin, human β subunit chorionic gonadotropin, HCG-β(109-145), HOG-β(112-145), HCG-β(123-145), HCG-β(128-145), HCG-β(Gly(88,90))82-101, hecate-chorionic gonadotropin β-subunit conjugate, human chorionic gonadotropin-tetanus toxoid, urinary gonadotropin fragment, Xanthomonas maltophilia chorionic gonadotropin, CTP37 peptide, gestagnost, a subunit glycoprotein hormone, glycosylated HCG, deglycosylated HCG, des(122-145)-HCG-β, hTSHβCTPα protein, human chorionic gonadotropin-cholera toxoid conjugate, human chorionic gonadotropin-diphtheria toxin fragment A, iodo-chorionic gonadotropin, nymfon-orion, Ovidrel, PMSG-HCG, Profasi, prostaglandin 600, selenomethionyl choriogonadotropin, recombinant yoked hormone receptor, prolactin-like protein-K, mouse, prolactin-like protein-N, mouse, prolactin-like protein-O, mouse, and salmon gonadotropin.

Pituitary gonadotropins include follicle stimulating hormone (FSH), 4-azidobenzoyl-FSH, 4-azidobenzoylglycyl-FSH, β-subunit FSH, β-subunit(1-15) FSH, human β-subunit(33-53) FSH, human β-subunit(33-53)-(81-95)-peptide amide FSH, β subunit(51-65) FSH, human β-subunit(81-95) FSH, porcine β subunit precursor FSH, human Ser(51)-FSH-β(33-53), human Ser(82,84,87,94)-FSH-β(81-95), deglycosylated FSH, DA-3801, human FSH with HCG C-terminal peptide, human chorionic gonadotropin-tetanus toxoid, Fundulus gonadotropin I β-subunit, bass gonadotropin I β-subunit, Katsuwonus gonadotropin I, tuna gonadotropin I, catfish gonadotropin II α-subunit, bass gonadotropin II β-subunit, catfish gonadotropin II β-subunit, Fundulus gonadotropin II β-subunit, Katsuwonus gonadotropin II, tuna gonadotropin II, salmon gonadotropin-pituitary,β-subunit I, luteinizing hormone (LH), big luteinizing hormone, FSH-α, β subunit LH, hecate-βLH, Phor14-βLH, deglycosylated LH, desialylated LH, diiodo LH, nitroguanidyl LH, plant LH-gelonin conjugate protein, menotropin, hMG-IBSA, menogonadyl, urofollitropin, prolactin, alanyl-seryl-(histidyl)6-isoleucyl-glutamyl-glycyl-arginyl-prolactin, mouse Dtprp protein, rat Dtprp protein, fluorescein-5-isothiocyanate-prolactin, methionylprolactin, rat PLP-I protein, preprolactin, Oreochromis niloticus PRL177 protein, Oreochromis niloticus PRL188 protein, human prolactin 16-kDa fragment, Arg(129)-prolactin, Asp(179)-prolactin, 0(1-9)-Arg(129)-prolactin, glycosylated prolactin, polymeric prolactin, and prolactin-daunomycin ligand.

Inhibins

In certain embodiments, the peptide therapeutic is an inhibin. Exemplary inhibins include inhibin, zebrafish activin βB, α-inhibin-92, β-inhibin(67-94), human inhibin-like peptide(1-31), inhibin A, inhibin a 1-26, inhibin B, inhibin-a subunit, inhibin-a subunit precursor, inhibin-βA subunit precursor, inhibin-β subunit, mouse erythroid differentiation and denucleation factor, human INHBB protein, mouse INHBB protein, rat INHBB protein, human INHBC protein, mouse INHBC protein, rat INHBC protein, human INHBE protein, mouse INHBE protein, rat INHBE protein, inhibin β A subunit, inhibin βD subunit, and Tyr85-Cys(Acm)87-seminal plasma inhibin(85-94).

Insulin-like Growth Factors

In certain embodiments, the peptide therapeutic is insulin-like growth factor I, insulin-like growth factor II, or an analog thereof. Such peptides include 14-kDa cementum-derived growth factor, human insulin-like-growth-factor-I (21-40), insulin like growth factor 1(1-27)-Gly₄-(38-70), A(27)-B-insulin-like growth factor I insulin, des(1-3)-insulin-like growth factor I, insulin-like growth factor 1A prohormone(91-103), insulin-like growth factor I(24-41), insulin-like growth factor I (30-41), insulin-like growth factor I (57-70), Gln(3)-Ala(4)-Tyr(15)-Leu(16)-insulin-like growth factor I, N-Ala-Glu-insulin-like growth factor I, N-methionyl-insulin-like growth factor I, Thr(59)-insulin-like growth factor I, Val(59)-insulin-like growth factor I, insulin-like growth factor I-Pseudomonas exotoxin A (40), insulin-like growth factor-1 D peptide, mouse insulin-like growth factor-1, long R(3)-insulin-like growth factor-I, LR(3)IGF-I, human mechano-growth factor E, mouse mechano-growth factor, rat mechano-growth factor, Na(Gly1)-((2-6-(biotinamido)-2-(4-azidobenzamido)hexanoamido)ethyl-1′-dithiopropionyl)-insulin-like growth factor-1, Nα(Gly1)-(4-azidobenzoyl)-insulin-like growth factor-I, preproinsulin-like growth factor I, pro-insulin-like growth factor I, 4-azidobenzoyliodo-insulin-like growth factor II, human IGF2 protein, mouse IGF2 protein, insulin-like growth factor II (33-40), Tyr(0)-insulin-like growth factor II(33-40), insulin-like growth factor II (69-84), Leu(27)-insulin-like growth factor II, preproinsulin-like growth factor II, preptin, proinsulin-like growth factor II, proinsulin-like growth factor II(117-156)

Natriuretic Peptides

In certain embodiments, the peptide is a natriuretic peptide. Exemplary natriuretic peptides include atrial natriuretic factor, (Cys18)-atrial natriuretic factor(4-23)-amide, A 68828, A 71915, Leu(8,18)-Ile(12)-Ala(20)-MePhe(26)-Tyr(28)-Pro(29)-ANF(4-28), asparaginyl-seryl-phenylalanyl-arginyl-tyrosinamide, atrial natriuretic factor(1-11), atrial natriuretic factor(1-16), atrial natriuretic factor(1-27), Ala(26)-atrial natriuretic factor(1-28), atrial natriuretic factor (101-105), Mpr105(3)-atrial natriuretic factor(105-126), atrial natriuretic factor(106-126), atrial natriuretic factor(3-28), atrial natriuretic factor(4-23), de-Gln(18)-de-Ser(19)-de-Gly(20,22)-de-Leu(21)-atrial natriuretic factor(4-23)NH₂, atrial natriuretic factor(4-28), atrial natriuretic factor(5-23)amide, I-Tyr(0)-atrial natriuretic factor(5-25), atrial natriuretic factor(5-27), atrial natriuretic factor(5-28), atrial natriuretic factor(7-23), Pro(10)-atrial natriuretic factor(7-23), atrial natriuretic factor(7-23)amide, Met-atrial natriuretic factor 26, rat atrial natriuretic factor 26, atrial natriuretic factor 270, atrial natriuretic factor 88, atrial natriuretic factor precursor(79-98), human atrial natriuretic factor prohormone(1-30), atrial natriuretic factor prohormone(1-98), atrial natriuretic factor prohormone(102-125), atrial natriuretic factor prohormone(102-126), atrial natriuretic factor prohormone(103-123), atrial natriuretic factor prohormone(103-125), atrial natriuretic factor prohormone(103-126), atrial natriuretic factor prohormone(31-67), atrial natriuretic factor prohormone(49-126), rat atrial natriuretic factor prohormone(6-33), atrial natriuretic factor prohormone(8-33), atrial natriuretic factor prohormone(95-126), desSer(5)-Ser(6)-atrial natriuretic factor, Ile(12)-atrial natriuretic peptide(101-126), atrial natriuretic peptide(3-33), rat atrial natriuretic peptide, Ala(8)-atrial natriuretic factor(1-28), atriopeptin analog I, azidobenzoyl-atrial natriuretic factor, cardiodilatin, dextronatrin, iso-atrial natriuretic peptide, iso-atrial natriuretic peptide(17-45), iso-atrial natriuretic peptide(23-39), MiniANP, N-terminal proatrial natriuretic peptide, NNC 70-0270, human NPPA protein, oxidized methionine-α-human atrial natriuretic factor, phospho-urodilatin, PL 058, preproatrial natriuretic factor(104-123), preproatrial natriuretic factor(26-55), preproatrial natriuretic factor(56-92), human RRP17 protein, mouse RRP17 protein, SC 46542, rainbow trout ventricular natriuretic factor, eel ventricular natriuretic peptide, X-atrial natriuretic factor, Salmo salar cardiac natriuretic peptide, Guanylin, brain natriuretic peptide, porcine brain natriuretic peptide, rat natriuretic peptide precursor type B, Pro-BNP1-108, pro-brain natriuretic peptide(1-76), C-type natriuretic peptide, C-type natriuretic peptide (1-53), human amino-terminal pro-C-type natriuretic peptide, mouse CIOR protein, mouse NPPA protein, and uroguanylin.

Parathyroid Hormone

In certain embodiments, the peptide therapeutic is PTH, PTHrP, or an analog thereof. Such peptides include amino-terminal PTH, BIM 44002, biotinyl-PTH, calciferin, carboxyl-terminal PTH, formyl-methionyl-hPTH(1-84), teriparatide, Ala(25,26,27)-PTH(1-34), Arg(2)-PTH(1-34), Leu(8),Asp(10),Lys(11),Ala(16),Gln(18),Thr(33),Ala(34)-PTH(1-34), PTH(1-34)amide, Nle(8,18)-Tyr(34)-PTH(1-34)amide, RS 66271, midcarboxylterminal PTH, p55-PTH(1-38) fusion protein, PTH(1-11), Ala(3)-Gln(10)-Har(11)-PTH(1-11)amide, PTH(1-14)amide, PTH(1-30), PTH(1-31), leucyl(27)-cyclo(glutamyl(22)-lysyl(26))-PTH(1-31)-NH₂, human PTH(1-31)amide, bovine PTH(1-34), chicken PTH(1-34), bovine 8,18-Nle-34-Tyr-PTH(1-34)amide, bovine Nle(8)-Lys(N-ε-4-azido-2-nitrophenyl)(13)-Nle(18)-Tyr(34)-PTH(1-34)amide, Nle(8,18)-Lys(13)(ε-pBz2)-2-Nal(23)-Tyr(34)-PTH(1-34)amide, bovine PTH(1-35), PTH(1-37), PTH(1-38), bovine PTH(1-41), Asp(76)-PTH(1-84), Tyr(34)-PTH(14-34)amide, PTH(19-38), PTH(2-34), PTH(24-48), PTH(28-48), PTH(28-54), PTH(3-34), Nle(8,18)-Nle(34)-PTH(3-34) amide, PTH(3-34)amide, PTH(3-84), formylmethionyl-PTH(3-84), bovine PTH(35-84), PTH(37-84), PTH(4-84), bovine PTH(41-84), 55-Tyr-PTH(42-55), 68-Tyr-PTH(43-68), PTH(44-68), 43-Tyr-PTH(44-68), PTH(46-84), PTH(53-68), PTH(53-84), PTH(65-84), PTH(68-84), PTH(7-34), Ahx(8,18)-Trp(12)-Tyr(34)-PTH(7-34)amide, Nle(8,18)-Trp(12)-Tyr(34)-PTH(7-34)amide, Tyr(34)-PTH(7-34)amide, bovine Trp(12)-Tyr(34)-PTH(7-34)amide, PTH(7-84), PTH(73-84), PTH(8-34), PTH(8-84), zebrafish PTH-1(1-34), zebrafish PTH-2(1-34), bovine 2-nitro-5-azidophenylsulfenyl-PTH, bovine PTH, Parathyroidin, Tyr(1)-Ala(14)-Nle(18,21,25)-pre-proPTH(29+1) amide, preproparathormone, proparathormone, formylmethionyl-proPTH(−6-+84), human PTH protein, RS 23581, 1-Bpa-PTHrP, 2-Bpa-PTHrP, PTHrP(1-36), PTHrP(38-141), PTHrP(38-64), PTHrP(1-108), PTHrP(1-139), PTHrP(1-141), PTHrP(1-16), PTHrP(1-173), PTHrP(1-23), PTHrP(1-34), Ala(26)-PTHrP(1-34)amide, TyrNH₂(36)-PTHrP(1-36), N(a)-(4-azido-2-nitrophenyl)-Ala(1)-Tyr(36)-PTHrP(1-36)amide, PTHrP(1-40), Tyr(40)-PTHrP(1-40), PTHrP(1-74), PTHrP(1-84), PTHrP(1-86), PTHrP(1-87), PTHrP(107-111), PTHrP(107-139), PTHrP(107-139)amide, PTHrP(109-138), PTHrP(109-141), PTHrP(14-34) amide, PTHrP(3-34), human Tyr(40)-PTHrP(3-40), PTHrP(37-67), PTHrP(53-84), PTHrP(67-84), PTHrP(67-86), PTHrP(7-34), Asn(10)-Leu(11)-PTHrP(7-34)amide, Leu(11)-Trp(12)-PTHrP(7-34)amide, PTHrP(1-38), PTHrP(100-114), and human PTHLH protein.

Peptide YY

In certain embodiments, the peptide therapeutic is peptide YY or an analog thereof. Such peptides include peptide YY(1-36), peptide YY(13-36), peptide YY(22-36), N-α-acetyl-Phe(27)-peptide YY(22-36), peptide YY(3-36), Leu(31)-Pro(34)-peptide YY, and Pro(34)-peptide YY.

Thymosin

In certain embodiments, the peptide therapeutic is thymosin or a thymosin analog. Such peptides include ((n-nitroveratryl)oxy)chlorocarbamate-caged thymosin β4, (Met(0)6,Phe(4F)12)deacetyl-thymosin β4, (Met(O)6,Tyr(Me)12)deacetyl-thymosin β4, deacetylthymosin β(10), deacetylthymosin β(11), deacetylthymosin β(12), deacetylthymosin β(4), deacetylthymosin β(4)(Xen), deacetylthymosin β(7), desacetylthymosin α(11), desacetylthymosine α(1), parathymosin α, prothymosin α, Arg(30)-prothymosin α(1-30), C elegans tetrathymosinβ, thymalfasin, thymosin α(1), thymosin α(1)(24-28), thymosin α(11), thymosin α(7), thymosin β(1), thymosin β(10), thymosin β(10)arginine, thymosin β(11), thymosin β(12), thymosin β(14), rat thymosin β(15), thymosin β(4), thymosin β(4)(11-19), thymosin β(4) sulfoxide, thymosin β(4)alanine, thymosin β(8), thymosin β(9), methionine thymosin β(9), human thymosin β-NB, rat thymosin β15, thymosin fraction 3, thymosin fraction 5, thymosin fraction 7, and timoptin.

Relaxin

In certain embodiments, the peptide therapeutic is relaxin or an analog thereof. Such peptides include N(α)-formyltyrosyl-relaxin, phenylalanyl relaxin, preprorelaxin, prorelaxin, human relaxin 3, relaxin C-peptide, mouse relaxin-3 protein, rat relaxin-3, human RLN1 protein, mouse Rln1 protein, human RLN2 protein, human RLN3 protein, and rat RLN3 protein.

### Other Peptides

Other peptides that may be used as a peptide therapeautic include disintegrins, endothelins, and secretory protein inhibitor proteins. Still other peptides include ((GRGDSGRKKRRQRRRPPQ)₂-K-epsilonAhx-C)₂, (asparaginyl-alanyl-asparaginyl-proline)₈, (ClCH₂CO)4K2KβA core peptide, (glycyl-glycyl)GLP-2, (GPGGA)₆-G, (Lys(40)(Ahx-DTPA-¹¹¹In)NH₂)exendin-14, (norleucyl-(succinyllysyl)4)(8)-norleucine, (OHCCO)4K2KβA core peptide, (FGE)₃-Y-(GEF)₂-GD, (POG)(4)POA(POG)(5) peptide, (prolyl-hydroxylprolyl-glycine)10, (prolyl-prolyl-glycine)10, (S)-alanyl-3-(α-(S)-chloro-3-(S)-hydroxy-2-oxo-3-azetidinylmethyl)-(S)-alanine, (T,G)-A-L, 1,3,5-benzene tricarbonyl ((aminoisobutyryl)(4)methyl ester)(3), 1,6-bis(N,N-dimethyl-2′,6′-dimethyltyrosyl-1,2,3,4-tetrahydro-3-isoquinolineamido)hexane, 1-(S)-hydroxy-2-(S,S)-valylamidocyclobutane-1-acetic acid, 101.10 peptide, ¹²³I-K31440 peptide, 27753R.P., 2G12.1 peptide, 3,6-bis(N,N-dimethyl-2′,6′-dimethyltyrosyl-1,2,3,4-tetrahydro-3-isoquinolineamidopropyl)-2(1H)-pyrazinone, 3104-V, 3K(I) peptide, 4-fluorobenzoyl-TN-14003, 4.2 kDa peptide, 5-fluorouracil-poly-α,β-(2-hydroxyethyl)asparamide, synthetic 5-helix protein, 61-26, A 10255, A 10947, A 21978C1, A-FF22, A2-binding peptide, AC 413, Ac-(Gly-Pro-Hyp)₃-Gly-Pro-Trp-(Gly-Pro-Hyp)₄-Gly-Gly-CONH₂, Ac-(Gly-Pro-Hyp)₃-Gly-Trp-Hyp-(Gly-Pro-Hyp)₄-Gly-Gly-CONH₂, LEHD-CHO, AC133 antigen, AC3-I peptide, Acanthophis acantoxin IVa, acetyl(leucyl-alanyl-arginyl-leucyl)3-6-alanyl-6-alanine, acetyl-(LSLLLSL)₃-CONH₂, acetyl-AAVALLPAVLLALLAP-DEVD-CHO, acetyl-AAVALLPAVLLALLAP-YVAD-CHO, NC100668, Ac-PEWLR(Aib)GVTFPGYIT-NH₂, Ac-WGHGHGHGPGHGHGH-NH₂, Ac-WEAQAREALAKEAQARA-NH₂, acetylated peptide A, acetylcysteine(asparaginyl-alanyl-asparaginyl-proline)₃, actinocarcin, actinoxanthine, Ser(3,11)-acyclic sunflower trypsin inhibitor, adipophilin, adolapin, Aek toxin, AF 10847, AF 12415, AF 12505, AF 18748, AF13948, AF15705, AFT-1 toxin, AFT-11 toxin, pig aglycin peptide, AH 111585, AI 3688, AI 409, aibellin, AIP-1-PEO3-ATP, AIP-2-PEO3-ATP, AIP-3-PEO3-ATP, AIPII peptide, ala inhibitor peptide, Ala(0)-actagardine, P+:ISP, Ala-MPSD, alahopcin, albolabrin, ALIN protein, ALL1 peptide, Alloferon, allotrap, α,β-poly((2-hydroxyethypaspartamide-co-(4-hydroxyphenethypaspartamide), α,β poly((2-hydroxyethyl)aspartamide)tyramine, α,β-poly(3-dimethylaminopropyl-D,L-aspartamide), α,β-poly((2-hydroxyethyl)-aspartamide), α-Glu-36 coiled coil, α1BAla, αtα protein, alveolar macrophage growth factor, alytesin, amandin, amastatin, AN 3, AN 7 peptide complex, Anal-R peptide, Anal-S peptide, angiohypotensin, Angiopep-2, anguibactin, annexin A1 peptide(1-25), annexin A1 peptide(2-26), antagonist G, antennapedia-CaMKIINtide, anthopleurin B, anthopleurin C, anthopleurin-A, anthopleurin-Q, anthrax LF protease inhibitor, antiamoebin, antiarrhytmic peptide, anticholecystokinin peptide, pineal antigonadotropin, antineoplaston A2, antineoplaston A3, antineoplaston A5, AP-4F peptide, AP1 peptide, ApC toxin, apstatin, peptide aptamer C1-1, ARAC peptide, Arg-Gly-Asp-Phe-Gly-Gly-Gly-Gly-AP26, arginine-alanine copolymer, arginine-serine polymer, Arg₄-Tyr-Gly-Ser-Arg_{y}-Tyr, RR-SRC, arginyl-leucyl-cysteinyl-arginyl-isoleucyl-valyl-valyl-isoleucyl-arginyl-valyl-cysteinyl-arginyl-aspartyl-aspartyl-aspartyl-aspartyl-glutamyl-glutamic acid(3-11)disulfide, arginyl-aspartyl-glutamyl-glutamic acid(3-11)disulfide, arietin, AS IBBR, ASK 753, TT1272-1284, aspartate carbamoyltransferse regulatory polypeptide, human-type tridecapeptide, AT 464, AT 744, Staphylococcus aureus aureocin A53, autocamptide-2-related inhibitory peptide II, autocamtide 3, autocamtide-2, Ay-AMP peptide, azotobactin, B 43, B2A2 peptide, B2A2-K-NS, Enterococcus faecalis BacA protein, bacteriocin MMFII, bacterioopsin(34-65) polypeptide, BE 22179, belactosin A, belactosin C, benzyloxycarbonyl-(glycyl-prolyl-proline)₈-methyl ester, bergofungin, β-adrenergic receptor kinase inhibitory peptide, Oryctolagus cuniculus β-globin readthrough protein, β-RTX, betabellin 12, BGIA protease inhibitor, bibrotoxin, BIM 23454, BIM 23627, BIM 43004-1, BIM 43073D, bitistatin, bivalirudin, Synechocystis blue-colored linker polypeptide L55, BmK AS polypeptide, BmKIM peptide, BmP02 peptide, BmP03 peptide, BMS 180742, BMS 184696, BMS 184697, BMS 205820, BMS 214572, BMY 28160, bogorol A, boletusin, bombolitins, BPI peptide, bresein, brevistin, bsp-RGD(15) peptide, butyrivibriocin OR79A, Fagopyrum esculentum BWI 3c peptide, BWI 4c peptide, C13-24DE peptide, C18G peptide, C28R2 peptide, cAChR ligand, human CADM-140 peptide, calcemic fraction A, human calcitermin peptide, califin, caloxin 1A1, caloxin 1b1, caloxin 1c2, caloxin 2A1, caloxin 3A1, CALP2 protein, CaMKII inhibitor AIP, carboxypeptidase B activation peptide, carboxypeptidase R intracellular inhibitor, cardiac antisecretory peptide, cardiomyopeptidin, carnocin UI49, carzinophilin, caseidin, CAT 1.6.1, CL22 cationic peptide, cavitein LG2, cavitein LG3, CBLB502, CC 1014, CC 1014B, CDA antibiotic, CDIP-2 peptide, cecropin P1-LI, cell differentiation agent II, cementoin, cepacidine A, cephaibol A, cephaibol B, cephaibol C, ceratitin, cerexins, Cerumenex, cervinin, red-Leu O-acetylated derivative of cervinin, CGP 78850, CGP 85793, chAβ30-16 peptide, Charybdotoxin, CTX-Clv, choroid plexus peptide, chromatophorotropin, chrysospermin A, chrysospermin B, chrysospermin C, chrysospermin D, chymodenin, chymotrypsin inhibitor 2, cicadapeptin I, cicadapeptin II, Cicerarin, cinropeptin, citropeptin, CJC 1131, CKS 17, CKS 25, clavaspirin, clonostachin, Cn 412, Phaseolus coccineus coccinin protein, COG112 peptide, colibiogen, collagen type I trimeric cross-linked peptide, collagen-related peptide, colostrinine, colutellin A, conantokin-L, conantokin-T, connective tissue-activating peptide, copoly(alanine, methionine), copolymer 1, cordialin, cortexin, corticostatin, corticostatin R4, lamprey corticostatin-related peptide, corticotensin, CP 14, CP 530, CREBtide, crotavirin, CS1 peptide, CS4 peptide, CS5 peptide, cupiennin 1a, human CVS995 protein, cyclic chimeric dodecapeptidyl multiple antigen peptide, cyclohexylalanine-prolyl-arginyl-ψCCOCH₂S)glycyl-glycyl-glycyl-glycyl-glycyl-aspartyl-tyrosyl-glutamyl-prolyl-isoleucyl-Prolyl-glutamyl-glutamyl-tyrosyl-cyclohexylalanine-glutamic acid, cystargin, C-εAhx-WKK(C₁₀)-KKK(C₁₀)-KKKK(C₁₀)-YKK(C₁₀)-KK, CYT 379, cytomedins, mouse D-JNKI-1, D2A21, D4E1 peptide, DAB(389)-GRP fusion protein, DAK 16, DAPD peptide, Lys(fluorescein)19-DB3 peptide, Geodia cydonium DD2 protein, debariocidine, DEFB106, deltibant, Dendroaspis natriuretic peptide, dendrotoxin A, dendrotoxin B, dendrotoxin K, β dendrotoxin, γ dendrotoxin, depelestat, deprimerones, dermcidin, DiaPep 277, diazepam binding inhibitor, diazepam binding inhibitor(33-50), diazepam binding inhibitor(39-75), dicynthaurin, dihydromycoplanecin A, dihydropacidamycin D, dimer E.P peptide, dopuin, Dox-penetratin conjugate, Dox-SynB1 conjugate, doxorubicin polyaspartic acid conjugate, doxorubicin-conjugated poly(ethylene glycol)-poly(aspartic acid) block copolymer, drosulfakinin II, DTK1 peptide, DTK2 peptide, DTS-108, dual altered peptide ligand, duodenin, DUP-1 peptide, dynorphin A-analogue kappa ligand, E1E2 peptide, E1K2 peptide, EAA26, EAK 16-IV peptide, ecallantide, echistatin, EF40 peptide, efrapeptin, efrapeptin C, efrapeptin F, efrapeptin G, mouse Egfl6 protein, elapherine A, elapherine B, elapherine C, elapherine D, elastin polypentapeptide, Ile(1)-elastin polypentapeptide, human elastin polypeptide 20-24-24, elegantin, embryonal carcinoma-derived growth factor, enamidonin, Endo-Porter, endosulfine, endothelin converting enzyme substrate, enterogastrone, eosinophilopoietin, EP-2104R, EP1873, Staphylococcus epidermidis EpiA protein, epiactins, epicidin 280, epidermin, epilancin 15X, epilancin K7, epiphisan, epithalamin, eptifibatide, eristostatin, erythrotropin, esein, estromedins, ETR-p1-f1 antisense peptide, exchanger inhibitory peptide, Achatina excitatory peptide 2, Achatina excitatory peptide 3, Fusinus excitatory peptide 4, exorphins, F2(Pmp)2-TAMzeta3 peptide, human F2L peptide, factor XIII activation peptide, FALL 39, FE 999024, FECO peptide, ferrocin A, ferrocin B, ferrocin C, ferrocin D, FGLL peptide, fibrin self-assembly inhibitor, fibrinogen binding inhibitor peptide, fibrinopeptide A, 5-tyrosine fibrinopeptide A, desaminotyrosylfibrinopeptide A, desAla1-fibrinopeptide A, phospho-Ser3-fibrinopeptide A, Fibrinopeptide B, desArg14-fibrinopeptide B, FLAG peptide, FLPIVGAKL, Fn-23 peptide, foroxymithine, FR 900490, FRAP-4 peptide, friulimicin A, friulimicin B, friulimicin C, friulimicin D, FROPDOTA compound, G10KHc peptide, G25 peptide, gaegurin 5, GALA peptide, GALAdel3E peptide, gallidermin, γ-Glu-36 coiled coil, gangliin, Gap 26 peptide, gastric releasing peptide, gastrin-releasing peptide precursor, GAT, GD1α-replica peptide, GE20372 factor A, Streptosporangium cinnabarinum GE82832 peptide, gene-drived bombinin H-like peptide, geninthiocin, gilatoxin, gliadin peptide B 3142, glidobactin D, glidobactin E, glidobactin F, glidobactin G, globopeptin, glucagon 29, glucagon releasing peptide, glucose utilization inhibitor, glucose-6-phosphate dehydrogenase inhibition controlling cofactor, glutamic acid-arginine-alanine polymer, glutamic acid-lysine-alanine polymer, glutamic acid-lysine-tyrosine terpolymer, glutamyl-phenylalanyl-threonine, EFW-NPSF, glutathione peroxidase-related selenopeptide, G1y14-humanin, glycine-proline-proline polymer, glycothiohexide α, GGG[Y²]-speract, GNRH precursor(14-26), goadsporin, gold keratinate, goniopora toxin, GR 83074, gratisin, GRF-PHI heptacosapeptide amide, griseoviridin, GsMTx-4 toxin, growth hormone-releasing peptide, growth hormone-releasing peptide-6, GSP-6 glycosulfopeptide, GTD-C protein, guamerin, guanylin 16, guanylin 94, GW395058, serine human angiotensin tetradecapeptide, H2K4bT protein, H5WYG peptide, halocidin, halocin S8, hanatoxin, harzianin HA V, HB-19 peptide, HBY 793, Hel 13-5 peptide, helical erythrocyte lysing peptide, heliodermin, helodermin(1-28) amide, helospectin, helospectin I, helospectin II, helothermine, hemalin, hematide, HepArrest, hepatic stimulator substance, herbicolin B, hexadecalysyl-glycyl-arginyl-glycyl-aspartyl-serinyl-prolyl-cysteine, hexamethylene diisocyanate cross-linked polypeptides, human immunodeficiency virus-1 HGP-30 peptide, HI peptide, hirullin P18, hirunorm, hirunorm IV, His6-tagged elastin-like peptide, HLP-1 polypeptide, HLP-2 polypeptide, HLP-3 polypeptide, HLP-6 peptide, Hoa-MIH, HRES-1 p25 protein, human X polypeptides, Huia versabilis HV-BBI peptide, HXP4 peptide, HXR9 peptide, Hym 346, Hym-323 peptide, hypeptin, hypertensive factor, hypotensin (snake), iberiotoxin, iberiotoxin-D19Y-Y36F, IC 101, ideal amphipathic peptide, imacidins, immunopeptide 1, Lactobacillus inducing factor, INF7 peptide, insulin resistance factor (uremia), insulin-glucagon liberin, human insulin-stimulating peptide, β-Interleukin I(163-171), β-Interleukin II(44-56), Interleukin II (60-70), iodo-Tyr5-Phe36-iberiotoxin, IP 20, isariin, JCP 405, JCP 410, Chilobrachys jingzhao jingzhaotoxin XI, Chilobrachys jingzhao jingzhaotoxin-I, Chilobrachys jingzhao jingzhaotoxin-III, Chilobrachys jingzhao jingzhaotoxin-V, joining segment peptide, K 582 A, K-MLC 11-23, K1E2 peptide, K1 K2 peptide, K4-M2GIyR protein, K5 peptide, KAI 9803, KALA amphipathic peptide, kassinakinin S, kassinatuerin-1, kawaguchipeptin B, KB-752 peptide, kedarcidin peptide, KIA7 protein, KID1-1 peptide, KIE1-1 peptide, KILT protein, kinetensin, kistamicin A, kistamicin B, kistrin, KL4 surfactant, KM 8, KT 199, plant Kunitz-type protease inhibitor, L 363714, L 689502, L 694746, L 733560, L-4F peptide, L-glutamic acid-L-tyrosine copolymer, L-JNKI-1, L-T6DP-1 peptide, lac α peptide, lactivicin, lactocin S, LAGA, LAH(4) protein, lambda Spi-1, lambda Spi-2, lanthiopeptin, lantibiotic PepS, LBMP1620 protein, leiuropeptide II, leiurutoxin III, Leu-Ser-Lys-Leu peptide, Leu3 blomhotin, leucinostatin A, leucinostatin B, leucinostatin C, leucinostatin D, leucinostatin H, leucinostatin K, leukocyte mobilizing factor, leupeptin, leuteonosticon, Limnoperna fortunei LF22 peptide, LH receptor binding inhibitor, lichenysin G, linaclotide, lipid mobilizing substance, lipid-associating peptides, lipopeptin A, lipoprotein lipase activators, LIQ 4, live yeast cell derivative, LL AF283β, LL AO341β1, longibrachin LGA I, longibrachin LGB II, longibrachin LGB III, LR9 peptide, lumbricin I, LY 295337, LY 315902, lymphoguanylin, lys-guanylin, lysoartrosi, lysometra, M-81, M1557 peptide, M2 delta, maduropeptin A1, maduropeptin A2, maduropeptin B, maduropeptin C, magainin-PGLa hybrid peptide, magaratensin, magnificalysin I, magnificalysin II, magnificalysin III, malantide, mamba intestinal toxin 1, mammastatin, MAP 1987, Mas-DP II, Mas7 protein, mast 21 peptide, mast cell degranulating peptide, mastoparan, mastoparan B, mastoparan M, mastoparan X, 11-dansyl-mastoparan, mating factor, Ala9-mating factor, maximin H1, MB21 peptide, MCP-4-EDTA-SH, MCR 14 peptide, MCR 4 peptide, MDL 27,367, melanophore-dispersing hormone, meliacin, Mer N5075A, mersacidin, methinin, methylenomycin A, michicarcin, microbial alkaline proteinase inhibitor, microbisporicin, microcin H47, microcin SF608, micrococcin, mitochondrial addressing peptide, mitomalcin, mitoparan, miyakamide A1, miyakamide A2, miyakamide B1, miyakamide B2, MJ347-81F4 A, MJ347-81F4 B, MLCK peptide, MMK-1 peptide, monocyto-angiotropin, monoketo-organomycin, motilin-associated peptide, MPGα peptide, MS-681a, MS05 peptide, MS09 peptide, MSI 511, MSI 594, MSI-99 peptide, MT-7 mamba toxin, multide, Leu7-multiple antigen peptide, leucyl(8)-lysyl(4)-lysyl(2)-lysyl-β-alanine multiple antigen peptide, muscarinic toxin 3, mutacin 1140, bacteria mutacin II prepeptide, MW167, mycoplanecin A, Myocardial Depressant Factor, myristoylated autocamtide-2-related inhibitory peptide, MGAIPAA, myroridin, myroridin K, myxovalargins, N-acetyl-gastrin releasing peptide ethyl ester, N-methyldepsipeptide, N-tert-butyloxycarbonyl-valyl-alanyl-leucyl-aminoisobutyryl-valyl-alanyl-leucyl(valyl-alanyl-leucyl-aminoisobutyryl)(2) methyl ester, Nano-1 peptide, mouse NBD peptide, neosulfakinin II, NeoTect, neotelomycin, neoviridogriseins, NK911, nocathiacin I, novospirin G-10, NSC 710295, NVP PDF 713, NVP-PDF386, octameric MYFGGGGG ligand, OS-3256-B, osteoclast stimulating factor, ovCNP-39 peptide, ovocystatin, oxaldie 1, oxaldie 2, p 230, P 498, P 500, P-LF II D, P.polypeptide, P596 peptide, P62 peptide, PA22-2, Paim I, Pam(3)CSK(4) peptide, Gila monster venom pancreatic secretory factor, pancreatic spasmolytic polypeptide, pandinin 1, pandinin 2, pantripin, Asp(12),Arg(13)-paotin-lysyl-GRP-27, PC 1038, PD-145065, PD 142893, mouse Pdcd1Ig1 protein, mouse Pdcd1lg2 protein, PEC-60 polypeptide, pedibin, PEG-DAPD peptide, PEG-PAsp(Dox), penaeidin 1, penaeidin 2, penaeidin 3, Pep-1 peptide, pep-1CF peptide, Pep-3 peptide, Pep-9 oligodeoxyribonucleotide-peptide conjugate, pepBs1-Ac peptide, PEPHC1 peptide, pepsanurin, DA RT1(A) peptide 1, peptide 106, peptide 18A, rat peptide 19, Fagopyrum esculentum peptide 4 kDa, peptide 5F, peptide 74, peptide 78, peptide 9M, peptide I, peptide KPR, peptide leucine arginine, peptide MB-35, peptide methionine-tyrosine, peptide NK-2, peptide P3, peptide Q, peptide S 42, peptide S-8300, peptide SC-R8A2, peptide SM-BC3, peptide stabilizing factor, peptide U6, peptidimer-c, peptilose, peptitergent PD1, Peri Coil 1, Perinerin, periodontal ligament chemotactic factor, permetin A, phenylalanyl-glycyl-glycyl-phenylalanyl-threonyl-glycyl-a-aminoisobutyryl-arginyl-lysyl-seryl-α-aminoisobutyryl-arginyl-lysyl-leucyl-alanyl-asparagyl-glutaminamide, PHM polymer, phosphofructokinase regulatory factors, phylloxin, PK1M peptide, PKL-lc peptide, plasmatocyte-spreading peptide 1, plauracin, plectasin, PNV2 peptide, PNV4 peptide, polistes mastoparan, poly(RGD), poly(RGDT), Poly 18 antigen, poly(1-benzylhistidine), poly(2-sulfoethyl aspartamide)silica, poly(3-hydroxypropyl)aspartamide, poly(3-hydroxypropyl-propyl)asparamide, poly(Ala)-poly(Lys), poly(alanyl-glutamyl-tyrosyl-glycine), poly(alanyl-tyrosyl-glutamyl-glycine), poly(alanyl-valyl-glycyl-valyl-prolyl), poly(alanylglycine), poly(arginyl-histidine), poly(aspartylhydrazide), poly(diethylaminoethylglutamine), poly(dimethylaminoethylglutamine), poly(ethylene oxide-co-β-benzyl-L-asparate), poly(γ-glutamylcysteinyl)clycine, poly(γ-methylglutamate)-grated polyallylamine, poly(Glu56-Lys35-Phe9)n, poly(glutamyl-alanyl-tyrosyl-glycine), poly(glutamyl-tyrosyl-alanyl-glycine), poly(glycyl-prolyl-serine), poly(glycyl-valyl-glycyl-valyl-proplyl), poly(glycyl-valyl-hydroxyproline), poly(histidyl-aspartyl-seryl-glycine) poly(hydroxybutylglutamine-co-proline), poly(hydroxyethylaspartamide-co-aspartic acid), poly(hyddroyxethylaspartamide-co-dimethylaminopropylaspartamide), poly(hydroxyprolyl-prolyl-glycine) (10, poly(L-aspartyl-L-phenylalanine), poly(L-tyrosyl-L-glutamyl-L-alanyl-glycyl)glycine ethyl ester, poly(leucyl-glycyl-glycyl-valyl-glycyl), poly(leucyl-phenylalanyl-proline), poly(lysyl-(glutamyl(i)-alanine(m))), poly(lysyl-poly-alanine)), poly(lysyl-seryl-glutamic acid), poly(lysyl-tyrosyl-tyrosyl-lysine), poly(N(β)-4-(phenylazo-benzoyl-α,β-diaminopropionic acid), poly(N(delta), poly(delta), poly(delta)-trimethylornithine), poly(N-(3-aminopropyl)glycine), poly(N-hydroxypropylglutamine-leucine), poly(O,O′-dicarbobenzoyx-L-β-3,4-dihydroxyphenyl-α-alanine), poly(phenylalanyl-alanyl-glutamyl-glycine), poly(phenylalanyl-glutamyl-alanyl-glycine), poly(prolyl-norleucyl-glycine), poly(prolylprolylglycine)15, poly(S-carboxymethylcysteine), poly(sarcoyl-glycl-phenylallanyl-leucyl-glycyl-aminoethylaminocarbonylmethyl(N-methyl)amino-co-α, omega-bis(oxiranylmethyl)poly(ethylene glycol)), poly(tyrosyl-alanyl-glutamyl-glycine), poly(tyrosyl-glutamic acid), poly(tyrosyl-glutamyl-ananyl-glycyl), poly(tyrosyl-isoleucyl-glycyl-seryl-arginine), poly(undecanoylvalinate), poly(valyl-glycyl-glycyl-valyl-glycine), poly-(His-Glu)-poly Ala-poly Lys, poly-β-benzyl-aspartate, poly-delta-L-arnithine, poly-DL-succinimide, poly-L-lysylphenylalanine, poly-L-lysyltyrosine, poly-N(5)-2-hydroxyethyl)glutamine, poly-N(5)-3-hydroxypropyl)-1-glutamide, poly-N(5)-(3-hydroxypropylglutamine)-prazosin carbamate, poly-O-acetylserine, poly-O-carbobenzoyserine, poly-S-benzylcysteine, poly-S-carbobenzoxycysteine, polyalanine, polyarginine, polyasparagine, polyaspartate, polyaspartoyl-L-arginine, polyaspartylglutamate, polychlorosubtilin, polycysteine, polyetherurethaneurea-polypeptide block copolymer, polyethylenimine-N-succinimidyl-3-(2-pyridyldithio)propionyl-MC11, Polygeline, polyglutamine, polyglycine, polyisoleucine, polyleucine, polymethionine, polymethionine, sulfoxide, polymorph, migration stimulator, polyoma peptide antigen MT162-176, polyornithine, polypeptide C, polypeptide oleate condensate, polypeptide pineal extract, polypeptide PPA-80, polypenylalanine, polyproline, polysarcosine, polyserine, polytryptophan, polytyrosine, polyvaline, prelacticin 481, probursin, procamine, progressin, proline-rich polypeptide, prolyl-lysyl-leucyl-leucyl-lysyl-threonyl-phenylalanyl-leucyl-seryl-lysyl-tryptophyl-iisoleucyl-glycine, prolyl-seryl-glycyl-phenylalanyl-tyrosyl-leucyl-lysyl-leucyl-aspartyl-prolyl-arginyl-asparaglinyl-phenylalanyl-asparagine, promoinducin, promothiocin, prostallin, rat prostate specific binding protein po9lypeptide C3, prostatilen, protein B23 antigenic, peptide, protein kinase inhibitor peptide, prtb peptide, pseudokonin KL III, pseudokonin KL VI, PTP-7S, peptide, pulmolin, pumilacidin, pVEC peptide, PW2 peptide, PYL(a), plyoricidin A, pyloricidin B, pyloricidin C, mouse Qdm protein, QK VEGF mimetic peptide, QRFP peptide, R18 peptide, R6A-1 peptide, rab3AL peptide, ranatensin R, ranatuerin, Raytide, RC 101, RCS-RF, resact, retilalamin, retro-bombolitin, I, retro-bombolitin III, retro-inverso-TAT p53C′ peptide, retro-nociceptin methylester, Rev peptide, Reb4 peptide, RH4 peptide, RHM1 peptide, RHM2 peptide, rhodostomin, RI-26 peptide, RK 699A, tetrahymena RNA inhibitor, RP 66453, RS 83277, S 862033, S 863390, S Ht31, S4(13)-PV peptide, S4K2Kr3A core peptide, S597 peptide, Sadat-Habdan mesenchymal stimulating peptide, samarosporin, sarafotoxin-c, saramycetin, saturnisporin SA II, saturnisporin SA IV, SC 40476, SC 42619, Sch 40832, Sch 419558, Sch 419559, SCH 466456, SCH 466457, schistosomin, scotophobin, Dictyostelium discoideum SDF-2 protein, semparatide acetate, SepOvotropin, sertolin, serum sodium transport inhibitor, mouse Sftpc protein, rat Sftpc protein, Shaker B inactivating peptide, short ragweed fraction A-D-glutamic acid-D-lysine polymer, siamycin I, siamycin II, sideromycin No. 216, sifuvirtide, silaffin 1A, silaffin 1B, sillucin, sinapultide, SM3-MUC1 peptide, SN50 peptide, SNK 863, SNP-1 protein, SNX 202, SNX 260, SNX 325, Conus striatus SO-3 conopeptide, somatostatin-like peptides, sorbin, spasmolytic polypeptide, sperm acrosomal peptide P23, spinigerin, rat Sponf protein, Streptomyces spore pigment, SQ 20858, SR 41476, stearated-Ht31 peptide, Streptococcus streptococcin A M49 protein, Styela clava styelin A peptide, Styela clava styelin B peptide, sublancin 168, substance P-like peptides, suzukacillin, sweet arrow peptide, synthetic peptide a27-50, syntide-2, systemin, T-activin, T1BP2 peptide, T1BT peptide, T22 peptide, human TAFII-17 protein, TAT-R7-LV1 peptide, TAT-TIJIP, tatumine, TC5b protein, TcapQ647 peptide, ^{99m}Tc-AGGCG, ^{99m}Tc-AGGCL, ^{99m}Tc-ASSCG, ^{99m}Tc-labeled α-M2 peptide, teduglutide, tendamistate, tessulin, testilin, Tet-p peptide, Eisenia tetradecapeptide, texenomycin A, mouse TFF1 protein, mouse TFF2 protein, human TFF3 protein, theonellapeptolide le, theromin, THG113.31 peptide, thioactin, thiocillin, thiopeptin, thiopeptin A, thiopeptin B, Thomsen-Friedeneich antigen-specific peptide P-30, Thr-Met-Lys-Ile-Ile-Pro-Phe-Asn-Arg-Thr-Leu-Ile-Gly-Gly, thrombopoietin mimetic peptide, thymocyte growth peptide, thymodepressin, thymohemin, thymone A, thymone B, thymone C, tick anticoagulant peptide, TIFI peptide, tigerinin 1, tissue polypeptide antigen, tissue polypeptide specific antigen, TL 119, TM11 peptide, TN14003, TNYL-RAW peptide, toxin FS2, TP10-PNA conjugate, transfecting peptide I, transporter peptide HGH6, trapoxin A, trapoxin B, trefoil factor, trichocellin, trichogin A IV, tricholongin BI, tricholongin BII, trichopolyn, trichorzin PA IV, trichorzin PA V, trichorzin PA VI, trichotoxin, trichotoxin A 50E, trichotoxin A40, trichovirin I IB, trichovirin I-4A, tridecaptins, triflavin, trifolitoxin, trigramin, trikoningin KB II, trimucrin, triwaglerin, trofopar, Trp-cage peptide, duck pancreas trypsin inhibitor, tuberoinfundibular peptide 38, turmerin, tylopeptin A, tylopeptin B, tyrosinase inhibitor, U 995, UK 156406, vaccinia growth factor, valosin, valyl-prolyl-glycyl-valyl-glycine polypeptide, VAP-map peptide, vascular factor, vasoactive intestinal constrictor, vasonin, ventriculine, vermilat, villikinin, vishnu, Vitaprost, Grammostola spatulata VSTX1 protein, VT5 peptide, Vueffe, Walsh peptide, WeiJia, WF 3161, Wheel-FKFE, WR-PAK18 peptide, Xen2174, xenin 25, xenopsin, XK-19-2, XR 586, xylocandin, Y(21) peptide, YALA peptide, YM 170320, YM 266183, YM 266184, YTA2 peptide, YTA4 peptide, Z 2685, Z28 peptide, zinc finger peptide Xfin-31, ZP10A peptide, Zwit-1F peptide, human chorionic thyrotropin protein, decidua inhibitory factor, placental antigen X-P2, placental lactogen, placental lactogen A-2, rat placental lactogen I, placental lactogen I-variant, placental lactogen II, human placental lactogen-3, placental ribonuclease inhibitor, placental uterotrophic factor, and prolactin-releasing factor (placenta).

Disintegrins

In certain embodiments, the peptide therapeutic is a disintegrin or an analog thereof. Such peptides include accutin, acostatin, rat Adam9 protein, Agkistrodon halys brevicaudus stejneger adinbitor protein, alternagin-C, bitisgabonin-1, bitisgabonin-2, Bothrops jararaca bothrostatin, contortrostatin, Echis carinatus EC3 protein, Echis carinatus sochureki EC6 protein, Eristocophis macmahoni EMF10 protein, flavorodin, flavostatin, jarastatin, jerdonin, Drosophila kuzbanian protein, C. elegans MIG-17 protein, ocellatusin, piscivostatin, saxatilin, Trimeresurus stejnegeri stejnin protein, Trimeresurus flavoviridis trimestatin protein, Gloydius ussuriensis ussuristatin 1 protein, and Gloydius ussuriensis ussuristatin 2 protein.

Endothelins

In certain embodiments, the peptide therapeutic is an endothelin or an endothelin analog. Such peptides include Phe(22)-big endothelin-1(19-37), Val(22)-big endothelin-1(16-38), big-endothelin(1-22), big-endothelin(16-32), BQ 3020, Cys(11)-Cys(15)-endothelin-1 (11-21), endothelin(16-21), endothelin(16-21) amide, Endothelin-1, (Sec(3)-Sec(11)-Nle(7))-endothelin-1, zebrafish edn1 protein, endothelin-1(1-21), (Cys,Acm(1,15),Aib(3,11),Leu(7))-endothelin-1(1-21), endothelin-1(1-31), (Aib(1,3,11,15),Nle(7))-endothelin-1, Aba(1,15)-endothelin-1, Ala(10)-endothelin-1, Cys(Acm)(1,15)-Ala(3)-Leu(7)-Aib(11)-endothelin-1, formylTrp(21)-endothelin-1, lysyl(-2)-arginyl(-1)-endothelin-1, Pen(1,11)-Nle(7)-Ala(18)-endothelin-1, Pen(1,11)-Nle(7)-Asn(18)-endothelin-1, Phe(16)-endothelin-1, Thr(18)-Cha(19)-endothelin-1, Thr(18)-Leu(19)-endothelin-1, ET-1(Cys(Acm)(1,15)-Ala(3)-Leu(7)-dAsp(8)-Aib(11)), proendothelin 1, Endothelin-3, Ala(1,15)-endothelin 1, Ala(1,3,11,15)-endothelin 1, Ala(3,11)-endothelin 1, Dpr(1)-Asp(15)-endothelin 1, Nle(7)-endothelin, Ala(9)-endothelin-1, Pro(12)-endothelin-1, Thr(18)-y-methyl-Leu(19)-endothelin-1, endothelin-1,2-6-keto-PGF1-α, endothelin7-21(Leu7,Aib11,Cys(Acm)15), Endothelin-2, IRL 1620, IRL 1720, N(E)-9-azidobenzoyliodoendothelin-1, preproendothelin 2, preproendothelin-3, proendothelin 2, proendothelin 3, proendothelin-1(22-39), and proendothelin-1(31-38).

Secretory Proteinase Inhibitory Protein

In certain embodiments, the peptide therapeutic is a secretory proteinase inhibitory protein or an analog thereof. Such peptides include α1-antichymotrypsin, α1-antitrypsin, S. cerevisiae A1PlZ protein, α1-antitrypsin Christchurch, α1-antitrypsin Pittsburgh, α1-antitrypsin Portland, α1-antitrypsin QOtrastevere, α1-antitrypsin Siiyama, α1-antitrypsin W(Bethesda), S α1-antitrypsin, α1-antitrypsin-leukocyte elastase complex, C105Y peptide, human serpin A1 (A1-C26), human SERPINA1 protein, human SERPINA2 protein, trypsin-2-α1-antitrypsin, human VIRIP peptide, Elafin, Human Pl3 protein, zebrafish Hai1 protein, human ITIH4 protein, human ITIH5 protein, mouse protease inhibitor 16, human secretory leukocyte peptidase inhibitor (SLPI) protein, mouse SLPI protein, rat SLPI protein, human SPINK5 protein, human SPINLW1 protein, and human SPINT1 protein.

### Modified Forms of Peptide Therapeutics

Any of the peptide therapeutics described herein (e.g., GLP-1 agonists) may be modified (e.g., as described herein or as known in the art). As described in U.S. Pat. No. 6,924,264, the polypeptide can be bound to a polymer to increase its molecular weight. Exemplary polymers include polyethylene glycol polymers, polyamino acids, albumin, gelatin, succinyl-gelatin, (hydroxypropyI)-methacrylamide, fatty acids, polysaccharides, lipid amino acids, and dextran.

In one case, the polypeptide is modified by addition of albumin (e.g., human albumin), or an analog or fragment thereof, or the Fc portion of an immunoglobulin. Such an approach is described, for example, in U.S. Pat. No. 7,271,149.

In one example, the polypeptide is modified by addition of a lipophilic substituent, as described in PCT Publication WO 98/08871. The lipophilic substituent may include a partially or completely hydrogenated cyclopentanophenathrene skeleton, a straight-chain or branched alkyl group; the acyl group of a straight-chain or branched fatty acid (e.g., a group including CH₃(CH₂)ₙCO— or HOOC(CH₂)ₘCO—, where norm is 4 to 38); an acyl group of a straight-chain or branched alkane α,ω-dicarboxylic acid; CH₃(CH₂)ₚ((CH₂)_{q}, COOH)CHNH—CO(CH₂)₂CO—, where p and q are integers and p+q is 8 to 33; CH₃(CH₂),CO—NHCH(COOH)(CH₂)₂CO—, where r is 10 to 24; CH₃(CH₂)ₛCO—NHCH((CH₂)₂COOH)CO—, where s is 8 to 24; COOH(CH₂)ₜCO—, where t is 8 to 24; —NHCH(COOH)(CH₂)₄NH—CO(CH₂)ᵤCH₃, where u is 8 to 18; —NHCH(COOH)(CH₂)₄NH—COCH((CH₂)₂COOH)NH—CO(CH₂)_{w}CH₃, where w is 10 to 16; —NHCH(COOH)(CH₂)₄NH—CO(CH₂)₂CH(COOH)NH—CO(CH₂)ₓCH₃, where x is 10 to 16; or —NHCH(COOH)(CH₂)₄NH—CO(CH₂)₂CH(COOH)NHCO(CH₂)_{y}CH₃, where y is 1 to 22.

In other embodiments, the peptide therapeuticis modified by addition of a chemically reactive group such as a maleimide group, as described in U.S. Patent No. 6,593,295. These groups can react with available reactive functionalities on blood components to form covalent bonds and can extending the effective therapeutic in vivo half-life of the modified insulinotropic peptides. To form covalent bonds with the functional group on a protein, one can use as a chemically reactive group a wide variety of active carboxyl groups (e.g., esters) where the hydroxyl moiety is physiologically acceptable at the levels required to modify the peptide. Particular agents include N-hydroxysuccinimide (NHS), N-hydroxy-sulfosuccinimide (sulfo-NHS), maleimide-benzoyl-succinimide (MBS), gamma-maleimido-butyryloxy succinimide ester (GMBS), maleimido propionic acid (MPA) maleimido hexanoic acid (MHA), and maleimido undecanoic acid (MUA).

Primary amines are the principal targets for NHS esters. Accessible a-amine groups present on the N-termini of proteins and the E-amine of lysine react with NHS esters. An amide bond is formed when the NHS ester conjugation reaction reacts with primary amines releasing N-hydroxysuccinimide. These succinimide containing reactive groups are herein referred to as succinimidyl groups. In certain embodiments of the invention, the functional group on the protein will be a thiol group and the chemically reactive group will be a maleimido-containing group such as gamma-maleimide-butrylamide (GMBA or MPA). Such maleimide containing groups are referred to herein as maleido groups.

The maleimido group is most selective for sulfhydryl groups on peptides when the pH of the reaction mixture is 6.5-7.4. At pH 7.0, the rate of reaction of maleimido groups with sulfhydryls (e.g., thiol groups on proteins such as serum albumin or IgG) is 1000-fold faster than with amines. Thus, a stable thioether linkage between the maleimido group and the sulfhydryl is formed, which cannot be cleaved under physiological conditions.

### Peptide Vectors

The compounds of the invention can feature any of polypeptides described herein, for example, any of the peptides described in Table 1 (e.g., Angiopep-1 or Angiopep-2), or a fragment or analog thereof as a peptide vector. In certain embodiments, the peptide vector may have at least 35%, 40%, 50%, 60%, 70%, 80%, 90%, 95%, 99%, or even 100% identity to a polypeptide described herein. The peptide vector may have one or more (e.g., 2, 3, 4, 5, 6, 7, 8, 9, 10, 11, 12, 13, 14, or 15) substitutions relative to one of the sequences described herein. Other modifications are described in greater detail below.

The invention also features fragments of these polypeptides (e.g., a functional fragment). In certain embodiments, the fragments are capable of efficiently being transported to or accumulating in a particular cell type (e.g., liver, eye, lung, kidney, or spleen) or are efficiently transported across the BBB. Truncations of the polypeptide may be 1, 2, 3, 4, 5, 6, 7, 8, 9, 10, 11, 12, or more amino acids from either the N-terminus of the polypeptide, the C-terminus of the polypeptide, or a combination thereof. Other fragments include sequences where internal portions of the polypeptide are deleted.

Additional peptide vectors may be identified by using one of the assays or methods described herein. For example, a candidate polypeptide may be produced by conventional peptide synthesis, conjugated with paclitaxel and administered to a laboratory animal. A biologically-active polypeptide conjugate may be identified, for example, based on its ability to increase survival of an animal injected with tumor cells and treated with the conjugate as compared to a control which has not been treated with a conjugate (e.g., treated with the unconjugated agent). For example, a biologically active polypeptide may be identified based on its location in the parenchyma in an in situ cerebral perfusion assay.

Assays to determine accumulation in other tissues may be performed as well. Labelled conjugates of a polypeptide can be administered to an animal, and accumulation in different organs can be measured. For example, a polypeptide conjugated to a detectable label (e.g., a near-IR fluorescence spectroscopy label such as Cy5.5) allows live in vivo visualization. Such a polypeptide can be administered to an animal, and the presence of the polypeptide in an organ can be detected, thus allowing determination of the rate and amount of accumulation of the polypeptide in the desired organ. In other embodiments, the polypeptide can be labelled with a radioactive isotope (e.g., ¹²⁵I). The polypeptide is then administered to an animal. After a period of time, the animal is sacrificed and the organs are extracted. The amount of radioisotope in each organ can then be measured using any means known in the art. By comparing the amount of a labeled candidate polypeptide in a particular organ relative to the amount of a labeled control polypeptide, the ability of the candidate polypeptide to access and accumulate in a particular tissue can be ascertained. Appropriate negative controls include any peptide or polypeptide known not to be efficiently transported into a particular cell type (e.g., a peptide related to Angiopep that does not cross the BBB, or any other peptide).

Additional sequences are described in U.S. Pat. No. 5,807,980 (e.g., SEQ ID NO:102 herein), U.S. Pat. No. 5,780,265 (e.g., SEQ ID NO:103), U.S. Pat. No. 5,118,668 (e.g., SEQ ID NO:105). An exemplary nucleotide sequence encoding an aprotinin analog atgagaccag atttctgcct cgagccgccg tacactgggc cctgcaaagc tcgtatcatc cgttacttct acaatgcaaa ggcaggcctg tgtcagacct tcgtatacgg cggctgcaga gctaagcgta acaacttcaa atccgcggaa gactgcatgc gtacttgcgg tggtgcttag; SEQ ID NO:6; Genbank accession No. X04666). Other examples of aprotinin analogs may be found by performing a protein BLAST (Genbank: www.ncbi.nlm.nih.gov/BLAST/) using the synthetic aprotinin sequence (or portion thereof) disclosed in International Application No. PCT/CA2004/000011. Exemplary aprotinin analogs are also found under accession Nos. CAA37967 (Gl:58005) and 1405218C (Gl:3604747).

### Modified Polypeptides

The peptide vectors and peptide therapeutics used in the invention may have a modified amino acid sequence. In certain embodiments, the modification does not destroy significantly a desired biological activity (e.g., ability to cross the BBB). The modification may reduce (e.g., by at least 5%, 10%, 20%, 25%, 35%, 50%, 60%, 70%, 75%, 80%, 90%, or 95%), may have no effect, or may increase (e.g., by at least 5%, 10%, 25%, 50%, 100%, 200%, 500%, or 1000%) the biological activity of the original polypeptide. The modified peptide may have or may optimize a characteristic of a polypeptide, such as in vivo stability, bioavailability, toxicity, immunological activity, immunological identity, and conjugation properties.

Modifications include those by natural processes, such as posttranslational processing, or by chemical modification techniques known in the art. Modifications may occur anywhere in a polypeptide including the polypeptide backbone, the amino acid side chains and the amino- or carboxy-terminus. The same type of modification may be present in the same or varying degrees at several sites in a given polypeptide, and a polypeptide may contain more than one type of modification. Polypeptides may be branched as a result of ubiquitination, and they may be cyclic, with or without branching. Cyclic, branched, and branched cyclic polypeptides may result from posttranslational natural processes or may be made synthetically. Other modifications include pegylation, acetylation, acylation, addition of acetomidomethyl (Acm) group, ADP-ribosylation, alkylation, amidation, biotinylation, carbamoylation, carboxyethylation, esterification, covalent attachment to fiavin, covalent attachment to a heme moiety, covalent attachment of a nucleotide or nucleotide derivative, covalent attachment of drug, covalent attachment of a marker (e.g., fluorescent or radioactive), covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination.

A modified polypeptide can also include an amino acid insertion, deletion, or substitution, either conservative or non-conservative (e.g., D-amino acids, desamino acids) in the polypeptide sequence (e.g., where such changes do not substantially alter the biological activity of the polypeptide). In particular, the addition of one or more cysteine residues to the amino or carboxy terminus of any of the polypeptides of the invention can facilitate conjugation of these polypeptides by, e.g., disulfide bonding. For example, Angiopep-1 (SEQ ID NO:67), Angiopep-2 (SEQ ID NO:97), or Angiopep-7 (SEQ ID NO:112) can be modified to include a single cysteine residue at the amino-terminus (SEQ ID NOS: 71, 113, and 115, respectively) or a single cysteine residue at the carboxy-terminus (SEQ ID NOS: 72, 114, and 116, respectively). Amino acid substitutions can be conservative (i.e., wherein a residue is replaced by another of the same general type or group) or non-conservative (i.e., wherein a residue is replaced by an amino acid of another type). In addition, a non-naturally occurring amino acid can be substituted for a naturally occurring amino acid (i.e., non-naturally occurring conservative amino acid substitution or a non-naturally occurring non-conservative amino acid substitution).

Polypeptides made synthetically can include substitutions of amino acids not naturally encoded by DNA (e.g., non-naturally occurring or unnatural amino acid). Examples of non-naturally occurring amino acids include D-amino acids, an amino acid having an acetylaminomethyl group attached to a sulfur atom of a cysteine, a pegylated amino acid, the omega amino acids of the formula NH₂(CH₂)ₙCOOH wherein n is 2-6, neutral nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties.

Analogs may be generated by substitutional mutagenesis and retain the biological activity of the original polypeptide. Examples of substitutions identified as “conservative substitutions” are shown in Table 2. If such substitutions result in a change not desired, then other type of substitutions, denominated “exemplary substitutions” in Table 3, or as further described herein in reference to amino acid classes, are introduced and the products screened.

Substantial modifications in function or immunological identity are accomplished by selecting substitutions that differ significantly in their effect on maintaining (a) the structure of the polypeptide backbone in the area of the substitution, for example, as a sheet or helical conformation. (b) the charge or hydrophobicity of the molecule at the target site, or (c) the bulk of the side chain. Naturally occurring residues are divided into groups based on common side chain properties:
(1) hydrophobic: norleucine, methionine (Met), Alanine (Ala), Valine (Val), Leucine (Leu), Isoleucine (Ile), Histidine (His), Tryptophan (Trp), Tyrosine (Tyr), Phenylalanine (Phe),
(2) neutral hydrophilic: Cysteine (Cys), Serine (Ser), Threonine (Thr)
(3) acidic/negatively charged: Aspartic acid (Asp), Glutamic acid (Glu)
(4) basic: Asparagine (Asn), Glutamine (Gin), Histidine (His), Lysine (Lys), Arginine (Arg)
(5) residues that influence chain orientation: Glycine (Gly), Proline (Pro);
(6) aromatic: Tryptophan (Trp), Tyrosine (Tyr), Phenylalanine (Phe), Histidine (His),
(7) polar: Ser, Thr, Asn, Gln
(8) basic positively charged: Arg, Lys, His, and;
(9) charged: Asp, Glu, Arg, Lys, His

Other amino acid substitutions are listed in Table 3.

TABLE 2
Amino acid substitutions

| Original | | Conservative |
|---|---|---|
| residue | Exemplary substitution | substitution |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, norleucine | Leu |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, norleucine | Leu |

Polypeptide Derivatives and Peptidomimetics

In addition to polypeptides consisting of naturally occurring amino acids, peptidomimetics or polypeptide analogs are also encompassed by the present invention and can form the peptide vectors or peptide therapeutics used in the compounds of the invention. Polypeptide analogs are commonly used in the pharmaceutical industry as non-peptide drugs with properties analogous to those of the template polypeptide. The non-peptide compounds are termed “peptide mimetics” or peptidomimetics (Fauchere et al., Infect. Immun. 54:283-287,1986 and Evans et al., J. Med. Chem. 30:1229-1239, 1987). Peptide mimetics that are structurally related to therapeutically useful peptides or polypeptides may be used to produce an equivalent or enhanced therapeutic or prophylactic effect. Generally, peptidomimetics are structurally similar to the paradigm polypeptide (i.e., a polypeptide that has a biological or pharmacological activity) such as naturally-occurring receptor-binding polypeptides, but have one or more peptide linkages optionally replaced by linkages such as —CH₂NH—, —CH₂S—, —CH₂CH₂—, —CH═CH— (cis and trans), —CH₂SO—, —CH(OH)CH₂—, —COCH₂— etc., by methods well known in the art (Spatola, Peptide Backbone Modifications, Vega Data, 1:267, 1983; Spatola et al., Life Sci. 38:1243-1249, 1986; Hudson et al., Int. J. Pept. Res. 14:177-185, 1979; and Weinstein, 1983, Chemistry and Biochemistry, of Amino Acids, Peptides and Proteins, Weinstein eds, Marcel Dekker, New York). Such polypeptide mimetics may have significant advantages over naturally occurring polypeptides including more economical production, greater chemical stability, enhanced pharmacological properties (e.g., half-life, absorption, potency, efficiency), reduced antigenicity, and others.

While the peptide vectors described herein may efficiently cross the BBB or target particular cell types (e.g., those described herein), their effectiveness may be reduced by the presence of proteases. Likewise, the effectiveness of GLP-1 agonists used in the invention may be similarly reduced. Serum proteases have specific substrate requirements, including L-amino acids and peptide bonds for cleavage. Furthermore, exopeptidases, which represent the most prominent component of the protease activity in serum, usually act on the first peptide bond of the polypeptide and require a free N-terminus (Powell et al., Pharm. Res. 10:1268-1273, 1993). In light of this, it is often advantageous to use modified versions of polypeptides. The modified polypeptides retain the structural characteristics of the original L-amino acid polypeptides, but advantageously are not readily susceptible to cleavage by protease and/or exopeptidases.

Systematic substitution of one or more amino acids of a consensus sequence with D-amino acid of the same type (e.g., an enantiomer; D-lysine in place of L-lysine) may be used to generate more stable polypeptides. Thus, a polypeptide derivative or peptidomimetic as described herein may be all L-, all D-, or mixed D, L polypeptides. The presence of an N-terminal or C-terminal D-amino acid increases the in vivo stability of a polypeptide because peptidases cannot utilize a D-amino acid as a substrate (Powell et al., Pharm. Res. 10:1268-1273, 1993). Reverse-D polypeptides are polypeptides containing D-amino acids, arranged in a reverse sequence relative to a polypeptide containing L-amino acids. Thus, the C-terminal residue of an L-amino acid polypeptide becomes N-terminal for the D-amino acid polypeptide, and so forth. Reverse D-polypeptides retain the same tertiary conformation and therefore the same activity, as the L-amino acid polypeptides, but are more stable to enzymatic degradation in vitro and in vivo, and thus have greater therapeutic efficacy than the original polypeptide (Brady and Dodson, Nature 368:692-693, 1994 and Jameson et al., Nature 368:744-746, 1994). In addition to reverse-D-polypeptides, constrained polypeptides comprising a consensus sequence or a substantially identical consensus sequence variation may be generated by methods well known in the art (Rizo et al., Ann. Rev. Biochem. 61:387-418, 1992). For example, constrained polypeptides may be generated by adding cysteine residues capable of forming disulfide bridges and, thereby, resulting in a cyclic polypeptide. Cyclic polypeptides have no free N- or C-termini. Accordingly, they are not susceptible to proteolysis by exopeptidases, although they are, of course, susceptible to endopeptidases, which do not cleave at polypeptide termini. The amino acid sequences of the polypeptides with N-terminal or C-terminal D-amino acids and of the cyclic polypeptides are usually identical to the sequences of the polypeptides to which they correspond, except for the presence of N-terminal or C-terminal D-amino acid residue, or their circular structure, respectively.

A cyclic derivative containing an intramolecular disulfide bond may be prepared by conventional solid phase synthesis while incorporating suitable S-protected cysteine or homocysteine residues at the positions selected for cyclization such as the amino and carboxy termini (Sah et al., J. Pharm. Pharmacol. 48:197, 1996). Following completion of the chain assembly, cyclization can be performed either (1) by selective removal of the S-protecting group with a consequent on-support oxidation of the corresponding two free SH-functions, to form a S—S bonds, followed by conventional removal of the product from the support and appropriate purification procedure or (2) by removal of the polypeptide from the support along with complete side chain de-protection, followed by oxidation of the free SH-functions in highly dilute aqueous solution.

The cyclic derivative containing an intramolecular amide bond may be prepared by conventional solid phase synthesis while incorporating suitable amino and carboxyl side chain protected amino acid derivatives, at the position selected for cyclization. The cyclic derivatives containing intramolecular —S-alkyl bonds can be prepared by conventional solid phase chemistry while incorporating an amino acid residue with a suitable amino-protected side chain, and a suitable S-protected cysteine or homocysteine residue at the position selected for cyclization.

Another effective approach to confer resistance to peptidases acting on the N-terminal or C-terminal residues of a polypeptide is to add chemical groups at the polypeptide termini, such that the modified polypeptide is no longer a substrate for the peptidase. One such chemical modification is glycosylation of the polypeptides at either or both termini. Certain chemical modifications, in particular N-terminal glycosylation, have been shown to increase the stability of polypeptides in human serum (Powell et al., Pharm. Res. 10:1268-1273, 1993). Other chemical modifications which enhance serum stability include, but are not limited to, the addition of an N-terminal alkyl group, consisting of a lower alkyl of from one to twenty carbons, such as an acetyl group, and/or the addition of a C-terminal amide or substituted amide group. In particular, the present invention includes modified polypeptides consisting of polypeptides bearing an N-terminal acetyl group and/or a C-terminal amide group.

Also included by the present invention are other types of polypeptide derivatives containing additional chemical moieties not normally part of the polypeptide, provided that the derivative retains the desired functional activity of the polypeptide. Examples of such derivatives include (1) N-acyl derivatives of the amino terminal or of another free amino group, wherein the acyl group may be an alkanoyl group (e.g., acetyl, hexanoyl, octanoyl) an aroyl group (e.g., benzoyl) or a blocking group such as F-moc (fluorenylmethyl-O—CO—); (2) esters of the carboxy terminal or of another free carboxy or hydroxyl group; (3) amide of the carboxy-terminal or of another free carboxyl group produced by reaction with ammonia or with a suitable amine; (4) phosphorylated derivatives; (5) derivatives conjugated to an antibody or other biological ligand and other types of derivatives.

Longer polypeptide sequences which result from the addition of additional amino acid residues to the polypeptides described herein are also encompassed in the present invention. Such longer polypeptide sequences can be expected to have the same biological activity and specificity (e.g., cell tropism) as the polypeptides described above. While polypeptides having a substantial number of additional amino acids are not excluded, it is recognized that some large polypeptides may assume a configuration that masks the effective sequence, thereby preventing binding to a target (e.g., a member of the LRP receptor family such as LRP or LRP2). These derivatives could act as competitive antagonists. Thus, while the present invention encompasses polypeptides or derivatives of the polypeptides described herein having an extension, desirably the extension does not destroy the cell targeting activity of the polypeptides or its derivatives.

Other derivatives included in the present invention are dual polypeptides consisting of two of the same, or two different polypeptides, as described herein, covalently linked to one another either directly or through a spacer, such as by a short stretch of alanine residues or by a putative site for proteolysis (e.g., by cathepsin, see e.g., U.S. Pat. No. 5,126,249 and European Patent No. 495 049). Multimers of the polypeptides described herein consist of a polymer of molecules formed from the same or different polypeptides or derivatives thereof.

The present invention also encompasses polypeptide derivatives that are chimeric or fusion proteins containing a polypeptide described herein, or fragment thereof, linked at its amino- or carboxy-terminal end, or both, to an amino acid sequence of a different protein. Such a chimeric or fusion protein may be produced by recombinant expression of a nucleic acid encoding the protein. For example, a chimeric or fusion protein may contain at least 6 amino acids shared with one of the described polypeptides which desirably results in a chimeric or fusion protein that has an equivalent or greater functional activity.

Assays to Identify Peptidomimetics

As described above, non-peptidyl compounds generated to replicate the backbone geometry and pharmacophore display (peptidomimetics) of the polypeptides described herein often possess attributes of greater metabolic stability, higher potency, longer duration of action, and better bioavailability.

Peptidomimetics compounds can be obtained using any of the numerous approaches in combinatorial library methods known in the art, including biological libraries, spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer, or small molecule libraries of compounds (Lam, Anticancer Drug Des. 12:145, 1997). Examples of methods for the synthesis of molecular libraries can be found in the art, for example, in: DeWitt et al. (Proc. Natl. Acad. Sci. USA 90:6909, 1993); Erb et al. (Proc. Natl. Acad. Sci. USA 91:11422, 1994); Zuckermann et al. (J. Med. Chem. 37:2678, 1994); Cho et al. (Science 261:1303, 1993); Carell et al. (Angew. Chem, Int. Ed. Engl. 33:2059, 1994 and ibid 2061); and in Gallop et al. (Med. Chem. 37:1233, 1994). Libraries of compounds may be presented in solution (e.g., Houghten, Biotechniques 13:412-421, 1992) or on beads (Lam, Nature 354:82-84, 1991), chips (Fodor, Nature 364:555-556, 1993), bacteria or spores (U.S. Pat. No. 5,223,409), plasmids (Cull et al., Proc. Natl. Acad. Sci. USA 89:1865-1869, 1992) or on phage (Scott and Smith, Science 249:386-390, 1990), or luciferase, and the enzymatic label detected by determination of conversion of an appropriate substrate to product.

Once a polypeptide as described herein is identified, it can be isolated and purified by any number of standard methods including, but not limited to, differential solubility (e.g., precipitation), centrifugation, chromatography (e.g., affinity, ion exchange, and size exclusion), or by any other standard techniques used for the purification of peptides, peptidomimetics, or proteins. The functional properties of an identified polypeptide of interest may be evaluated using any functional assay known in the art. Desirably, assays for evaluating downstream receptor function in intracellular signaling are used (e.g., cell proliferation).

For example, the peptidomimetics compounds of the present invention may be obtained using the following three-phase process: (1) scanning the polypeptides described herein to identify regions of secondary structure necessary for targeting the particular cell types described herein; (2) using conformationally constrained dipeptide surrogates to refine the backbone geometry and provide organic platforms corresponding to these surrogates; and (3) using the best organic platforms to display organic pharmocophores in libraries of candidates designed to mimic the desired activity of the native polypeptide. In more detail the three phases are as follows. In phase 1, the lead candidate polypeptides are scanned and their structure abridged to identify the requirements for their activity. A series of polypeptide analogs of the original are synthesized. In phase 2, the best polypeptide analogs are investigated using the conformationally constrained dipeptide surrogates. Indolizidin-2-one, indolizidin-9-one and quinolizidinone amino acids (I²aa, I⁹aa and Qaa respectively) are used as platforms for studying backbone geometry of the best peptide candidates. These and related platforms (reviewed in Halab et al., Biopolymers 55:101-122, 2000 and Hanessian et al., Tetrahedron 53:12789-12854, 1997) may be introduced at specific regions of the polypeptide to orient the pharmacophores in different directions. Biological evaluation of these analogs identifies improved lead polypeptides that mimic the geometric requirements for activity. In phase 3, the platforms from the most active lead polypeptides are used to display organic surrogates of the pharmacophores responsible for activity of the native peptide. The pharmacophores and scaffolds are combined in a parallel synthesis format. Derivation of polypeptides and the above phases can be accomplished by other means using methods known in the art.

Structure function relationships determined from the polypeptides, polypeptide derivatives, peptidomimetics or other small molecules described herein may be used to refine and prepare analogous molecular structures having similar or better properties. Accordingly, the compounds of the present invention also include molecules that share the structure, polarity, charge characteristics and side chain properties of the polypeptides described herein.

In summary, based on the disclosure herein, those skilled in the art can develop peptides and peptidomimetics screening assays which are useful for identifying compounds for targeting an agent to particular cell types (e.g., those described herein). The assays of this invention may be developed for low-throughput, high-throughput, or ultra-high throughput screening formats. Assays of the present invention include assays amenable to automation.

### Linkers

The peptide therapeutic may be bound to the vector peptide either directly (e.g., through a covalent bond such as a peptide bond) or may be bound through a linker. Linkers include chemical linking agents (e.g., cleavable linkers) and peptides.

In some embodiments, the linker is a chemical linking agent. The peptide therapeutic and vector peptide may be conjugated through sulfhydryl groups, amino groups (amines), and/or carbohydrates or any appropriate reactive group. Homobifunctional and heterobifunctional cross-linkers (conjugation agents) are available from many commercial sources. Regions available for cross-linking may be found on the polypeptides of the present invention. The cross-linker may comprise a flexible arm, e.g., 2, 3, 4, 5, 6, 7, 8, 9, 10, 11, 12, 13, 14, or 15 carbon atoms. Exemplary cross-linkers include BS3 ([Bis(sulfosuccinimidyl)suberate]; BS3 is a homobifunctional N-hydroxysuccinimide ester that targets accessible primary amines), NHS/EDC (N-hydroxysuccinimide and N-ethyl-(dimethylaminopropyl)carbodimide; NHS/EDC allows for the conjugation of primary amine groups with carboxyl groups), sulfo-EMCS ([N-e-Maleimidocaproic acid]hydrazide; sulfo-EMCS are heterobifunctional reactive groups (maleimide and NHS-ester) that are reactive toward sulfhydryl and amino groups), hydrazide (most proteins contain exposed carbohydrates and hydrazide is a useful reagent for linking carboxyl groups to primary amines), and SATA (N-succinimidyl-S-acetylthioacetate; SATA is reactive towards amines and adds protected sulfhydryls groups).

To form covalent bonds, one can use as a chemically reactive group a wide variety of active carboxyl groups (e.g., esters) where the hydroxyl moiety is physiologically acceptable at the levels required to modify the peptide. Particular agents include N-hydroxysuccinimide (NHS), N-hydroxy-sulfosuccinimide (sulfo-NHS), maleimide-benzoyl-succinimide (MBS), gamma-maleimido-butyryloxy succinimide ester (GMBS), maleimido propionic acid (MPA) maleimido hexanoic acid (MHA), and maleimido undecanoic acid (MUA).

Primary amines are the principal targets for NHS esters. Accessible a-amine groups present on the N-termini of proteins and the E-amine of lysine react with NHS esters. An amide bond is formed when the NHS ester conjugation reaction reacts with primary amines releasing N-hydroxysuccinimide. These succinimide containing reactive groups are herein referred to as succinimidyl groups. In certain embodiments of the invention, the functional group on the protein will be a thiol group and the chemically reactive group will be a maleimido-containing group such as gamma-maleimide-butrylamide (GMBA or MPA). Such maleimide containing groups are referred to herein as maleido groups.

The maleimido group is most selective for sulfhydryl groups on peptides when the pH of the reaction mixture is 6.5-7.4. At pH 7.0, the rate of reaction of maleimido groups with sulfhydryls (e.g., thiol groups on proteins such as serum albumin or IgG) is 1000-fold faster than with amines. Thus, a stable thioether linkage between the maleimido group and the sulfhydryl can be formed.

In other embodiments, the linker includes at least one amino acid (e.g., a peptide of at least 2, 3, 4, 5, 6, 7, 10, 15, 20, 25, 40, or 50 amino acids). In certain embodiments, the linker is a single amino acid (e.g., any naturally occurring amino acid such as Cys). In other embodiments, a glycine-rich peptide such as a peptide having the sequence [Gly-Gly-Gly-Gly-Ser]ₙ where n is 1, 2, 3, 4, 5 or 6 is used, as described in U.S. Pat. No. 7,271,149. In other embodiments, a serine-rich peptide linker is used, as described in U.S. Pat. No. 5,525,491. Serine rich peptide linkers include those of the formula [X-X-X-X-Gly]_{y}, where up to two of the X are Thr, and the remaining X are Ser, and y is 1 to 5 (e.g., Ser-Ser-Ser-Ser-Gly, where y is greater than 1). In some cases, the linker is a single amino acid (e.g., any amino acid, such as Gly or Cys).

Examples of suitable linkers are succinic acid, Lys, Glu, and Asp, or a dipeptide such as Gly-Lys. When the linker is succinic acid, one carboxyl group thereof may form an amide bond with an amino group of the amino acid residue, and the other carboxyl group thereof may, for example, form an amide bond with an amino group of the peptide or substituent. When the linker is Lys, Glu, or Asp, the carboxyl group thereof may form an amide bond with an amino group of the amino acid residue, and the amino group thereof may, for example, form an amide bond with a carboxyl group of the substituent. When Lys is used as the linker, a further linker may be inserted between the ε-amino group of Lys and the substituent. In one particular embodiment, the further linker is succinic acid which, e.g., forms an amide bond with the ε-amino group of Lys and with an amino group present in the substituent. In one embodiment, the further linker is Glu or Asp (e.g., which forms an amide bond with the E-amino group of Lys and another amide bond with a carboxyl group present in the substituent), that is, the substituent is a N^{ε}-acylated lysine residue.

### GLP-1 Agonist Activity Assay

Determination of whether a compound has GLP-1 agonist activity can be performed using any method known in the art. Cyclic AMP (cAMP) production from cells expressing a GLP-1 receptor (e.g., a human receptor) can be measured in the presence and in the absence of a compound, where an increase in cAMP production indicates the compound to be a GLP-1 agonist.

In one example described in U.S. Patent Application Publication No. 2008/0207507, baby hamster kidney (BHK) cells expressing the cloned human GLP-1 receptor (BHK-467-12A) were grown in DMEM media with the addition of 100 IU/ml penicillin, 100 μg/ml streptomycin, 5% fetal calf serum, and 0.5 mg/mL Geneticin G-418 (Life Technologies). The cells were washed twice in phosphate buffered saline and harvested with Versene. Plasma membranes were prepared from the cells by homogenisation with an Ultraturrax in buffer 1 (20 mM HEPES-Na, 10 mM EDTA, pH 7.4). The homogenate was centrifuged at 48,000×g for 15 min at 4° C. The pellet was suspended by homogenization in buffer 2 (20 mM HEPES-Na, 0.1 mM EDTA, pH 7.4), then centrifuged at 48,000×g for 15 min at 4° C. The washing procedure was repeated one more time. The final pellet was suspended in buffer 2 and used immediately for assays or stored at 80° C.

The functional receptor assay was carried out by measuring cAMP as a response to stimulation by the insulinotropic agent. cAMP formed was quantified by the AlphaScreen™ cAMP Kit (Perkin Elmer Life Sciences). Incubations were carried out in half-area 96-well microtiter plates in a total volume of 50 μ/L buffer 3 (50 mM Tris-HCl, 5 mM HEPES, 10 mM MgCl₂, pH 7.4) and with the following additions: 1 mM ATP, 1 μM GTP, 0.5 mM 3-isobutyl-1-methylxanthine (IBMX), 0.01% Tween-20, 0.1% BSA, 6 pg membrane preparation, 15 pg/ml acceptor beads, 20 μg/ml donor beads preincubated with 6 nM biotinyl-cAMP. Compounds to be tested for agonist activity were dissolved and diluted in buffer 3. GTP was freshly prepared for each experiment. The plate was incubated in the dark with slow agitation for three hours at room temperature followed by counting in the Fusion™ instrument (Perkin Elmer Life Sciences). Concentration-response curves were plotted for the individual compounds and EC₅₀ values estimated using a four-parameter logistic model with Prism v. 4.0 (GraphPad, Carlsbad, Calif.).

### Therapeutic Applications

The compounds of the invention can be used in any appropriate therapeutic application where the activity of the peptide therapeutic is beneficial. The compounds of the invention can be used to treat infections (e.g., where the peptide therapeutic is antimicrobial or antibiotic peptide), to treat neoplasms such as a cancer (e.g., using a agent having antiproliferative activity, such as a tumor antibiotic or thyrotropin), for treating pain (e.g., using an opioid), to treat metabolic disorders (e.g., using a GLP-1 agonist, gastric inhibitory polypeptide, insulin, growth hormone-releasing hormone, or an analog thereof), neurological disorder such as seizures (e.g., using galanin or an analog therof), for bone diseases such as osteoporosis, Paget's disease (e.g., using PTH, PTHrP, calcintonin, or an analog thereof), and hypertension (e.g., using bradykinin or an analog thereof). Compounds containing any of the human peptides described herein (or analogs or fragments thereof) as a peptide thereapeutic may be used to treat a subject suffering a deficiency of that peptide. Additional indications are described below.

Cancer

The compounds of the invention can be used to treat any cancer, but, in the case of conjugates including a vector that is efficiently transported across the BBB, are particularly useful for the treatment of brain cancers and other cancers protected by the BBB. These cancers include astrocytoma, pilocytic astrocytoma, dysembryoplastic neuroepithelial tumor, oligodendrogliomas, ependymoma, glioblastoma multiforme, mixed gliomas, oligoastrocytomas, medulloblastoma, retinoblastoma, neuroblastoma, germinoma, and teratoma. Other types of cancer include hepatocellular carcinoma, breast cancer, cancers of the head and neck including various lymphomas such as mantle cell lymphoma, non-Hodgkins lymphoma, adenoma, squamous cell carcinoma, laryngeal carcinoma, cancers of the retina, cancers of the esophagus, multiple myeloma, ovarian cancer, uterine cancer, melanoma, colorectal cancer, bladder cancer, prostate cancer, lung cancer (including non-small cell lung carcinoma), pancreatic cancer, cervical cancer, head and neck cancer, skin cancers, nasopharyngeal carcinoma, liposarcoma, epithelial carcinoma, renal cell carcinoma, gallbladder adenocarcinoma, parotid adenocarcinoma, endometrial sarcoma, multidrug resistant cancers; and proliferative diseases and conditions, such as neovascularization associated with tumor angiogenesis, macular degeneration (e.g., wet/dry AMD), comeal neovascularization, diabetic retinopathy, neovascular glaucoma, myopic degeneration and other proliferative diseases and conditions such as restenosis and polycystic kidney disease.

Neurological Disease

Because polypeptides described herein are capable of transporting an agent across the BBB, the compounds of the invention are also useful for the treatment of neurological diseases such as neurodegenerative diseases or other conditions of the central nervous system (CNS), the peripheral nervous system, or the autonomous nervous system (e.g., where neurons are lost or deteriorate). Many neurodegenerative diseases are characterized by ataxia (i.e., uncoordinated muscle movements) and/or memory loss. Neurodegenerative diseases include Alexander disease, Alper disease, Alzheimer's disease, amyotrophic lateral sclerosis (ALS; i.e., Lou Gehrig's disease), ataxia telangiectasia, Batten disease (Spielmeyer-Vogt-Sjogren-Batten disease), bovine spongiform encephalopathy (BSE), Canavan disease, Cockayne syndrome, corticobasal degeneration, Creutzfeldt-Jakob disease, Huntington's disease, HIV-associated dementia, Kennedy's disease, Krabbé disease, Lewy body dementia, Machado-Joseph disease (Spinocerebellar ataxia type 3), multiple sclerosis, multiple system atrophy, narcolepsy, neuroborreliosis, Parkinson's disease, Pelizaeus-Merzbacher disease, Pick's disease, primary lateral sclerosis, prion diseases, Refsum's disease, Schilder's disease (i.e., adrenoleukodystrophy), schizophrenia, spinocerebellar ataxia, spinal muscular atrophy, Steele-Richardson, Olszewski disease, and tabes dorsalis.

Lysosomal Storage Disorders

The conjugates of the invention may also be used to treat a lysosomal storage disease or disorder, many of which affect the central nervous system (CNS) and cause or exacerbate neurodegenerative disease. Lysosomal storage diseases include any of the mucopolysaccharidoses (MPS; including MPS-I (Hurler syndrome, Scheie syndrome), MPS-II (Hunter syndrome), MPS-IIIA (Sanfilippo syndrome A), MPS-IIIB (Sanfilippo syndrome B), MPS-IIIC (Sanfilippo syndrome C), MPS-IIID (Sanfilippo syndrome D), MPS-IV (Morquio syndrome), MPS-VI (Maroteaux-Lamy syndrome), MPS-VII (Sly syndrome), and MPS-IX (hyaluronidase deficiency)), lipidoses (including Gaucher’ disease, Niemann-Pick disease, Fabry disease, Farber's disease, and Wolman's disease), gangliosidoses (including GM1 and GM2 gangliosidoses, Tay-Sachs disease, and Sandhoff disease), leukodystrophies (including adrenoleukodystrophy (i.e., Schilder's disease), Alexander disease, metachromatic leukodystrophy, Krabbe disease, Pelizaeus-Merzbacher disease, Canavan disease, childhood ataxia with central hypomyelination (CACH), Refsum's disease, and cerebrotendineous xanthomatosis), mucolipidoses (ML; including ML-I (sialidosis), ML-II (1-cell disease), ML-III (pseudo-Hurler polydystrophy), and ML-IV), and glycoproteinoses (including aspartylglucosaminuria, fucosidosis, and mannosidosis).

GLP-1 Related Disorders

In certain embodiments, the peptide therapeutic is a GLP-1 agonist. Such compounds can be used in any therapeutic application where a GLP-1 agonist activity in the brain, or in particular tissues, is desired. GLP-1 agonist activity is associated with stimulation of insulin secretion (i.e., to act as an incretin hormone) and inhibition glucagon secretion, thereby contributing to limit postprandial glucose excursions. GLP-1 agonists can also inhibit gastrointestinal motility and secretion, thus acting as an enterogastrone and part of the “ileal brake” mechanism. GLP-1 also appears to be a physiological regulator of appetite and food intake. Because of these actions, GLP-1 and GLP-1 receptor agonists can be used for therapy of metabolic disorders, as reviewed in, e.g., Kinzig et al., J Neurosci 23:6163-6170, 2003. Such disorders include obesity, hyperglycemia, dyslipidemia, hypertriglyceridemia, syndrome X, insulin resistance, IGT, diabetic dyslipidemia, hyperlipidemia, a cardiovascular disease, and hypertension.

GLP-1 is also has neurological effects including sedative or anti-anxiolytic effects, as described in U.S. Pat. No. 5,846,937. Thus, GLP-1 agonists can be used in the treatment of anxiety, aggression, psychosis, seizures, panic attacks, hysteria, or sleep disorders. GLP-1 agonists can also be used to treat Alzheimer's disease, as GLP-1 agonists have been shown to protect neurons against amyloid-β peptide and glutamate-induced apoptosis (Perry et al., Curr Alzheimer Res 2:377-85, 2005).

Other therapeutic uses for GLP-1 agonists include improving learning, enhancing neuroprotection, and alleviating a symptom of a disease or disorder of the central nervous system, e.g., through modulation of neurogenesis, and e.g., Parkinson's Disease, Alzheimer's Disease, Huntington's Disease, ALS, stroke, ADD, and neuropsychiatric syndromes (U.S. Pat. No. 6,969,702 and U.S. Patent Application No. 2002/0115605). Stimulation of neurogenesis using GLP-1 agonists has been described, for example, in Bertilsson et al., J Neurosci Res 86:326-338, 2008.

Still other therapeutic uses include converting liver stem/progenitor cells into functional pancreatic cells (U.S. Patent Application Publication No. 2005/0053588); preventing beta-cell deterioration (U.S. Pat. Nos. 7,259,233 and 6,569,832) and stimulation of beta-cell proliferation (U.S. Patent Application Publication No. 2003/0224983); treating obesity (U.S. Pat. No. 7,211,557); suppressing appetite and inducing satiety (U.S. Patent Application Publication No. 2003/0232754); treating irritable bowel syndrome (U.S. Pat. No. 6,348,447); reducing the morbidity and/or mortality associated with myocardial infarction (US Pat. No. 6,747,006) and stroke (PCT Publication No. WO 00/16797); treating acute coronary syndrome characterized by an absence of Q-wave myocardial infarction (U.S. Pat. No. 7,056,887); attenuating post-surgical catabolic changes (U.S. Pat. No. 6,006,753); treating hibernating myocardium or diabetic cardiomyopathy (U.S. Pat. No. 6,894,024); suppressing plasma blood levels of norepinepherine (U.S. Pat. No. 6,894,024); increasing urinary sodium excretion, decreasing urinary potassium concentration (U.S. Pat. No. 6,703,359); treating conditions or disorders associated with toxic hypervolemia, e.g., renal failure, congestive heart failure, nephrotic syndrome, cirrhosis, pulmonary edema, and hypertension (U.S. Pat. No. 6,703,359); inducing an inotropic response and increasing cardiac contractility (U.S. Pat. No. 6,703,359); treating polycystic ovary syndrome (U.S. Pat. No. 7,105,489); treating respiratory distress (U.S. Patent Application Publication No. 2004/0235726); improving nutrition via a non-alimentary route, i.e., via intravenous, subcutaneous, intramuscular, peritoneal, or other injection or infusion (U.S. Pat. No. 6,852,690); treating nephropathy (U.S. Patent Application Publication No. 2004/0209803); treating left ventricular systolic dysfunction, e.g., with abnormal left ventricular ejection fraction (U.S. Pat. No. 7,192,922); inhibiting antro-duodenal motility, e.g., for the treatment or prevention of gastrointestinal disorders such as diarrhea, postoperative dumping syndrome and irritable bowel syndrome, and as premedication in endoscopic procedures (U.S. Pat. No. 6,579,851); treating critical illness polyneuropathy (CIPN) and systemic inflammatory response syndrome (SIRS) (U.S. Patent Application Publication No. 2003/0199445); modulating triglyceride levels and treating dyslipidemia (U.S. Patent Application Publication Nos. 2003/0036504 and 2003/0143183); treating organ tissue injury caused by reperfusion of blood flow following ischemia (U.S. Pat. No. 6,284,725); treating coronary heart disease risk factor (CHDRF) syndrome (U.S. Pat. No. 6,528,520); and others.

Additional Indications

The conjugates of the invention can also be used to treat diseases found in other organs or tissues. For example, Angiopep-7 (SEQ ID NO:112) is efficiently transported into liver, lung, kidney, spleen, and muscle cells, allowing for the preferential treatment of diseases associated with these tissues (e.g., hepatocellular carcinoma and lung cancer). The compounds of the presents invention may also be used to treat genetic disorders, such as Down syndrome (i.e., trisomy 21), where down-regulation of particular gene transcripts may be useful.

Administration and Dosage

The present invention also features pharmaceutical compositions that contain a therapeutically effective amount of a compound of the invention. The composition can be formulated for use in a variety of drug delivery systems. One or more physiologically acceptable excipients or carriers can also be included in the composition for proper formulation. Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

The pharmaceutical compositions are intended for parenteral, intranasal, topical, oral, or local administration, such as by a transdermal means, for prophylactic and/or therapeutic treatment. The pharmaceutical compositions can be administered parenterally (e.g., by intravenous, intramuscular, or subcutaneous injection), or by oral ingestion, or by topical application or intraarticular injection at areas affected by the vascular or cancer condition. Additional routes of administration include intravascular, intra-arterial, intratumor, intraperitoneal, intraventricular, intraepidural, as well as nasal, ophthalmic, intrascleral, intraorbital, rectal, topical, or aerosol inhalation administration. Sustained release administration is also specifically included in the invention, by such means as depot injections or erodible implants or components. Thus, the invention provides compositions for parenteral administration that comprise the above mention agents dissolved or suspended in an acceptable carrier, preferably an aqueous carrier, e.g., water, buffered water, saline, PBS, and the like. The compositions may contain pharmaceutically acceptable auxiliary substances as required to approximate physiological conditions, such as pH adjusting and buffering agents, tonicity adjusting agents, wetting agents, detergents and the like. The invention also provides compositions for oral delivery, which may contain inert ingredients such as binders or fillers for the formulation of a tablet, a capsule, and the like. Furthermore, this invention provides compositions for local administration, which may contain inert ingredients such as solvents or emulsifiers for the formulation of a cream, an ointment, and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

The compositions containing an effective amount can be administered for prophylactic or therapeutic treatments. In prophylactic applications, compositions can be administered to a subject with a clinically determined predisposition or increased susceptibility to a metabolic disorder or neurological disease. Compositions of the invention can be administered to the subject (e.g., a human) in an amount sufficient to delay, reduce, or preferably prevent the onset of clinical disease. In therapeutic applications, compositions are administered to a subject (e.g., a human) already suffering from disease (e.g., a metabolic disorder such as those described herein, or a neurological disease) in an amount sufficient to cure or at least partially arrest the symptoms of the condition and its complications. An amount adequate to accomplish this purpose is defined as a “therapeutically effective amount,” an amount of a compound sufficient to substantially improve some symptom associated with a disease or a medical condition. For example, in the treatment of a metabolic disorder (e.g., those described herein), an agent or compound which decreases, prevents, delays, suppresses, or arrests any symptom of the disease or condition would be therapeutically effective. A therapeutically effective amount of an agent or compound is not required to cure a disease or condition but will provide a treatment for a disease or condition such that the onset of the disease or condition is delayed, hindered, or prevented, or the disease or condition symptoms are ameliorated, or the term of the disease or condition is changed or, for example, is less severe or recovery is accelerated in an individual.

The compounds of the invention may be administered in equivalent doses of as specified for the unconjugated peptide therapeutic, may be administered in higher equivalent doses (e.g., 10%, 25%, 50%, 100%, 200%, 500%, 1000% greater doses), or can be administered in lower equivalent doses (e.g., 90%, 75%, 50%, 40%, 30%, 20%, 15%, 12%, 10%, 8%, 7%, 6%, 5%, 4%, 3%, 2%, 1%, 0.5%, or 0.1% of the equivalent dose). Amounts effective for this use may depend on the severity of the disease or condition and the weight and general state of the subject, but generally range from about 0.05 μg to about 10,000 μg (e.g., 0.5-1000 μg) of an equivalent amount of the peptide therapeutic the agent or agents per dose per subject. Suitable regimes for initial administration and booster administrations are typified by an initial administration followed by repeated doses at one or more hourly, daily, weekly, or monthly intervals by a subsequent administration. The total effective amount of an agent present in the compositions of the invention can be administered to a mammal as a single dose, either as a bolus or by infusion over a relatively short period of time, or can be administered using a fractionated treatment protocol, in which multiple doses are administered over a more prolonged period of time (e.g., a dose every 4-6, 8-12, 14-16, or 18-24 hours, or every 2-4 days, 1-2 weeks, once a month). Alternatively, continuous intravenous infusion sufficient to maintain therapeutically effective concentrations in the blood are contemplated.

The therapeutically effective amount of one or more agents present within the compositions of the invention and used in the methods of this invention applied to mammals (e.g., humans) can be determined by the ordinarily-skilled artisan with consideration of individual differences in age, weight, and the condition of the mammal. Because certain compounds of the invention exhibit an enhanced ability to cross the BBB, the dosage of the compounds of the invention can be lower than (e.g., less than or equal to about 90%, 75%, 50%, 40%, 30%, 20%, 15%, 12%, 10%, 8%, 7%, 6%, 5%, 4%, 3%, 2%, 1%, 0.5%, or 0.1% of) the equivalent dose of required for a therapeutic effect of the unconjugated peptide therapeutic. The agents of the invention are administered to a subject (e.g. a mammal, such as a human) in an effective amount, which is an amount that produces a desirable result in a treated subject (e.g. reduction in glycemia, reduced weight gain, increased weight loss, and reduced food intake). Therapeutically effective amounts can also be determined empirically by those of skill in the art.

The subject may also receive an agent in the range of about 0.05 to 10,000 μg equivalent dose as compared to peptide therapeutic per dose one or more times per week (e.g., 2, 3, 4, 5, 6, or 7 or more times per week), 0.1 to 2,500 (e.g., 2,000, 1,500, 1,000, 500, 100, 10, 1, 0.5, or 0.1) μg dose per day, more than once per day, or per week. A subject may also receive an agent of the composition in the range of 0.1 to 3,000 μg per dose once every two or three weeks.

Single or multiple administrations of the compositions of the invention comprising an effective amount can be carried out with dose levels and pattern being selected by the treating physician. The dose and administration schedule can be determined and adjusted based on the severity of the disease or condition in the subject, which may be monitored throughout the course of treatment according to the methods commonly practiced by clinicians or those described herein.

The compounds of the present invention may be used in combination with either conventional methods of treatment or therapy or may be used separately from conventional methods of treatment or therapy.

When the compounds of this invention are administered in combination therapies with other agents, they may be administered sequentially or concurrently to an individual. Alternatively, pharmaceutical compositions according to the present invention may be comprised of a combination of a compound of the present invention in association with a pharmaceutically acceptable excipient, as described herein, and another therapeutic or prophylactic agent known in the art.

### EXAMPLE 1

### Synthesizing GLP-1 agonist-Angiopep Conjugates

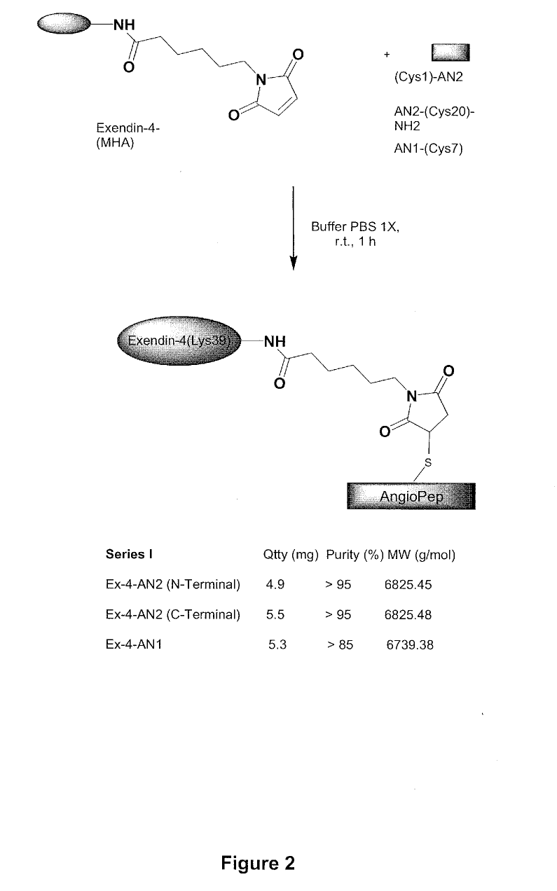

The exemplary GLP-1 conjugates, exendin-4-cysAn2 N-terminal, and Exendin-4-cysAn2 C-terminal, and Angiopep-1/Exendin 4 conjugates were made by conjugating [Lys(maleimido hexanoic acid)³⁹]exendin-4 to the sulfide in cys-An2 (SEQ ID NO:113), in An2-cys (SEQ ID NO:114), or in Angiopep-1 (SEQ ID NO:67) in lx PBS buffer for 1 hour. This resulted in production of exendin-4/Angiopep conjugates, as shown in FIG. 2.

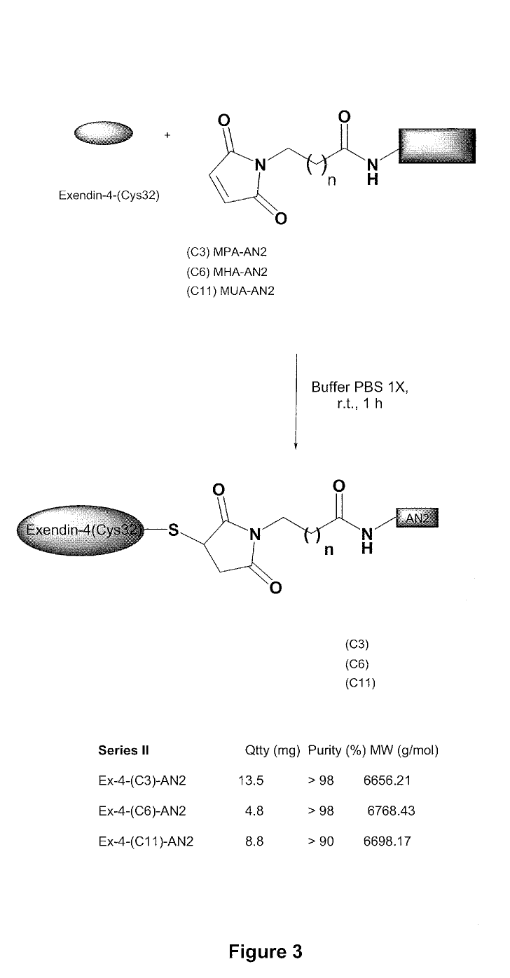

A second set of exendin-4/Angiopep conjugates was made by reacting Angiopep-2 having maleimido propionic acid (MPA), maleimido hexanoic acid (MHA), or maleimido undecanoic acid (MUA) bound to its N-terminus with [Cys³²]Exendin-4 to form a conjugate, as shown in FIG. 3.

### EXAMPLE 2

### Brain Uptake of Exendin-4/Angiopep-2 Conjugates In Situ

To measure brain uptake of the exendin-4/Angiopep-2 conjugates, we used an in situ perfusion assay. The assay, which is described in U.S. Patent Application Publication No. 2006/0189515, is performed as follows. The uptake of labeled exendin-4 and the exendin-4/Angiopep-2 conjugates was measured using the in situ brain perfusion method adapted in our laboratory for the study of drug uptake in the mouse brain (Dagenais et al., J Cereb Blood Flow Metab. 20:381-6, 2000; Cisternino et al., Pharm Res 18, 183-190, 2001). Briefly, the right common carotid artery of mice anesthetized with ketamine/xylazine (140/8 mg/kg i.p.) was exposed and ligated at the level of the bifurcation of the common carotid, rostral to the occipital artery. The common carotid was then catheterized rostrally with polyethylene tubing filled with heparin (25 U/ml) and mounted on a 26-gauge needle. The syringe containing the perfusion fluid ([¹²⁵I]-proteins or [¹²⁵I]-peptides in Krebs/bicarbonate buffer at pH 7.4, gassed with 95% O₂ and 5% CO₂) was placed in an infusion pump (Harvard pump PHD 2000; Harvard Apparatus) and connected to the catheter. Prior to the perfusion, the contralateral blood flow contribution was eliminated by severing the heart ventricles. The brain was perfused for 5 min at a flow rate of 1.15 ml/min. After perfusion of radiolabeled molecules, the brain was further perfused for 60 s with Krebs buffer, to wash away excess [¹²⁵I]-proteins. Mice were then decapitated to terminate perfusion and the right hemisphere was isolated on ice before being subjected to capillary depletion. Aliquots of homogenates, supernatants, pellets, and perfusates were taken to measure their contents and to evaluate the apparent volume of distribution.

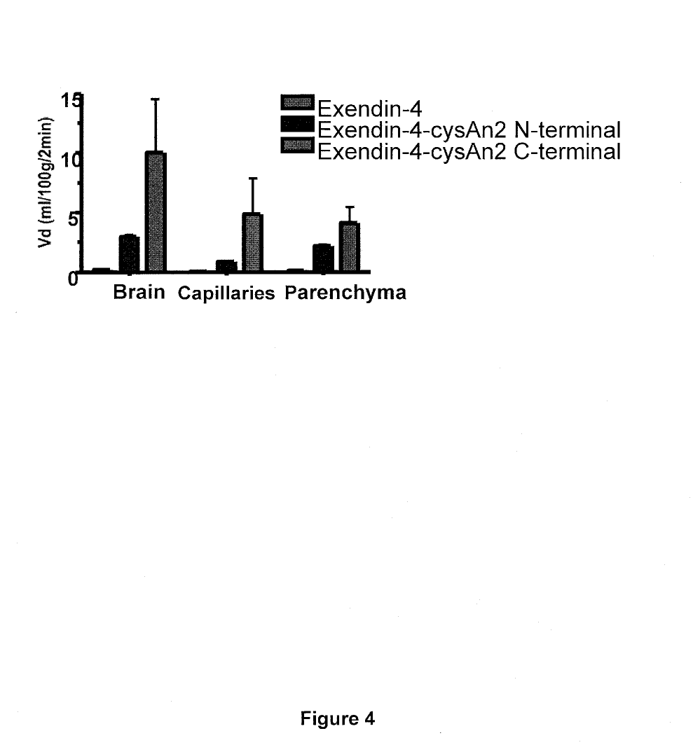

From these experiments, brain distribution of both exendin-4/Angiopep-2 conjugates was increased 15-50 fold over that of unconjugated exendin-4. The brain distribution of exendin-4 was observed at 0.2 ml/100 g/2 min, whereas the conjugate modified at its N-terminal was observed at 3 ml/100 g/2 min, and the conjugate modified at its C-terminal was observed at 10 ml/100 g/2 min. Results are shown in FIG. 4.

### EXAMPLE 3

### Treatment of Obese Mice with Exendin-4/Angiopep-2 Conjugates

Obese mice (ob/ob mice) were administered the [Lys³⁹-MHA]exendin-4/Angiopep-2-Cys-NH₂ conjugate (Exen-An2).

In vivo study to determine the efficacy
of Exendin-4-Angiopep-2 conjugate

| | Dose | Dose | Dose | mice/ | Q1Dx 28 days |
|---|---|---|---|---|---|
| Groups | (μg/kg) | (nmol/kg) | (μg/mouse) | group | (Total amount μg) |
| Control | 0 | 0 | 0 | 5 | 0 |
| Exendin-4 | 3 | 0.72 | 0.18 | 5 | 20.16 |
| | 30 | 7.2 | 1.8 | 5 | 201.6 |
| Exen-An2 | 4.8 | 0.72 | 0.288 | 5 | 32.256 |
| | 48 | 7.2 | 2.88 | 5 | 322.56 |

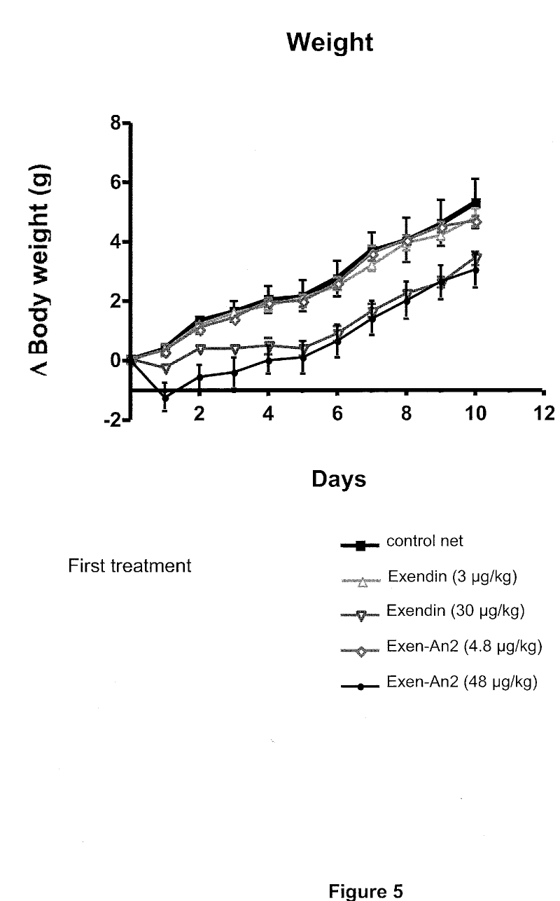

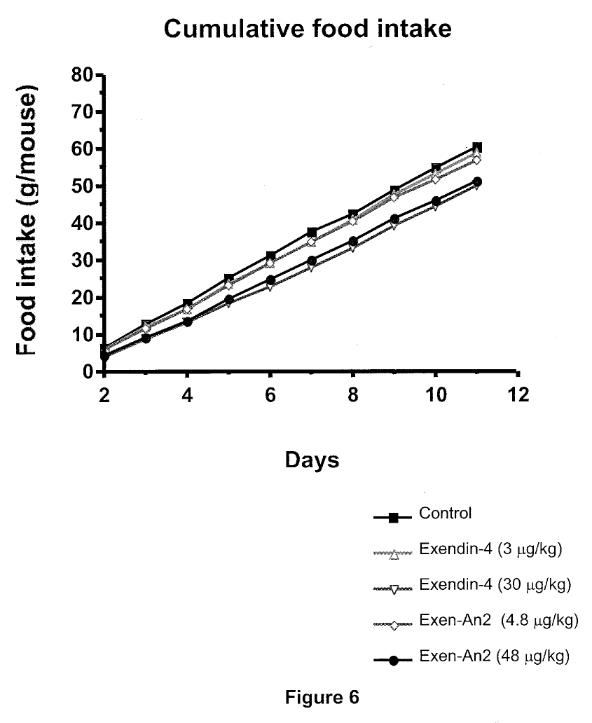

A 1.6 μg/kg dose of Exen-An2 is equivalent to a 1 μg/kg dose of exendin-4. The body weight of each mouse was measured daily. Food intake was estimated based on the mean values for each group, and glycemia was measured one hour following treatment. After 10 days of treatment, body weight gain and food intake of mice treated at the higher doses of either exendin-4 or the conjugate are lower than the control (FIG. 5). Food intake was also reduced in the mice receiving the higher doses of either exendin-4 or the conjugate (FIG. 6) as compared to the control.

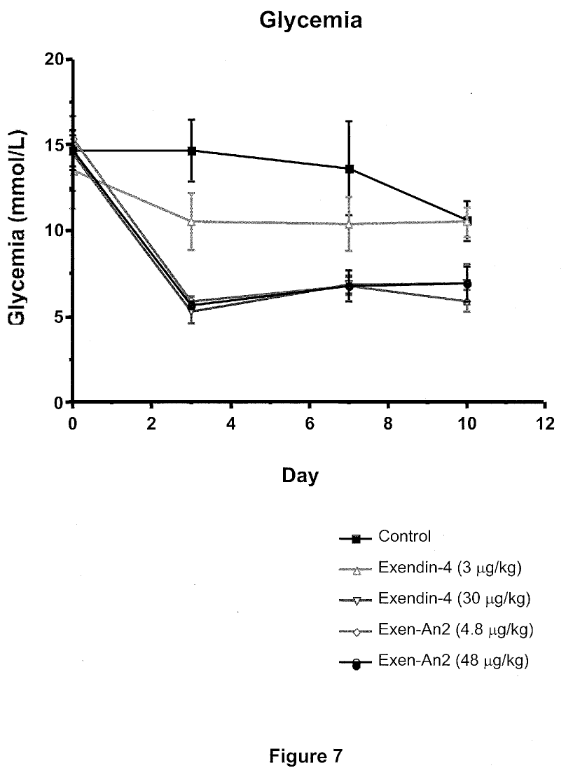

Glycemia measurements showed that the lower dose of the conjugate had the same effect as the higher doses of either exendin-4 or Exen-An2 (FIG. 7). Suprisingly, a similar effect of 1/10 the dosage on glycemia is observed using the conjugate, as compared to exendin-4.

### EXAMPLE 4

### Generation of an Exendin-4-Angioep-2 Dimer Conjugate

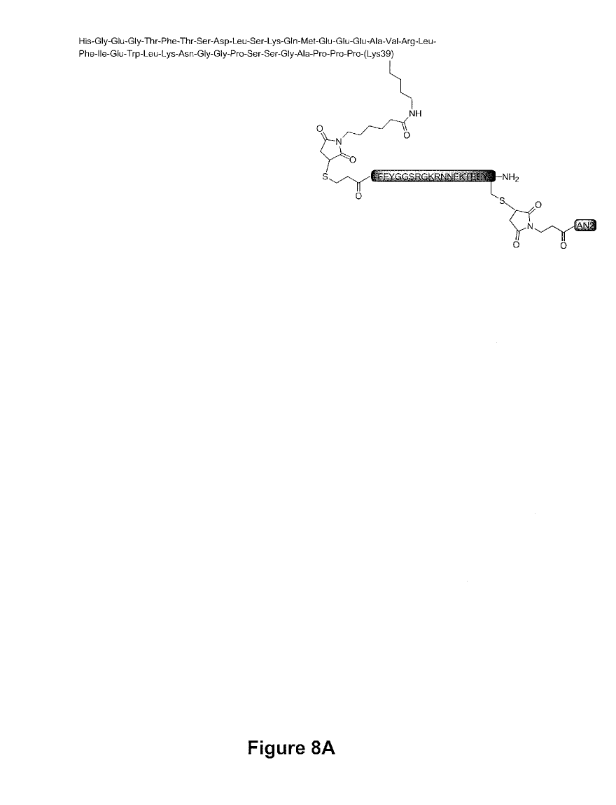

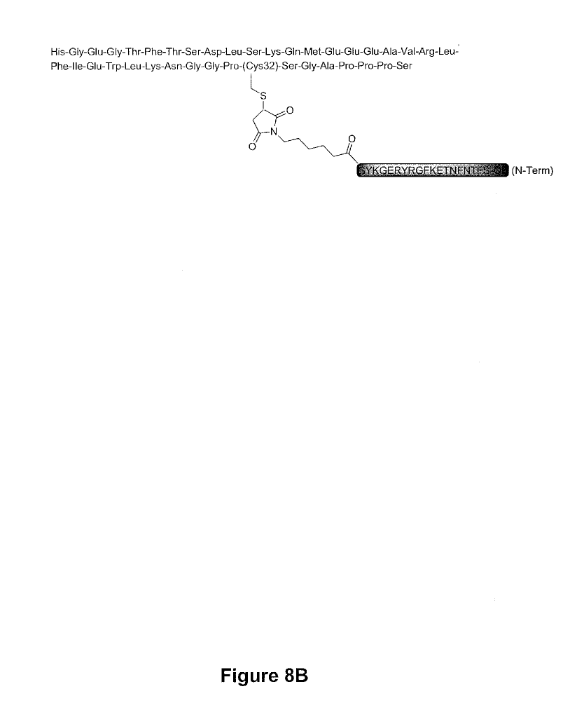

Using the conjugation chemistry described herein or similar chemistry, an Exendin-4-Angiopep-2 dimer was generated having the structure shown in FIG. 8A. Briefly, the amine group in the C-terminal lysine of [Lys³⁹]Exendin-4 was conjugated to an Angiopep-2 dimer through an MHA linker at the N-terminal threonine of the first Angiopep-2 peptide. A N-Succinimidyl-S-acetylthiopropionate (SATP) linker was attached to an Angiopep-2-Cys peptide at its N-terminus. Through this cysteine, the Angiopep-2-Cys peptide was conjugated to a second Angiopep-2 peptide, which had been modified to contain an MPA linker. The dimer was the linked to the [Lys³⁹]Exendin-4 through an MHA linker A control molecule (Exen-S4) was also generated using a scrambed form of Angiopep-2 conjugated at its N-terminal to the cysteine of [Cys³²]Exendin-4 through an MHA linker (FIG. 8B). These conjugates were prepared as trifluoroacetate (TFA) salts.

### EXAMPLE 5

### Characterization of an Exendin-4-Angiopep-2 Dimer Conjugate

Brain uptake of the exemplary GLP-1 agonist, exendin-4, was measured in situ when unconjugated, conjugated to a single Angiopep-2, conjugated to a scrambled Angiopep-2 (S4), or conjugated to a dimeric form of Angiopep-2. The experiments were performed as described in Example 2 above.

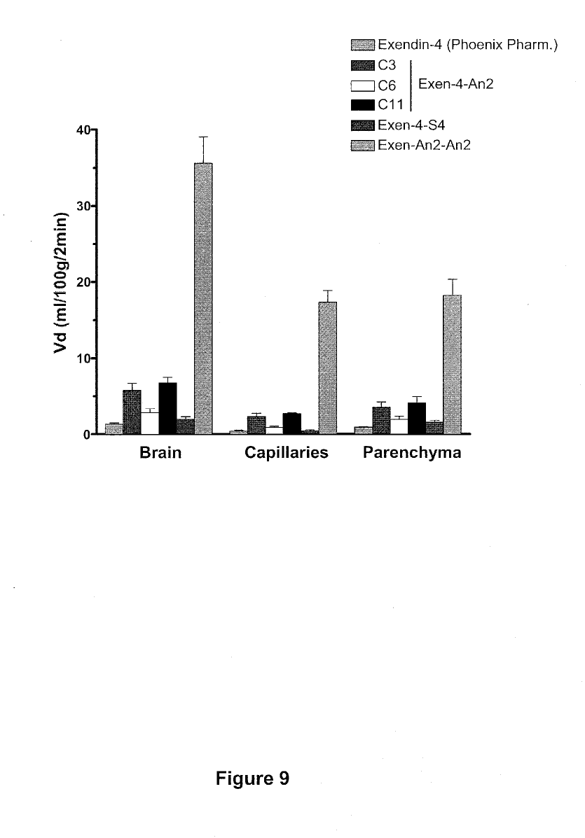

From these results, we observed that conjugation of the exendin-4 analog to the dimeric form of Angiopep-2 results in a conjugate with a surprisingly greater ability to cross the BBB as compared to either the unconjugated exendin-4 or to the exendin-4 conjugated to a single Angiopep-2 (FIG. 9).

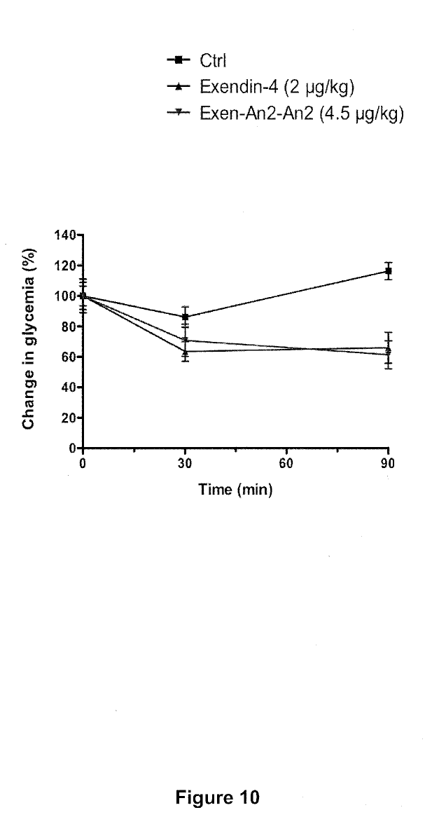

We also tested the ability of the exendin-4-Angiopep-2 dimer conjugate to reduce glycemia in DIO mice. Mice were injected with a bolus containing a control, exendin-4, or the exendin-4-Angiopep-2 dimer conjugate. Mice receiving either exendin-4 or the conjugate exhibited reduced glycemia as compared to mice receiving the control (FIG. 10).

### EXAMPLE 6

### Characterization of an Exendin-4-Angiopep-2 Dimer Conjugate

Brain uptake of the exemplary GLP-1 agonist, exendin-4, was measured in situ when unconjugated, conjugated to a single Angiopep-2, conjugated to S4, or conjugated to a dimeric form of Angiopep-2. The experiments were performed as described in Example 2 above.

From these results, we observed that conjugation of the exendin-4 analog to the dimeric form of Angiopep-2 results in a conjugate with a surprisingly greater ability to cross the BBB as compared to either the unconjugated exendin-4 or to the exendin-4 conjugated to a single Angiopep-2 (FIG. 8).

We also tested the ability of the exendin-4-Angiopep-2 dimer conjugate to reduce glycemia in DIO mice. Mice were injected with a bolus containing a control, exendin-4, or the exendin-4-Angiopep-2 dimer conjugate. Mice receiving either exendin-4 or the conjugate exhibited reduced glycemia as compared to mice receiving the control (FIG. 9).

### EXAMPLE 7

### Pancreatic Uptake and Insulin Response of Exen-4-An2 Conjugate

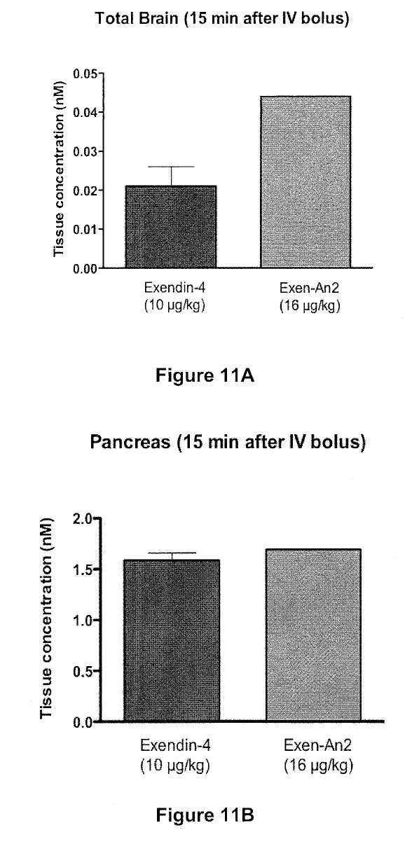

We also tested the brain and pancreatic uptake of both Exendin-4 and the Exen-4-An2 conjugate in mice 15 minutes following an intravenous bolus injection of either compound. As seen in FIG. 11A, brain uptake was enhanced in the Exen-4-An2 as compared to the unconjugated Exendin-4 peptide, whereas similar levels of pancreatic concentration were observed with both compounds.

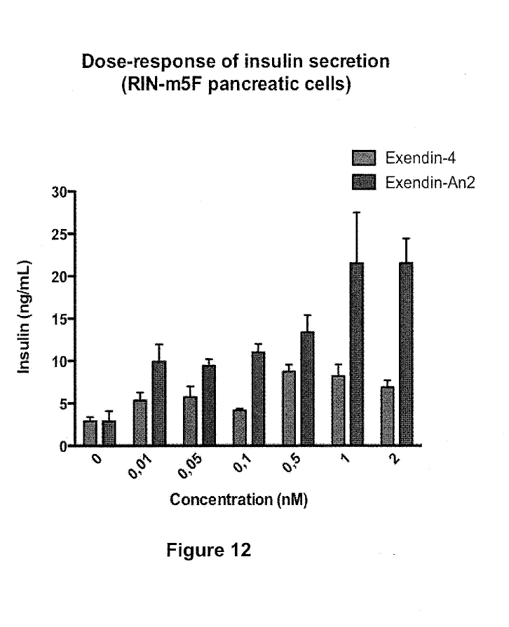

The ability of either Exendin-4 or Exen-4-An2 to induce an increase in insulation secretion was measured using RIN-m5F pancreatic cells. As shown in FIG. 12, the conjugate, at all concentrations tested, surprisingly exhibited a stronger level of insulin secretion, as compared to Exendin-4.

### EXAMPLE 8

### Synthesis of a Leptin Conjugate

The following procedure was used to generate a Leptin-(C11)-AN2 conjugate.

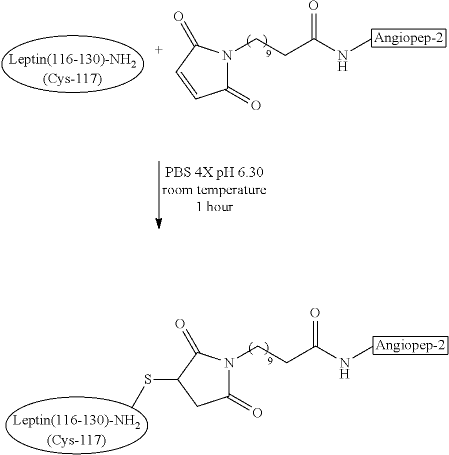

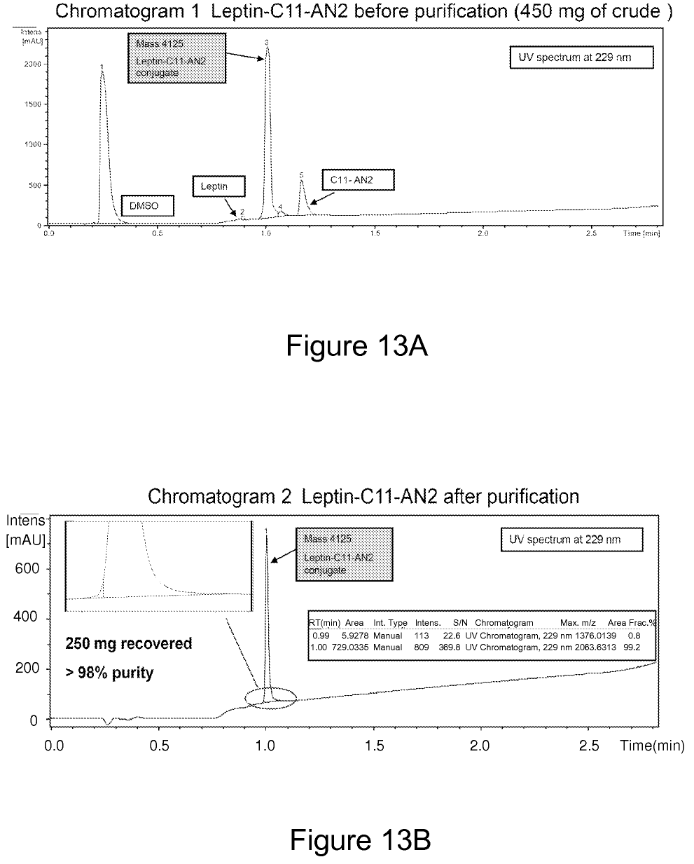

MUA-AN2 (264.6 mg, 91.5 μmol, 1.2 eq., 82% peptide content) was dissolved in H₂O/ACN (9/1) (14 ml) by adjusting pH from 3.9 to 5.00 with addition of a 0.1 N NaOH solution (1.5 ml). This solution was added to a solution of Leptin₁₁₆₋₁₃₀-NH₂ (156.5 mg, 76.2 μmol, 1 eq., 76% peptide content) in PBS 4× (pH 6.61, 7 mL). Monitoring of the reaction was done with the analytical method described below. Results are shown in FIGS. 13A and 13B (chromatograms 1 and 2).

A cloudy suspension was observed as the reaction went to completion. After 1 h at room temperature, the reaction (3.62 mM) was complete and the mixture was purified immediately by FPLC chromatography (AKTAexplorer, see chromatogram 3, Table 1). Purification was performed on a GE Healthcare AKTA explorer column (GE Healthcare) 30 RPC resin (polystyrene/divinylbenzene), 95 ml, sample load: 450 mg in reaction buffer (21 ml), 10% ACN in H₂O, 0.05% TFA (60 ml), DMSO.HCl (pH 2.87, 6 ml), Solution A: H₂O, 0.05% TFA, Solution B: ACN, 0.05% TFA, Flow: 5-17 ml/min, Gradient: 10-30% B.

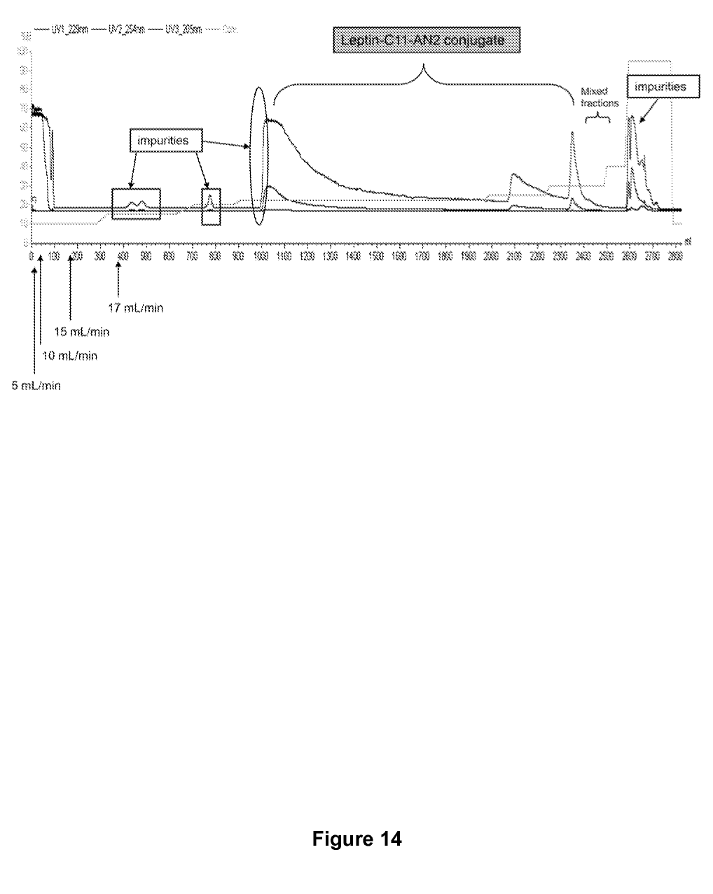

Purification results are shown in FIG. 14 (chromatogram 3). The gradient used to purify the compound is shown in the table below.

| Volume | Column volume | Flow rate | |
|---|---|---|---|
| (ml) | (C.V.) | (ml/min) | % Solvent B |
| 0 | 0 | 5 | 10 |
| 33.58 | 0.35 | 10 | 10 |
| 186.98 | 1.61 | 15 | 10 |
| 282.51 | 1.01 | 15 | 15.0 (over 3 min) |
| 346.26 | 0.67 | 16 | 15 |
| 366.68 | 0.21 | 17 | 15 |
| 625.3 | 2.72 | 17 | 20.0 (over 5 min) |
| 876.28 | 2.64 | 17 | 22.5 (over 2 min) |
| 1970.49 | 11.52 | 17 | 25.0 (over 1 min) |
| 2233.45 | 2.77 | 17 | 30.0 (over 1 min) |
| 2488.68 | 2.69 | 17 | 40.0 (over 0.5 min) |
| 2577.28 | 0.93 | 17 | 95.0 (over 1 min) |
| 2777.41 | 2.11 | 17 | 10.0 (over 0.5 min) |

After evaporation of acetonitrile and lyophilization, a white solid (250 mg, 79%, purity >98%) was obtained. The mass was checked by ESI-TOFMS (Bruker Daltonics). To avoid the possibility that some remaining Leptin(116-130)-NH₂ might dimerize 5%, cysteine peptide Mw =3119.44), immediate purification was performed and an 1.2 equivalent excess of maleimido-(C11)-AN2 was used.

To monitor the reaction, the following analytical method was used. A Waters Acquity UPLC system with a Waters Acquity UPLC BEH phenyl column was used (1.7 μm, 2.1×50 mm). Detection was performed at 229 nm. Solution A was H₂O, 0.1% FA, and Solution B was acetonitrile (ACN), 0.1% formic acid (FA). Flow and gradient are shown in the Table below.

| Time | Flow | | | |
|---|---|---|---|---|
| (min) | (ml/min) | % A | % B | Curve |
| | 0.5 | 90 | 10 | |
| 0.4 | 0.5 | 90 | 10 | 6 |
| 0.7 | 0.5 | 70 | 30 | 6 |
| 2.2 | 0.5 | 30 | 70 | 6 |
| 2.4 | 0.5 | 10 | 90 | 6 |
| 2.7 | 0.5 | 10 | 90 | 6 |
| 2.8 | 0.5 | 90 | 10 | 6 |
| 2.81 | 0.5 | 90 | 10 | 6 |

From mass spectroscopy (ESI-TOF-MS; Bruker Daltonics): calculated 4125.53; found 4125.06, m/z 1376.01 (+3), 1032.26 (+4), 826.02 (+5), 688.52 (+6).

The conjugate was stored under nitrogen atmosphere, in a dark room, below −20° C.

The leptin conjugate generated using the procedure is called Leptin-AN2 (C11), due its 11-carbon linker. Other length carbon linker conjugates, were also generated, including Leptin-AN2 (C3) and Leptin AN2 (C6) using similar procedures.

### EXAMPLE 9

### In Situ Brain Perfursion of Leptin ₁₁₆₋₁₃₀ Angiopep-2 Conjugates

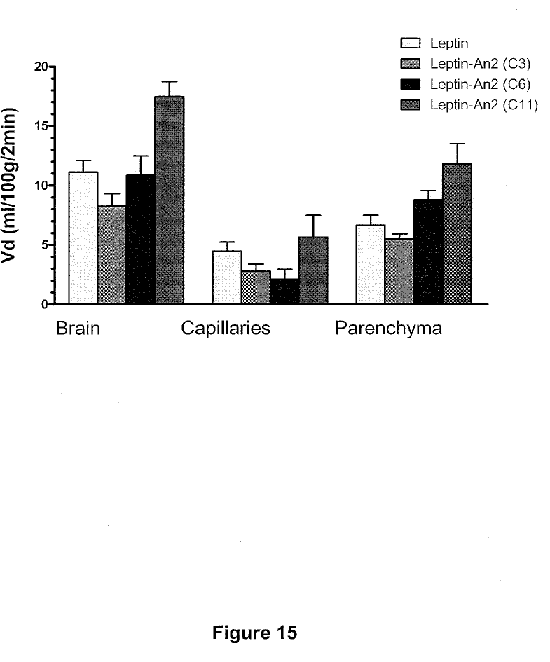

To determine which of the leptin conjugates most effectively crossed the blood-brain barrier, we tested each conjugate in the in situ brain perfusion assay. This assay is or a similar assay is described, for example, in U.S. Patent Publication No. 20060189515, which was based on a method described in Dagenais et al., 2000, J. Cereb. Blood Flow Metab. 20(2):381-386. The BBB transport constants were determined as previously described by Smith (1996, Pharm. Biotechnol. 8:285-307). From these experiments, Leptin-AN2 (C11) exhibed the greatest transport across the BBB as compared to the conjugates having C3 or a C6 linker and was thus selected for further experimentation (FIG. 15).

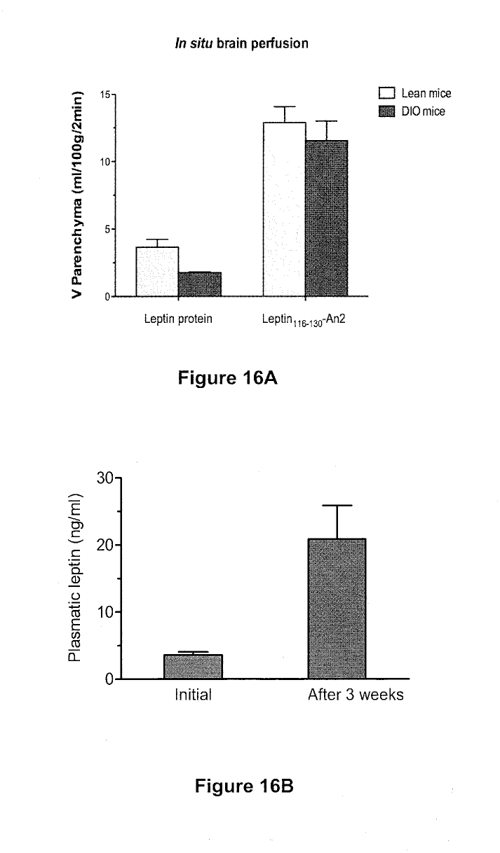

Transport of leptin was compared to the Leptin-AN2 (C11) conjugate using the in situ perfusion assay in lean and diet-induced obese (D10) mice (available, e.g., from the Jackson laboratories). From these results, transport of leptin across the BBB in DIO mice was reduced as compared to in lean mice. By contrast, the Leptin-AN2 (C11) conjugate crossed the brain much more efficiently in both lean and DIO mice, and no statistically significant difference between the lean and DIO mice in transport of the conjugate was observed (FIG. 16A). Plasma leptin levels were observed to increase after 3 weeks on a high fat (60%) diet, suggesting that the mice were becoming leptin resistant (FIG. 16B).

### EXAMPLE 10

### Effect of Leptin Conjugates on Food Intake and Weight Gain

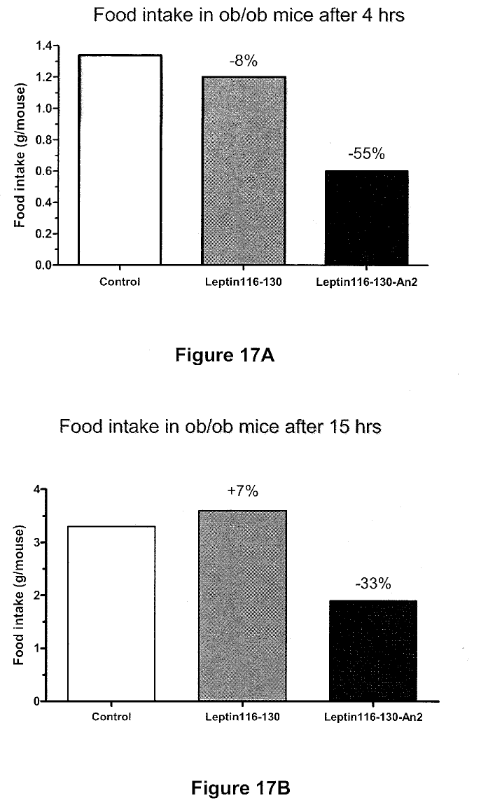

Mice were injected with an intravenous bolus of either Leptin-AN2 (C11) (eq. of 1 mg of leptin₁₁₆₋₁₃₀ per mouse), leptin₁₁₆₋₁₃₀ (1 mg/mouse), or a control (saline) (n=5 per group). Food intake of the mice was monitored at 4 hours (FIG. 17A) and at 15 hours (FIG. 17B). In both cases, the conjugate exhibited significantly greater reduction in food intake, as compared to either the control mice, or mice receiving leptin₁₁₆₋₁₃₀.

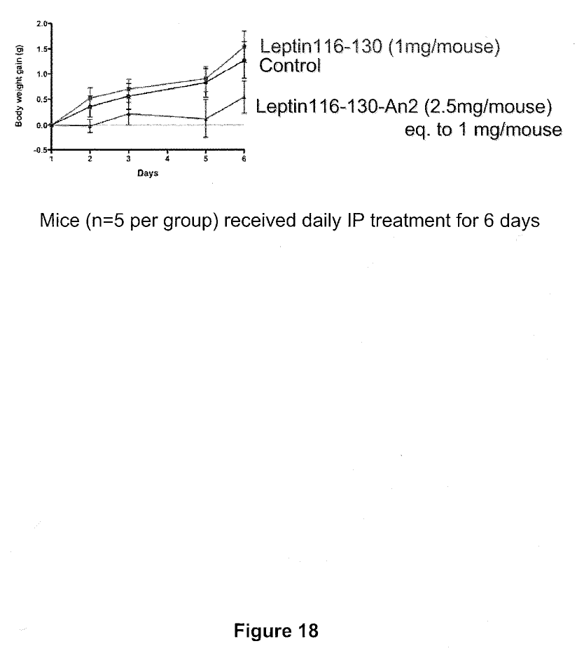

We also compared weight changes in DIO mice receiving the conjugate (2.5 mg/mouse; equivalent of 1 mg ₗₑₚₜᵢₙ₁₁₆₋₁₃₀ mg/mouse), leptin₁₁₆₋₁₃₀ (1 mg/mouse), and a control over a period of six days. Each mouse received daily treatment by intraperitoneal injection. Mice receiving leptin or the control exhibited similar amounts of weight gain over the six days, whereas mice receiving the conjugate showed marked reduction in weight gain (FIG. 18) as compared to the control mice and mice receiving leptin₁₁₆₋₁₃₀.

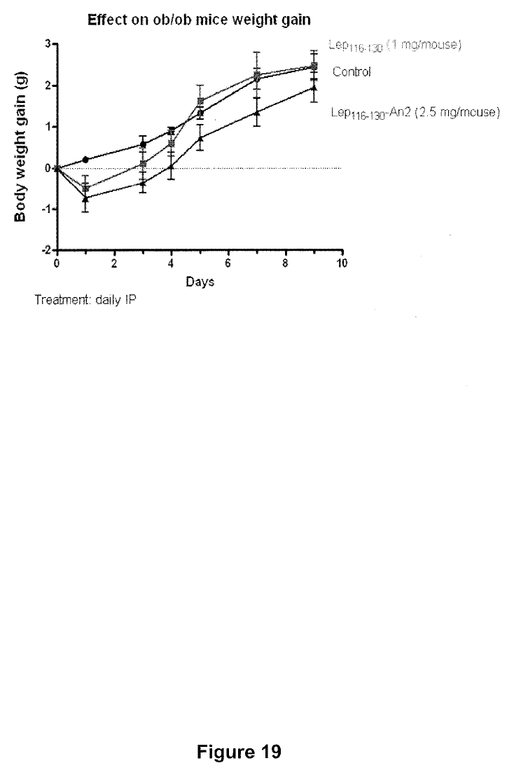

We further compared weight changes in leptin-deficient ob/ob mice receiving the conjugate (2.5 mg/mouse; equivalent of 1 mg leptin₁₁₆₋₁₃₀ mg/mouse), leptin₁₁₆₋₁₃₀ (1 mg/mouse), and a control over a period of six days. Each mouse (n=5 per group) received daily treatment by intraperitoneal injection. The mice receiving the conjugate exhibited lower weight gain than the mice receiviing either leptin₁₁₆₋₁₃₀ or the control (FIG. 19) during the period of administration.

### EXAMPLE 11

### Development of Recombinant Angiopep-2 and Angiopep-2 Leptin Fusion Proteins

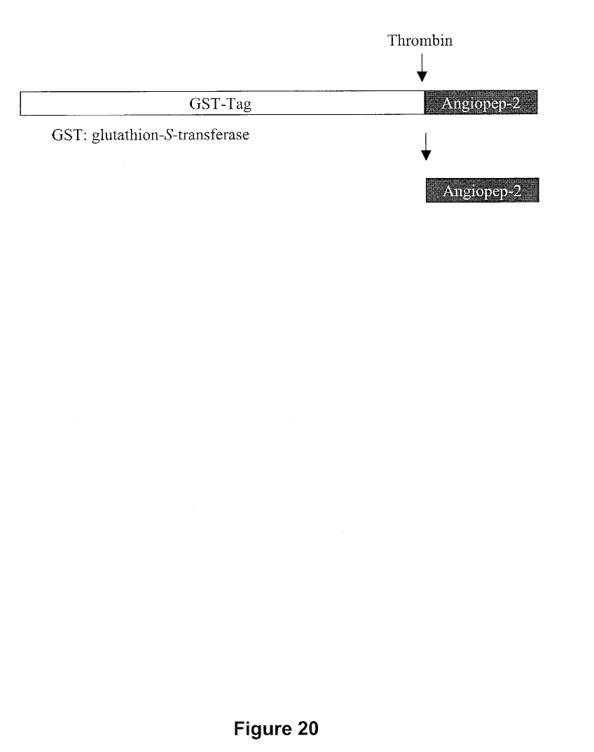

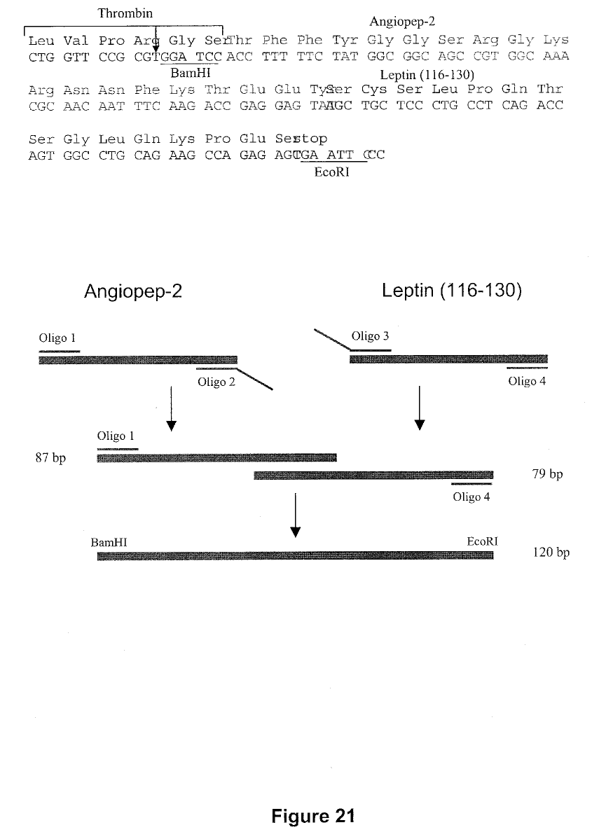

We also developed an Angiopep-2 fusion protein. As an initial step, a cDNA (ACC TTT TTC TAT GGC GGC AGC CGT GGC AAA CGC AAC AAT TTC AAG ACC GAG GAG TAT; SEQ ID NO:117) was created. This sequence was inserted into a pGEX vector system for bacterial expression, and sequence of the insert was verified (FIG. 20). The GST-An2-Leptin₁₁₆₋₁₃₀ construct was made using an overlap extension PCR strategy (FIG. 21).

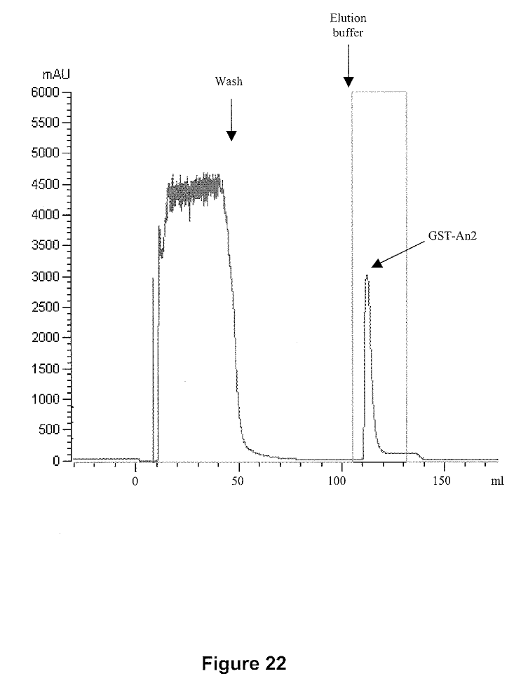

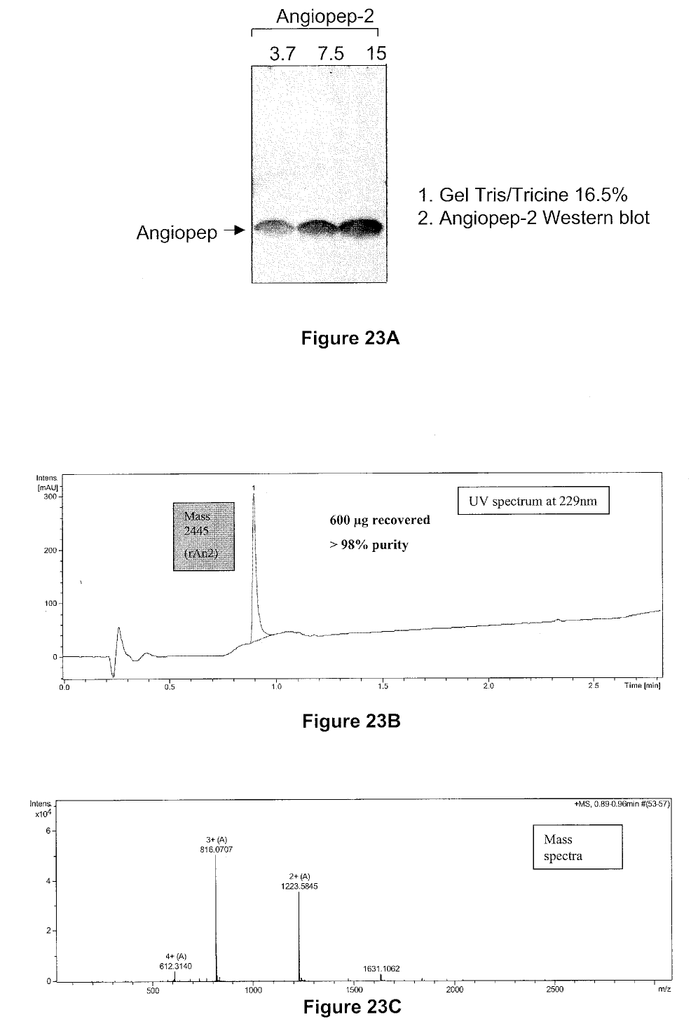

The recombinant Angiopep-2 was expressed in a bacterial expression system and purified using a GSH-Sepharose column. A chromatogram from this procedure is shown (FIG. 22). The purified Angiopep-2 was analyzed by Western blot using an Angiopep-2 antibody (FIG. 23A), by liquid chromatography (FIG. 23B), and by mass spectroscopy (FIG. 23C).

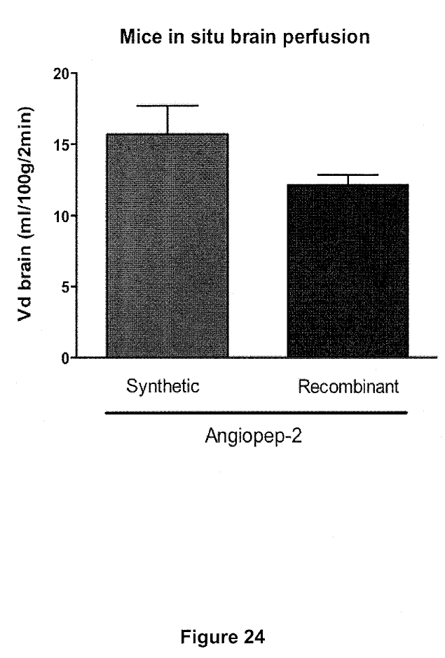

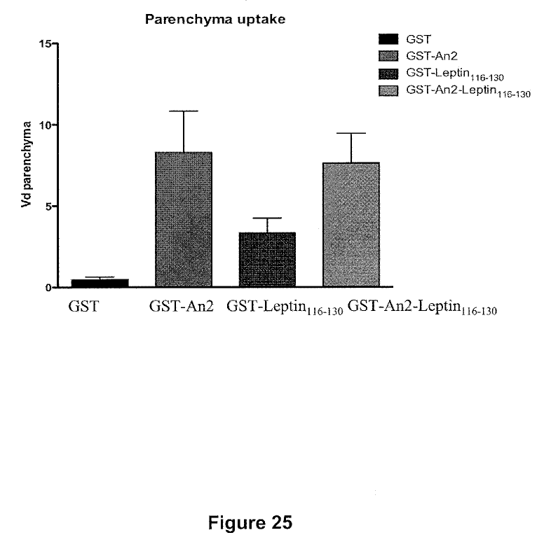

The in situ brain perfusion assay was performed using recombinant Angiopep-2. The results were compared to synthetic Angiopep-2 (FIG. 24). Similar levels of uptake were observed with both forms of Angiopep-2. Uptake into the parenchyma between GST, GST-Angiopep-2, GST-Leptin₁₁₆₋₁₃₀, and GST-Angiopep-2-Leptin₁₁₆₋₁₃₀ was compared (FIG. 25). These results show that fusion proteins containing the Angiopep-2 sequence are efficiently taken up into the parenchyma, whereas proteins lacking the Angiopep-2 sequence are taken up much less efficiently.

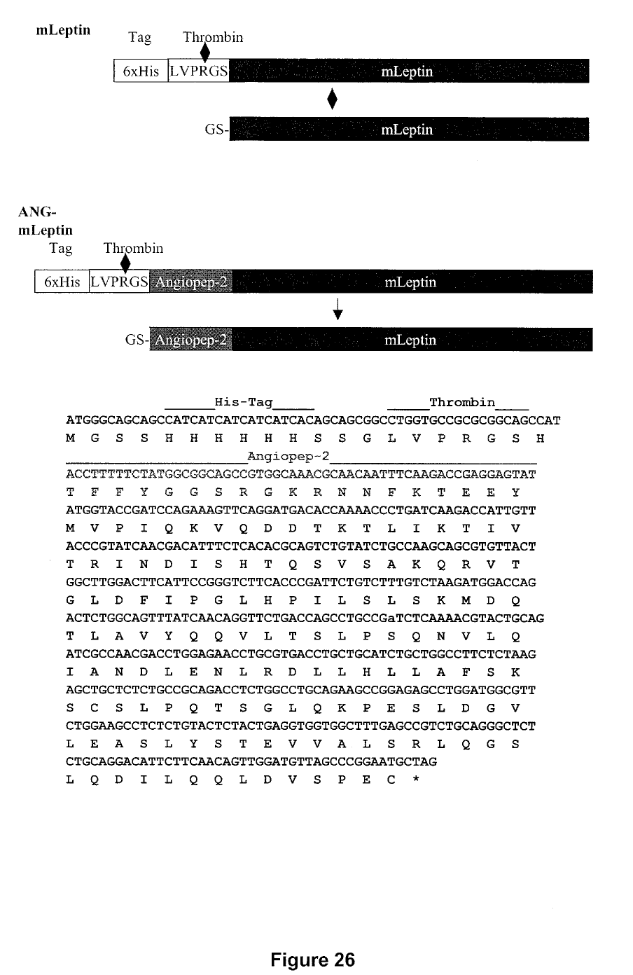

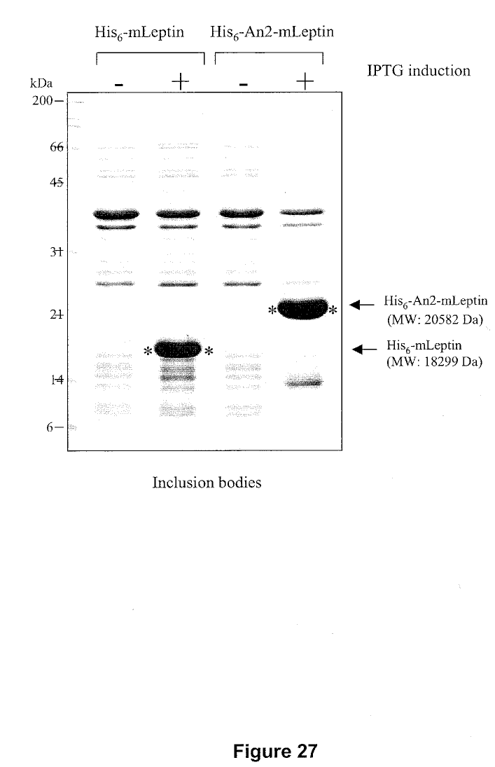

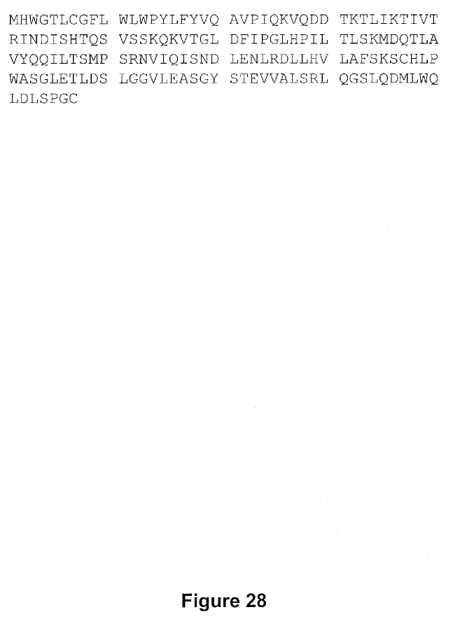

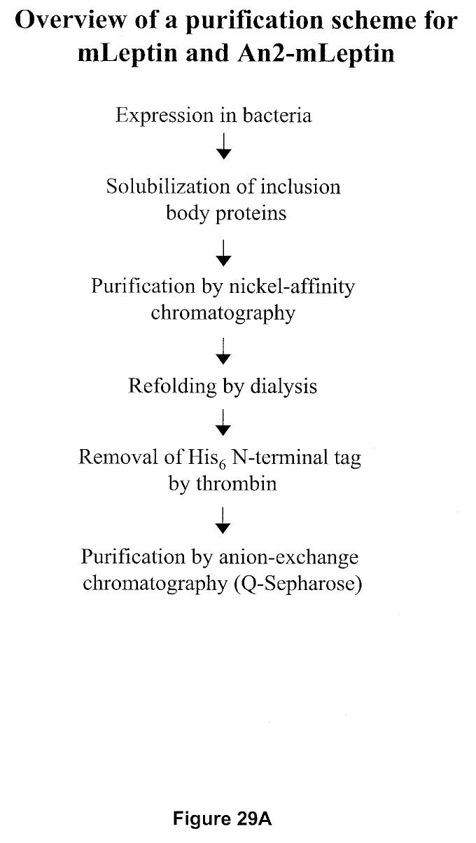

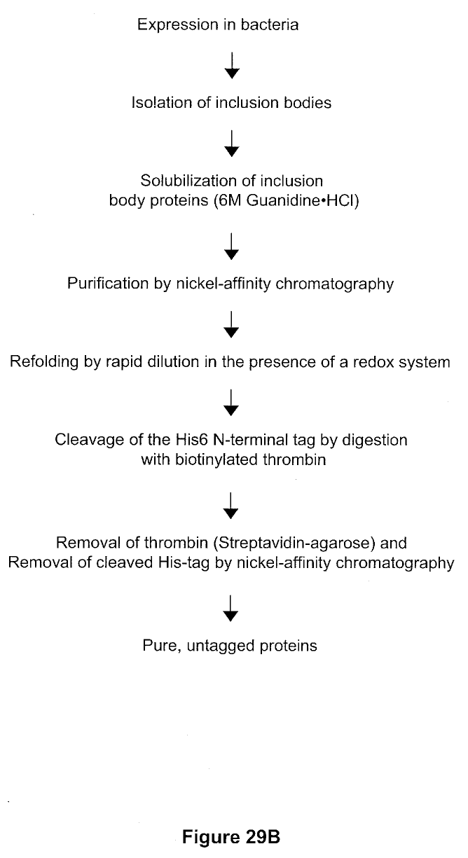

A His-tagged Angiopep-2/mouse leptin fusion protein containing the full length leptin sequence has been generated (FIG. 26). This fusion protein has been expressed in a bacterial expression system (FIG. 27). Exemplary purification schemes for the fusion protein are shown in FIGS. 29A and 29B. Results from a small scale purification are shown in FIG. 30.

The thrombin cleavage step resulted in production of two products, suggesting the possibility that the Angiopep-2 sequence contains a low-affinity thrombin cleavage site, as showin in FIG. 31. As the leptin-Angiopep-2 has a propensity to agregate in solution, purification conditions to reduce the aggregation and improve yields are being tested.

### EXAMPLE 12

### Brain Uptake and Activity of Leptin Fusion Proteins

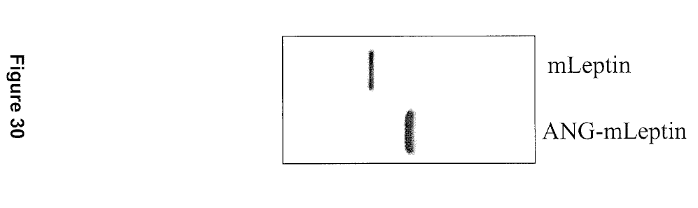

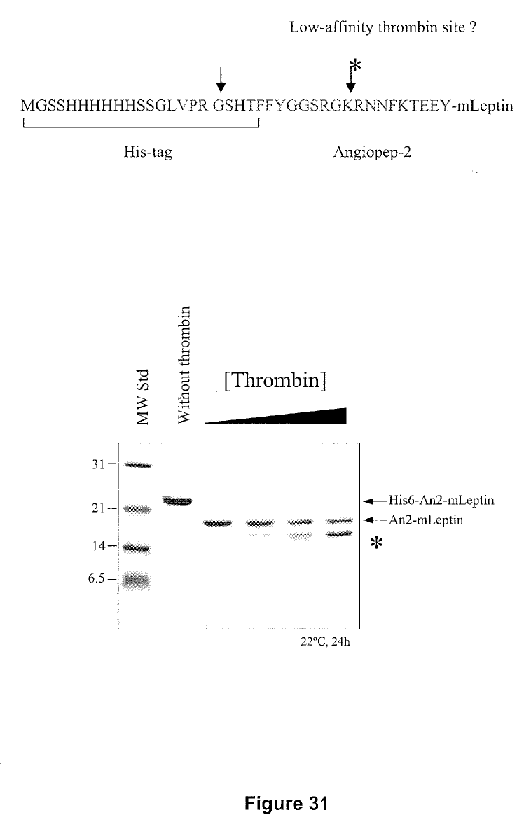

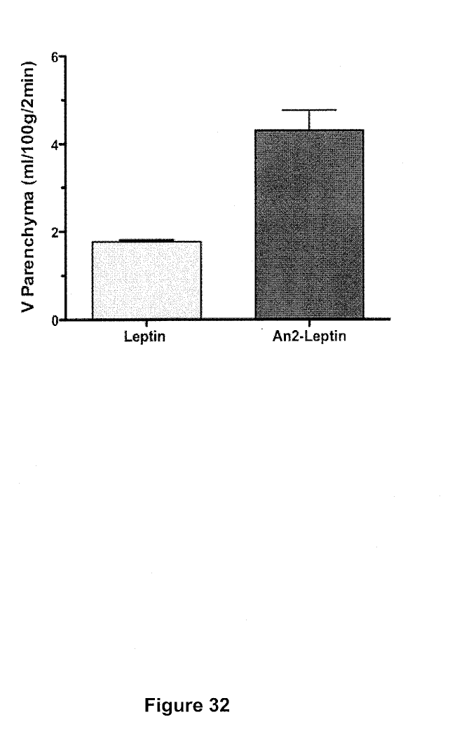

We then examined the ability of the Angiopep-2-leptin fusion protein to be taken up into the parenchyma of the brain of DIO mice as compared to leptin using the in situ brain perfusion assay (FIG. 32). From this experiment, we observed that the fusion protein exhibited increased uptake as compared to leptin.

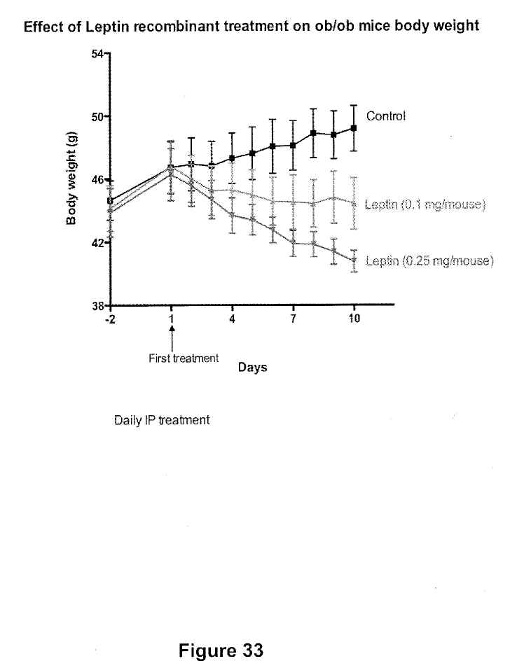

As a control, we tested the ability of recombinant leptin to reduce body weight in ob/ob mice using either 0.1 mg/mouse or 0.25 mg/mouse daily. As shown in FIG. 33, leptin did indeed reduce body weight in these mice in a dose-dependent manner.

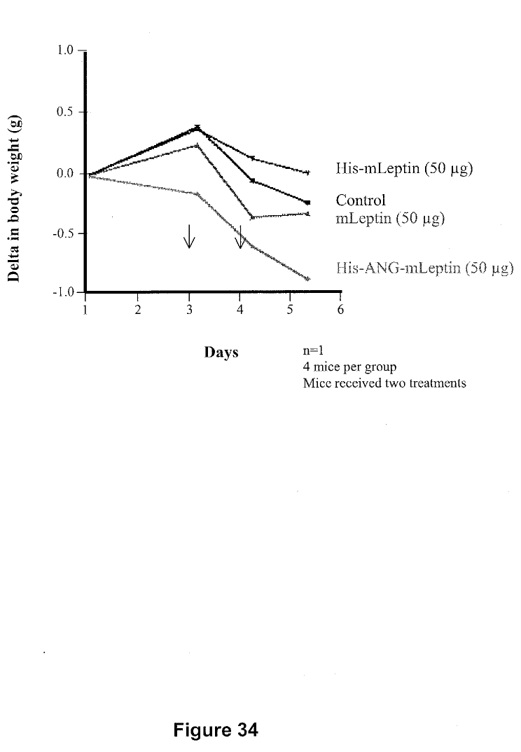

DIO mice were also treated with a control or with 50 pg his-tagged fusion protein, leptin, or the his-tagged leptin. Mice received two treatments, on days three and four as indicated. Based on these results, the greatest weight loss was observed in mice receiving the fusion protein (FIG. 34).

### Example 13

### Synthesis of a Neurotensin-Angiopep-2 Conjugate

An exemplary neurotensin-Angiopep-2 conjugate was synthesized using the scheme described below. As used in these examples, the abbreviation NT refers to the pE-substituted neurotensin peptide described below.

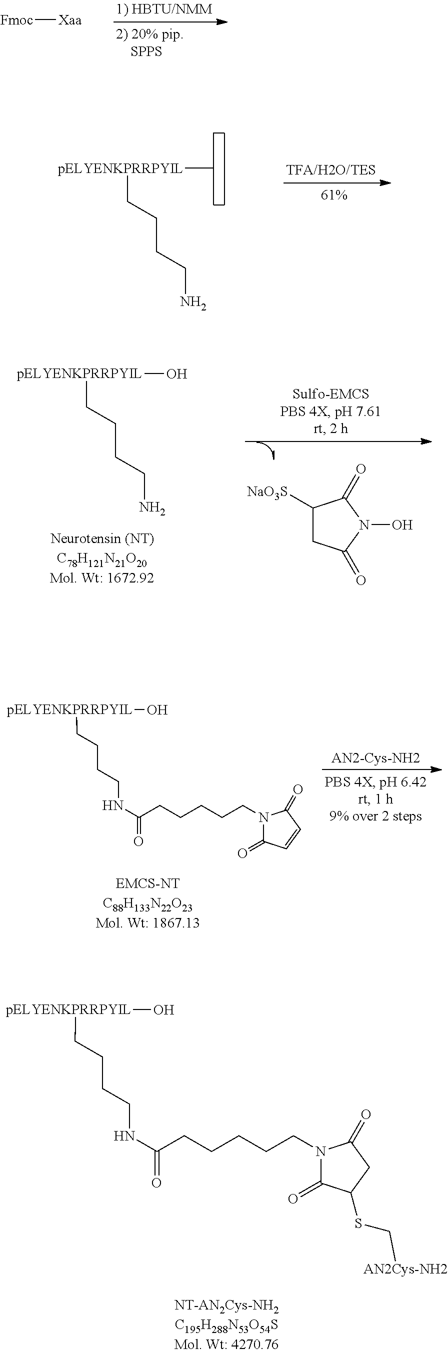

Neurotensin Peptide Synthesis

pELYENKPRRPYIL-OH, where the unusual amino acid L-pyroglutamic acid (pE) is used, was synthesized using SPPS (Solid phase peptide synthesis). SPPS was carried out on a Protein Technologies, Inc. Symphony® peptide synthesizer using Fmoc (9-fluorenylmethyloxycarbonyl) amino-terminus protection. The peptide was synthesized on a 100 pmol scale using a 5-fold excess of Fmoc-amino acids (200 mM) relative to the resin. Coupling was performed by a pre-loaded Fmoc-Leu-Wang resin (0.48 mmol/g) for carboxyl-terminus acids using 1:1:2 amino acid/activator/NMM in DMF with HBTU (2-(1H-benzotriazol-1-yl)-1,1,3,3-tetramethyluronium hexafluorophosphate) and NMM (N-methylmorpholine). Deprotection was carried out using 20% piperidine/DMF. The resin-bound product was routinely cleaved using a solution comprised of TFA/water/TES: 95/2.5/2.5 for 2 hours at room temperature.

Pre-loaded Fmoc-Leu-Wang resin (0.48 mmol/g) was purchased from ChemPep, Fmoc-amino acids, HBTU from Chemlmpex, and the unusual L-pyroglutamic acid from Sigma-Aldrich. Side protecting groups for amino acids were Trt (trityl) for aspargine, tBu (ter-butyl) for glutamic acid and tyrosine, Pbf (pentamethyldihydrobenzofuran-5-sulfonyl) for arginine, and tBoc (tButyloxycarbonyl) for lysine.

The crude peptide was precipitated using ice-cold ether, and purified by RP-HPLC chromatography (Waters Delta Prep 4000). Acetonitrile was evaporated from the collected fractions and lyophilized to give a pure white solid (204 mg, 61%, purity >98%). The mass was confirmed by ESI-TOF MS (Bruker Daltonics; calculated 1672.92; found 1671.90, m/z 558.31 (+3), 836.96 (+2)).

EMCS-NT

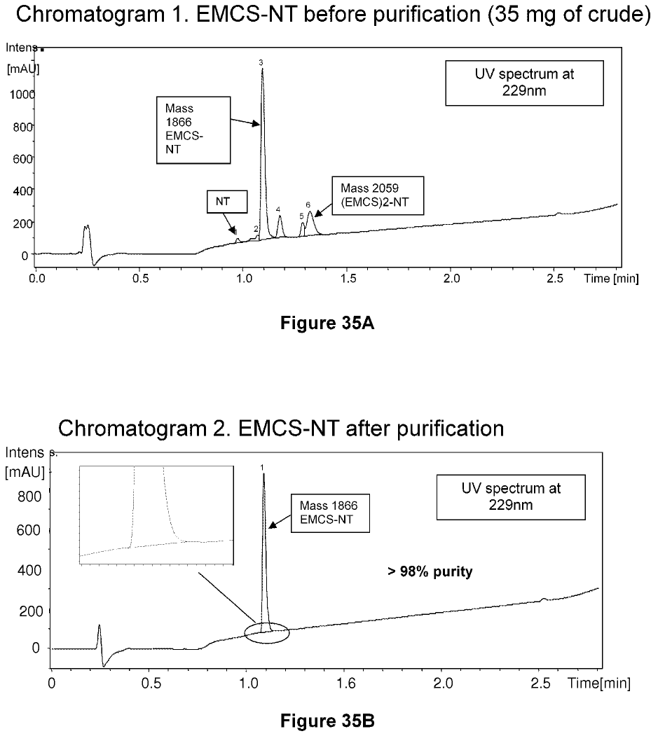

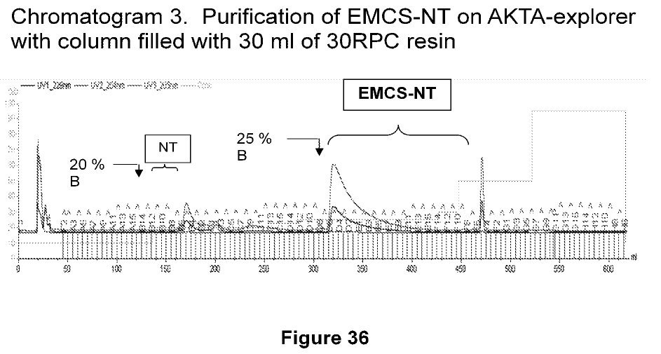

The N-lysine primary amine of NT was activated by treating a solution of NT (25 mg, 14.9 pmol, 1 eq. in 3.5 ml of PBS 4×, pH 7.64), with a solution of sulfo-EMCS (N-[L-maleimidocaproyloxy]sulfosuccinimide ester) (Pierce Biotechnology) (6.1 mg, 14.9 μmol, 1 eq. in 1 ml of PBS 4×). Monitoring of the reaction was done with the analytical method described below (see chromatograms 1-2 in FIGS. 35A and 35B). The reaction (3.32 mM, pH 7.61) allowed proceeding at room temperature for 1 h. The modification was repeated once for 1 h with addition of sulfo-EMCS (4.5 mg, 10.9 pmol, 0.73 eq. in 1 ml of PBS 4×). The mixture was purified by FPLC chromatography (AKTA explorer, see chromatogram 3 in FIG. 36). Purification of EMCS-NT was performed on a column containing 30 RPC resin (polystyrene/divinyl benzene), 30 ml. Sample was loaded as 35 mg in reaction buffer (4 ml), 10% acetonitrile (ACN) in H₂O, 0.05% TFA (200 μl). Solution A was H₂O, 0.05% TFA, and Solution B was ACN, 0.05% TFA. Flow rate 5-9 ml/min with a gradient of 10-25% of Solution B.

After the acetonitrile was evaporated, the volume of water was reduced to 5 ml for the next step. A colorless solution of the pure EMCS-modified NT (purity>98%) was obtained. The mass was checked by ESI-TOF MS (Bruker Daltonics), was calculated to be 1867.13, and was found to be 1866.00, m/z 623.01 (+3), 934.00 (+2).

NT-AN2Cys-NH2

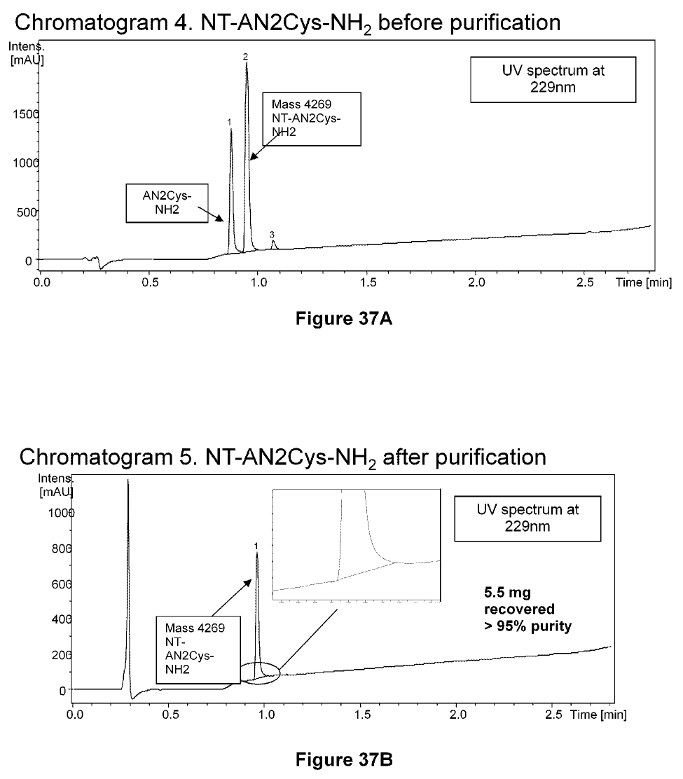

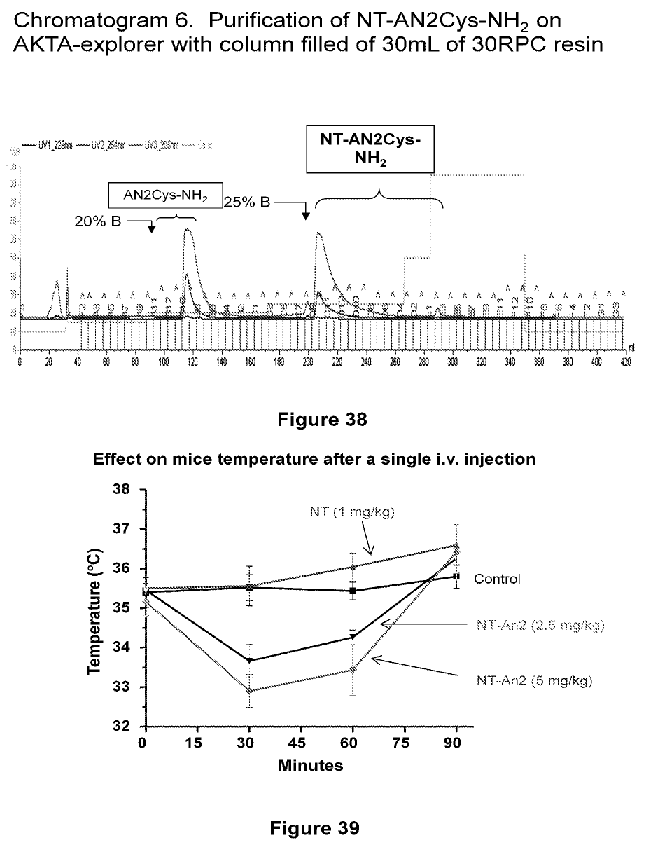

Conjugation was performed with the maleimido-containing EMCS-NT and the free thiol residue of AN2Cys-NH₂. The pH of the solution of EMCS-NT was adjusted from 1.65 to 6.42 by a slow addition of a 0.1N NaOH solution. A hydrolysis side reaction can occur during adjustment of pH (5%, hydrolyzed EMCS-NT Mw=1833). A solution of AN2Cys-NH₂ (46.4 mg, 14.9 pmol, 1 eq. in 2.5 ml of PBS 4×, pH 7.64) was added to the solution of EMCS-NT. The analytical method below was used to monitoring the reaction (see chromatograms 4-5 in FIGS. 37A and 37B). The reaction (1.9 mM, pH 6.3) was allowed to proceed at room temperature for 30 minutes. The mixture was purified by FPLC chromatography (AKTA explorer, see chromatogram 6 in FIG. 38). Purification of NT-AN2Cys-NH2 was performed using a column (GE Healthcare) containing 30 RPC resin (Polystyrene/divinyl benzene), 30 ml, Sample was loaded in the amount of 74 mg in 4 ml reaction buffer (10% ACN in H₂O, 0.05% TFA (200 ul)). Solution A was H₂O, 0.05% TFA, and Solution B was ACN, 0.05% TFA. The flow rate was 5-9 ml/min, using a gradient of 10% to 25% of Solution B.

After evaporation of acetonitrile and lyophilization, the conjugated NT-AN2Cys-NH₂ was obtained as a pure white solid (5.5 mg, 9% over 2 steps, purity>95%). The mass was confirmed by ESI-TOF MS (Bruker Daltonics); MW was calculated to be 4270.76 and was found to be 4269.17 (m/z 712.54 (+6), 854.84 (+5), 1068.29 (+4), 1424.04 (+3)).

The conjugate was stored under nitrogen atmosphere, below −20° C.

Analytical Method

The following method was used as described above. To analyze samples during purification, a Waters Acquity UPLC system was employed with a BEH phenyl column, 1.7 μm, 2.1×50 mm Detection was performed at 229 nm. Solution A was H₂O, 0.1% FA, and Solution B was acetonitrile (ACN), 0.1% FA. Flow rate was 0.5 ml/min with a gradient of 10-90% B, as shown in the table below.

| Time | Flow | | | |
|---|---|---|---|---|
| (min) | (mL/min) | % A | % B | Curve |
| | 0.5 | 90 | 10 | |
| 0.40 | 0.5 | 90 | 10 | 6 |
| 0.70 | 0.5 | 70 | 30 | 6 |
| 2.20 | 0.5 | 30 | 70 | 6 |
| 2.40 | 0.5 | 10 | 90 | 6 |
| 2.70 | 0.5 | 10 | 90 | 6 |
| 2.80 | 0.5 | 90 | 10 | 6 |
| 2.81 | 0.5 | 90 | 10 | 6 |

### EXAMPLE 14

### Characterization of the NT-AN2Cys-NH₂ Conjugate

To investigate the pharmacological efficacy and brain penetration of the NT-AN2Cys-NH₂ (NT-An2) conjugate, we monitored its effect on the body temperature of mice (FIG. 39). The temperature of mice was unaffected by intravenous administration of 1 mg/kg NT or the saline control. By contrast, intravenous administration of an equivalent dose of the conjugate (2.5 mg/kg) resulted in a rapid decrease in the body temperature, leading to hypothermia. The injection of a higher dose (5 mg/kg) of NT-An2 caused a stronger decrease in body temperature indicating that the effect of NT-An2 is dose dependent.

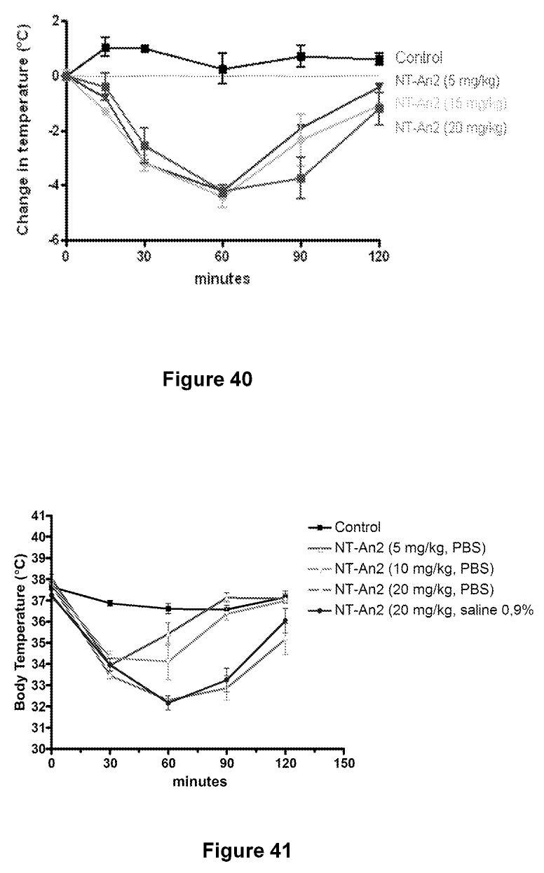

We also tested whether higher doses of the conjugate would results in greater induction of hypothermia. Mice were administered 5, 15, or 20 mg/kg of the conjugate, and the reduction in body temperature following administration was monitored for 120 minutes following administration. Small differences between these higher doses were observed (FIG. 40).

This experiment was repeated again with a second small batch of the NT-An2 compound, which resulted in similar activity. A third batch, which was produced as a part of an attempt to scale up the production, exhibited similar but somewhat lower activity, as shown in FIG. 41.

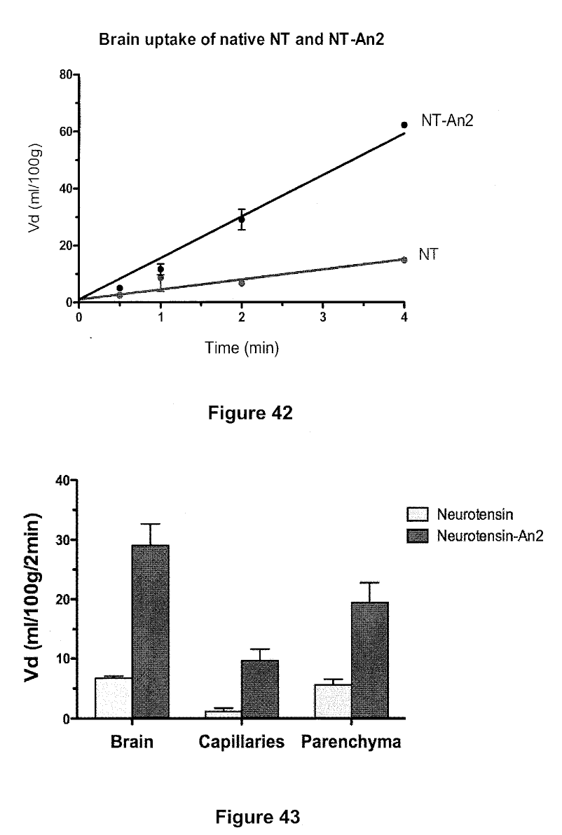

To confirm that the NT-An2 conjugate crosses the BBB, both NT and the conjugate were iodinated using standard procedures, and in situ brain perfusion was performed using methods standard in the art. The initial transport was measured as a function of time (FIG. 42). Results clearly indicate that the initial brain uptake for the NT-An2 conjugate is higher than for the unconjugated NT. Furthermore, after a 2 min in situ perfusion, capillary depletion was done to quantify the amount of NT-An2 found in the brain parenchyma (FIG. 43). Higher levels of NT-An2 were found in the brain parenchyma when compared to NT. In addition, these results indicate that NT-An2 is not trapped in the brain capillaries. Overall, our results demonstrate that the new NT-An2 derivative crosses the BBB at a sufficient concentration required to activate its receptors involved in the control of the body temperature.

### EXAMPLE 15

### Induction of Sustained Hypothermia using Angiopep-NT Conjugates

We performed an additional experiments to test whether the conjugates were able to induce sustained hypothermia in mice and rats.

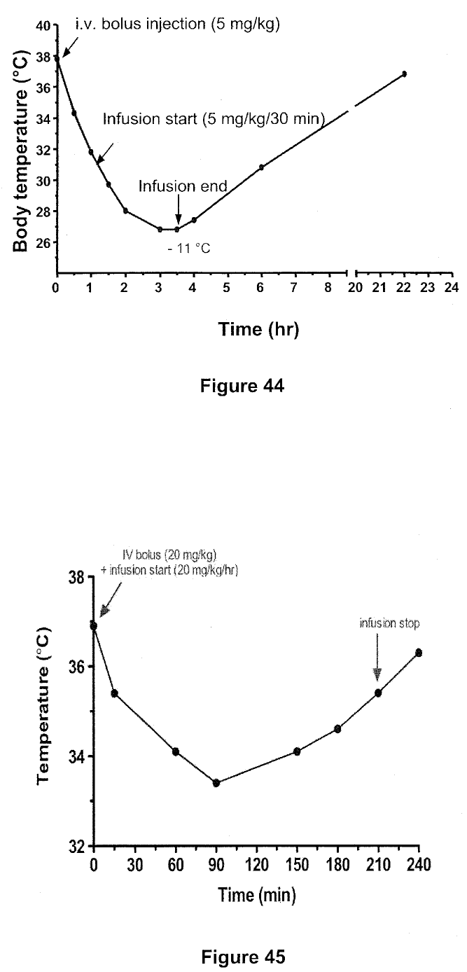

In a first experiment, mice first received an intravenous 5 mg/kg bolus injection of NT-An2, followed by an intravenous infusion (10 mg/kg/hr) 1 hour later for a duration of 2.5 hours. The body temperature continued to decrease during the infusion, reaching a nadir of −11° C. (FIG. 44). After the end of the infusion, body temperature slowly returned to 37° C., and the animals recovered.

A similar experiment was performed in rats. Here the rats were administered an intravenous bolus injection of 20 mg/kg NT-An2 immediately followed by a 20 mg/kg/hr infusion for 3.5 hours. This resulted in a maximal temperature drop of about 3.5° C. after 90 minutes (FIG. 45).

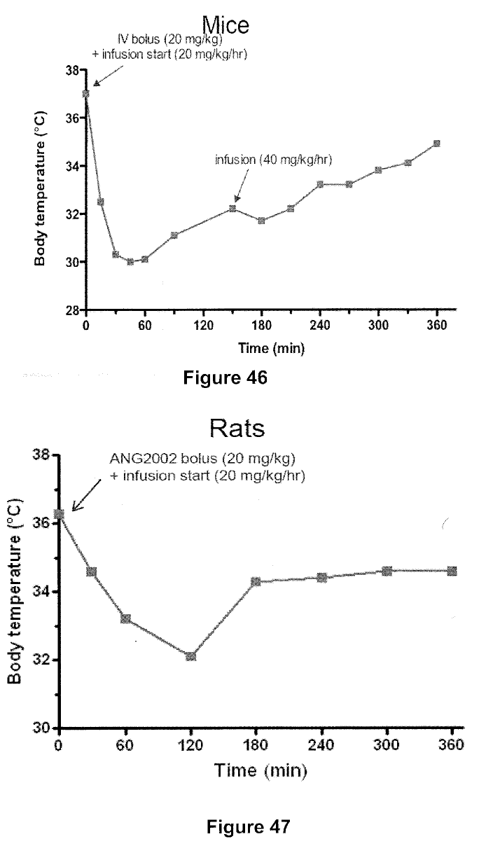

Sustained hypothermia experiments were performed using a intravenous bolus injection of 20 mg/kg of NT-An2 immediately followed by a 20 mg/kg/hr infusion for 2.5 hours. At this time, the infusion was increased to 40 mg/kg/hr. A reduction in body temperature for the initial 37° C. was observed over the 360 minute time course of the experiment (FIG. 46).

A similar experiment was conducted in rats. In this experiment, rats were injected intravenously with 20 mg/kg of NT-An2 immediately followed by a 20 mg/kg/hr infusion. A sustained reduction in body temperature was also observed during the 360-minute time course of this experiment (FIG. 47).

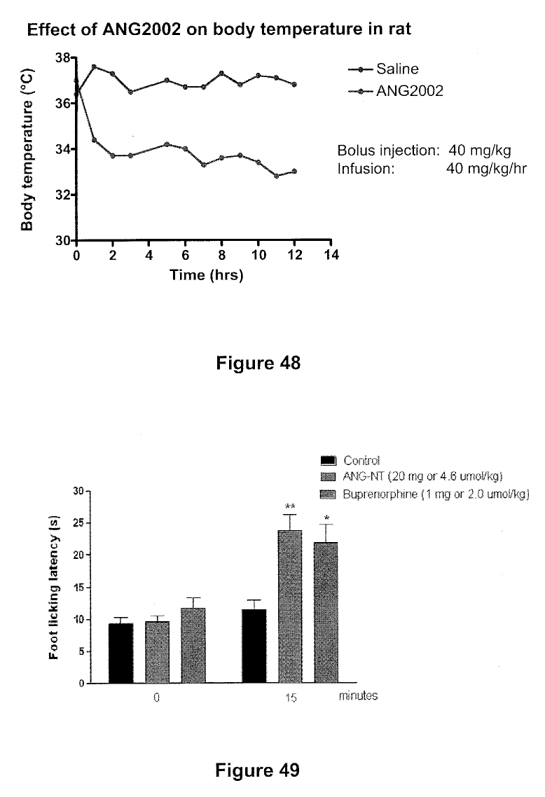

A further experiment, conducted over a 12-hour period, was also performed in rats. This experiment involved a 40 mg/kg intravenous bolus injection of NT-An2 followed immediately by a 20 mg/kg/hr infusion of NT-An2. As shown in FIG. 48, this resulted in a prolonged reduction of body temperature over the course of the experiment.

### EXAMPLE 16

### Analgesia Induction by NT-An2

We also tested the ability of NT-An2 to induce analgesia in mice. We tested the latency between hot plate foot exposure and foot licking behavior in control mice, mice receiving 20 mg/kg NT-An2, and mice receiving 1 mg/kg of buprenorphine (an opiate analgesic) as a positive control. Both the NT-An2 and the buprenorphine increased the latency of foot licking behavior in a statistically significant manner 15 minutes following injection, thus indicating that NT-An2 can act as an analgesic (FIG. 49).

### EXAMPLE 17

### Generation of Shorter Neurotensin Analogs

We further generated several shorter neurotensin analogs. These analogs include NT(8-13) (RRPYIL), Ac-LysNT(8-13), Ac-Lys-[D-Tyr¹¹]NT(8-13), pGlu-LysNT(8-13), MHA-NT(8-13), and β-mercaptoMHA-NT(8-13) (see below).

| Name | Sequence | Mw (g/mol) | Qtty (mg) |
|---|---|---|---|
| NT native | pELYENKPRRPYIL | 1672.97 | 800, ≧95% |
| NT(8-13) | RRPYIL | 816.99 | 45, ≧95% |
| Ac-LysNT(8-13) | 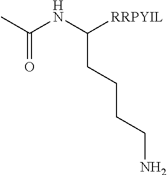 | 987.20 | 78, ≧95% |
| Ac-Lys-[D- Tyr¹¹]NT(8-13) | 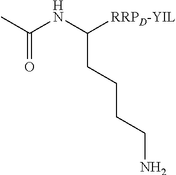 | 987.20 | 55, ≧95% |
| pGlu-LysNT(8-13) | 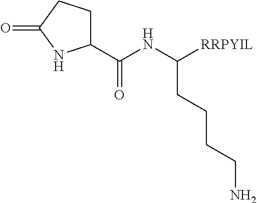 | 1056.26 | 86, ≧95% |
| MHA-NT(8-13) | 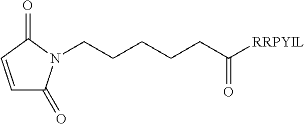 | 1010.19 | 55, ≧95% |
| □-mercaptoMHA- NT(8-13) (desactive) | 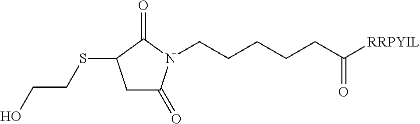 | 1088.32 | 12, ≧95% |

NT and the NT(8-13) analogs were synthesized by using a SPPS method on a Protein Technologies, Inc. Symphony® peptide synthesizer and Fmoc chemistry. Pre-loaded Fmoc-Leu-Wang resin (0.48 mmol/g) was purchased from ChemPep, Fmoc-amino acids, HBTU from Chemlmpex, the unusual pE from Sigma-Aldrich, unnatural D-Tyrosine from Chemlmpex, Sulfo-EMCS from Pierce Biotechnology. Side protecting groups for amino acids were Trt for aspargine, tBu for glutamic acid and tyrosine, Pbf for arginine, and tBoc for lysine. Mass was confirmed by ESI-TOF MS (MicroTof, Bruker Daltonics).

General procedure—Synthesis of Neurotensin (NT) (pELYENKPRRPYIL-OH). NT was synthesized using the unusual L-pyroglutamic acid (pE) and a 5 fold excess of Fmoc-AA (200 mM) relative to the resin. Coupling was performed from a pre-loaded Fmoc-Leu-Wang resin (0.48 mmol/g) for carboxyl-terminus acids using 1:1:4 AA/HBTU/NMM in DMF. Deprotection was carried out using 20% piperidine/DMF. The resin-bound product was routinely cleaved using a cocktail solution comprised of TFA/water/TES: 95/2.5/2.5 for 2 h at room temperature.

The crude peptide was precipitated using ice-cold ether and was purified by RP-HPLC chromatography, Waters Delta Prep 4000, Kromasil 100-10-C18, H₂O/ACN with 0.05%TFA (“Method A”). Acetonitrile was evaporated from the collected fractions and lyophilized. This resulted in the formation of a white and fluffy solid, 800 mg, 80% yield, purity HPLC>98%, calc. 1672.92, found 1671.90, m/z 558.31 (+3), 836.96 (+2).

Synthesis of MHA-NT(8-13) (MHA-RRPYIL-OH). The same procedure was used as for NT. A 100mM Fmoc-AA solution, and TBTU were used. Prior to cleavage, the N-terminal MHA group was introduced on SPPS by treating the free N-terminal amino peptide bound to the resin with an 18 mM solution of Sulfo-EMCS (1.2 eq. in DMF) for 1.5 h at room temperature. The crude peptide was purified by RP-HPLC chromatography, Waters Delta Prep 4000, Waters BEH Phenyl, H₂O/ACN with 0.05%TFA (“Method B”). This generated 55 mg of product, 73% yield, purity HPLC 95%, calc. 1010.19, found 1010.59, m/z 505.81 (+2).

Synthesis of Ac-Lys-p-Tyr¹¹]NT(8-13) (Ac-KRRPD-YIL-OH). The same procedure was used as for NT. D-Tyrosine, a 100 mM Fmoc-AA solution, and TBTU were used. Before cleavage, a subsequent capping reaction was carried out using a large excess of 1:1:3 v/v/v acetic anhydride/DIEA/DMF for 10 min at room temperature. The peptide was purified by Method A. This resulted in the formation of a white and fluffy solid, 426 mg, 82% yield, purity HPLC 95%, calc. 987.20, found 987.58, m/z 494.30 (+2).

Synthesis of ANG-Cys-NH2 (H-T¹FFYGG⁶S⁷RGKRNNFKTEEYC-NH2). ANG-Cys-NH2 was synthesized using a 5-fold excess of Fmoc-AA (200 mM) relative to the resin. G⁶S⁷ is coupled as the pseudoproline dipeptide GS. Coupling was performed from a Rink amide MBHA resin with Nle (0.40 mmol/g) for carboxyl-terminus amides using 1:1:4 AA/HCTU/NMM in DMF. Cleavage of the resin-bound product was carried out using TFA/water/EDT/TES: 94/2.5/2.5/1 for 2 h at room temperature.

The crude peptide was precipitated using ice-cold ether, and purified by RP-HPLC chromatography twice successively, Waters Delta Prep 4000, Kromasil 100-10-C18 and Waters BEH Phenyl, H₂O/ACN with 0.05%TFA (“Method C”). Acetonitrile was evaporated from the collected fractions and lyophilized. This resulted in formation of a white and fluffy solid, 565 mg, 28% yield, purity HPLC>90%, calc. 2403.63; found 2402.05, m/z 1202.53 (+2), 802.04 (+3), 601.78 (+4).

General procedure—Synthesis of MHA-NT. NT (1 eq.) was dissolved in PBS 4× (pH 7.3), and the solution pH was adjusted to 7.1 by addition of NaOH 0.1 N solution. To this solution was added a solution of Sulfo-EMCS (1 eq. in PBS 4×). Monitoring of the reaction was done with an analytical method. The reaction (9.0 mM, pH 7.1) allowed proceeding at RT for 2 h. The pH of reaction was adjusted from 5.2 to 7 with addition of NaOH 0.1 N solution.

After a second addition of sulfo-EMCS (0.3 eq. in PBS 4×), the reaction was repeated for 1 h. The mixture was purified by FPLC chromatography, AKTA explorer, 30RPC resin, H₂O/ACN without acid (“Method D”). Before evaporation, the resulting pure pooled fractions were acidified to pH 4 with a solution of water, 0.1%TFA.

After acetonitrile was evaporated, the volume of water was reduced to a minimum volume to be engaged directly in the subsequent conjugation step. This resulted in a colorless solution, estimated to be 278 mg, 83% yield, purity HPLC>98%, calc. 1867.13, found 1866.00, m/z 623.01 (+3), 934.00 (+2).

Synthesis of AcLys(MHA)NT(8-13)(D-Tyr11). The same procedure as MHA-NT from AcLysNT(8-13)(D-Tyr11). After the first addition of the solution of Sulfo-EMCS (1 eq. in PBS 4×), the reaction (5.0 mM, pH 6.8) allowed proceeding at room temperature for 2 h. This produced a colorless solution, estimated to be 24 mg, 67% yield, purity HPLC calc. 1180.40, found 1180.64, m/z 590.83 (+2).

The following abbreviations are used in the description of the above synthetic methods.
AA Amino acid
Ac Acetyl group
ANG Angiopep-2
DIEA Diisopropylethylamine
DMF Dimethylformamide
DMSO Dimethylsulfoxide
Fmoc 9-Fluorenylmethyloxycarbonyl
HBTU 2-(1H-Benzotriazol-1-yl)-1,1,3,3-TetramethylUronium Hexafluorophosphate
HCTU 2-(1H-6-Chlorobenzotriazol-1-yl)-1,1,3,3-TetramethylUronium Hexafluorophosphate
MBHA 4-Methylbenzhydrylamine
MHA Maleimidohexanoic acyl group
NMM N-MethylMorpholine
NT Neurotensin
Pbf Pentamethyldihydrobenzofuran-5-sulfonyl
pE L-pyroglutamic acid
SPPS Solid Phase Peptide Synthesis
Sulfo-EMCS N-[L-maleimidocaproyloxy]sulfosuccinimide ester
tBoc tButyloxycarbonyl
TBTU 2-(1H-Benzotriazol-1-yl)-1,1,3,3-TetramethylUronium Tetrafluoroborate
tBu ter-butyl group
TES Triethylsilane
TFA Trifluoroacetic acid
Trt Trityl group

### EXAMPLE 18

### Characterization of Neurotensin Analogs

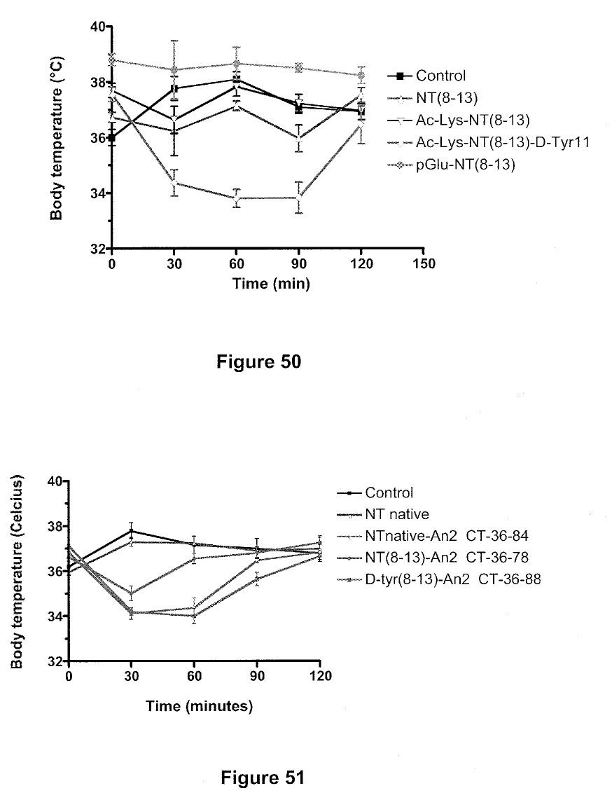

To determine which NT analog or analogs would be best suited for conjugation to Angiopep-2, we evaluated the ability of each analog to induce hypothermia in mice. Bolus intravenous injections of 7.5 mg/kg of NT(8-13), Ac-Lys-NT(8-13), Ac-Lys-[D-Tyr¹¹]NT(8-13), pGlu-NT(8-13), and a control were performed (FIG. 50) and body temperature was measured over a period of 120 minutes. Ac-Lys-[D-Tyr¹¹]NT(8-13) exhibited the greatest reduction in body temperature of the analogs tested. This analog was therefore selected for conjugation and further experimentation.

### EXAMPLE 19

### Generation of Neurotensin Analog Conjugates

Three neurotensin and NT analog conjugates were generated, NT-AN2 (as described above), NT(8-13)-AN2, and Ac-Lys-[D-Tyr¹¹]NT(8-13)-AN2. The structure of each of these conjugates is shown in the table below.

| | | Mw | |
|---|---|---|---|
| Name | Sequence | (g/mol) | Qtty (mg) |
| NT-An2 | 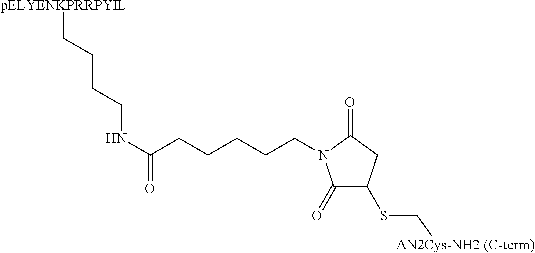 | 4270.76 | 18, ≧95% |
| NT(8-13)-AN2 | 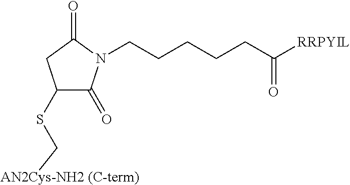 | 3413.82 | 59, ≧95% |
| Ac-Lys-[D- Tyr¹¹]NT(8- 13)-AN2 | 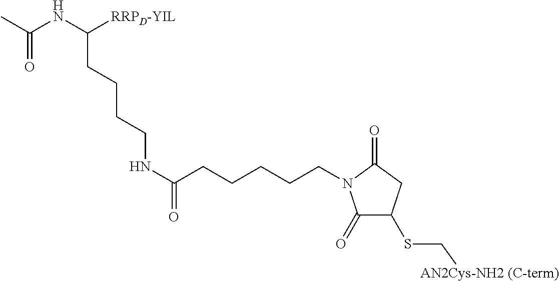 | 3584.03 | 17, ≧95% |

The conjugates analogs were synthesized by using a SPPS method on a Protein Technologies, Inc. Symphony® peptide synthesizer and Fmoc chemistry. Pre-loaded Fmoc-Leu-Wang resin (0.48 mmol/g) was purchased from ChemPep, Fmoc-amino acids, HBTU from Chemlmpex, the unusual pE from Sigma-Aldrich, unnatural D-Tyrosine from Chemlmpex, Sulfo-EMCS from Pierce Biotechnology.

Side protecting groups for amino acids were Trt for aspargine, tBu for glutamic acid and tyrosine, Pbf for arginine, and tBoc for lysine. Mass was confirmed by ESI-TOF MS (MicroTof, Bruker Daltonics). All abbreviations used in the following methods are defined in Example 17 above.

General procedure—Synthesis of ANG-NT. Conjugation was performed with the maleimido-containing MHA-NT and the free thiol residue of ANG-Cys-NH2.

The pH of the previously prepared solution of MHA-NT was adjusted from 4.2 to 5 by slow addition of a 0.1N NaOH solution. To this solution of MHA-NT was added a solution of ANG-Cys-NH2 (1 eq. in PBS 4×, pH 7.3). Monitoring of the reaction was done with an analytical method. The reaction (2.5 mM, pH 5.1) was allowed to proceed at room temperature for 1 h. The mixture was purified by FPLC chromatography, AKTA explorer, 30RPC resin, H₂O/ACN with 0.05% TFA (“Method E”).

After evaporation of acetonitrile and lyophilization, the conjugated ANG-NT was obtained as a pure white solid, 412 mg, 65% yield, 54% over 2 steps, purity HPLC>95%, calc. 4270.76, found 4269.17, m/z 712.54 (+6), 854.84 (+5), 1068.29 (+4), 1424.04 (+3).

Synthesis of ANG-NT(8-13). The same procedure as ANG-NT was used for MHA-NT(8-13). MHA-NT(8-13) (1 eq.) was dissolved in DMSO (19 mM). The mixture was purified by Method B (see above). This resulted in a white and fluffy solid, 597 mg, 88% yield, purity HPLC 95%, calc. 3413.82, found 3413.46, m/z 683.75 (+5), 854.19 (+4), 1138.91 (+3).

Synthesis of ANG-AcLys-p-Tyr¹¹NT(8-13). The same procedure as ANG-NT was used for AcLys(MHA)- [D-Tyr¹¹]NT(8-13). The peptide was purified by Method E. This resuled in a white solid, 17 mg, 24% yield, purity HPLC 95%, calc. 3584.03, found 3583.79, m/z 598.30 (+6), 717.76 (+5), 896.70 (+4), 1195 (+3).

### EXAMPLE 20

### Characterization of NT Analog Conjugates

To determine the ability of the NT analog conjugates to induce hypothermia, a bolus of a control, unconjugated NT, NT-An2, NT(8-13)-An2, and Ac-Lys-[D-Tyr¹¹]NT(8-13)-An2 were each injected intravenously into mice, and body temperature was monitored over a period of 120 minutes. Little difference between the control and the unconjugated NT, some effect was observed with the NT(8-13)-An2 conjugate, and a larger effect was observed with both the NT-An2 and Ac-Lys-[D-Tyr¹¹]NT(8-13)-An2 conjugates (FIG. 51).

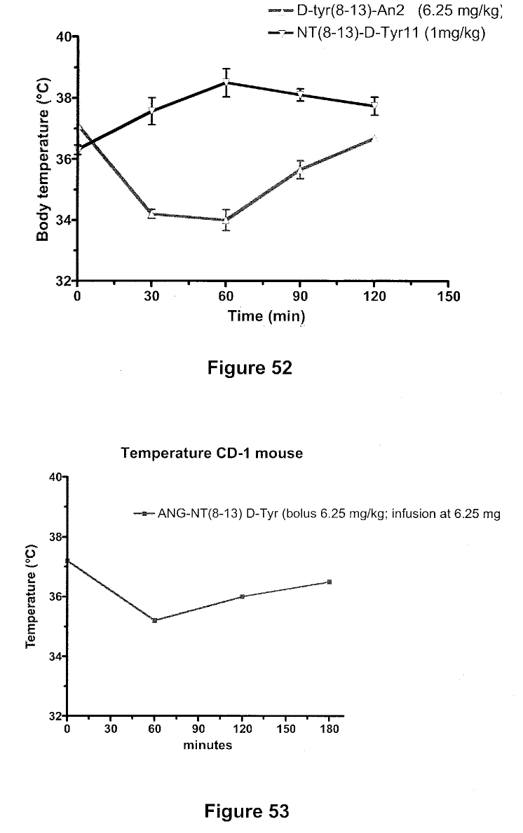

We also compared the ability of unconjugated Ac-Lys-[D-Tyr¹¹]NT(8-13) at 1 mg/kg to the Ac-Lys-[D-Tyr¹¹]NT(8-13)-An2 conjugate at 6.25 mg/kg to reduce body temperature. From these experiments, it was observed that the conjugate reduced body temperature to a greater extent than the unconjugated compound (FIG. 52).

A bolus injection (6.25 mg/kg) of the Ac-Lys-[D-Tyr¹¹]NT(8-13)-An2 conjugate followed by a 6.25 mg/kg/hr infusion of this conjugate after one hour was also performed (FIG. 53).

### EXAMPLE 21

### Binding of NT and NT Analogs and Conjugates Thereof to the NT Receptor NTSR1

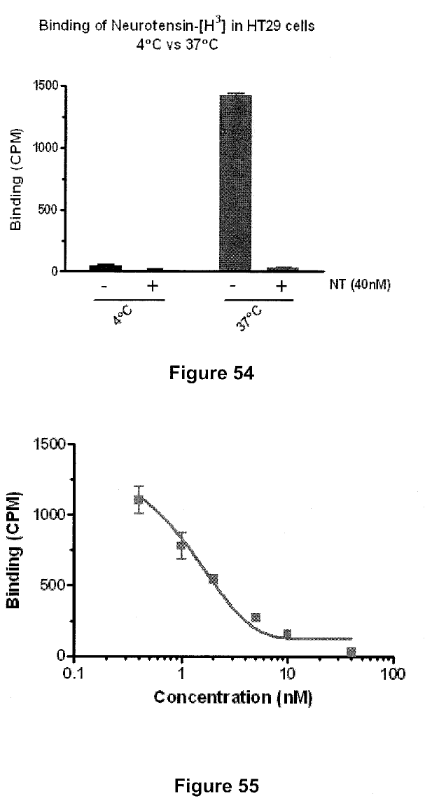

To further characterize NT, the NT analogs, and conjugates of NT, or NT analogs, a competitive binding assay using HT29 cells (human colon adenocarcinoma grade II cell line) that express the high affinity NTSR1 receptor was employed. As an initial test, we were able to demonstrate that [³H]-neurotensin could be completely displaced from the cells by 40 nM of unlabeled NT (FIG. 54). We then performed a dose response test between 0.4 nM and 40 nM. From these results, we determined that NT has an 10₅₀ of 1.4 nM in this system (FIG. 55).

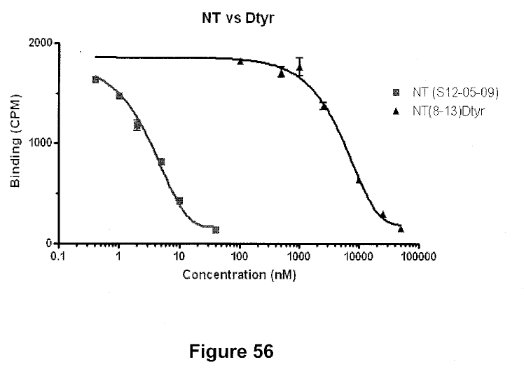

We then compared the binding of NT to that of Ac-Lys-[D-Tyr¹¹]NT(8-13)-An2. From this experiment, the binding of this analog was observed to be over 1000-fold weaker than the native NT (IC₅₀ of 3.5 nM vs. 5389 nM, as shown in FIG. 56).

Using these methods, we compared both the binding and the induced body temperature reduction between neurotensin, NT analogs, and the conjugates. These results are presented in the table below. The different results for NT and ANG-NT (i.e., NT-An2) represent results from different production batches of each compound.

| | | | |
|---|---|---|---|
| | IC50 | Δ Max temp | Sustained |
| Molecules | (nM) | (° C.) | hypothermia |

NT

| | | | |
|---|---|---|---|
| prep #1 | 1.6 | 0 | n.d. |
| prep #2 | 1.2-3.5 | 0 | n.d. |
| BACHEM | 4.0 | 0 | n.d. |
| Phoenix Pharmaceutials | 3.1 | 0 | n.d. |

NT analogs

| | | | |
|---|---|---|---|
| NT(8-13) | <1 | 0 | n.d. |
| AcLysNT(8-13)D-tyr11 | 5389 | −3 (at | n.d. |
| | | high dose) | |
| AcLys-NT(8-13) | 1 | 0 | n.d. |
| pGlu-Lys-NT(8-13) | 1.3 | 0 | n.d. |
| β-mercapto-MHA-NT(8-13) | 5 | 0 | n.d. |

ANG-NT conjugates

| | | | |
|---|---|---|---|
| ANG-NT (prep #1) | n.a. | −4 to −5 | n.a. |
| ANG-NT (prep #2) | 23.5 (glass) | −4 to −5 | +++ |
| ANG-NT (prep #3) | 10 | −3 to −4 | + |
| ANG-NT (prep #4) | 6.8 | −2 to −4 | − |
| ANG-NT(8-13) | 4 | 0 | n.d. |
| ANG-NT(8-13)(D-Tyr) | >100 | −3 to −4 | + |

### Other Embodiments

All patents, patent applications, including U.S. Provisional Application No. 61/200,947, filed Dec. 5, 2008 and publications mentioned in this specification are herein incorporated by reference to the same extent as if each independent patent, patent application, or publication was specifically and individually indicated to be incorporated by reference.

What is claimed is:

1. A compound having the formula
A-X-B
wherein
A is a peptide vector comprising an amino acid sequence at least 70% identical to a sequence selected from the group consisting of SEQ ID NO:1-105 and 107-114, or a fragment thereof;
X is a linker; and
B is a peptide therapeutic.

2. The compound of claim 1, where said peptide therapeutic is selected from the group consisting of antimicrobial or antibiotic peptides, gastrointestinal peptides, pancreatic peptides, peptide hormones, hypothalamic hormones, pituitary hormones, and neuropeptides.

3. The compound of claim 1, wherein A is a polypeptide has an amino acid sequence at least 70% identical to a sequence selected from the group consisting of Angiopep-1 (SEQ ID NO:67), Angiopep-2 (SEQ ID NO:97), cys-Angiopep-2 (SEQ ID NO:113), and Angiopep-2-cys (SEQ ID NO:114).

4. The compound of claim 3, wherein said polypeptide comprises or consists of an amino acid sequence selected from the group consisting of Angiopep-1 (SEQ ID NO:67), Angiopep-2 (SEQ ID NO:97), cys-Angiopep-2 (SEQ ID NO:113), and Angiopep-2-cys (SEQ ID NO:114).

5. The compound of claim 1 wherein X has the formula: 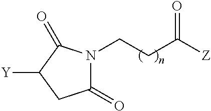
where n is an integer between 2 and 15; and either Y is a thiol on A and Z is a primary amine on B or Y is a thiol on B and Z is a primary amine on A.

6. The compound of claim 5, wherein n is 3, 6, or 11.

7. The compound of claim 1, wherein X is peptide bond.

8. The compound of claim 1, wherein X is at least one amino acid; and A and B are each covalently bonded to X by a peptide bond.

9. A nucleic acid molecule encoding the compound of claim 7.

10. A method of treating cancer, a neurological disease, or a lysosomal storage disorder in a subject, said method comprising administering to said subject a compound of claim 1 to a subject in an amount sufficient to treat said cancer, disease, or disorder.

11. The method of claim 10, wherein said cancer is a brain cancer or other cancer protected by the blood-brain barrier (BBB), and said peptide vector is efficiently transported across the BBB.

12. The method of claim 11, wherein said cancer is selected from the group consisting of astrocytoma, pilocytic astrocytoma, dysembryoplastic neuroepithelial tumor, oligodendroglioma, ependymoma, glioma, glioblastoma multiforme, mixed glioma, oligoastrocytoma, medulloblastoma, retinoblastoma, neuroblastoma, germinoma, and teratoma.

13. The method of claim 10, wherein said cancer is selected from the group consisting of hepatocellular carcinoma, breast cancer, cancers of the head and neck including various lymphomas such as mantle cell lymphoma, non-Hodgkins lymphoma, adenoma, squamous cell carcinoma, laryngeal carcinoma, cancers of the retina, cancers of the esophagus, multiple myeloma, ovarian cancer, uterine cancer, melanoma, colorectal cancer, bladder cancer, prostate cancer, lung cancer (including non-small cell lung carcinoma), pancreatic cancer, cervical cancer, head and neck cancer, skin cancers, nasopharyngeal carcinoma, liposarcoma, epithelial carcinoma, renal cell carcinoma, gallbladder adenocarcinoma, parotid adenocarcinoma, endometrial sarcoma, and multidrug resistant cancers.

14. The method of claim 10, wherein said neurological disease is selected from the group consisting of Alexander disease, Alper disease, Alzheimer's disease, amyotrophic lateral sclerosis (ALS), ataxia telangiectasia, Batten disease (Spielmeyer-Vogt-Sjogren-Batten disease), bovine spongiform encephalopathy (BSE), Canavan disease, Cockayne syndrome, corticobasal degeneration, Creutzfeldt-Jakob disease, Huntington's disease, HIV-associated dementia, Kennedy's disease, Krabbe disease, Lewy body dementia, Machado-Joseph disease (Spinocerebellar ataxia type 3), multiple sclerosis, multiple system atrophy, narcolepsy, neuroborreliosis, Parkinson's disease, Pelizaeus-Merzbacher disease, Pick's disease, primary lateral sclerosis, prion diseases, Refsum's disease, Schilder's disease (i.e., adrenoleukodystrophy), schizophrenia, spinocerebellar ataxia, spinal muscular atrophy, Steele-Richardson, Olszewski disease, and tabes dorsalis.
